(19) United States
(12) Patent Application Publication
(10) Pub. No.: US 2009/0093464 A1
(43) Pub. Date: Apr. 9, 2009

(54) Naphthalene Derivatives as Modulators of the Glucocorticoid Receptor

(75) Inventors: Stephen William Rafferty, Durham, NC (US); Philip Stewart Turnbull, Durham, NC (US); Eugene Lee Stewart, Durham, NC (US); Richard Dana Caldwell, Durham, NC (US)

Correspondence Address: GLAXOSMITHKLINE;CORPORATE INTELLECTUAL PROPERTY, MAI B482, FIVE MOORE DR., PO BOX 13398, RESEARCH TRIANGLE PARK, NC, 27709-3398, US

(73) Assignee: SMITHKLINE BEECHMA CORPORATION, Philadelphia, PA (US)

(21) Appl. No.: 11/816,889

(22) PCT Filed: Feb. 21, 2006
(86) PCT No.: PCT/US06/06096

§ 371 Date: Aug. 22, 2007

Related U.S. Application Data
(60) Provisional application No. 60/655,603, filed on Feb. 23, 2005.

(51) Int. Cl. A61K 31/551; C07D 237/02; A61K 31/50; A61K 31/501; C07D 231/02; A61P 25/18; A61P 25/30; A61P 3/10; A61K 31/415; C07D 403/00; C07D 243/02

(52) U.S. Cl. 514/218; 544/224; 514/247; 514/252.06; 544/238; 514/252.03; 514/252.01; 540/553; 514/403; 548/356.1

(57) ABSTRACT

The present invention relates to naphthalene derivatives of formula (I) that are modulators of the glucocorticoid receptor, and to processes for the preparation and use of the same.

### FIELD OF THE INVENTION

The present invention relates to naphthalene derivatives that are modulators of the glucocorticoid receptor, and to processes for the preparation and use of the same.

### BACKGROUND OF THE INVENTION

Nuclear receptors are a class of structurally related gene expression modulators that act as ligand-dependent transcription factors (R. M. Evans, (1988) Science 240, 889). The steroid receptors, namely the androgen receptor (AR), the estrogen receptor (ER), the glucocorticoid receptor (GR), the mineralocorticoid receptor (MR), and the progesterone receptor (PR) represent a subclass of the nuclear receptor superfamily. Nuclear receptor ligands in this subclass exert their effects by binding to an intracellular steroid hormone receptor. After the receptor-ligand complex is translocated to the nucleus of the cell, the complex binds to recognition sites on DNA, which allows for the modulation of certain genes.

Certain ligands have demonstrated the ability to exhibit their activity in a tissue selective manner. In other words, tissue selectivity allows a nuclear receptor ligand to function as an agonist in some tissues, while having no effect or even an antagonist effect in other tissues. The term “selective receptor modulator” (SRM) has been given to these molecules. A synthetic compound that binds to an intracellular receptor and mimics the effects of the native hormone is referred to as an agonist. A compound that inhibits the effect of the native hormone is called an antagonist. The term “modulators” refers to compounds that have a spectrum of activities ranging activation to inhibition of a cellular function (M. Coghlan, (2003) Curr. Topics Med. Chem. 3, 1617-1635).

Glucocorticoids (GCs) exert several effects in tissues that express GR. They regulate the expression of several genes either positively or negatively and in a direct or indirect manner. GCs have been identified as a key player in glucose regulation. Hepatic glucose production is regulated by two main mechanisms: glycogenolysis and gluconeogenesis. Glycogenolysis occurs within 2-3 hours after feeding in humans, while gluconeogenesis, a major source of glucose production in diabetic patients, becomes more important after prolonged fasting (Nordlie & Foster, (1999) Annu. Rev. Nutr. 19, 379-406 and Pilkis & Granner (1992) Annu. Rev. Physiol. 54, 885-909). Hormones such as insulin, glucagon, and GCs have been shown to control the expression of genes that encode proteins important in gluconeogenesis regulation (Spiegelman et al., (2001) Nature 413, 131-138).

There is much evidence that the GR is directly involved in glucose regulation. In one study, mice containing a homodimerizing-deficient GR (GR-dim mice) demonstrated an inability to regulate genes involved in gluconeogenesis (Reichardt et al., (1998) Cell 93, 531-541). Also, RU-486, a non-selective steroidal GR antagonist, significantly lowered glucose in a type 2 diabetes animal model (Friedman et al., (1997) J. Biol. Chem. 272, 31475-31481). The reduction in glucose was mainly due to a reduction in hepatic enzyme gene expression. However, the use of steroidal GR antagonists is limited by their side-effects.

Glucocorticoids are the end product of the HPA axis. Under normal conditions their secretion into the systemic circulation is tightly regulated by a negative feedback mechanism and marked increases in serum cortisol occur only in accord with a well-established circadian rhythm and in conditions of stress. It has been hypothesized that dysregulation of the HPA axis is a cause of depression (see, for example, Holsboer, F., (2000) Neuropsychopharmacol, 23, 477-501). Hyperactivity of the HPA axis in patients with major depression is one of the most consistent findings in biological psychiatry (see, for example, Holsboer, F & Barden, N. (1996). Endocr Rev, 17, 187-205). Patients with major depression have been shown to exhibit increased concentrations of cortisol in the plasma, urine and cerebrospinal fluid, exaggerated cortisol responses to exogenous ACTH and an enlargement of both the pituitary and the adrenal glands. In addition, a multitude of studies have demonstrated that the GC-mediated feedback inhibition of the HPA axis is impaired in depression; thus, unlike normal patients, approximately 50% of depressed patients fail to respond to synthetic GCs with a reduction in serum cortisol (Holsboer, F & Barden, N. (1996) Endocr Rev, 17, 187-205; Endocr Rev, 17, 187-205).

Evidence that disregulation of the HPA axis is a cause of depression has emerged from studies in patients with Cushing's syndrome (a condition of excessive cortisol secretion) and subjects in whom the HPA axis has been stimulated pharmacologically, for example with interferon-α (IFN-α) (J Psychopharmacol 16, 230-234) and that the severity of the symptoms correlates directly with the cytokine induced rise in serum cortisol (Am J Psychiatry, 160, 1342-1345). Further evidence of a role for the HPA axis in the pathogenesis of depression has emerged from the use of drugs to relieve depression which inhibit GC synthesis (such as ketoconazole, metyrapone and aminoglutethimide) or directly block the glucocorticoid receptor (such as RU38486 or Org-34517) (see, for example, Wolkowitz, O. et al., (1999) Psychosom Med, 61, 698-711; Belanoff, J. et al., (2002) Biol Psychiatry, 52, 386-392; Int J Neuropsychopharmacoll, 5 (Suppl. 1): Abst P.3.E.044). These and other data support a primary role for HPA dysfunction as a crucial biological mechanism in the pathogenesis of depression.

### SUMMARY OF THE INVENTION

Briefly, in one aspect, the present invention provides compounds of formula (I)

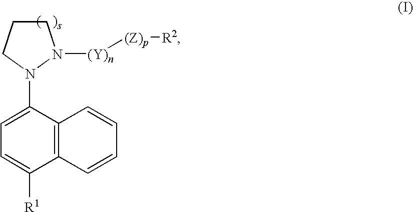

or a salt or solvate thereof, wherein

s is 1, 2, 3 or 4;

R¹ is cyano or nitro;

Y is —C(O)—;

Z is alkylene or —(R^{a})ₘO—;

R^{a} is alkylene;

m is 0 or 1;

n is 0 or 1;

p is 0 or 1;

R² is alkyl, cyano, cycloalkyl, substituted cycloalkyl, heterocycle, substituted heterocycle, aryl, substituted aryl, heteroaryl, substituted heteroaryl, haloalkyl, diphenylalkyl, alkylsilyl, amino, hydroxyl, —C(O)OCH₃, —CH(CN)CH₂Ph, —CH(OCH₂CH₃)Ph, or —NH(CH₂)₂Ph, wherein

when R² is substituted cycloalkyl, substituted aryl, substituted heteroaryl, or substituted heterocycle, each substituent is independently selected from the group consisting of alkyl, alkenyl, aryl, alkylaryl, alkylthio, alkoxy, alkenoxy, aryloxy, aralkoxy, halo, haloalkyl, haloaryl, haloalkoxy, haloalkylthio, haloalkylaryl, alkylsulfonyl, cyano, nitro, heterocycle, heteroaryl, cycloalkyl-alkylene, CH₃C(O)—, CH₃C(O)OCH₂—, and CH₃C(O)NH—.

Another aspect of the present invention provides a compound substantially as hereinbefore defined with reference to any one of the Examples.

Another aspect of the present invention provides a pharmaceutical composition comprising a compound of the present invention.

Another aspect of the present invention provides a compound of the present invention for use as an active therapeutic substance.

Another aspect of the present invention provides a compound of the present invention for use in the treatment of conditions or disorders that respond to glucocorticoid receptor modulation.

Another aspect of the present invention provides a compound of the present invention for use in the treatment of type 2 diabetes, type 1 diabetes, hyperglycemia, insulin resistance, metabolic syndrome X, diabetic dyslipidemia, bipolar disorder (manic depression), drug dependency, sleep disorders, schizophrenia, obsessive-compulsive disorder, post-traumatic stress disorder, social anxiety disorder, and generalized anxiety disorder.

Another aspect of the present invention provides a method for the treatment of type 2 diabetes, type 1 diabetes, hyperglycemia, insulin resistance, metabolic syndrome X, diabetic dyslipidemia, bipolar disorder (manic depression), drug dependency, sleep disorders, schizophrenia, obsessive-compulsive disorder, post-traumatic stress disorder, social anxiety disorder, and generalized anxiety disorder comprising the administration of a compound of the present invention.

Another aspect of the present invention provides a method for the treatment of conditions or disorders that respond to glucocorticoid receptor modulation comprising the administration of a compound of the present invention.

Another aspect of the present invention provides the use of a compound of the present invention in the manufacture of a medicament for use in the treatment of type 2 diabetes, type 1 diabetes, hyperglycemia, insulin resistance, metabolic syndrome X, diabetic dyslipidemia, bipolar disorder (manic depression), drug dependency, sleep disorders, schizophrenia, obsessive-compulsive disorder, post-traumatic stress disorder, social anxiety disorder, and generalized anxiety disorder.

Another aspect of the present invention provides the use of a compound of the present invention in the manufacture of a medicament for use in the treatment of conditions or disorders that respond to glucocorticoid receptor modulation.

### DETAILED DESCRIPTION OF THE INVENTION

Terms are used within their accepted meanings. The following definitions are meant to clarify, but not limit, the terms defined.

As used herein, “a compound of formula (I)” means a compound of formula (I) or a salt or solvate thereof.

As used herein the term “alkyl” refers to a straight or branched chain hydrocarbon, preferably having from one to twelve carbon atoms. Examples of “alkyl” as used herein include, but are not limited to, methyl, ethyl, propyl, isopropyl, isobutyl, n-butyl, tert-butyl, isopentyl, and n-pentyl.

As used throughout this specification, the preferred number of atoms, such as carbon atoms, will be represented by, for example, the phrase “Cₓ₋C_{y} alkyl,” which refers to an alkyl group, as herein defined, containing the specified number of carbon atoms. Similar terminology will apply for other preferred terms and ranges as well.

As used herein the term “alkenyl” refers to a straight or branched chain aliphatic hydrocarbon containing one or more carbon-to-carbon double bonds. Examples include, but are not limited to, vinyl and the like.

As used herein, the term “alkylene” refers to a straight or branched chain divalent hydrocarbon radical, preferably having from one to ten carbon atoms. Examples of “alkylene” as used herein include, but are not limited to, methylene (—CH₂—), ethylene (—CH₂—CH₂—), and branched versions thereof such as (—CH(CH₃)—) and the like.

As used herein, the term “cycloalkyl” refers to a non-aromatic cyclic hydrocarbon ring. Exemplary “cycloalkyl” groups include, but are not limited to, cyclopropyl, cyclobutyl, cyclopentyl, cyclohexyl, cycloheptyl, and the like.

As used herein, the term “heterocycle” or “heterocyclyl” refers to a mono- or poly-cyclic ring system containing one or more heteroatoms and optionally containing one or more degrees of unsaturation. Preferred heteroatoms include N, O, and/or S, including N-oxides, sulfur oxides, and dioxides. Preferably the ring is three to ten-membered and is saturated. Such rings may be optionally fused to one or more of another “heterocyclic” ring(s), heteroaryl ring(s), aryl ring(s), or cycloalkyl ring(s). Examples of “heterocyclic” groups include, but are not limited to, tetrahydrofuran, pyran, piperidine, pyrrolidine, pyrrolidinone, and morpholine.

The term “aryl” refers to an aromatic ring system, such as an benzene ring system, such as phenyl. The term encompasses fused systems where one or more benzene rings form, for example, anthracene, phenanthrene, or naphthalene ring systems. The term also includes an optional alkylene linker, such as C₁-C₆ alkylene, through which the aryl group may be attached. Examples of “aryl” groups include, but are not limited to phenyl, benzyl, 2-naphthyl, 1-naphthyl, biphenyl, as well as substituted derivatives thereof.

As used herein the term “aralkyl” refers to a group —RₐR_{b}, where Rₐ is an alkylene group and R_{b} is an aryl group as each is herein defined.

As used herein, the term “heteroaryl” refers to a monocyclic five to seven membered aromatic ring, or to a fused bicyclic aromatic ring system comprising two of such aromatic rings, which contain one or more nitrogen, sulfur, and/or oxygen atoms,

where N-oxides, sulfur oxides, and dioxides are permissible heteroatom substitutions. Examples of “heteroaryl” groups used herein include, but should not be limited to, furan, thiophene, pyrrole, imidazole, pyrazole, triazole, tetrazole, thiazole, oxazole, isoxazole, oxadiazole, thiadiazole, isothiazole, pyridine, pyridazine, pyrazine, pyrimidine, quinoline, isoquinoline, benzofuran, benzothiophene, indole, indazole, and the like. Preferred heteroaryl groups include furyl, thienyl, pyrrolyl, imidazolyl, pyrazolyl, triazolyl, isooxazolyl, pyridyl, piperonyl, and indolyl.

As used herein the term “alkylaryl” refers to a group —RₐR_{b}, where Rₐ is an arylene group and R_{b} is an alkyl group as each is herein defined.

As used herein the term “halo” or “halogen” refers to fluorine, chlorine, bromine, or iodine.

As used herein the term “haloalkyl” refers to an alkyl group, as defined herein that is substituted with at least one halogen. Examples of branched or straight chained “haloalkyl” groups useful in the present invention include, but are not limited to, methyl, ethyl, propyl, isopropyl, n-butyl, and t-butyl substituted independently with one or more halogens, e.g., fluoro, chloro, bromo, and iodo. The term “haloalkyl” should be interpreted to include such substituents such as —CF₃, —CH₂—CH₂—F, —CH₂—CF₃, and the like.

As used herein the term “haloaryl” refers to an aryl group, as defined herein that is substituted with at least one halogen. Examples of “haloaryl” groups useful in the present invention include, but are not limited to, phenyl and naphthyl, substituted independently with one or more halogens, e.g., fluoro, chloro, bromo, and iodo.

As used herein the term “haloalkylaryl” refers to an aryl group, as defined herein, substituted with one or more haloalkyl groups, as defined herein.

As used herein the term “hydroxy” or “hydroxyl” refers to a group —OH.

As used herein the term “alkoxy” refers to a group —ORₐ, where Rₐ is alkyl as herein defined.

As used herein the term “alkenoxy” refers to a group —ORₐ, where Rₐ is alkenyl as herein defined.

As used herein the term “aryloxy” refers to a group —ORₐ, where Rₐ is aryl as herein defined.

As used herein the term “aralkoxy” refers to a group —ORₐ, where Rₐ is aralkyl as herein defined.

As used herein the term “haloalkoxy” refers to a group —ORₐ, where Rₐ is haloalkyl as defined herein.

As used herein the term “alkylthio” refers to a group —SRₐ, where Rₐ is alkyl as herein defined.

As used herein the term “haloalkylthio” refers to a group —SRₐ, where Rₐ is haloalkyl as defined herein.

As used herein the term “alkylsulfonyl” refers to a group —SO₂Rₐ, where Rₐ is an alkyl group as herein defined.

As used herein the term “alkylsilyl” refers to a group —Si(Rₐ)₃, where Rₐ is an alkyl group as herein defined.

As used herein the term “nitro” refers to a group —NO₂.

As used herein the term “cyano” refers to a group —CN.

As used herein the term “amino” refers to a group —NH₂, and also refers to a group —N(Rₐ)(R_{b}), where one or both of Rₐ and R_{b} are other than H. For example, the term includes groups such as —N(CH₃)(CH₃), —N(CH₃)(CH₂—CH₃), and the like.

In one embodiment, the present invention provides compounds of formula (I) wherein s is 1, 2, or 3.

In another embodiment, s is 1, R¹ is cyano or nitro, Y is —C(O)—, Z is alkylene, n is 0 or 1, p is 0 or 1, and R² is alkyl, cycloalkyl, heteroaryl, aryl, substituted aryl, haloalkyl, or amino, wherein when R² is substituted aryl, each substituent is independently selected from the group consisting of alkyl, alkoxy, halo, haloalkyl, haloalkoxy, cyano, and CH₃C(O)—.

In another embodiment, s is 3, R¹ is cyano or nitro, Y is —C(O)—, Z is alkylene, n is 0 or 1, p is 0 or 1, and R² is alkyl, cycloalkyl, substituted cycloalkyl, heterocycle, substituted heterocycle, aryl, substituted aryl, heteroaryl, substituted heteroaryl, or haloalkyl, wherein when R² is substituted cycloalkyl, substituted aryl, substituted heteroaryl, or substituted heterocycle, each substituent is independently selected from the group consisting of alkyl, alkenyl, aryl, alkylaryl, alkoxy, halo, haloalkyl, haloalkoxy, and cyano.

In another embodiment, s is 2, R¹ is cyano or nitro, Y is —C(O)—, Z is alkylene or —(R^{a})ₘO—, R^{a} is alkylene, m is 0 or 1, n is 0 or 1, p is 0 or 1, and R² is alkyl, cycloalkyl, substituted cycloalkyl, heterocycle, substituted heterocycle, aryl, substituted aryl, heteroaryl, substituted heteroaryl haloalkyl, diphenylalkyl, amino, hydroxyl, or —C(O)OCH₃, —CH(CN)CH₂Ph, —CH(OCH₂CH₃)Ph, —NH(CH₂)₂Ph, wherein when R² is substituted cycloalkyl, substituted aryl, substituted heteroaryl, or substituted heterocycle, each substituent is independently selected from the group consisting of alkyl, alkenyl, aryl, alkylaryl, alkylthio, alkoxy, alkenoxy, aryloxy, aralkoxy, halo, haloalkyl, haloaryl, haloalkoxy, haloalkylthio, haloalkylaryl, alkylsulfonyl, cyano, nitro, heterocyclyl, cycloalkyl-alkylene, CH₃C(O)—, CH₃C(O)OCH₂—, and CH₃C(O)NH—.

In another embodiment, s is 2, R¹ is cyano or nitro, Y is —C(O)—, Z is alkylene, n is 0 or 1, p is 1, and R² is heterocycle, substituted aryl, or substituted heteroaryl, wherein when R² is substituted aryl or substituted heteroaryl, each substituent is independently selected from the group consisting of alkyl, aryl, haloalkyl, haloalkoxy, cyano, and heteroaryl.

Preferably, R¹ is cyano.

Preferably, s is 2.

Preferably, n is 0.

Preferably, Y is —C(O)— and n is 1.

Preferably, Z is alkylene and p is 1.

Preferably, Z is methylene and p is 1.

Preferably, R² is heterocycle, substituted aryl, or substituted heteroaryl, wherein when R² is substituted aryl or substituted heteroaryl, each substituent is independently selected from the group consisting of alkyl, aryl, haloalkyl, haloalkoxy, cyano, and heteroaryl.

In another embodiment, the present invention provides compounds of formula (IA)

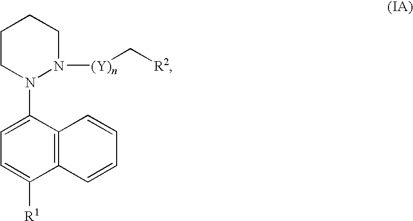

or a salt or solvate thereof, wherein R¹ is cyano or nitro, Y is —C(O)—, n is 0 or 1, and R² is substituted aryl, heterocycle, or substituted heteroaryl, wherein when R² is substituted aryl or substituted heteroaryl, each substituent, is independently selected from the group consisting of alkyl, haloalkyl, haloalkoxy, heteroaryl, cyano and aryl.

While the preferred groups for each variable have generally been listed above separately for each variable, preferred compounds of this invention include those in which several of each variable in Formula (I) is selected from the preferred, more preferred, or most preferred groups for each variable. Therefore, this invention is intended to include all combinations of preferred, more preferred, and most preferred groups.

The compounds of formula (I) may crystallize in more than one form, a characteristic known as polymorphism, and such polymorphic forms (“polymorphs”) are within the scope of formulas (I). Polymorphism generally can occur as a response to changes in temperature, pressure, or both. Polymorphism can also result from variations in the crystallization process. Polymorphs can be distinguished by various physical characteristics known in the art such as x-ray diffraction patterns, solubility, and melting point.

Certain of the compounds described herein contain one or more chiral centers, or may otherwise be capable of existing as multiple stereoisomers. The scope of the present invention includes mixtures of stereoisomers as well as purified enantiomers or enantiomerically/diastereomerically enriched mixtures. Also included within the scope of the invention are the individual isomers of the compounds represented by formulas (I), as well as any wholly or partially equilibrated mixtures thereof. The present invention also includes the individual isomers of the compounds represented by the formulas above as mixtures with isomers thereof in which one or more chiral centers are inverted.

Typically, but not absolutely, the salts of the present invention are pharmaceutically acceptable salts. Salts encompassed within the term “pharmaceutically acceptable salts” refer to non-toxic salts of the compounds of this invention. Salts of the compounds of the present invention may comprise acid addition salts. Representative salts include acetate, benzenesulfonate, benzoate, bicarbonate, bisulfate, bitartrate, borate, bromide, calcium edetate, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isethionate, lactate, lactobionate, laurate, malate, maleate, mandelate, mesylate, methylbromide, methylnitrate, methylsulfate, monopotassium maleate, mucate, napsylate, nitrate, N-methylglucamine, oxalate, pamoate (embonate), palmitate, pantothenate, phosphate/diphosphate, polygalacturonate, potassium, salicylate, sodium, stearate, subacetate, succinate, sulfate, tannate, tartrate, teoclate, tosylate, triethiodide, trimethylammonium, and valerate salts. Other salts, which are not pharmaceutically acceptable, may be useful in the preparation of compounds of this invention and these should be considered to form a further aspect of the invention.

As used herein, the term “solvate” refers to a complex of variable stoichiometry formed by a solute (in this invention, a compound of Formula I, Formula II, or a salt or physiologically functional derivative thereof) and a solvent. Such solvents, for the purpose of the invention, should not interfere with the biological activity of the solute. Non-limiting examples of suitable solvents include, but are not limited to water, methanol, ethanol, and acetic acid. Preferably the solvent used is a pharmaceutically acceptable solvent. Non-limiting examples of suitable pharmaceutically acceptable solvents include water, ethanol, and acetic acid. Most preferably the solvent used is water.

As used herein, the term “physiologically functional derivative” refers to any pharmaceutically acceptable derivative of a compound of the present invention that, upon administration to a mammal, is capable of providing (directly or indirectly) a compound of the present invention or an active metabolite thereof. Such derivatives, for example, esters and amides, will be clear to those skilled in the art, without undue experimentation. Reference may be made to the teaching of Burger's Medicinal Chemistry And Drug Discovery, 5^{th} Edition, Vol 1: Principles and Practice, which is incorporated herein by reference to the extent that it teaches physiologically functional derivatives.

As used herein, the term “effective amount” means that amount of a drug or pharmaceutical agent that will elicit the biological or medical response of a tissue, system, animal, or human that is being sought, for instance, by a researcher or clinician. The biological or medical response may be considered a prophylactic response or a treatment response. The term “therapeutically effective amount” means any amount which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function. For use in therapy, therapeutically effective amounts of a compound of formula (I) may be administered as the raw chemical. Additionally, the active ingredient may be presented as a pharmaceutical composition.

Accordingly, the invention further provides pharmaceutical compositions that include compounds of the formula (I) and one or more pharmaceutically acceptable carriers, diluents, or excipients. The compounds of formula (I) are as herein described. The carrier(s), diluent(s) or excipient(s) must be acceptable, in the sense of being compatible with the other ingredients of the formulation and not deleterious to the recipient of the pharmaceutical composition.

In accordance with another aspect of the invention there is also provided a process for the preparation of a pharmaceutical formulation including admixing a compound of the formula (I) with one or more pharmaceutically acceptable carriers, diluents or excipients.

A therapeutically effective amount of a compound of the present invention will depend upon a number of factors. For example, the species, age, and weight of the recipient, the precise condition requiring treatment and its severity, the nature of the formulation, and the route of administration are all factors to be considered. The therapeutically effective amount ultimately should be at the discretion of the attendant physician or veterinarian. More usually, the effective amount should be in the range of 0.1 to 10 mg/kg body weight per day. Thus, for a 70 kg adult mammal the actual amount per day would usually be from 7 to 700 mg. This amount may be given in a single dose per day or in a number (such as two, three, four, five, or more) of sub-doses per day such that the total daily dose is the same. An effective amount of a salt, solvate, or physiologically functional derivative thereof, may be determined as a proportion of the effective amount of the compound of formula (I) per se. Similar dosages should be appropriate for treatment or prophylaxis of the other conditions referred to herein.

Pharmaceutical formulations may be presented in unit dose forms containing a predetermined amount of active ingredient per unit dose. Such a unit may contain, as a non-limiting example, 0.5 mg to 1 g of a compound of the formula (I), depending on the condition being treated, the route of administration, and the age, weight, and condition of the patient. Preferred unit dosage formulations are those containing a daily dose or sub-dose, as herein above recited, or an appropriate fraction thereof, of an active ingredient. Such pharmaceutical formulations may be prepared by any of the methods well known in the pharmacy art.

Pharmaceutical formulations may be adapted for administration by any appropriate route, for example by an oral (including buccal or sublingual), rectal, nasal, topical (including buccal, sublingual or transdermal), vaginal, or parenteral (including subcutaneous, intramuscular, intravenous or intradermal) route. Such formulations may be prepared by any method known in the art of pharmacy, for example by bringing into association the active ingredient with the carrier(s) or excipient(s).

Pharmaceutical formulations adapted for oral administration may be presented as discrete units such as capsules or tablets; powders or granules; solutions or suspensions, each with aqueous or non-aqueous liquids; edible foams or whips; or oil-in-water liquid emulsions or water-in-oil liquid emulsions. For instance, for oral administration in the form of a tablet or capsule, the active drug component can be combined with an oral, non-toxic pharmaceutically acceptable inert carrier such as ethanol, glycerol, water, and the like. Generally, powders are prepared by comminuting the compound to a suitable fine size and mixing with an appropriate pharmaceutical carrier such as an edible carbohydrate, as, for example, starch or mannitol. Flavorings, preservatives, dispersing agents, and coloring agents can also be present.

Capsules may be made by preparing a powder, liquid, or suspension mixture and encapsulating with gelatin or some other appropriate shell material. Glidants and lubricants such as colloidal silica, talc, magnesium stearate, calcium stearate, or solid polyethylene glycol can be added to the mixture before the encapsulation. A disintegrating or solubilizing agent such as agar-agar, calcium carbonate or sodium carbonate can also be added to improve the availability of the medicament when the capsule is ingested. Moreover, when desired or necessary, suitable binders, lubricants, disintegrating agents, and coloring agents can also be incorporated into the mixture. Examples of suitable binders include starch, gelatin, natural sugars such as glucose or beta-lactose, corn sweeteners, natural and synthetic gums such as acacia, tragacanth, or sodium alginate, carboxymethylcellulose, polyethylene glycol, waxes, and the like. Lubricants useful in these dosage forms include, for example, sodium oleate, sodium stearate, magnesium stearate, sodium benzoate, sodium acetate, sodium chloride, and the like. Disintegrators include, without limitation, starch, methyl cellulose, agar, bentonite, xanthan gum, and the like.

Tablets may be formulated, for example, by preparing a powder mixture, granulating or slugging, adding a lubricant and disintegrant, and pressing into tablets. A powder mixture may be prepared by mixing the compound, suitably comminuted, with a diluent or base as described above. Optional ingredients include binders such as carboxymethylcellulose, aliginates, gelatins, or polyvinyl pyrrolidone, solution retardants such as paraffin, resorption accelerators such as a quaternary salt, and/or absorption agents such as bentonite, kaolin, or dicalcium phosphate. The powder mixture can be wet-granulated with a binder such as syrup, starch paste, acadia mucilage or solutions of cellulosic or polymeric materials, and forcing through a screen. As an alternative to granulating, the powder mixture can be run through the tablet machine and the result is imperfectly formed slugs broken into granules. The granules can be lubricated to prevent sticking to the tablet forming dies by means of the addition of stearic acid, a stearate salt, talc or mineral oil. The lubricated mixture is then compressed into tablets. The compounds of the present invention can also be combined with a free flowing inert carrier and compressed into tablets directly without going through the granulating or slugging steps. A clear or opaque protective coating consisting of a sealing coat of shellac, a coating of sugar or polymeric material, and a polish coating of wax can be provided. Dyestuffs can be added to these coatings to distinguish different unit dosages.

Oral fluids such as solutions, syrups, and elixirs can be prepared in dosage unit form so that a given quantity contains a predetermined amount of the compound. Syrups can be prepared, for example, by dissolving the compound in a suitably flavored aqueous solution, while elixirs are prepared through the use of a non-toxic alcoholic vehicle. Suspensions can be formulated generally by dispersing the compound in a non-toxic vehicle. Solubilizers and emulsifiers such as ethoxylated isostearyl alcohols and polyoxy ethylene sorbitol ethers, preservatives; flavor additives such as peppermint oil, or natural sweeteners, saccharin, or other artificial sweeteners; and the like can also be added.

Where appropriate, dosage unit formulations for oral administration can be microencapsulated. The formulation can also be prepared to prolong or sustain the release as for example by coating or embedding particulate material in polymers, wax or the like.

The compounds of formula (I) can also be administered in the form of liposome delivery systems, such as small unilamellar vesicles, large unilamellar vesicles, and multilamellar vesicles. Liposomes can be formed from a variety of phospholipids, such as cholesterol, stearylamine, or phosphatidylcholines.

The compounds of formula (I) may also be delivered by the use of monoclonal antibodies as individual carriers to which the compound molecules are coupled.

The compounds may also be coupled with soluble polymers as targetable drug carriers. Such polymers can include polyvinylpyrrolidone (PVP), pyran copolymer, polyhydroxypropylmethacrylamide-phenol, polyhydroxyethyl-aspartamidephenol, or polyethyleneoxidepolylysine substituted with palmitoyl residues. Furthermore, the compounds may be coupled to a class of biodegradable polymers useful in achieving controlled release of a drug; for example, polylactic acid, polyepsilon caprolactone, polyhydroxy butyric acid, polyorthoesters, polyacetals, polydihydropyrans, polycyanoacrylates, and cross-linked or amphipathic block copolymers of hydrogels.

Pharmaceutical formulations adapted for transdermal administration may be presented as discrete patches intended to remain in intimate contact with the epidermis of the recipient for a prolonged period of time. For example, the active ingredient may be delivered from the patch by iontophoresis as generally described in Pharmaceutical Research, 3(6), 318 (1986), incorporated herein by reference as related to such delivery systems.

Pharmaceutical formulations adapted for topical administration may be formulated as ointments, creams, suspensions, lotions, powders, solutions, pastes, gels, sprays, aerosols, or oils.

For treatments of the eye or other external tissues, for example mouth and skin, the formulations may be applied as a topical ointment or cream. When formulated in an ointment, the active ingredient may be employed with either a paraffinic or a water-miscible ointment base. Alternatively, the active ingredient may be formulated in a cream with an oil-in-water cream base or a water-in-oil base.

Pharmaceutical formulations adapted for topical administrations to the eye include eye drops wherein the active ingredient is dissolved or suspended in a suitable carrier, especially an aqueous solvent.

Pharmaceutical formulations adapted for topical administration in the mouth include lozenges, pastilles, and mouthwashes.

Pharmaceutical formulations adapted for nasal administration, where the carrier is a solid, include a coarse powder having a particle size for example in the range 20 to 500 microns. The powder is administered in the manner in which snuff is taken, i.e., by rapid inhalation through the nasal passage from a container of the powder held close up to the nose. Suitable formulations wherein the carrier is a liquid, for administration as a nasal spray or as nasal drops, include aqueous or oil solutions of the active ingredient.

Pharmaceutical formulations adapted for administration by inhalation include fine particle dusts or mists, which may be generated by means of various types of metered dose pressurized aerosols, nebulizers, or insufflators.

Pharmaceutical formulations adapted for rectal administration may be presented as suppositories or as enemas.

Pharmaceutical formulations adapted for vaginal administration may be presented as pessaries, tampons, creams, gels, pastes, foams, or spray formulations.

Pharmaceutical formulations adapted for parenteral administration include aqueous and non-aqueous sterile injection solutions which may contain anti-oxidants, buffers, bacteriostats, and solutes that render the formulation isotonic with the blood of the intended recipient; and aqueous and non-aqueous sterile suspensions which may include suspending agents and thickening agents. The formulations may be presented in unit-dose or multi-dose containers, for example sealed ampules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules, and tablets.

In addition to the ingredients particularly mentioned above, the formulations may include other agents conventional in the art having regard to the type of formulation in question. For example, formulations suitable for oral administration may include flavoring or coloring agents.

The present invention provides methods for the treatment of several conditions or diseases, all of which comprise the step of administering a compound of formula (I). As used herein, the term “treatment” refers to alleviating the specified condition, eliminating or reducing the symptoms of the condition, slowing or eliminating the progression of the condition and preventing or delaying the initial occurrence of the condition in a subject, or reoccurrence of the condition in a previously afflicted subject.

A further aspect of the invention provides a method of treatment of a mammal requiring the treatment of a variety of disorders including, but not limited to type 2 diabetes, type 1 diabetes, hyperglycemia, insulin resistance, metabolic syndrome X, diabetic dyslipidemia, hyperlipidemia, hypercholesterolemia, hypertension, obesity, cardiovascular disease/atherosclerosis, bipolar disorder (manic depression), drug dependency, sleep disorders, schizophrenia, obsessive-compulsive disorder, post-traumatic stress disorder, social anxiety disorder, generalized anxiety disorder, hypogonadism, sexual dysfunction, Cushing's Syndrome, inflammation, liver fibrosis, tissue rejection, auto-immunity, various malignancies, such as leukemias and lymphomas, rheumatic fever, polyarteritis nodosa, granulomatous polyarteritis, immune proliferation/apoptosis, chronic kidney disease, stroke and spinal cord injury, hypercalcemia, acute and chronic adrenal insufficiency, cerebral edema, Little's syndrome, inflammatory bowel disease, rheumatoid arthritis, osteoarthritis, rhinitis, asthma, tendonitis, and Crohn's disease, which use includes administering to a subject a compound of formula (I). The mammal requiring treatment with a compound of the present invention is typically a human being.

The compounds of the present invention and their salts and solvates thereof, may be employed alone or in combination with other therapeutic agents for the treatment of the above-mentioned conditions. For example, in type 2 diabetes, combination may be had with other glucose lowering therapeutic agents. As one example, type 2 diabetes combination therapies according to the present invention would thus comprise the administration of at least one compound of formula (I) and the use of at least one other glucose lowering therapy. As a further example, combination therapies according to the present invention include the administration of at least one compound of formula (I) and at least one other glucose lowering treatment agent, for example, a sulfonourea. The compound(s) of formula (I) and the other pharmaceutically active agent(s) may be administered together or separately and, when administered separately, administration may occur simultaneously or sequentially, in any order. The amounts of the compound(s) of formula (I) and the other pharmaceutically active agent(s) and the relative timings of administration will be selected in order to achieve the desired combined therapeutic effect. The administration in combination of a compound of formula (I) with other treatment agents may be in combination by administration concomitantly in: (1) a unitary pharmaceutical composition including both compounds; or (2) separate pharmaceutical compositions each including one of the compounds. Alternatively, the combination may be administered separately in a sequential manner wherein one treatment agent is administered first and the other second or vice versa. Such sequential administration may be close in time or remote in time.

Another embodiment of the present invention includes compounds that are selective for GR. As used herein, the term “selective” means a compound having an IC50 in one receptor assay that is at least 10 fold lower than the IC50 in the other receptor assay, as described below. For example, a GR selective compound is a compound that would have an IC50 in the GR fluorescence polarization assay that is at least 10 fold less than the IC50 of that same compound in the AR fluorescence polarization assay.

The compounds of the present invention may be used in the treatment of a variety of disorders and conditions and, as such, the compounds of the present invention may be used in combination with a variety of other suitable therapeutic agents useful in the treatment of those disorders or conditions. Non-limiting examples include combinations of the present invention with anti-diabetic agents, anti-osteoporosis agents, anti-obesity agents, anti-inflammatory agents, anti-anxiety agents, anti-depressants, anti-hypertensive agents, anti-platelet agents, anti-thrombotic and thrombolytic agents, cardiac glycosides, cholesterol or lipid lowering agents, mineralocorticoid receptor antagonists, phosphodiesterase inhibitors, kinase inhibitors, thyroid mimetics, anabolic agents, viral therapies, cognitive disorder therapies, sleeping disorder therapies, sexual dysfunction therapies, contraceptives, cytotoxic agents, radiation therapy, anti-proliferative agents, and anti-tumor agents. Additionally, the compounds of the present invention may be combined with nutritional supplements such as amino acids, triglycerides, vitamins, minerals, creatine, piloic acid, carnitine, or coenzyme Q10.

The compounds of this invention may be made by a variety of methods. Illustrative general synthetic methods are set out below and then specific compounds of the invention are prepared in the working Examples.

In all of the examples described below, protecting groups for sensitive or reactive groups are employed where necessary in accordance with general principles of synthetic chemistry. Protecting groups are manipulated according to standard methods of organic synthesis (T. W. Green and P. G. M. Wuts (1991) Protecting Groups in Organic Synthesis, John Wiley & Sons, incorporated by reference with regard to protecting groups). These groups are removed at a convenient stage of the compound synthesis using methods that are readily apparent to those skilled in the art. The selection of processes as well as the reaction conditions and order of their execution shall be consistent with the preparation of compounds of formula (I).

Those skilled in the art will recognize if a stereocenter exists in compounds of formula (I). Accordingly, the present invention includes all possible stereoisomers and includes not only racemic compounds but the individual enantiomers as well. When a compound is desired as a single enantiomer, such may be obtained by stereospecific synthesis, by resolution of the final product or any convenient intermediate, or by chiral chromatographic methods as are known in the art. Resolution of the final product, an intermediate, or a starting material may be effected by any suitable method known in the art. See, for example, Stereochemistry of Organic Compounds by E. L. Eliel, S. H. Wilen, and L. N. Mander (Wiley-Interscience, 1994), incorporated by reference with regard to stereochemistry.

Suitable compounds of the present invention include:
4-[2-(phenylmethyl)tetrahydro-1(2H)-pyridazinyl]-1-naphthalenecarbonitrile;
4-[2-{[4-(methyloxy)phenyl]methyl}tetrahydro-1(2H)-pyridazinyl]-1-naphthalenecarbonitrile;
4-[2-{[3-(ethyloxy)phenyl]methyl}tetrahydro-1(2H)-pyridazinyl]-1-naphthalenecarbonitrile;
4-[2-{[4-(ethyloxy)phenyl]methyl}tetrahydro-1(2H)-pyridazinyl]-1-naphthalenecarbonitrile;
4-[2-{[4-(butyloxy)phenyl]methyl}tetrahydro-1(2H)-pyridazinyl]-1-naphthalenecarbonitrile;
4-[2-[(4-ethylphenyl)methyl]tetrahydro-1(2H)-pyridazinyl]-1-naphthalenecarbonitrile;
4-[2-{[4-(1-methylethyl)phenyl]methyl}tetrahydro-1(2H)-pyridazinyl]-1-naphthalenecarbonitrile;
4-[2-[(2-bromophenyl)methyl]tetrahydro-1(2H)-pyridazinyl]-1-naphthalenecarbonitrile;
4-[2-[(3-bromophenyl)methyl]tetrahydro-1(2H)-pyridazinyl]-1-naphthalenecarbonitrile;
4-[2-[(4-bromophenyl)methyl]tetrahydro-1(2H)-pyridazinyl]-1-naphthalenecarbonitrile;
4-[2-[(2-chlorophenyl)methyl]tetrahydro-1(2H)-pyridazinyl]-1-naphthalenecarbonitrile;
4-[2-[(2-methylphenyl)methyl]tetrahydro-1(2H)-pyridazinyl]-1-naphthalenecarbonitrile;
4-[2-[(3-chlorophenyl)methyl]tetrahydro-1(2H)-pyridazinyl]-1-naphthalenecarbonitrile;
4-[2-[(4-chlorophenyl)methyl]tetrahydro-1(2H)-pyridazinyl]-1-naphthalenecarbonitrile;
4-[2-[(2-fluorophenyl)methyl]tetrahydro-1(2H)-pyridazinyl]-1-naphthalenecarbonitrile;
4-[2-[(3-fluorophenyl)methyl]tetrahydro-1(2H)-pyridazinyl]-1-naphthalenecarbonitrile;
4-[2-[(4-fluorophenyl)methyl]tetrahydro-1(2H)-pyridazinyl]-1-naphthalenecarbonitrile;
4-[2-[(2-cyanophenyl)methyl]tetrahydro-1(2H)-pyridazinyl]-1-naphthalenecarbonitrile;
4-[2-[(3-cyanophenyl)methyl]tetrahydro-1(2H)-pyridazinyl]-1-naphthalenecarbonitrile;
4-[2-[(4-cyanophenyl)methyl]tetrahydro-1(2H)-pyridazinyl]-1-naphthalenecarbonitrile;
4-[2-{[4-(methylthio)phenyl]methyl}tetrahydro-1(2H)-pyridazinyl]-1-naphthalenecarbonitrile;
4-[2-[(3,4-dichlorophenyl)methyl]tetrahydro-1(2H)-pyridazinyl]-1-naphthalenecarbonitrile;
4-[2-[(3-methylphenyl)methyl]tetrahydro-1(2H)-pyridazinyl]-1-naphthalenecarbonitrile;
4-[2-[(4-acetylphenyl)methyl]tetrahydro-1(2H)-pyridazinyl]-1-naphthalenecarbonitrile;
4-[2-{[4-(methylsulfonyl)phenyl]methyl}tetrahydro-1(2H)-pyridazinyl]-1-naphthalenecarbonitrile;
4-[2-{[4-(phenyloxy)phenyl]methyl}tetrahydro-1(2H)-pyridazinyl]-1-naphthalenecarbonitrile;
4-[2-[(4-butylphenyl)methyl]tetrahydro-1(2H)-pyridazinyl]-1-naphthalenecarbonitrile;
4-[2-({2-[(1,1-dimethylethyl)thio]phenyl}methyl)tetrahydro-1(2H)-pyridazinyl]-1-naphthalenecarbonitrile;
4-[2-({3-[(trifluoromethyl)thio]phenyl}methyl)tetrahydro-1(2H)-pyridazinyl]-1-naphthalenecarbonitrile;
4-[2-{[2,4-bis(trifluoromethyl)phenyl]methyl}tetrahydro-1(2H)-pyridazinyl]-1-naphthalenecarbonitrile;
4-[2-{[3,5-bis(trifluoromethyl)phenyl]methyl}tetrahydro-1(2H)-pyridazinyl]-1-naphthalenecarbonitrile;
4-[2-[(4-methylphenyl)methyl]tetrahydro-1(2H)-pyridazinyl]-1-naphthalenecarbonitrile;
4-[2-(1-naphthalenylmethyl)tetrahydro-1(2H)-pyridazinyl]-1-naphthalenecarbonitrile;
4-[2-(2-naphthalenylmethyl)tetrahydro-1(2H)-pyridazinyl]-1-naphthalenecarbonitrile;
4-[2-(3-thienylmethyl)tetrahydro-1(2H)-pyridazinyl]-1-naphthalenecarbonitrile;
4-[2-{[2,4-bis(methyloxy)phenyl]methyl}tetrahydro-1(2H)-pyridazinyl]-1-naphthalenecarbonitrile;
4-[2-{[2-(trifluoromethyl)phenyl]methyl}tetrahydro-1(2H)-pyridazinyl]-1-naphthalenecarbonitrile;
4-[2-{[4-(trifluoromethyl)phenyl]methyl}tetrahydro-1(2H)-pyridazinyl]-1-naphthalenecarbonitrile;
4-[2-{[2-(methyloxy)phenyl]methyl}tetrahydro-1(2H)-pyridazinyl]-1-naphthalenecarbonitrile;
4-[2-{[3-(methyloxy)phenyl]methyl}tetrahydro-1(2H)-pyridazinyl]-1-naphthalenecarbonitrile;
4-[2-{[3-(phenyloxy)phenyl]methyl}tetrahydro-1(2H)-pyridazinyl]-1-naphthalenecarbonitrile;
4-[2-(cyclohexylmethyl)tetrahydro-1(2H)-pyridazinyl]-1-naphthalenecarbonitrile;
4-[2-{[2-(2-propen-1-yloxy)phenyl]methyl}tetrahydro-1(2H)-pyridazinyl]-1-naphthalenecarbonitrile;
4-[2-(2,1,3-benzoxadiazol-4-ylmethyl)tetrahydro-1(2H)-pyridazinyl]-1-naphthalenecarbonitrile;
4-[2-{[1-(1,1-dimethylethyl)-5-methyl-1H-pyrazol-3-yl]methyl}tetrahydro-1(2H)-pyridazinyl]-1-naphthalenecarbonitrile;
4-[2-{[5-(2-pyridinyl)-2-thienyl]methyl}tetrahydro-1(2H)-pyridazinyl]-1-naphthalenecarbonitrile;
4-[2-[(1-phenyl-1H-pyrazol-5-yl)methyl]tetrahydro-1(2H)-pyridazinyl]-1-naphthalenecarbonitrile;
4-[2-[(2-phenyl-1H-imidazol-4-yl)methyl]tetrahydro-1(2H)-pyridazinyl]-1-naphthalenecarbonitrile;
4-[2-[(3-phenyl-1H-pyrazol-4-yl)methyl]tetrahydro-1(2H)-pyridazinyl]-1-naphthalenecarbonitrile;
N-(5-{[2-(4-cyano-1-naphthalenyl)tetrahydro-1(2H)-pyridazinyl]methyl}-1,3-thiazol-2-yl)acetamide;
4-[2-(2-pyridinylmethyl)tetrahydro-1(2H)-pyridazinyl]-1-naphthalenecarbonitrile;
(5-{[2-(4-cyano-1-naphthalenyl)tetrahydro-1(2H)-pyridazinyl]methyl}-2-furanyl)methyl acetate;
4-[2-[(1-phenyl-1H-imidazol-2-yl)methyl]tetrahydro-1(2H)-pyridazinyl]-1-naphthalenecarbonitrile;
4-[2-{[6-(methyloxy)-2-pyridinyl]methyl}tetrahydro-1(2H)-pyridazinyl]-1-naphthalenecarbonitrile;
4-[2-(1H-pyrazol-3-ylmethyl)tetrahydro-1(2H)-pyridazinyl]-1-naphthalenecarbonitrile;
4-[2-[(2-ethyl-4-methyl-1H-imidazol-5-yl)methyl]tetrahydro-1(2H)-pyridazinyl]-1-naphthalenecarbonitrile;
5-{[2-(4-cyano-1-naphthalenyl)tetrahydro-1(2H)-pyridazinyl]methyl}-3-pyridinecarbonitrile;
4-[2-[(1-phenyl-1H-pyrazol-4-yl)methyl]tetrahydro-1(2H)-pyridazinyl]-1-naphthalenecarbonitrile;
4-[2-[(3-methyl-2-thienyl)methyl]tetrahydro-1(2H)-pyridazinyl]-1-naphthalenecarbonitrile;
4-[2-[(5-methyl-2-thienyl)methyl]tetrahydro-1(2H)-pyridazinyl]-1-naphthalenecarbonitrile;
4-[2-(1H-imidazol-4-ylmethyl)tetrahydro-1(2H)-pyridazinyl]-1-naphthalenecarbonitrile;
4-[2-[(3,5-dichlorophenyl)methyl]tetrahydro-1(2H)-pyridazinyl]-1-naphthalenecarbonitrile;
4-[2-[(5-chloro-1,3-dimethyl-1H-pyrazol-4-yl)methyl]tetrahydro-1(2H)-pyridazinyl]-1-naphthalenecarbonitrile;
4-[2-[(3,5-dibromophenyl)methyl]tetrahydro-1(2H)-pyridazinyl]-1-naphthalenecarbonitrile;
4-[2-({5-[3-(trifluoromethyl)phenyl]-2-furanyl}methyl)tetrahydro-1(2H)-pyridazinyl]-1-naphthalenecarbonitrile;
4-[2-{[1-(3,5-dichlorophenyl)-1H-pyrrol-2-yl]methyl}tetrahydro-1(2H)-pyridazinyl]-1-naphthalenecarbonitrile;
4-[2-{[3-fluoro-5-(trifluoromethyl)phenyl]methyl}tetrahydro-1(2H)-pyridazinyl]-1-naphthalenecarbonitrile;
4-[2-[(3,5-difluorophenyl)methyl]tetrahydro-1(2H)-pyridazinyl]-1-naphthalenecarbonitrile;
4-[2-{[3,5-bis(methyloxy)phenyl]methyl}tetrahydro-1(2H)-pyridazinyl]-1-naphthalenecarbonitrile;
4-[2-[(5-ethyl-2-furanyl)methyl]tetrahydro-1(2H)-pyridazinyl]-1-naphthalenecarbonitrile;
4-[2-[(5-bromo-2-furanyl)methyl]tetrahydro-1(2H)-pyridazinyl]-1-naphthalenecarbonitrile;
4-[2-[(4,5-dimethyl-2-furanyl)methyl]tetrahydro-1(2H)-pyridazinyl]-1-naphthalenecarbonitrile;
4-[2-(3-pyridinylmethyl)tetrahydro-1(2H)-pyridazinyl]-1-naphthalenecarbonitrile
4-[2-[(6-methyl-2-pyridinyl)methyl]tetrahydro-1(2H)-pyridazinyl]-1-naphthalenecarbonitrile;
4-[2-[(4-bromo-1H-pyrazol-3-yl)methyl]tetrahydro-1(2H)-pyridazinyl]-1-naphthalenecarbonitrile;
4-[2-[(5-bromo-2-thienyl)methyl]tetrahydro-1(2H)-pyridazinyl]-1-naphthalenecarbonitrile;
4-[2-{[5-chloro-1-methyl-3-(trifluoromethyl)-1H-pyrazol-4-yl]methyl}tetrahydro-1(2H)-pyridazinyl]-1-naphthalenecarbonitrile;
4-[2-[(1-methyl-1H-imidazol-2-yl)methyl]tetrahydro-1(2H)-pyridazinyl]-1-naphthalenecarbonitrile;
4-[2-[(4-methyl-1H-imidazol-5-yl)methyl]tetrahydro-1(2H)-pyridazinyl]-1-naphthalenecarbonitrile;
4-[2-{[4-chloro-2-(cyclopropylmethyl)-1H-imidazol-5-yl]methyl}tetrahydro-1(2H)-pyridazinyl]-1-naphthalenecarbonitrile;
4-[2-[(2-butyl-1H-imidazol-4-yl)methyl]tetrahydro-1(2H)-pyridazinyl]-1-naphthalenecarbonitrile;
4-[2-[(3-bromo-4-pyridinyl)methyl]tetrahydro-1(2H)-pyridazinyl]-1-naphthalenecarbonitrile;
4-[2-({6-[(1,1-dimethylethyl)oxy]-2-pyridinyl}methyl)tetrahydro-1(2H)-pyridazinyl]-1-naphthalenecarbonitrile;
4-[2-[(2-ethyl-1H-imidazol-4-yl)methyl]tetrahydro-1(2H)-pyridazinyl]-1-naphthalenecarbonitrile;
4-[2-[(4-bromo-2-thienyl)methyl]tetrahydro-1(2H)-pyridazinyl]-1-naphthalenecarbonitrile;
4-[2-(1H-pyrazol-4-ylmethyl)tetrahydro-1(2H)-pyridazinyl]-1-naphthalenecarbonitrile;
4-[2-[(5-methyl-2-furanyl)methyl]tetrahydro-1(2H)-pyridazinyl]-1-naphthalenecarbonitrile;
4-[2-[(5-bromo-3-thienyl)methyl]tetrahydro-1(2H)-pyridazinyl]-1-naphthalenecarbonitrile;
4-[2-[(1,3-dimethyl-1H-pyrazol-5-yl)methyl]tetrahydro-1(2H)-pyridazinyl]-1-naphthalenecarbonitrile;
4-[2-[(6-bromo-3-pyridinyl)methyl]tetrahydro-1(2H)-pyridazinyl]-1-naphthalenecarbonitrile;
4-[2-(1H-1,2,3-triazol-4-ylmethyl)tetrahydro-1(2H)-pyridazinyl]-1-naphthalenecarbonitrile;
4-[2-(1H-imidazol-2-ylmethyl)tetrahydro-1(2H)-pyridazinyl]-1-naphthalenecarbonitrile;
4-(2-{[1-(1,1-dimethylethyl)-6-methyl-1H-pyrazol-3-yl]methyl}hexahydro-1H-1,2-diazepin-1-yl)-1-naphthalenecarbonitrile;
4-[2-({2-[(trifluoromethyl)oxy]phenyl}methyl)hexahydro-1H-1,2-diazepin-1-yl]-1-naphthalenecarbonitrile;
4-[2-(1H-1,2,3-triazol-4-ylmethyl)hexahydro-1H-1,2-diazepin-1-yl]-1-naphthalenecarbonitrile;
4-[2-(phenylmethyl)hexahydro-1H-1,2-diazepin-1-yl]-1-naphthalenecarbonitrile;
4-{2-[(4-phenyl-1H-imidazol-5-yl)methyl]hexahydro-1H-1,2-diazepin-1-yl}-1-naphthalenecarbonitrile;
4-(2-hexylhexahydro-1H-1,2-diazepin-1-yl)-1-naphthalenecarbonitrile;
4-{2-[(3-ethenylphenyl)methyl]hexahydro-1H-1,2-diazepin-1-yl}-1-naphthalenecarbonitrile;
4-{2-[(2-fluorophenyl)methyl]hexahydro-1H-1,2-diazepin-1-yl}-1-naphthalenecarbonitrile;
4-{2-[(1-phenyl-1H-pyrazol-4-yl)methyl]hexahydro-1H-1,2-diazepin-1-yl}-1-naphthalenecarbonitrile;
4-[2-(2-pyridinylmethyl)hexahydro-1H-1,2-diazepin-1-yl]-1-naphthalenecarbonitrile;
4-{2-[(3,5-dimethylphenyl)methyl]hexahydro-1H-1,2-diazepin-1-yl}-1-naphthalenecarbonitrile;
4-{2-[(3-methyl-5-phenyl-4-isoxazolyl)methyl]hexahydro-1H-1,2-diazepin-1-yl}-1-naphthalenecarbonitrile;
4-{2-[(2-methylphenyl)methyl]hexahydro-1H-1,2-diazepin-1-yl}-1-naphthalenecarbonitrile;
4-(2-{[2-(trifluoromethyl)phenyl]methyl}hexahydro-1H-1,2-diazepin-1-yl)-1-naphthalenecarbonitrile;
4-[2-(3-pyridinylmethyl)hexahydro-1H-1,2-diazepin-1-yl]-1-naphthalenecarbonitrile;
4-{2-[(3-phenyl-1H-pyrazol-5-yl)methyl]hexahydro-1H-1,2-diazepin-1-yl}-1-naphthalenecarbonitrile;
5-{[2-(4-cyano-1-naphthalenyl)hexahydro-1H-1,2-diazepin-1-yl]methyl}-3-pyridinecarbonitrile;
4-{2-[(2,2,3,3-tetramethylcyclopropyl)methyl]hexahydro-1H-1,2-diazepin-1-yl}-1-naphthalenecarbonitrile;
4-{2-[(1,3-dimethyl-1H-pyrazol-5-yl)methyl]hexahydro-1H-1,2-diazepin-1-yl}-1-naphthalenecarbonitrile;
4-{2-[(1-phenyl-1H-pyrazol-5-yl)methyl]hexahydro-1H-1,2-diazepin-1-yl}-1-naphthalenecarbonitrile;
4-{2-[(4-bromo-1H-pyrazol-3-yl)methyl]hexahydro-1H-1,2-diazepin-1-yl}-1-naphthalenecarbonitrile;
4-{2-[(3-methylphenyl)methyl]hexahydro-1H-1,2-diazepin-1-yl}-1-naphthalenecarbonitrile;
4-{2-[(2-cyanophenyl)methyl]hexahydro-1H-1,2-diazepin-1-yl}-1-naphthalenecarbonitrile;
4-(2-{[3-(4-methylphenyl)-1H-pyrazol-4-yl]methyl}hexahydro-1H-1,2-diazepin-1-yl)-1-naphthalenecarbonitrile;
4-{2-[(6-methyl-2-pyridinyl)methyl]hexahydro-1H-1,2-diazepin-1-yl}-1-naphthalenecarbonitrile;
4-{2-[(4-cyanophenyl)methyl]hexahydro-1H-1,2-diazepin-1-yl}-1-naphthalenecarbonitrile;
4-[2-(1H-pyrazol-3-ylmethyl)hexahydro-1H-1,2-diazepin-1-yl]-1-naphthalenecarbonitrile;
4-[2-(4-pyridinylmethyl)hexahydro-1H-1,2-diazepin-1-yl]-1-naphthalenecarbonitrile;
4-[2-(2-cyclopentylethyl)hexahydro-1H-1,2-diazepin-1-yl]-1-naphthalenecarbonitrile;
4-{2-[(3-cyanophenyl)methyl]hexahydro-1H-1,2-diazepin-1-yl}-1-naphthalenecarbonitrile;
4-(2-{[4-(trifluoromethyl)phenyl]methyl}hexahydro-1H-1,2-diazepin-1-yl)-1-naphthalenecarbonitrile;
4-(2-{[2-fluoro-3-(trifluoromethyl)phenyl]methyl}hexahydro-1H-1,2-diazepin-1-yl)-1-naphthalenecarbonitrile;
4-(2-{[3-(ethyloxy)phenyl]methyl}hexahydro-1H-1,2-diazepin-1-yl)-1-naphthalenecarbonitrile;
4-{2-[(3-fluorophenyl)methyl]hexahydro-1H-1,2-diazepin-1-yl}-1-naphthalenecarbonitrile;
4-{2-[(3-methyl-2-thienyl)methyl]hexahydro-1H-1,2-diazepin-1-yl}-1-naphthalenecarbonitrile;
4-(2-{[4-fluoro-2-(trifluoromethyl)phenyl]methyl}hexahydro-1H-1,2-diazepin-1-yl)-1-naphthalenecarbonitrile;
4-[2-({3-[(trifluoromethyl)oxy]phenyl}methyl)hexahydro-1H-1,2-diazepin-1-yl]-1-naphthalenecarbonitrile;
4-[2-({3-[(trifluoromethyl)oxy]phenyl}methyl)hexahydro-1H-1,2-diazepin-1-yl]-1-naphthalenecarbonitrile;
4-{2-[(5-methyl-2-thienyl)methyl]hexahydro-1H-1,2-diazepin-1-yl}-1-naphthalenecarbonitrile;
4-[2-(2-naphthalenylmethyl)hexahydro-1H-1,2-diazepin-1-yl]-1-naphthalenecarbonitrile;
4-{2-[(3-phenyl-1H-pyrazol-4-yl)methyl]hexahydro-1H-1,2-diazepin-1-yl}-1-naphthalenecarbonitrile;
4-(2-{[3-(trifluoromethyl)phenyl]methyl}hexahydro-1H-1,2-diazepin-1-yl)-1-naphthalenecarbonitrile;
4-(2-{[6-(methyloxy)-2-pyridinyl]methyl}hexahydro-1H-1,2-diazepin-1-yl)-1-naphthalenecarbonitrile;
4-{2-[(3,5-difluorophenyl)methyl]hexahydro-1H-1,2-diazepin-1-yl}-1-naphthalenecarbonitrile;
4-{2-[(4,5-dimethyl-2-furanyl)methyl]hexahydro-1H-1,2-diazepin-1-yl}-1-naphthalenecarbonitrile;
4-(2-{[3,5-bis(trifluoromethyl)phenyl]methyl}hexahydro-1H-1,2-diazepin-1-yl)-1-naphthalenecarbonitrile;
4-[2-(1-naphthalenylmethyl)hexahydro-1H-1,2-diazepin-1-yl]-1-naphthalenecarbonitrile;
4-(2-{[4-(methyloxy)phenyl]methyl}hexahydro-1H-1,2-diazepin-1-yl)-1-naphthalenecarbonitrile;
4-(2-{[3-(methyloxy)phenyl]methyl}hexahydro-1H-1,2-diazepin-1-yl)-1-naphthalenecarbonitrile;
4-(2-{[2-(methyloxy)phenyl]methyl}hexahydro-1H-1,2-diazepin-1-yl)-1-naphthalenecarbonitrile;
4-{[2-(4-nitro-1-naphthalenyl)tetrahydro-1(2H)-pyridazinyl]methyl}benzonitrile;
1-{[4-(1-methylethyl)phenyl]methyl}-2-(4-nitro-1-naphthalenyl)hexahydropyridazine;
1-(4-nitro-1-naphthalenyl)-2-(phenylmethyl)hexahydropyridazine;
1-(cyclohexylmethyl)-2-(4-nitro-1-naphthalenyl)hexahydropyridazine;
4-[2-(3-phenylbutyl)tetrahydro-1(2H)-pyridazinyl]-1-naphthalenecarbonitrile;
4-[2-(3-phenylpropyl)tetrahydro-1(2H)-pyridazinyl]-1-naphthalenecarbonitrile;
1-(4-nitro-1-naphthalenyl)-2-(3-phenylbutyl)hexahydropyridazine;
1-(4-nitro-1-naphthalenyl)-2-(3-phenylpropyl)hexahydropyridazine;
1-methyl-2-(4-nitro-1-naphthalenyl)hexahydropyridazine;
Methyl 6-[2-(4-nitro-1-naphthalenyl)tetrahydro-1(2H)-pyridazinyl]hexanoate;
4-{2-[(3-bromophenyl)methyl]-1-pyrazolidinyl}-1-naphthalenecarbonitrile;
4-[2-(3-thienylmethyl)-1-pyrazolidinyl]-1-naphthalenecarbonitrile;
4-(2-{[2,4-bis(trifluoromethyl)phenyl]methyl}-1-pyrazolidinyl)-1-naphthalenecarbonitrile;
4-{2-[(3-fluorophenyl)methyl]-1-pyrazolidinyl}-1-naphthalenecarbonitrile;
4-{2-[(2-bromophenyl)methyl]-1-pyrazolidinyl}-1-naphthalenecarbonitrile;
4-{2-[(4-acetylphenyl)methyl]-1-pyrazolidinyl}-1-naphthalenecarbonitrile;
4-(2-{[2-(trifluoromethyl)phenyl]methyl}-1-pyrazolidinyl)-1-naphthalenecarbonitrile;
4-(2-{[4-(1-methylethyl)phenyl]methyl}-1-pyrazolidinyl)-1-naphthalenecarbonitrile;
4-{2-[(4-fluorophenyl)methyl]-1-pyrazolidinyl}-1-naphthalenecarbonitrile;
4-(2-{[4-(trifluoromethyl)phenyl]methyl}-1-pyrazolidinyl)-1-naphthalenecarbonitrile;
4-[2-({3-[(trifluoromethyl)oxy]phenyl}methyl)-1-pyrazolidinyl]-1-naphthalenecarbonitrile;
4-{2-[(2-fluorophenyl)methyl]-1-pyrazolidinyl}-1-naphthalenecarbonitrile;
4-(2-{[3-(methyloxy)phenyl]methyl}-1-pyrazolidinyl)-1-naphthalenecarbonitrile;
4-(2-{[3,5-bis(trifluoromethyl)phenyl]methyl}-1-pyrazolidinyl)-1-naphthalenecarbonitrile;
4-(2-{[3-fluoro-5-(trifluoromethyl)phenyl]methyl}-1-pyrazolidinyl)-1-naphthalenecarbonitrile;
4-(2-{[3-(trifluoromethyl)phenyl]methyl}-1-pyrazolidinyl)-1-naphthalenecarbonitrile;
4-{2-[(4-methylphenyl)methyl]-1-pyrazolidinyl}-1-naphthalenecarbonitrile;
4-[2-(1-naphthalenylmethyl)-1-pyrazolidinyl]-1-naphthalenecarbonitrile;
4-[2-(phenylmethyl)-1-pyrazolidinyl]-1-naphthalenecarbonitrile;
4-{2-[(3,4-dichlorophenyl)methyl]-1-pyrazolidinyl}-1-naphthalenecarbonitrile;
4-(2-{[4-(methyloxy)phenyl]methyl}-1-pyrazolidinyl)-1-naphthalenecarbonitrile;
4-(2-{[3-(ethyloxy)phenyl]methyl}-1-pyrazolidinyl)-1-naphthalenecarbonitrile;
4-{2-[(4-cyanophenyl)methyl]-1-pyrazolidinyl}-1-naphthalenecarbonitrile;
4-{2-[(3-cyanophenyl)methyl]-1-pyrazolidinyl}-1-naphthalenecarbonitrile;
4-{2-[(2-methylphenyl)methyl]-1-pyrazolidinyl}-1-naphthalenecarbonitrile;
4-[2-[(4-phenyl-1H-imidazol-5-yl)methyl]tetrahydro-1(2H)-pyridazinyl]-1-naphthalenecarbonitrile;
4-[2-{[3-(1-methylethyl)-1H-pyrazol-4-yl]methyl}tetrahydro-1(2H)-pyridazinyl]-1-naphthalenecarbonitrile;
4-[2-{[3-(1-methylethyl)-1H-pyrazol-4-yl]methyl}tetrahydro-1(2H)-pyridazinyl]-1-naphthalenecarbonitrile;
4-[2-[(3-ethyl-1H-pyrazol-4-yl)methyl]tetrahydro-1(2H)-pyridazinyl]-1-naphthalenecarbonitrile;
4-[2-[(3-propyl-1H-pyrazol-4-yl)methyl]tetrahydro-1(2H)-pyridazinyl]-1-naphthalenecarbonitrile;
4-[2-{[3-(2-pyridinyl)-1H-pyrazol-4-yl]methyl}tetrahydro-1(2H)-pyridazinyl]-1-naphthalenecarbonitrile;
4-[2-{[3-(3-methylphenyl)-1H-pyrazol-4-yl]methyl}tetrahydro-1(2H)-pyridazinyl]-1-naphthalenecarbonitrile;
1-(2-{[(1,1-dimethylethyl)(dimethyl)silyl]oxy}ethyl)-2-(4-nitro-1-naphthalenyl)hexahydropyridazine;
4-[2-(2-{[(1,1-dimethylethyl)(dimethyl)silyl]oxy}ethyl)tetrahydro-1(2H)-pyridazinyl]-1-naphthalenecarbonitrile;
4-[2-{2-[2-(trifluoromethyl)phenyl]ethyl}tetrahydro-1(2H)-pyridazinyl]-1-naphthalenecarbonitrile;
4-[2-[2-(3,4-dichlorophenyl)ethyl]tetrahydro-1(2H)-pyridazinyl]-1-naphthalenecarbonitrile;
4-[2-[2-(3-fluorophenyl)ethyl]tetrahydro-1(2H)-pyridazinyl]-1-naphthalenecarbonitrile;
4-[2-[2-(2-methylphenyl)ethyl]tetrahydro-1(2H)-pyridazinyl]-1-naphthalenecarbonitrile;
4-[2-{2-[3-(methyloxy)phenyl]ethyl}tetrahydro-1(2H)-pyridazinyl]-1-naphthalenecarbonitrile;
4-[2-[2-(4-fluorophenyl)ethyl]tetrahydro-1(2H)-pyridazinyl]-1-naphthalenecarbonitrile;
4-[2-[2-(4-methylphenyl)ethyl]tetrahydro-1(2H)-pyridazinyl]-1-naphthalenecarbonitrile;
4-[2-[2-(2-bromophenyl)ethyl]tetrahydro-1(2H)-pyridazinyl]-1-naphthalenecarbonitrile;
4-[2-[2-(3-methylphenyl)ethyl]tetrahydro-1(2H)-pyridazinyl]-1-naphthalenecarbonitrile;
4-[2-[2-(2,4-dichlorophenyl)ethyl]tetrahydro-1(2H)-pyridazinyl]-1-naphthalenecarbonitrile;
4-[2-(2-phenylethyl)tetrahydro-1(2H)-pyridazinyl]-1-naphthalenecarbonitrile;
4-[2-[2-(1-naphthalenyl)ethyl]tetrahydro-1(2H)-pyridazinyl]-1-naphthalenecarbonitrile;
4-[2-{2-[3-(trifluoromethyl)phenyl]ethyl}tetrahydro-1(2H)-pyridazinyl]-1-naphthalenecarbonitrile;
4-[2-[2-(3-bromophenyl)ethyl]tetrahydro-1(2H)-pyridazinyl]-1-naphthalenecarbonitrile;
4-[2-[2-(2,6-dichlorophenyl)ethyl]tetrahydro-1(2H)-pyridazinyl]-1-naphthalenecarbonitrile;
4-[2-[2-(2-chloro-4-fluorophenyl)ethyl]tetrahydro-1(2H)-pyridazinyl]-1-naphthalenecarbonitrile;
4-[2-[2-(2-chlorophenyl)ethyl]tetrahydro-1(2H)-pyridazinyl]-1-naphthalenecarbonitrile;
4-[2-[2-(2,4,6-trimethylphenyl)ethyl]tetrahydro-1(2H)-pyridazinyl]-1-naphthalenecarbonitrile;
1-[2-(2,6-dichlorophenyl)ethyl]-2-(4-nitro-1-naphthalenyl)hexahydropyridazine;
1-[2-(2-chloro-4-fluorophenyl)ethyl]-2-(4-nitro-1-naphthalenyl)hexahydropyridazine;
1-[2-(2-chlorophenyl)ethyl]-2-(4-nitro-1-naphthalenyl)hexahydropyridazine;
1-(4-nitro-1-naphthalenyl)-2-[2-(2,4,6-trimethylphenyl)ethyl]hexahydropyridazine;
1-[2-(2-methylphenyl)ethyl]-2-(4-nitro-1-naphthalenyl)hexahydropyridazine;
1-{2-[3-(methyloxy)phenyl]ethyl}-2-(4-nitro-1-naphthalenyl)hexahydropyridazine;
1-(4-nitro-1-naphthalenyl)-2-{2-[2-(trifluoromethyl)phenyl]ethyl}hexahydropyridazine;
1-[2-(2-bromophenyl)ethyl]-2-(4-nitro-1-naphthalenyl)hexahydropyridazine;
1-(4-nitro-1-naphthalenyl)-2-[2-(3-nitrophenyl)ethyl]hexahydropyridazine;
1-[2-(3-methylphenyl)ethyl]-2-(4-nitro-1-naphthalenyl)hexahydropyridazine;
1-[2-(2,4-dichlorophenyl)ethyl]-2-(4-nitro-1-naphthalenyl)hexahydropyridazine;
1-[2-(1-naphthalenyl)ethyl]-2-(4-nitro-1-naphthalenyl)hexahydropyridazine;
1-(4-nitro-1-naphthalenyl)-2-{2-[3-(trifluoromethyl)phenyl]ethyl}hexahydropyridazine;
1-[2-(3-bromophenyl)ethyl]-2-(4-nitro-1-naphthalenyl)hexahydropyridazine;
1-[2-(4-fluorophenyl)ethyl]-2-(4-nitro-1-naphthalenyl)hexahydropyridazine;
4-{2-[2-(4-nitro-1-naphthalenyl)tetrahydro-1(2H)-pyridazinyl]ethyl}benzonitrile;
1-[2-(4-chlorophenyl)ethyl]-2-(4-nitro-1-naphthalenyl)hexahydropyridazine;
1-{2-[4-(ethyloxy)phenyl]ethyl}-2-(4-nitro-1-naphthalenyl)hexahydropyridazine;
1-(4-nitro-1-naphthalenyl)-2-(2-phenylethyl)hexahydropyridazine;
4-[2-(cyclohexylcarbonyl)tetrahydro-1(2H)-pyridazinyl]-1-naphthalenecarbonitrile;
4-(2-pentanoyltetrahydro-1(2H)-pyridazinyl)-1-naphthalenecarbonitrile;
4-[2-({3-[(phenylmethyl)oxy]phenyl}acetyl)tetrahydro-1(2H)-pyridazinyl]-1-naphthalenecarbonitrile;
4-[2-(cyclopentylacetyl)tetrahydro-1(2H)-pyridazinyl]-1-naphthalenecarbonitrile;
4-[2-(phenylacetyl)tetrahydro-1(2H)-pyridazinyl]-1-naphthalenecarbonitrile;
4-[2-(2-phenylbutanoyl)tetrahydro-1(2H)-pyridazinyl]-1-naphthalenecarbonitrile;
4-[2-{[(1S,2S)-2-phenylcyclopropyl]carbonyl}tetrahydro-1(2H)-pyridazinyl]-1-naphthalenecarbonitrile;
4-[2-(3-phenylpropanoyl)tetrahydro-1(2H)-pyridazinyl]-1-naphthalenecarbonitrile;
4-[2-(3,3-dimethylbutanoyl)tetrahydro-1(2H)-pyridazinyl]-1-naphthalenecarbonitrile;
4-[2-{[4-(methyloxy)phenyl]carbonyl}tetrahydro-1(2H)-pyridazinyl]-1-naphthalenecarbonitrile;
4-[2-({3-[(trifluoromethyl)oxy]phenyl}carbonyl)tetrahydro-1(2H)-pyridazinyl]-1-naphthalenecarbonitrile;
4-[2-{[3-(methyloxy)phenyl]carbonyl}tetrahydro-1(2H)-pyridazinyl]-1-naphthalenecarbonitrile;
4-[2-{[2-(methyloxy)phenyl]carbonyl}tetrahydro-1(2H)-pyridazinyl]-1-naphthalenecarbonitrile;
4-[2-{[2-(trifluoromethyl)phenyl]carbonyl}tetrahydro-1(2H)-pyridazinyl]-1-naphthalenecarbonitrile;
4-[2-{[3-(trifluoromethyl)phenyl]carbonyl}tetrahydro-1(2H)-pyridazinyl]-1-naphthalenecarbonitrile;
4-[2-({4-[(trifluoromethyl)oxy]phenyl}carbonyl)tetrahydro-1(2H)-pyridazinyl]-1-naphthalenecarbonitrile;
4-[2-[(3-cyanophenyl)carbonyl]tetrahydro-1(2H)-pyridazinyl]-1-naphthalenecarbonitrile;
4-[2-{[4-(trifluoromethyl)phenyl]carbonyl}tetrahydro-1(2H)-pyridazinyl]-1-naphthalenecarbonitrile;
4-[2-({2-[(trifluoromethyl)oxy]phenyl}carbonyl)tetrahydro-1(2H)-pyridazinyl]-1-naphthalenecarbonitrile;
1-(4-nitro-1-naphthalenyl)-2-pentanoylhexahydropyridazine;
1-(4-nitro-1-naphthalenyl)-2-{[3-(trifluoromethyl)phenyl]carbonyl}hexahydropyridazine;
1-(4-nitro-1-naphthalenyl)-2-{[4-(trifluoromethyl)phenyl]carbonyl}hexahydropyridazine;
4-{[2-(4-nitro-1-naphthalenyl)tetrahydro-1(2H)-pyridazinyl]carbonyl}benzonitrile;
1-{[3-(methyloxy)phenyl]carbonyl}-2-(4-nitro-1-naphthalenyl)hexahydropyridazine;
1-{[4-(methyloxy)phenyl]carbonyl}-2-(4-nitro-1-naphthalenyl)hexahydropyridazine;
1-(1-naphthalenylcarbonyl)-2-(4-nitro-1-naphthalenyl)hexahydropyridazine;
1-(2-naphthalenylcarbonyl)-2-(4-nitro-1-naphthalenyl)hexahydropyridazine;
1-(4-nitro-1-naphthalenyl)-2-(2-thienylcarbonyl)hexahydropyridazine;
1-(2-methylpropanoyl)-2-(4-nitro-1-naphthalenyl)hexahydropyridazine;
1-[(2-bromophenyl)carbonyl]-2-(4-nitro-1-naphthalenyl)hexahydropyridazine;
1-[(3-bromophenyl)carbonyl]-2-(4-nitro-1-naphthalenyl)hexahydropyridazine;
1-[(4-bromophenyl)carbonyl]-2-(4-nitro-1-naphthalenyl)hexahydropyridazine;
1-[(2,4-dichlorophenyl)carbonyl]-2-(4-nitro-1-naphthalenyl)hexahydropyridazine;
1-{[4-(1,1-dimethylethyl)phenyl]carbonyl}-2-(4-nitro-1-naphthalenyl)hexahydropyridazine;
1-(4-nitro-1-naphthalenyl)-2-[(phenyloxy)acetyl]hexahydropyridazine;
1-(4-nitro-1-naphthalenyl)-2-(2-phenylbutanoyl)hexahydropyridazine;
1-(4-nitro-1-naphthalenyl)-2-(phenylacetyl)hexahydropyridazine;
1-(cyclopropylcarbonyl)-2-(4-nitro-1-naphthalenyl)hexahydropyridazine;
1-(3-methylbutanoyl)-2-(4-nitro-1-naphthalenyl)hexahydropyridazine;
1-(cyclohexylcarbonyl)-2-(4-nitro-1-naphthalenyl)hexahydropyridazine;
1-[(3-methylphenyl)carbonyl]-2-(4-nitro-1-naphthalenyl)hexahydropyridazine;
1-[(4-methylphenyl)carbonyl]-2-(4-nitro-1-naphthalenyl)hexahydropyridazine;
1-(4-nitro-1-naphthalenyl)-2-{[2-(trifluoromethyl)phenyl]carbonyl}hexahydropyridazine;
1-{[3-(methyloxy)phenyl]acetyl}-2-(4-nitro-1-naphthalenyl)hexahydropyridazine;
1-{[4-(butyloxy)phenyl]carbonyl}-2-(4-nitro-1-naphthalenyl)hexahydropyridazine;
1-(cyclobutylcarbonyl)-2-(4-nitro-1-naphthalenyl)hexahydropyridazine;
1-(cyclopentylcarbonyl)-2-(4-nitro-1-naphthalenyl)hexahydropyridazine;
1-(cyclopentylacetyl)-2-(4-nitro-1-naphthalenyl)hexahydropyridazine;
1-(2-methylpentanoyl)-2-(4-nitro-1-naphthalenyl)hexahydropyridazine;
1-(3-cyclopentylpropanoyl)-2-(4-nitro-1-naphthalenyl)hexahydropyridazine;
1-[(2-bromophenyl)acetyl]-2-(4-nitro-1-naphthalenyl)hexahydropyridazine;
1-(3,3-dimethylbutanoyl)-2-(4-nitro-1-naphthalenyl)hexahydropyridazine;
1-(4-nitro-1-naphthalenyl)-2-{[(1S,2S)-2-phenylcyclopropyl]carbonyl}hexahydropyridazine;
1-(4-nitro-1-naphthalenyl)-2-(3-phenylpropanoyl)hexahydropyridazine;
1-[(4-chlorophenyl)acetyl]-2-(4-nitro-1-naphthalenyl)hexahydropyridazine;
1-(diphenylacetyl)-2-(4-nitro-1-naphthalenyl)hexahydropyridazine;
1-{[4-(methyloxy)phenyl]acetyl}-2-(4-nitro-1-naphthalenyl)hexahydropyridazine;
1-(4-nitro-1-naphthalenyl)-2-({3-[(trifluoromethyl)oxy]phenyl}carbonyl)hexahydropyridazine;
4-(2-acetyl-1-pyrazolidinyl)-1-naphthalenecarbonitrile;
4-[2-(2-methylpropanoyl)-1-pyrazolidinyl]-1-naphthalenecarbonitrile;
4-[2-(3-phenylpropanoyl)-1-pyrazolidinyl]-1-naphthalenecarbonitrile;
4-(2-butanoyl-1-pyrazolidinyl)-1-naphthalenecarbonitrile;
4-[2-(cyclopropylcarbonyl)-1-pyrazolidinyl]-1-naphthalenecarbonitrile;
4-[2-(3-methylbutanoyl)-1-pyrazolidinyl]-1-naphthalenecarbonitrile;
4-[2-(phenylcarbonyl)-1-pyrazolidinyl]-1-naphthalenecarbonitrile;
4-(2-{[3-(trifluoromethyl)phenyl]carbonyl}-1-pyrazolidinyl)-1-naphthalenecarbonitrile;
4-{2-[(2-fluorophenyl)carbonyl]-1-pyrazolidinyl}-1-naphthalenecarbonitrile;
4-{2-[(3-methylphenyl)carbonyl]-1-pyrazolidinyl}-1-naphthalenecarbonitrile;
4-[2-(2-furanylcarbonyl)-1-pyrazolidinyl]-1-naphthalenecarbonitrile;
4-{2-[(3-fluorophenyl)carbonyl]-1-pyrazolidinyl}-1-naphthalenecarbonitrile;
4-{2-[(2-methylphenyl)carbonyl]-1-pyrazolidinyl}-1-naphthalenecarbonitrile;
4-[2-(cyclopentylcarbonyl)-1-pyrazolidinyl]-1-naphthalenecarbonitrile;
4-[2-(2-phenylbutanoyl)-1-pyrazolidinyl]-1-naphthalenecarbonitrile;
4-[2-({2-[(trifluoromethyl)oxy]phenyl}acetyl)tetrahydro-1(2H)-pyridazinyl]-1-naphthalenecarbonitrile;
4-[2-{[2-(methyloxy)phenyl]acetyl}tetrahydro-1(2H)-pyridazinyl]-1-naphthalenecarbonitrile;
4-[2-{[4-(ethyloxy)phenyl]acetyl}tetrahydro-1(2H)-pyridazinyl]-1-naphthalenecarbonitrile;
4-[2-{[2-(ethyloxy)phenyl]acetyl}tetrahydro-1(2H)-pyridazinyl]-1-naphthalenecarbonitrile;
4-[2-{[3-(methyloxy)phenyl]acetyl}tetrahydro-1(2H)-pyridazinyl]-1-naphthalenecarbonitrile;
4-[2-{[3-(ethyloxy)phenyl]acetyl}tetrahydro-1(2H)-pyridazinyl]-1-naphthalenecarbonitrile,
4-[2-({4-[(trifluoromethyl)oxy]phenyl}acetyl)tetrahydro-1(2H)-pyridazinyl]-1-naphthalenecarbonitrile;
4-[2-{[2-fluoro-6-(trifluoromethyl)phenyl]acetyl}tetrahydro-1(2H)-pyridazinyl]-1-naphthalenecarbonitrile;
4-[2-[(2-fluorophenyl)acetyl]tetrahydro-1(2H)-pyridazinyl]-1-naphthalenecarbonitrile;
4-[2-[(2,3-difluorophenyl)acetyl]tetrahydro-1(2H)-pyridazinyl]-1-naphthalenecarbonitrile;
4-[2-{[2-(phenyloxy)phenyl]acetyl}tetrahydro-1(2H)-pyridazinyl]-1-naphthalenecarbonitrile;
4-[2-[(2-iodophenyl)acetyl]tetrahydro-1(2H)-pyridazinyl]-1-naphthalenecarbonitrile;
4-[2-[(pentafluorophenyl)acetyl]tetrahydro-1(2H)-pyridazinyl]-1-naphthalenecarbonitrile;
4-[2-(2-naphthalenylacetyl)tetrahydro-1(2H)-pyridazinyl]-1-naphthalenecarbonitrile;
4-[2-[(2-bromophenyl)acetyl]tetrahydro-1(2H)-pyridazinyl]-1-naphthalenecarbonitrile;
4-[2-{[3-(trifluoromethyl)phenyl]acetyl}tetrahydro-1(2H)-pyridazinyl]-1-naphthalenecarbonitrile;
4-[2-[(2,4-difluorophenyl)acetyl]tetrahydro-1(2H)-pyridazinyl]-1-naphthalenecarbonitrile;
4-[2-({2-[(phenylmethyl)oxy]phenyl}acetyl)tetrahydro-1(2H)-pyridazinyl]-1-naphthalenecarbonitrile;
4-[2-{[2-(trifluoromethyl)phenyl]acetyl}tetrahydro-1(2H)-pyridazinyl]-1-naphthalenecarbonitrile;
4-[2-[(2,6-difluorophenyl)acetyl]tetrahydro-1(2H)-pyridazinyl]-1-naphthalenecarbonitrile;
4-[2-[(2-chloro-4-fluorophenyl)acetyl]tetrahydro-1(2H)-pyridazinyl]-1-naphthalenecarbonitrile;
4-[2-[(4-fluorophenyl)acetyl]tetrahydro-1(2H)-pyridazinyl]-1-naphthalenecarbonitrile;
4-[2-[(3-nitrophenyl)acetyl]tetrahydro-1(2H)-pyridazinyl]-1-naphthalenecarbonitrile;
4-[2-[(3,4-dichlorophenyl)acetyl]tetrahydro-1(2H)-pyridazinyl]-1-naphthalenecarbonitrile;
4-[2-[(2-chloro-6-fluorophenyl)acetyl]tetrahydro-1(2H)-pyridazinyl]-1-naphthalenecarbonitrile;
4-[2-[(2-methylphenyl)acetyl]tetrahydro-1(2H)-pyridazinyl]-1-naphthalenecarbonitrile;
4-[2-[(3-methylphenyl)acetyl]tetrahydro-1(2H)-pyridazinyl]-1-naphthalenecarbonitrile;
4-[2-({3-[(trifluoromethyl)oxy]phenyl}acetyl)tetrahydro-1(2H)-pyridazinyl]-1-naphthalenecarbonitrile;
4-[2-({3-[(trifluoromethyl)oxy]phenyl}acetyl)-1-pyrazolidinyl]-1-naphthalenecarbonitrile;
4-[2-({3-[(trifluoromethyl)oxy]phenyl}acetyl)-1-pyrazolidinyl]-1-naphthalenecarbonitrile;
4-{2-[(2-fluorophenyl)acetyl]-1-pyrazolidinyl}-1-naphthalenecarbonitrile;
4-(2-{[3-(trifluoromethyl)phenyl]acetyl}-1-pyrazolidinyl)-1-naphthalenecarbonitrile;
4-{2-[(2-bromophenyl)acetyl]-1-pyrazolidinyl}-1-naphthalenecarbonitrile;
4-{2-[(3-methylphenyl)acetyl]-1-pyrazolidinyl}-1-naphthalenecarbonitrile;
4-{2-[(2-methylphenyl)acetyl]-1-pyrazolidinyl}-1-naphthalenecarbonitrile;
4-{2-[(3,4-dichlorophenyl)acetyl]-1-pyrazolidinyl}-1-naphthalenecarbonitrile;
4-[2-({2-[(trifluoromethyl)oxy]phenyl}acetyl)-1-pyrazolidinyl]-1-naphthalenecarbonitrile;
4-[2-(3-isoxazolylacetyl)tetrahydro-1(2H)-pyridazinyl]-1-naphthalenecarbonitrile;
4-[2-[(1-methyl-1H-indol-3-yl)acetyl]tetrahydro-1(2H)-pyridazinyl]-1-naphthalenecarbonitrile;
4-[2-[(ethyloxy)(phenyl)acetyl]tetrahydro-1(2H)-pyridazinyl]-1-naphthalenecarbonitrile;
4-[2-(2-thienylacetyl)tetrahydro-1(2H)-pyridazinyl]-1-naphthalenecarbonitrile;
4-[2-(3-thienylacetyl)tetrahydro-1(2H)-pyridazinyl]-1-naphthalenecarbonitrile;
4-[2-(2-furanylacetyl)tetrahydro-1(2H)-pyridazinyl]-1-naphthalenecarbonitrile;
4-[2-[3-(4-fluorophenyl)propanoyl]tetrahydro-1(2H)-pyridazinyl]-1-naphthalenecarbonitrile;
4-[2-[3-(3-bromophenyl)propanoyl]tetrahydro-1(2H)-pyridazinyl]-1-naphthalenecarbonitrile;
4-[2-[3-(3,4-dichlorophenyl)propanoyl]tetrahydro-1(2H)-pyridazinyl]-1-naphthalenecarbonitrile;
4-[2-[3-(1,3-benzodioxol-5-yl)propanoyl]tetrahydro-1(2H)-pyridazinyl]-1-naphthalenecarbonitrile;
4-[2-{3-[3,5-bis(trifluoromethyl)phenyl]propanoyl}tetrahydro-1(2H)-pyridazinyl]-1-naphthalenecarbonitrile;
4-[2-[(3R)-3-phenylbutanoyl]tetrahydro-1(2H)-pyridazinyl]-1-naphthalenecarbonitrile;
4-[2-{3-[4-(trifluoromethyl)phenyl]propanoyl}tetrahydro-1(2H)-pyridazinyl]-1-naphthalenecarbonitrile;
4-[2-(3-{4-[(trifluoromethyl)oxy]phenyl}propanoyl)tetrahydro-1(2H)-pyridazinyl]-1-naphthalenecarbonitrile;
4-[2-[3-(3,4-difluorophenyl)propanoyl]tetrahydro-1(2H)-pyridazinyl]-1-naphthalenecarbonitrile;
4-[2-{3-[2-(methyloxy)phenyl]propanoyl}tetrahydro-1(2H)-pyridazinyl]-1-naphthalenecarbonitrile;
4-[2-[3-(2-bromophenyl)propanoyl]tetrahydro-1(2H)-pyridazinyl]-1-naphthalenecarbonitrile;
4-[2-[3-(3-chlorophenyl)propanoyl]tetrahydro-1(2H)-pyridazinyl]-1-naphthalenecarbonitrile;
4-[2-(3,3-diphenylpropanoyl)tetrahydro-1(2H)-pyridazinyl]-1-naphthalenecarbonitrile
4-[2-{3-[3-(methyloxy)phenyl]propanoyl}tetrahydro-1(2H)-pyridazinyl]-1-naphthalenecarbonitrile;
4-[2-[3-(2-chlorophenyl)propanoyl]tetrahydro-1(2H)-pyridazinyl]-1-naphthalenecarbonitrile;
4-[2-[3-(4-methylphenyl)propanoyl]tetrahydro-1(2H)-pyridazinyl]-1-naphthalenecarbonitrile;
4-[2-{3-[3-(trifluoromethyl)phenyl]propanoyl}tetrahydro-1(2H)-pyridazinyl]-1-naphthalenecarbonitrile;
4-[2-{3-[4-(methyloxy)phenyl]propanoyl}tetrahydro-1(2H)-pyridazinyl]-1-naphthalenecarbonitrile;
4-[2-(3-phenylbutanoyl)tetrahydro-1(2H)-pyridazinyl]-1-naphthalenecarbonitrile;
4-[2-[3-(2-fluorophenyl)propanoyl]tetrahydro-1(2H)-pyridazinyl]-1-naphthalenecarbonitrile;
4-[2-{[4-fluoro-2-(trifluoromethyl)phenyl]acetyl}tetrahydro-1(2H)-pyridazinyl]-1-naphthalenecarbonitrile;
4-[2-[3-(2-methylphenyl)propanoyl]tetrahydro-1(2H)-pyridazinyl]-1-naphthalenecarbonitrile;
4-[2-[3-(3-fluorophenyl)propanoyl]tetrahydro-1(2H)-pyridazinyl]-1-naphthalenecarbonitrile;
4-[2-[3-(4-bromophenyl)propanoyl]tetrahydro-1(2H)-pyridazinyl]-1-naphthalenecarbonitrile;
4-[2-[2-(phenyloxy)propanoyl]tetrahydro-1(2H)-pyridazinyl]-1-naphthalenecarbonitrile;
4-[2-(2-cyano-3-phenylpropanoyl)tetrahydro-1(2H)-pyridazinyl]-1-naphthalenecarbonitrile;
4-[2-[3-methyl-3-(1H-pyrrol-1-yl)butanoyl]tetrahydro-1(2H)-pyridazinyl]-1-naphthalenecarbonitrile;
4-[2-[3-(2-furanyl)propanoyl]tetrahydro-1(2H)-pyridazinyl]-1-naphthalenecarbonitrile;
4-[2-[3-(4-methyl-1,3-thiazol-5-yl)propanoyl]tetrahydro-1(2H)-pyridazinyl]-1-naphthalenecarbonitrile;
4-[2-[2-(phenyloxy)butanoyl]tetrahydro-1(2H)-pyridazinyl]-1-naphthalenecarbonitrile;
1-[(4-methylphenyl)acetyl]-2-(4-nitro-1-naphthalenyl)hexahydropyridazine;
1-(4-nitro-1-naphthalenyl)-2-{[4-(trifluoromethyl)phenyl]acetyl}hexahydropyridazine;
1-(4-nitro-1-naphthalenyl)-2-({2-[(trifluoromethyl)oxy]phenyl}acetyl)hexahydropyridazine;
1-(4-nitro-1-naphthalenyl)-2-{[3-(trifluoromethyl)phenyl]acetyl}hexahydropyridazine;
1-[(3-bromophenyl)acetyl]-2-(4-nitro-1-naphthalenyl)hexahydropyridazine;
1-(4-nitro-1-naphthalenyl)-2-({2-[(phenylmethyl)oxy]phenyl}acetyl)hexahydropyridazine;
1-{[2-(ethyloxy)phenyl]acetyl}-2-(4-nitro-1-naphthalenyl)hexahydropyridazine;
1-(4-nitro-1-naphthalenyl)-2-{[2-(trifluoromethyl)phenyl]acetyl}hexahydropyridazine;
1-[(3-methylphenyl)acetyl]-2-(4-nitro-1-naphthalenyl)hexahydropyridazine;
1-{[3-(ethyloxy)phenyl]acetyl}-2-(4-nitro-1-naphthalenyl)hexahydropyridazine;
1-(2-naphthalenylacetyl)-2-(4-nitro-1-naphthalenyl)hexahydropyridazine;
1-{[2-(methyloxy)phenyl]acetyl}-2-(4-nitro-1-naphthalenyl)hexahydropyridazine;
1-{[4-(ethyloxy)phenyl]acetyl}-2-(4-nitro-1-naphthalenyl)hexahydropyridazine;
1-[(4-bromophenyl)acetyl]-2-(4-nitro-1-naphthalenyl)hexahydropyridazine;
1-[(2-methylphenyl)acetyl]-2-(4-nitro-1-naphthalenyl)hexahydropyridazine;
1-(4-nitro-1-naphthalenyl)-2-{[3-(phenyloxy)phenyl]acetyl}hexahydropyridazine;
1-{[3,4-bis(methyloxy)phenyl]acetyl}-2-(4-nitro-1-naphthalenyl)hexahydropyridazine;
4-[2-(2-hydroxyethyl)tetrahydro-1(2H)-pyridazinyl]-1-naphthalenecarbonitrile;
2-[2-(4-nitro-1-naphthalenyl)tetrahydro-1(2H)-pyridazinyl]ethanol;
4-[2-[2-(phenyloxy)ethyl]tetrahydro-1(2H)-pyridazinyl]-1-naphthalenecarbonitrile;
4-[2-{2-[(3-chlorophenyl)oxy]ethyl}tetrahydro-1(2H)-pyridazinyl]-1-naphthalenecarbonitrile;
1-(4-nitro-1-naphthalenyl)-2-[2-(phenyloxy)ethyl]hexahydropyridazine;
1-{2-[(2-methylphenyl)oxy]ethyl}-2-(4-nitro-1-naphthalenyl)hexahydropyridazine;
1-{2-[(3-chlorophenyl)oxy]ethyl}-2-(4-nitro-1-naphthalenyl)hexahydropyridazine;
1-{2-[(4-methylphenyl)oxy]ethyl}-2-(4-nitro-1-naphthalenyl)hexahydropyridazine;
2-(4-cyano-1-naphthalenyl)tetrahydro-1(2H)-pyridazinecarbonitrile;
2-(4-nitro-1-naphthalenyl)tetrahydro-1(2H)-pyridazinecarbonitrile;
4-[2-(2,2,2-trifluoroethyl)hexahydro-1H-1,2-diazepin-1-yl]-1-naphthalenecarbonitrile;
4-[2-({3-[4-(trifluoromethyl)phenyl]-1H-pyrazol-4-yl}methyl)tetrahydro-1(2H)-pyridazinyl]-1-naphthalenecarbonitrile;
4-[2-({3-[2-(trifluoromethyl)phenyl]-1H-pyrazol-4-yl}methyl)tetrahydro-1(2H)-pyridazinyl]-1-naphthalenecarbonitrile;
4-[2-{[3-(4-chlorophenyl)-1H-pyrazol-4-yl]methyl}tetrahydro-1(2H)-pyridazinyl]-1-naphthalenecarbonitrile;
4-[2-{[3-(4-fluorophenyl)-1H-pyrazol-4-yl]methyl}tetrahydro-1(2H)-pyridazinyl]-1-naphthalenecarbonitrile;
4-[2-({3-[3-(trifluoromethyl)phenyl]-1H-pyrazol-4-yl}methyl)tetrahydro-1(2H)-pyridazinyl]-1-naphthalenecarbonitrile;
   or a salt or solvate thereof.

### ABBREVIATIONS

As used herein the symbols and conventions used in these processes, schemes and examples are consistent with those used in the contemporary scientific literature, for example, The Journal of the American Chemical Society or the Journal of Biological Chemistry. Specifically, the following abbreviations may be used in the examples and throughout the specification:

| | |
|---|---|
| g (grams); | mg (milligrams); |
| L (liters); | ml (milliliters); |
| μL (microliters); | psi (pounds per square inch); |
| M (molar); | mM (millimolar); |
| Hz (Hertz); | MHz (megahertz); |
| mol (mol(s)); | mmol (millimol(s)); |
| rt (room temperature); | eq (equivalent); |
| min (minutes); | h (hours); |
| mp (melting point); | TLC (thin layer chromatography); |
| Tᵣ (retention time); | RP (reverse phase); |
| TEA (triethylamine); | TFA (trifluoroacetic acid); |
| THF (tetrahydrofuran); | CDCl₃ (deuterated chloroform); |
| CD₃OD (deuterated methanol); | SiO₂ (silica); |
| H₂O₂ (hydrogen peroxide); | H₂SO₄ (sulfuric acid); |
| DMSO (dimethylsulfoxide); | EtOAc (ethyl acetate); |
| HCl (hydrochloric acid); | CH₂Cl₂ (methylene chloride); |
| LiAlH₄ (lithium aluminum hydride); | CHCl₃ (chloroform); |
| DMF (N,N-dimethylformamide); | HOAc (acetic acid); |
| BOC (tert-butyloxycarbonyl); | LiOH (lithium hydroxide); |
| Ac (acetyl); | atm (atmosphere); |
| TBS (t-butyldimethylsilyl); | Me (methyl); |
| Et (ethyl); | EtOH (ethanol); |
| MeOH (methanol); | tBu (tert-butyl); |
| PtO₂ (platinum dioxide). | NaH (sodium hydride); |
| w/w (weight/weight); | m (multiplet); |
| ppm (parts-per-million): | d (doublet); |
| t (triplet); | q (quartet); |
| J (coupling constant); | dd (doublet of doublets); |
| ESI (electrospray injection); | N (normal); |
| ES⁺(electrospray ionization | m/z (mass-charge ratio); |
| in positive mode); | |
| MS (mass spectrometry); | wt % (weight percent); |
| HPLC (high pressure liquid | mm (millimeters); |
| chromatography); | |
| mBar (millibar); | NaOH (sodium hydroxide); |

HATU (O-(7-Azabenzotriazol-1-yl)-N,N,N′,N′-tetramethyluronium
hexafluorophosphate);

| | |
|---|---|
| PS (polystyrene); | |
| DMEM = Dulbecco's modified | FBS = fetal calf serum; |
| Eagle's medium; | |
| Pen/Strep = penicillin and | PBS = phosphate-buffered saline; |
| streptomycin; | |
| DTT = Dithiothreitol; | ip = intraperitoneal. |

Unless otherwise indicated, all temperatures are expressed in ° C. (degrees Centigrade). All reactions conducted under an inert atmosphere at room temperature unless otherwise noted.

¹H NMR spectra were recorded on a Varian VXR-300, a Varian Unity-300, a Varian Unity-400 instrument, or a General Electric QE-300. Chemical shifts are expressed in parts per million (ppm, δ units). Coupling constants are in units of hertz (Hz). Splitting patterns describe apparent multiplicities and are designated as s (singlet), d (doublet), t (triplet), q (quartet), m (multiplet), or br (broad).

Compounds were analyzed on a Micromass ZMD LC/MS using either Conditions I or Conditions II (below). Retention times were recorded for each compound.

Conditions I: The column was a C18 Phenomenex Luna, 20×4.0 mm, 3-micron column 90% H2O, 10% MeOH to 100% MeOH in 3 minutes, holding at 100% MeOH for final 1 minute. Water contained 0.1% v/v formic acid, MeOH contains 0.075% v/v formic acid. The flow rate was 2 ml/min with 3 uL of solution injected. Mass spectra were recorded on a Micromass ZMD utilizing electrospray ionization or atmospheric pressure chemical ionization (APCI) switching between positive and negative modes with DAD (Waters 996 DAD) scanning from 210 to 400 nm.
Conditions II: The column was a C18 Phenomenex Luna, 20×4.0 mm, 3-micron column 98% H2O, 2% MeOH to 100% MeOH in 3 minutes, holding at 100% MeOH for final 1 minute. Water contained 0.1% v/v formic acid, MeOH contains 0.075% v/v formic acid. The flow rate was 2 ml/min with 3 uL of solution injected. Mass spectra were recorded on a Micromass ZMD utilizing electrospray ionization or atmospheric pressure chemical ionization (APCI) switching between positive and negative modes with DAD (Waters 996 DAD) scanning from 210 to 400 nm.

Compounds were purified on an Agilent 1100 HPLC using a Phenomenex Luna C-18(2), 150×21.2 mm, 5 micron column; a linear gradient of 10-90% ACN/H2O/0.1% TFA or 30% ACN/H2O/0.1% TFA was run over 10 minutes, followed by 2 minutes at 100% ACN. The flow rate was 20 mL/min with DAD at 254 nm or 214 mm.

The syntheses of compounds of formula (I) proceeded via the formation of the mono-substituted cyclic hydrazine B which is derived from the nucleophilic addition of the cyclic hydrazine dihydrochloride salt to various electron deficient naphthalenes (Scheme 1, n=0-2). Intermediate B can then be used in reductive amination reactions to produce compounds C (Scheme 1, PS=polystyrene) or amide coupling transformations utilizing the three methods illustrated in Scheme 2 to furnish compounds D.

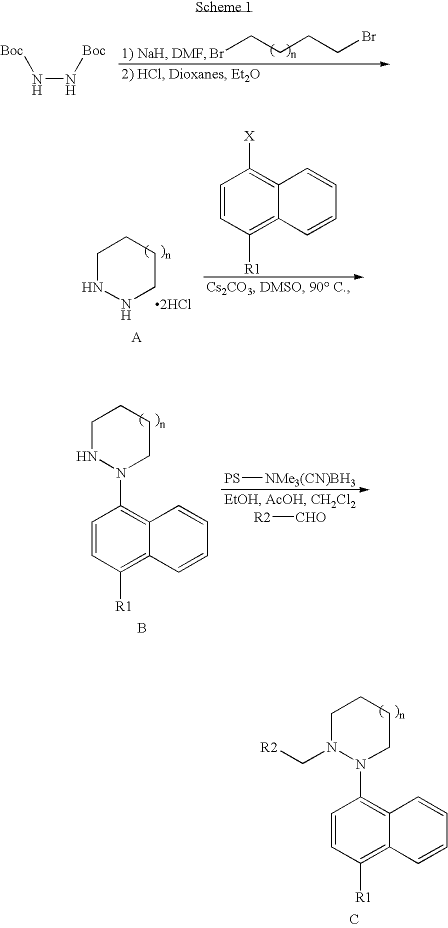

The first illustrated method in Scheme 2, Method A, involves coupling of intermediate B with various carboxylic acid chlorides. Method B proceeds via a HATU mediated amide coupling, while Method C utilizes a mixed-anhydride approach to furnish compounds D.

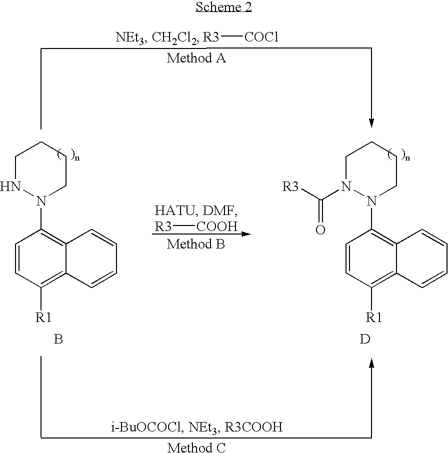

Intermediate B has also been used to install aryloxyethyl-sidechains as shown in Scheme 3. This method utilizes similar reductive amination methodology as illustrated in Scheme 1 to arrive at intermediate E. After subsequent TBDMS deprotection with refluxing ethanol, standard Mitsonobu conditions were employed to yield compounds G.

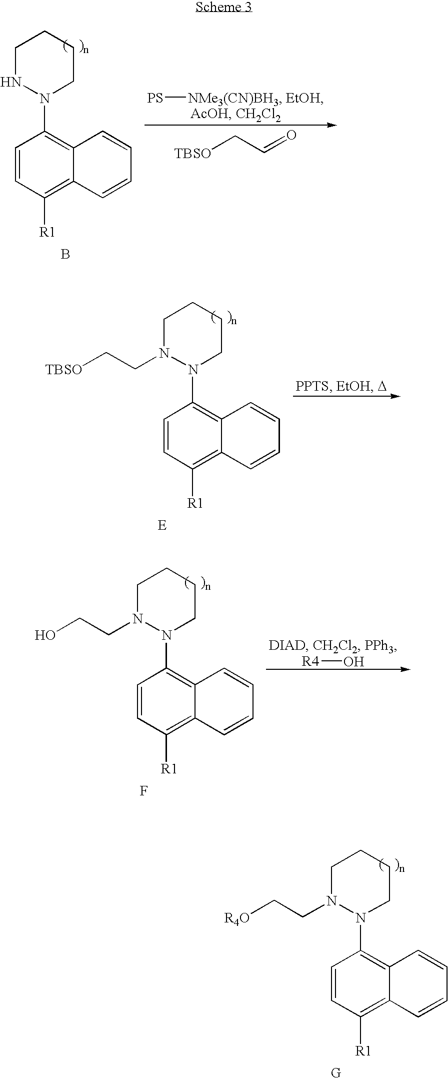

### EXAMPLES

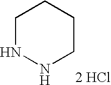

### A Hexahydropyridazine Dihydrochloride (A1)

To a 1-L 3-neck round bottom flask equipped with a magnetic stirbar, an addition funnel, and nitrogen flow, was added 7.6 g NaH (60% w/w dispersion in mineral oil, 0.19 moles, 2.2 eq). The NaH was washed three times with hexanes after which were added 350 ml of dry DMF. The reaction mixture was cooled to 0° C. via an ice bath, then 20 g t-butyl carbazate (0.086 moles, 1 eq) in 50 ml dry DMF was added dropwise via the addition funnel. After the addition was complete, the reaction was allowed to warm to room temperature and stirred for 30 minutes. Then 10.27 ml of 1,4-dibromobutane (0.086 moles, 1 eq) was added all at once at room temperature and stirred overnight. The reaction mixture was quenched with water until gas evolution ceased, then partitioned between diethyl ether and water. The phases were separated and the organic fraction was washed twice with water. The ether layer was concentrated in vacuo. The residue was dissolved in 300 ml of 4N HCl in dioxanes to which was added 300 ml of diethyl ether. This mixture was stirred at room temperature for 1 hour, at which time a white solid precipitated. The solids were isolated (13.7 g, 100% yield) by filtration and dried under vacuum.

1H NMR (400 MHz, DMSO-D6) 8 ppm 1.6 (m, 4H) 3.0 (m, 4H)

The following compounds were synthesized according to the same general procedure as used for intermediate A1:

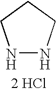

### Pyrazolidine Dihydrochloride (A2)

From 1,3-dibromopropane the title compound was obtained.

1H NMR (300 MHz, DMSO-D6) δ ppm 2.0 (m, 2H) 3.0 (m, 4H)

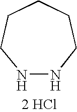

### Hexahydro-1H-1,2-diazepine dihydrochloride (A3)

From 1,5-dibromopentane the title compound was obtained.

1H NMR (400 MHz, DMSO-D6) δ ppm 1.6 (m, 6H) 3.0 (m, 2H) 3.7 (m, 2H)

### Cyclic Hydrazine Additions

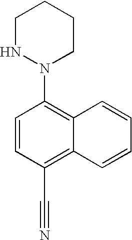

### B. 4-(tetrahydro-1(2H)-pyridazinyl)-1-naphthalenecarbonitrile (B1)

To a 500 ml round bottom flask equipped with a magnetic stirbar, an addition funnel, and nitrogen flow was added 7.0 g hexahydropyridazine dihydrochloride (43.18 mmoles, 1.5 eq) in 100 ml dry DMSO, then 33 g cesium carbonate (0.102 moles, 3.5 eq) was added all at once. The reaction mixture was heated to 80° C. via an oil bath, at which point, 5 g 4-fluoro-1-naphthalenecarbonitrile (29.21 mmoles, 1 eq) in 20 ml DMSO was added dropwise. The reaction was stirred at 80° C. overnight. After cooling to ambient temperature, 100 ml distilled water was added, which resulted in the precipitation of a 5.6 g (80% yield) of the title compound as a yellow solid.

1H NMR (400 MHz, DMSO-D6) δ ppm 1.6 (m, 2H) 1.9 (m, 2H) 3.1 (m, 2H) 3.4 (m, 2H) 4.5 (m, 1H) 7.2 (d, J=8.6 Hz, 1H) 7.6 (m, 1H) 7.7 (m, 1H) 8.3 (m, 2H) 8.5 (d, J=8.1 Hz, 1H)

The following compounds were synthesized according to the same general procedure as used for intermediate B1:

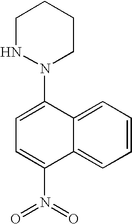

### 1-(4-nitro-1-naphthalenyl)hexahydropyridazine (B2)

From 1-chloro-4-nitronaphthalene and A1 the title compound was isolated as an orange solid.

1H NMR (400 MHz, DMSO-D6) 8 ppm 1.6 (m, 2H) 1.9 (m, 2H) 3.1 (m, 2H) 3.4 (m, 2H) 4.5 (m, 1H) 7.2 (d, J=8.6 Hz, 1H) 7.6 (m, 1H) 7.7 (m, 1H) 8.3 (m, 2H) 8.5 (d, J=8.1 Hz, 1H)

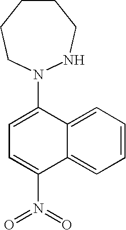

### 4-(hexahydro-1H-1,2-diazepin-1-yl)-1-naphthalenecarbonitrile (B3)

From 1-chloro-4-nitronaphthalene and A3 the title compound was obtained.

MS (m/z) ESI ES⁺=272

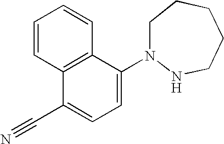

### 4-(hexahydro-1H-1,2-diazepin-1-yl)-1-naphthalenecarbonitrile (B4)

From 4-fluoro-1-naphthalenecarbonitrile and A3 the title compound was obtained.

MS (m/z) ESI ES⁺=252

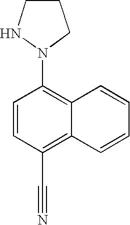

### 4-(1-pyrazolidinyl)-1-naphthalenecarbonitrile (B5)

From 4-fluoro-1-naphthalenecarbonitrile and A2 the title compound was obtained.

1H NMR (400 MHz, CHLOROFORM-D) δ ppm 2.1 (m, 2H) 3.2 (m, J=7.1, 7.1 Hz, 2H) 3.6 (m, 2H) 7.5 (m, 2H) 7.6 (m, 1H) 7.8 (d, J=8.1 Hz, 1H) 8.2 (m, 1H) 8.2 (m, 1H)

### Cyclic Hydrazine Reductive Aminations Excluding Phenylacetaldehydes

### Example 1

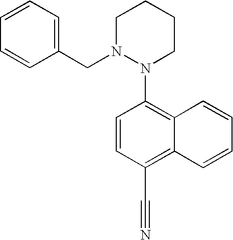

### C. 4-[2-(phenylmethyl)tetrahydro-1(2H)-pyridazinyl]-1-naphthalenecarbonitrile (C1)

To a 40 ml scintillation vial was added 75 mg B1 (0.316 mmoles, 1 eq), 1 ml ethanol, 0.5 ml acetic acid, 1 ml dichloromethane, and 0.042 ml benzaldehyde (0.411 mmoles, 1.3 eq). Then 400 mg (polystyrylmethyl)trimethylammoniumcyanoborohydride resin (Novabiochem, 4.3 mmol/g, 5 eq) was added and the reaction was shaken on an orbital shaker overnight. The reaction was filtered and concentrated in vacuo. The residue was purified via preparative HPLC (Phenomenex column Luna C18, 75 mm×30 mm, 5 micron), 50% acetonitrile/water (0.01% TFA) to 100% acetonitrile, to yield 15 mg of the title compound as a yellow solid.

1H NMR (500 MHz, DMSO-D6) 8 ppm 1.8 (m, 4H) 3.0 (m, 2H) 3.5 (m, 2H) 3.9 (m, 2H) 4.1 (m, 2H) 6.9 (m, 2H) 7.0 (d, J=7.8 Hz, 1H) 7.1 (m, 3H) 7.6 (t, J=7.3 Hz, 1H) 7.7 (t, J=7.1 Hz, 1H) 7.9 (d, J=8.3 Hz, 1H) 8.0 (d, J=8.3 Hz, 1H) 8.4 (d, J=8.8 Hz, 1H)

The following compounds were synthesized according to a similar general procedure as used for C1:

### Example 2

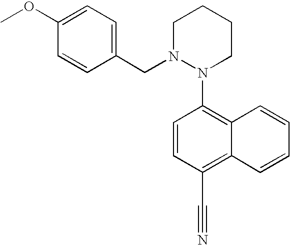

### 4-[2-{[4-(methyloxy)phenyl]methyl}tetrahydro-1(2H)-pyridazinyl]-1-naphthalenecarbonitrile (C2)

From B1 and 4-(methyloxy)benzaldehyde the title compound was isolated as an orange oil.

1H NMR (500 MHz, DMSO-D6) δ ppm 1.7 (m, 4H) 3.0 (m, 2H) 3.6 (s, 3H) 3.8 (m, 2H) 6.6 (d, J=8.3 Hz, 2H) 6.8 (d, J=8.3 Hz, 2H) 7.0 (d, J=8.3 Hz, 1H) 7.6 (t, J=7.6 Hz, 1H) 7.7 (t, J=7.6 Hz, 1H) 7.9 (d, J=8.3 Hz, 1H) 8.0 (d, J=8.3 Hz, 1H) 8.4 (d, J=8.8 Hz, 1H)

### Example 3

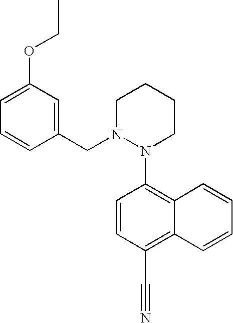

### 4-[2-{[3-(ethyloxy)phenyl]methyl}tetrahydro-1(2H)-pyridazinyl]-1-naphthalenecarbonitrile (C3)

From B1 and 3-(ethyloxy)benzaldehyde the title compound was isolated as a yellow solid.

1H NMR (500 MHz, DMSO-D6) δ ppm 1.1 (t, J=7.1 Hz, 3H) 1.8 (m, 4H) 3.1 (m, 2H) 3.5 (q, J=7.1 Hz, 2H) 3.8 (m, 2H) 6.3 (s, 1H) 6.5 (d, J=7.3 Hz, 1H) 6.6 (m, J=8.3 Hz, 1H) 7.0 (m, 2H) 7.6 (t, J=7.6 Hz, 1H) 7.7 (t, J=7.1 Hz, 1H) 7.9 (d, J=8.3 Hz, 1H) 8.0 (d, J=8.3 Hz, 1H) 8.4 (d, J=8.3 Hz, 1H)

### Example 4

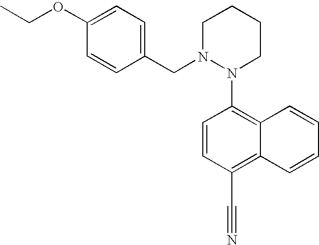

### 4-[2-{[4-(ethyloxy)phenyl]methyl}tetrahydro-1(2H)-pyridazinyl]-1-naphthalenecarbonitrile (C4)

From B1 and 4-(ethyloxy)benzaldehyde the title compound was isolated as an orange oil.

1H NMR (500 MHz, DMSO-D6) δ ppm 1.2 (t, J=7.1 Hz, 3H) 1.7 (m, 4H) 3.0 (m, 3H) 3.5 (m, 2H) 3.8 (m, 2H) 3.9 (q, J=6.8 Hz, 2H) 6.6 (d, J=8.8 Hz, 2H) 6.8 (d, J=8.3 Hz, 2H) 7.0 (d, J=8.3 Hz, 1H) 7.6 (t, J=7.8 Hz, 1H) 7.7 (t, J=7.6 Hz, 1H) 7.9 (d, J=8.3 Hz, 1H) 8.0 (d, J=8.3 Hz, 1H) 8.4 (d, J=8.3 Hz, 1H)

### Example 5

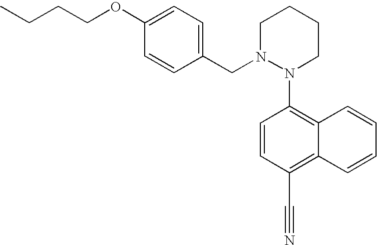

### 4-[2-{[4-(butyloxy)phenyl]methyl}tetrahydro-1(2H)-pyridazinyl]-1-naphthalenecarbonitrile (C5)

From B1 and 4-(butyloxy)benzaldehyde the title compound was isolated as a yellow oil.

MS (m/z) ESI ES⁺=400

### Example 6

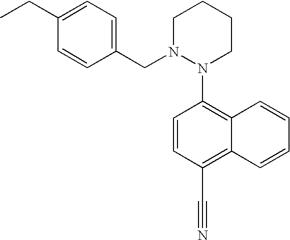

### 4-[2-[(4-ethylphenyl)methyl]tetrahydro-1(2H)-pyridazinyl]-1-naphthalenecarbonitrile (C6)

From B1 and 4-ethybenzaldehyde the title compound was isolated as a yellow solid.

1H NMR (500 MHz, DMSO-D6) δ ppm 1.0 (t, J=7.6 Hz, 3H) 1.8 (m, 4H) 2.4 (q, J=7.3 Hz, 2H) 3.0 (m, 2H) 3.4 (m, 2H) 3.8 (m, 2H) 6.8 (d, J=7.8 Hz, 2H) 6.9 (d, J=7.8 Hz, 2H) 7.0 (d, J=8.3 Hz, 1H) 7.6 (t, J=7.3 Hz, 1H) 7.7 (t, J=7.6 Hz, 1H) 7.9 (d, J=8.3 Hz, 1H) 8.0 (d, J=8.3 Hz, 1H) 8.4 (d, J=8.8 Hz, 1H)

### Example 7

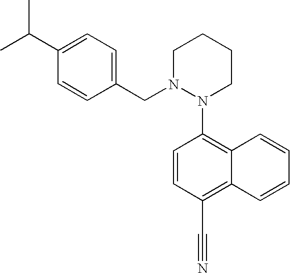

### 4-[2-{[4-(1-methylethyl)phenyl]methyl}tetrahydro-1(2H)-pyridazinyl]-1-naphthalenecarbonitrile (C7)

From B1 and 4-(1-methylethyl)benzaldehyde the title compound was isolated as a yellow solid.

1H NMR (500 MHz, DMSO-D6) δ ppm 1.0 (d, J=6.8 Hz, 6H) 1.8 (m, 4H) 2.7 (m, 1H) 3.0 (m, 2H) 3.4 (m, 2H) 3.8 (m, 2H) 6.8 (d, J=7.8 Hz, 2H) 6.9 (d, J=7.8 Hz, 2H) 7.0 (d, J=8.3 Hz, 1H) 7.6 (t, J=7.8 Hz, 1H) 7.7 (t, J=7.3 Hz, 1H) 7.9 (d, J=8.3 Hz, 1H) 8.0 (d, J=8.3 Hz, 1H) 8.4 (d, J=8.3 Hz, 1H)

### Example 8

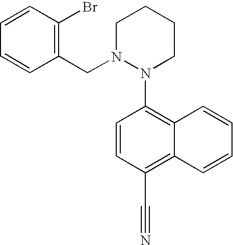

### 4-[2-[(2-bromophenyl)methyl]tetrahydro-1(2H)-pyridazinyl]-1-naphthalenecarbonitrile (C8)

From B1 and 2-bromobenzaldehyde the title compound was isolated as a yellow solid.

1H NMR (500 MHz, DMSO-D6) 8 ppm 1.8 (m, 4H) 3.4 (m, 2H) 3.5 (m, 2H) 4.0 (m, 2H) 6.9 (dd, J=6.8, 2.4 Hz, 1H) 7.0 (m, 2H) 7.1 (d, J=8.3 Hz, 1H) 7.3 (m, 1H) 7.5 (t, J=7.1 Hz, 1H) 7.7 (t, J=7.1 Hz, 1H) 7.9 (m, 2H) 8.3 (d, J=8.3 Hz, 1H)

### Example 9

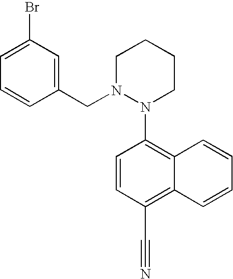

### 4-[2-[(3-bromophenyl)methyl]tetrahydro-1(2H)-pyridazinyl]-1-naphthalenecarbonitrile (C9)

From B1 and 3-bromobenzaldehyde the title compound was isolated as a yellow solid.

1H NMR (500 MHz, DMSO-D6) δ ppm 1.8 (m, 4H) 3.4 (m, 2H) 3.9 (m, 2H) 6.9 (d, J=7.8 Hz, 1H) 6.9 (s, 1H) 7.0 (t, J=7.6 Hz, 2H) 7.2 (d, J=7.8 Hz, 1H) 7.6 (t, J=7.3 Hz, 1H) 7.7 (t, J=7.1 Hz, 1H) 7.9 (d, J=8.3 Hz, 1H) 8.0 (d, J=8.3 Hz, 1H) 8.4 (d, J=8.8 Hz, 1H)

### Example 10

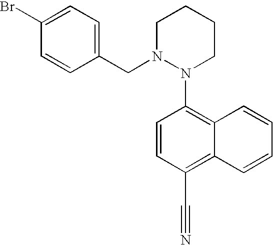

### 4-[2-[(4-bromophenyl)methyl]tetrahydro-1(2H)-pyridazinyl]-1-naphthalenecarbonitrile (C10)

From B1 and 4-bromobenzaldehyde the title compound was isolated as a yellow oil.

1H NMR (500 MHz, DMSO-D6) δ ppm 1.7 (m, 4H) 3.0 (m, 2H) 3.4 (m, 2H) 3.8 (m, 2H) 6.8 (d, J=8.3 Hz, 2H) 7.0 (d, J=7.8 Hz, 1H) 7.2 (d, J=8.3 Hz, 2H) 7.6 (t, J=7.8 Hz, 1H) 7.7 (t, J=7.1 Hz, 1H) 7.9 (d, J=8.3 Hz, 1H) 8.0 (d, J=8.3 Hz, 1H) 8.3 (d, J=8.3 Hz, 1H)

### Example 11

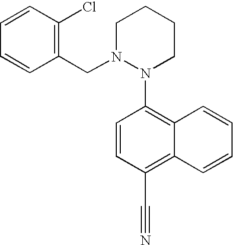

### 4-[2-[(2-chlorophenyl)methyl]tetrahydro-1(2H)-pyridazinyl]-1-naphthalenecarbonitrile (C11)

From B1 and 2-chlorobenzaldehyde the title compound was isolated as a yellow solid.

1H NMR (500 MHz, DMSO-D6) δ ppm 1.8 (m, 4H) 3.5 (m, 2H) 4.0 (m, 2H) 4.1 (m, 2H) 6.9 (m, 2H) 7.0 (m, J=9.0, 9.0 Hz, 2H) 7.1 (d, J=7.8 Hz, 1H) 7.5 (t, J=7.6 Hz, 1H) 7.7 (t, J=7.6 Hz, 1H) 7.9 (dd, J=7.8, 3.9 Hz, 2H) 8.3 (d, J=8.8 Hz, 1H)

### Example 12

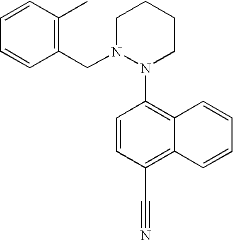

### 4-[2-[(2-methylphenyl)methyl]tetrahydro-1(2H)-pyridazinyl]-1-naphthalenecarbonitrile (C12)

From B1 and 2-methylbenzaldehyde the title compound was isolated as a yellow solid.

1H NMR (500 MHz, DMSO-D6) δ ppm 1.6 (s, 3H) 1.8 (m, 4H) 3.1 (m, 2H) 3.5 (m, 2H) 3.9 (m, 2H) 6.8 (d, J=7.3 Hz, 1H) 7.0 (m, 3H) 7.0 (d, J=7.8 Hz, 1H) 7.5 (t, J=7.6 Hz, 1H) 7.7 (t, J=7.6 Hz, 1H) 7.9 (t, J=8.3 Hz, 2H) 8.3 (d, J=8.3 Hz, 1H)

### Example 13

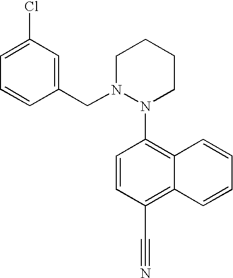

### 4-[2-[(3-chlorophenyl)methyl]tetrahydro-1(2H)-pyridazinyl]-1-naphthalenecarbonitrile (C13)

From B1 and 3-chlorobenzaldehyde the title compound was isolated as a yellow solid.

1H NMR (500 MHz, DMSO-D6) δ ppm 1.8 (m, 4H) 3.1 (m, 2H) 3.4 (m, 2H) 3.9 (m, 2H) 6.8 (s, 1H) 6.9 (d, J=6.3 Hz, 1H) 7.0 (d, J=8.3 Hz, 1H) 7.1 (m, 2H) 7.6 (t, J=7.8 Hz, 1H) 7.7 (t, J=7.1 Hz, 1H) 7.9 (d, J=7.8 Hz, 1H) 8.0 (d, J=7.8 Hz, 1H) 8.4 (d, J=8.8 Hz, 1H)

### Example 14

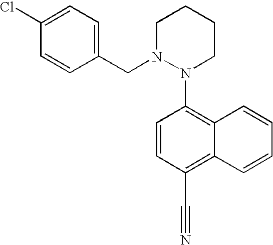

### 4-[2-[(4-chlorophenyl)methyl]tetrahydro-1(2H)-pyridazinyl]-1-naphthalenecarbonitrile (C14)

From B1 and 4-chlorobenzaldehyde the title compound was isolated as a yellow oil.

1H NMR (500 MHz, DMSO-D6) δ ppm 1.8 (m, 4H) 3.0 (m, 2H) 3.5 (m, 2H) 3.9 (m, 2H) 6.9 (d, J=8.3 Hz, 2H) 7.0 (d, J=8.3 Hz, 1H) 7.1 (d, J=8.3 Hz, 2H) 7.6 (t, J=7.6 Hz, 1H) 7.7 (t, J=7.3 Hz, 1H) 7.9 (d, J=7.8 Hz, 1H) 8.0 (d, J=8.3 Hz, 1H) 8.3 (d, J=8.3 Hz, 1H)

### Example 15

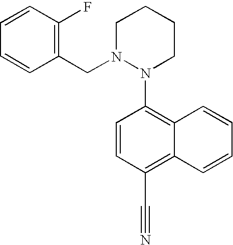

### 4-[2-[(2-fluorophenyl)methyl]tetrahydro-1(2H)-pyridazinyl]-1-naphthalenecarbonitrile (C15)

From B1 and 2-fluorobenzaldehyde the title compound was isolated as a yellow solid.

1H NMR (500 MHz, DMSO-D6) δ ppm 1.8 (m, 4H) 3.1 (m, 2H) 3.5 (m, 2H) 3.9 (m, 2H) 6.8 (m, 2H) 6.9 (m, 1H) 7.0 (d, J=8.3 Hz, 1H) 7.1 (m, 1H) 7.5 (t, J=7.8 Hz, 1H) 7.7 (t, J=7.6 Hz, 1H) 7.9 (m, 2H) 8.3 (d, J=8.8 Hz, 1H)

### Example 16

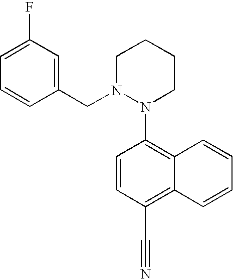

### 4-[2-[(3-fluorophenyl)methyl]tetrahydro-1(2H)-pyridazinyl]-1-naphthalenecarbonitrile (C16)

From B1 and 3-fluorobenzaldehyde the title compound was isolated as a yellow solid.

MS (m/z) ESI ES⁺=346

### Example 17

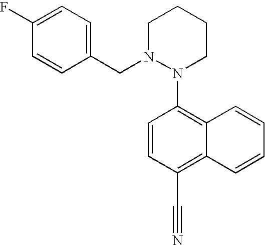

### 4-[2-[(4-fluorophenyl)methyl]tetrahydro-1(2H)-pyridazinyl]-1-naphthalenecarbonitrile (C17)

From B1 and 4-fluorobenzaldehyde the title compound was isolated as an orange oil.

1H NMR (500 MHz, DMSO-D6) δ ppm 1.7 (m, 4H) 3.0 (m, 2H) 3.5 (m, 2H) 3.9 (m, 2H) 6.9 (m, 4H) 7.0 (d, J=7.8 Hz, 1H) 7.6 (t, J=7.3 Hz, 1H) 7.7 (t, J=7.6 Hz, 1H) 7.9 (d, J=7.8 Hz, 1H) 8.0 (d, J=8.3 Hz, 1H) 8.4 (d, J=8.8 Hz, 1H)

### Example 18

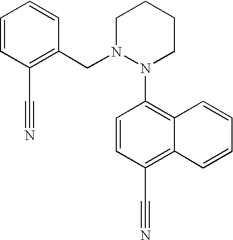

### 4-[2-[(2-cyanophenyl)methyl]tetrahydro-1(2H)-pyridazinyl]-1-naphthalenecarbonitrile (C18)

From B1 and 2-cyanobenzaldehyde the title compound was isolated as a yellow solid.

1H NMR (500 MHz, DMSO-D6) δ ppm 1.8 (m, 4H) 3.2 (m, 2H) 3.4 (m, 2H) 3.5 (m, 2H) 6.9 (d, J=7.8 Hz, 1H) 7.0 (d, J=7.8 Hz, 1H) 7.1 (t, J=7.6 Hz, 1H) 7.2 (t, J=7.8 Hz, 1H) 7.4 (d, J=7.8 Hz, 1H) 7.5 (t, J=7.6 Hz, 1H) 7.6 (t, J=7.6 Hz, 1H) 7.8 (d, J=8.3 Hz, 2H) 8.2 (d, J=8.3 Hz, 1H)

### Example 19

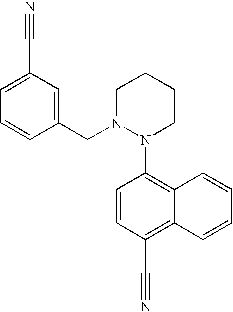

### 4-[2-[(3-cyanophenyl)methyl]tetrahydro-1(2H)-pyridazinyl]-1-naphthalenecarbonitrile (C19)

From B1 and 3-cyanobenzaldehyde the title compound was isolated as a yellow oil.

1H NMR (500 MHz, DMSO-D6) δ ppm 1.8 (m, 4H) 3.1 (m, 2H) 3.5 (m, 2H) 3.9 (m, 2H) 7.0 (d, J=7.8 Hz, 1H) 7.2 (m, 3H) 7.4 (d, J=7.3 Hz, 1H) 7.6 (t, J=7.3 Hz, 1H) 7.7 (t, J=7.1 Hz, 1H) 7.9 (d, J=7.8 Hz, 1H) 7.9 (d, J=8.3 Hz, 1H) 8.3 (d, J=8.3 Hz, 1H)

### Example 20

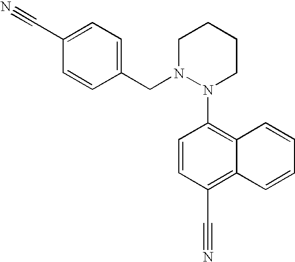

### 4-[2-[(4-cyanophenyl)methyl]tetrahydro-1(2H)-pyridazinyl]-1-naphthalenecarbonitrile (C20)

From B1 and 4-cyanobenzaldehyde the title compound was isolated as a yellow solid.

1H NMR (500 MHz, DMSO-D6) 8 ppm 1.8 (m, 4H) 3.1 (m, 2H) 3.5 (m, 3H) 4.0 (m, 2H) 7.0 (d, J=8.3 Hz, 1H) 7.1 (d, J=8.3 Hz, 2H) 7.5 (d, J=8.3 Hz, 2H) 7.6 (t, J=7.3 Hz, 1H) 7.7 (t, J=7.1 Hz, 1H) 7.9 (d, J=7.8 Hz, 1H) 8.0 (d, J=8.3 Hz, 1H) 8.3 (d, J=8.3 Hz, 1H)

### Example 21

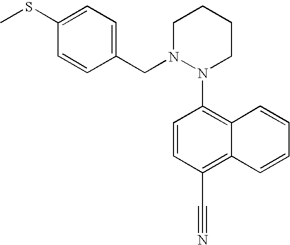

### 4-[2-{[4-(methylthio)phenyl]methyl}tetrahydro-1(2H)-pyridazinyl]-1-naphthalenecarbonitrile (C21)

From B1 and 4-(methylthio)benzaldehyde the title compound was isolated as a yellow oil.

1H NMR (500 MHz, DMSO-D6) δ ppm 1.7 (m, 4H) 2.3 (s, 3H) 3.0 (m, 2H) 3.5 (m, 2H) 3.8 (m, 2H) 6.8 (d, J=8.3 Hz, 2H) 6.9 (d, J=8.3 Hz, 2H) 7.0 (d, J=8.3 Hz, 1H) 7.6 (t, J=7.3 Hz, 1H) 7.7 (t, J=7.6 Hz, 1H) 7.9 (d, J=8.3 Hz, 1H) 8.0 (d, J=8.3 Hz, 1H) 8.4 (d, J=8.3 Hz, 1H)

### Example 22

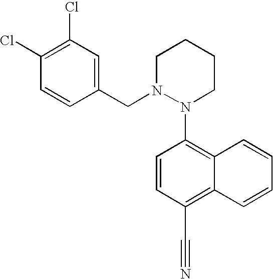

### 4-[2-[(3,4-dichlorophenyl)methyl]tetrahydro-1(2H)-pyridazinyl]-1-naphthalenecarbonitrile (C22)

From B1 and 3,4-dichlorobenzaldehyde the title compound was isolated as a yellow oil.

1H NMR (500 MHz, DMSO-D6) δ ppm 1.7 (m, 4H) 3.1 (m, 2H) 3.5 (m, 2H) 3.9 (m, 2H) 6.8 (dd, J=8.3, 2.0 Hz, 1H) 7.0 (m, 2H) 7.2 (d, J=8.3 Hz, 1H) 7.6 (t, J=7.3 Hz, 1H) 7.7 (t, J=7.6 Hz, 1H) 7.9 (d, J=7.8 Hz, 1H) 7.9 (d, J=8.3 Hz, 1H) 8.3 (d, J=8.8 Hz, 1H)

### Example 23

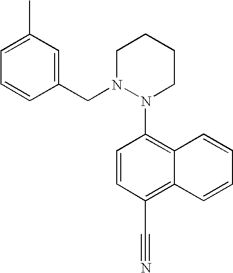

### 4-[2-[(3-methylphenyl)methyl]tetrahydro-1(2H)-pyridazinyl]-1-naphthalenecarbonitrile (C23)

From B1 and 3-methylbenzaldehyde the title compound was isolated as a yellow solid.

1H NMR (500 MHz, DMSO-D6) δ ppm 1.7 (m, 2H) 1.9 (m, 2H) 2.0 (s, 3H) 3.0 (s, 2H) 3.4 (m, 2H) 3.8 (s, 2H) 6.6 (s, 1H) 6.7 (d, J=7.3 Hz, 1H) 6.9 (d, J=7.8 Hz, 1H) 6.9 (d, J=7.6 Hz, 1H) 7.0 (d, J=8.3 Hz, 1H) 7.6 (t, J=7.3 Hz, 1H) 7.7 (t, J=7.1 Hz, 1H) 7.9 (d, J=8.3 Hz, 1H) 8.0 (d, J=7.8 Hz, 1H) 8.4 (d, J=8.8 Hz, 1H)

### Example 24

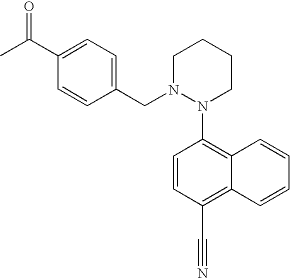

### 4-[2-[(4-acetylphenyl)methyl]tetrahydro-1(2H)-pyridazinyl]-1-naphthalenecarbonitrile (C24)

From B1 and 4-acetylbenzaldehyde the title compound was isolated as an orange oil.

1H NMR (500 MHz, DMSO-D6) δ ppm 1.7 (m, 4H) 2.4 (s, 3H) 3.1 (m, 2H) 3.5 (m, 2H) 3.9 (m, 2H) 7.0 (m, 3H) 7.6 (m, 3H) 7.7 (t, J=7.3 Hz, 1H) 7.9 (d, J=7.8 Hz, 1H) 8.0 (d, J=8.3 Hz, 1H) 8.4 (d, J=8.3 Hz, 1H)

### Example 25

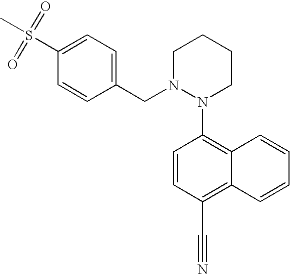

### 4-[2-{[4-(methylsulfonyl)phenyl]methyl}tetrahydro-1(2H-pyridazinyl]-1-naphthalenecarbonitrile (C25)

From B1 and 4-(methylsulfonyl)benzaldehyde the title compound was isolated as an orange oil.

1H NMR (500 MHz, DMSO-D6) δ ppm 1.8 (m, 4H) 3.0 (s, 3H) 3.1 (m, 2H) 3.5 (m, 2H) 4.0 (m, 2H) 7.0 (d, J=7.8 Hz, 1H) 7.1 (d, J=8.3 Hz, 2H) 7.5 (d, J=8.3 Hz, 2H) 7.6 (t, J=7.6 Hz, 1H) 7.7 (t, J=7.6 Hz, 1H) 7.9 (d, J=8.3 Hz, 1H) 7.9 (d, J=8.3 Hz, 1H) 8.3 (d, J=8.3 Hz, 1H)

### Example 26

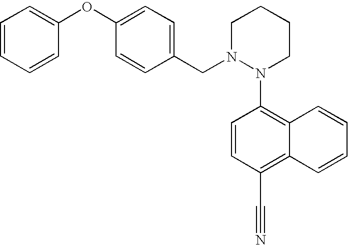

### 4-[2-{[4-(phenyloxy)phenyl]methyl}tetrahydro-1(2H)-pyridazinyl]-1-naphthalenecarbonitrile (C26)

From B1 and 4-phenyloxybenzaldehyde the title compound was isolated as a yellow oil.

1H NMR (500 MHz, DMSO-D6) δ ppm 1.8 (m, 4H) 3.1 (m, 2H) 3.5 (m, 2H) 3.9 (m, 2H) 6.6 (d, J=8.3 Hz, 2H) 6.8 (d, J=7.8 Hz, 2H) 6.9 (d, J=8.3 Hz, 2H) 7.0 (d, J=8.3 Hz, 1H) 7.1 (t, J=7.3 Hz, 1H) 7.3 (t, J=8.1 Hz, 2H) 7.6 (t, J=7.3 Hz, 1H) 7.7 (t, J=7.6 Hz, 1H) 7.9 (d, J=8.3 Hz, 1H) 8.0 (d, J=8.3 Hz, 1H) 8.4 (d, J=8.3 Hz, 1H)

### Example 27

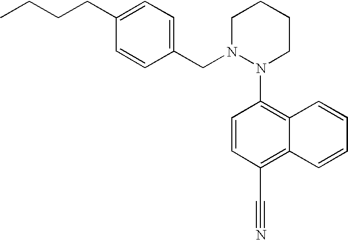

### 4-[2-[(4-butylphenyl)methyl]tetrahydro-1(2H)-pyridazinyl]-1-naphthalenecarbonitrile (C27)

From B1 and 4-butylbenzaldehyde the title compound was isolated as a yellow oil.

1H NMR (500 MHz, DMSO-D6) δ ppm 0.8 (t, J=7.3 Hz, 3H) 1.2 (m, 2H) 1.4 (m, 2H) 1.7 (m, 4H) 2.4 (t, J=7.6 Hz, 2H) 3.0 (m, 2H) 3.4 (m, 2H) 3.8 (m, 2H) 6.7 (d, J=7.8 Hz, 2H) 6.8 (d, J=7.8 Hz, 2H) 7.0 (d, J=7.8 Hz, 1H) 7.6 (t, J=7.6 Hz, 1H) 7.7 (t, J=7.6 Hz, 1H) 7.9 (d, J=7.8 Hz, 1H) 8.0 (d, J=8.3 Hz, 1H) 8.4 (d, J=8.8 Hz, 1H)

### Example 28

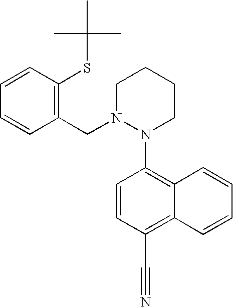

### 4-[2-({2-[(1,1-dimethylethyl)thio]phenyl}methyl)tetrahydro-1(2H)-pyridazinyl]-1-naphthalenecarbonitrile (C28)

From B1 and 2-[(1,1-dimethylethyl)thio]benzaldehyde the title compound was isolated as a yellow solid.

1H NMR (500 MHz, DMSO-D6) δ ppm 1.0 (m, 9H) 1.8 (m, 4H) 3.0 (m, 2H) 3.5 (m, 2H) 4.1 (m, 2H) 6.9 (d, J=5.9 Hz, 1H) 7.0 (m, 3H) 7.3 (d, J=7.3 Hz, 1H) 7.5 (t, J=7.3 Hz, 1H) 7.7 (t, J=7.1 Hz, 1H) 8.3 (d, J=8.8 Hz, 1H)

### Example 29

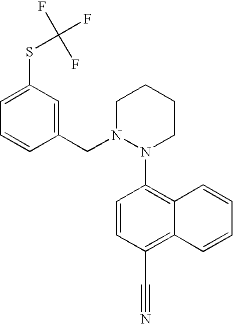

### 4-[2-({3-[(trifluoromethyl)thio]phenyl}methyl)tetrahydro-1(2H)-pyridazinyl]-1-naphthalenecarbonitrile (C29)

From B1 and 3-[(trifluoromethyl)thio]benzaldehyde the title compound was isolated as a yellow oil.

1H NMR (500 MHz, DMSO-D6) δ ppm 1.7 (m, 4H) 3.1 (m, 2H) 3.5 (m, 2H) 4.0 (m, 2H) 7.0 (d, J=7.8 Hz, 1H) 7.1 (m, 2H) 7.2 (t, J=7.6 Hz, 1H) 7.3 (d, J=7.8 Hz, 1H) 7.5 (t, J=7.6 Hz, 1H) 7.7 (t, J=7.1 Hz, 1H) 7.9 (d, J=7.8 Hz, 1H) 7.9 (d, J=8.3 Hz, 1H) 8.3 (d, J=8.3 Hz, 1H)

### Example 30

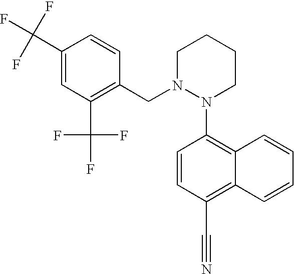

### 44-[2-{[2,4-bis(trifluoromethyl)phenyl]methyl}tetrahydro-1(2H)-pyridazinyl]-1-naphthalenecarbonitrile (C30)

From B1 and 2,4-bis(trifluoromethyl)benzaldehyde the title compound was isolated as a yellow solid.

1H NMR (500 MHz, DMSO-D6) δ ppm 1.8 (m, 4H) 3.2 (m, 2H) 3.5 (m, 2H) 4.2 (m, 2H) 7.1 (d, J=8.3 Hz, 1H) 7.3 (d, J=8.3 Hz, 1H) 7.5 (d, J=8.3 Hz, 1H) 7.6 (t, J=8.3 Hz, 1H) 7.7 (m, 2H) 7.9 (m, 2H) 8.2 (d, J=8.3 Hz, 1H)

### Example 31

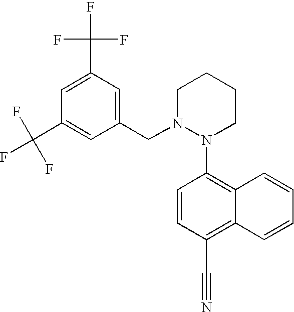

### 4-[2-{[3,5-bis(trifluoromethyl)phenyl]methyl}tetrahydro-1(2H)-pyridazinyl]-1-naphthalenecarbonitrile (C31)

From B1 and 3,5-bis(trifluoromethyl)benzaldehyde the title compound was isolated as a white solid.

1H NMR (500 MHz, DMSO-D6) δ ppm 1.8 (m, 4H) 3.3 (m, 2H) 3.5 (m, 2H) 4.1 (m, 2H) 6.9 (d, J=8.3 Hz, 1H) 7.4 (s, 2H) 7.5 (m, 1H) 7.6 (s, 1H) 7.6 (t, J=7.6 Hz, 1H) 7.8 (d, J=7.8 Hz, 1H) 7.8 (d, J=8.3 Hz, 1H) 8.3 (d, J=8.3 Hz, 1H)

### Example 32

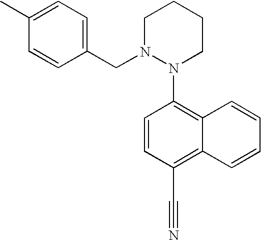

### 4-[2-[(4-methylphenyl)methyl]tetrahydro-1(2H)-pyridazinyl]-1-naphthalenecarbonitrile (C32)

From B1 and 4-methylbenzaldehyde the title compound was isolated as a yellow solid.

1H NMR (500 MHz, DMSO-D6) δ ppm 1.7 (m, 4H) 2.1 (s, 3H) 3.0 (m, 2H) 3.5 (m, 2H) 3.8 (m, 2H) 6.7 (d, J=7.8 Hz, 2H) 6.9 (d, J=7.8 Hz, 2H) 7.0 (d, J=8.3 Hz, 1H) 7.6 (t, J=7.6 Hz, 1H) 7.7 (t, J=7.3 Hz, 1H) 7.9 (d, J=7.8 Hz, 1H) 8.0 (d, J=8.3 Hz, 1H) 8.4 (d, J=8.3 Hz, 1H)

### Example 33

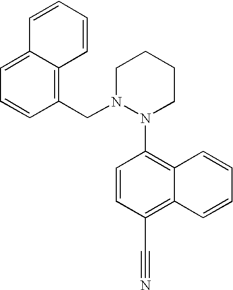

### 4-[2-(1-naphthalenylmethyl)tetrahydro-1(2H)-pyridazinyl]-1-naphthalenecarbonitrile (C33)

From B1 and 1-naphthalenecarbaldehyde the title compound was isolated as a yellow solid.

1H NMR (500 MHz, DMSO-D6) δ ppm 1.8 (m, 4H) 3.0 (m, 2H) 3.6 (m, 2H) 4.3 (m, 2H) 6.7 (t, J=7.6 Hz, 1H) 7.0 (d, J=8.3 Hz, 1H) 7.1 (d, J=8.3 Hz, 1H) 7.3 (m, 2H) 7.4 (d, J=6.8 Hz, 1H) 7.5 (t, J=7.6 Hz, 1H) 7.7 (t, J=9.3 Hz, 3H) 7.9 (d, J=7.8 Hz, 1H) 8.0 (d, J=8.3 Hz, 1H) 8.3 (d, J=8.8 Hz, 1H)

### Example 34

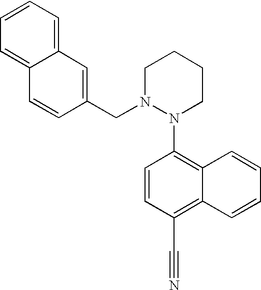

### 4-[2-(2-naphthalenylmethyl)tetrahydro-1(2H)-pyridazinyl]-1-naphthalenecarbonitrile (C34)

From B1 and 2-naphthalenecarbaldehyde the title compound was isolated as a yellow solid.

1H NMR (500 MHz, DMSO-D6) δ ppm 1.8 (m, 4H) 3.1 (m, 2H) 3.5 (m, 2H) 4.0 (m, 2H) 6.8 (d, J=8.8 Hz, 1H) 7.0 (d, J=8.3 Hz, 1H) 7.4 (m, 2H) 7.5 (s, 1H) 7.5 (d, J=8.8 Hz, 1H) 7.6 (m, 2H) 7.7 (m, J=7.3, 7.3 Hz, 2H) 7.9 (d, J=7.8 Hz, 1H) 8.0 (d, J=8.8 Hz, 1H) 8.5 (d, J=8.8 Hz, 1H)

### Example 35

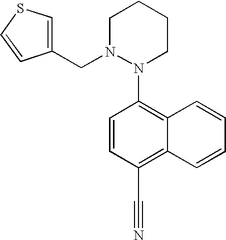

### 4-[2-(3-thienylmethyl)tetrahydro-1(2H)-pyridazinyl]-1-naphthalenecarbonitrile (C35)

From B1 and 3-thiophenecarbaldehyde the title compound was isolated as a yellow solid.

1H NMR (500 MHz, DMSO-D6) δ ppm 1.7 (m, 4H) 3.0 (m, 2H) 3.4 (m, 2H) 3.9 (m, 2H) 6.4 (d, J=3.9 Hz, 1H) 7.0 (d, J=8.3 Hz, 1H) 7.1 (s, 1H) 7.2 (dd, J=4.9, 2.9 Hz, 1H) 7.6 (t, J=7.3 Hz, 1H) 7.7 (t, J=7.3 Hz, 1H) 7.9 (d, J=8.3 Hz, 1H) 8.0 (d, J=8.3 Hz, 1H) 8.4 (d, J=8.3 Hz, 1H)

### Example 36

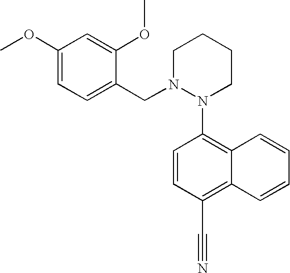

### 4-[2-{[2,4-bis(methyloxy)phenyl]methyl}tetrahydro-1(2H)-pyridazinyl]-1-naphthalenecarbonitrile (C36)

From B1 and 2,4-bis(methyloxy)benzaldehyde the title compound was isolated as a yellow solid.

1H NMR (500 MHz, DMSO-D6) δ ppm 1.8 (m, 4H) 3.0 (m, 2H) 3.3 (s, 3H) 3.4 (m, 2H) 3.6 (s, 3H) 3.8 (m, 2H) 6.2 (dd, J=8.3, 2.4 Hz, 1H) 6.2 (d, J=2.4 Hz, 1H) 6.6 (d, J=8.3 Hz, 1H) 6.9 (d, J=7.8 Hz, 1H) 7.5 (t, J=7.1 Hz, 1H) 7.7 (t, J=7.3 Hz, 1H) 7.9 (d, J=7.8 Hz, 1H) 7.9 (d, J=8.3 Hz, 1H) 8.4 (d, J=8.3 Hz, 1H)

### Example 37

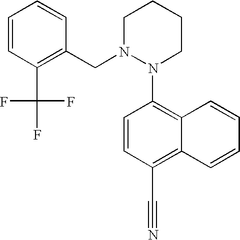

### 4-[2-{[2-(trifluoromethyl)phenyl]methyl}tetrahydro-1(2H)-pyridazinyl]-1-naphthalenecarbonitrile (C37)

From B1 and 2-(trifluoromethyl)benzaldehyde the title compound was isolated as a yellow solid.

1H NMR (500 MHz, DMSO-D6) δ ppm 1.7 (m, 4H) 3.4 (m, 2H) 3.5 (m, 2H) 4.1 (m, 2H) 7.1 (m, 2H) 7.2 (m, 2H) 7.5 (m, 1H) 7.6 (t, J=7.3 Hz, 1H) 7.7 (t, J=7.3 Hz, 1H) 7.9 (m, 2H) 8.3 (d, J=8.8 Hz, 1H)

### Example 38

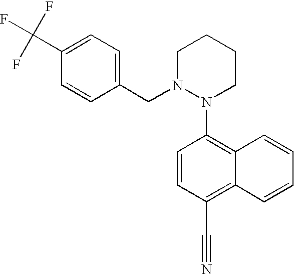

### 4-{[2-([4-(trifluoromethyl)phenyl]methyl}tetrahydro-1(2H)-pyridazinyl]-1-naphthalenecarbonitrile (C38)

From B1 and 4-(trifluoromethyl)benzaldehyde the title compound was isolated as a yellow solid.

1H NMR (500 MHz, DMSO-D6) δ ppm 1.8 (m, 4H) 3.1 (m, 2H) 3.5 (m, 2H) 4.0 (m, 2H) 7.0 (d, J=7.8 Hz, 1H) 7.1 (d, J=8.3 Hz, 1H) 7.3 (d, J=7.8 Hz, 1H) 7.6 (t, J=7.8 Hz, 1H) 7.7 (t, J=7.1 Hz, 1H) 7.9 (d, J=8.3 Hz, 1H) 8.3 (d, J=8.8 Hz, 1H)

### Example 39

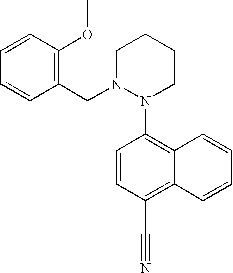

### 4-[2-{[2-(methyloxy)phenyl]methyl}tetrahydro-1(2H)-pyridazinyl]-1-naphthalenecarbonitrile (C39)

From B1 and 2-(methyloxy)benzaldehyde the title compound was isolated as a yellow solid.

1H NMR (500 MHz, DMSO-D6) δ ppm 1.8 (m, 4H) 3.1 (m, 2H) 3.3 (s, 3H) 3.5 (m, 2H) 3.8 (m, 2H) 6.6 (t, J=7.3 Hz, 1H) 6.7 (d, J=8.3 Hz, 1H) 6.8 (d, J=7.3 Hz, 1H) 7.0 (d, J=7.8 Hz, 1H) 7.0 (t, J=7.8 Hz, 1H) 7.5 (t, J=7.6 Hz, 1H) 7.7 (t, J=7.6 Hz, 1H) 7.9 (d, J=8.3 Hz, 1H) 7.9 (d, J=8.3 Hz, 1H) 8.4 (d, J=8.3 Hz, 1H)

### Example 40

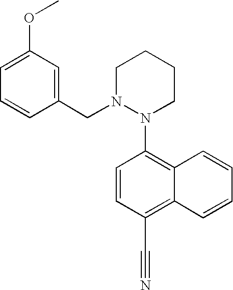

### 4-[2-{[3-(methyloxy)phenyl]methyl}tetrahydro-1(2H)-pyridazinyl]-1-naphthalenecarbonitrile (C40)

From B1 and 3-(methyloxy)benzaldehyde the title compound was isolated as a yellow solid.

1H NMR (500 MHz, DMSO-D6) 8 ppm 1.8 (m, 4H) 3.1 (m, 2H) 3.4 (s, 3H) 3.8 (m, 2H) 6.3 (s, 1H) 6.5 (d, J=7.3 Hz, 1H) 6.6 (dd, J=8.3, 2.4 Hz, 1H) 7.0 (m, 2H) 7.6 (t, J=7.8 Hz, 1H) 7.7 (t, J=7.6 Hz, 1H) 7.9 (d, J=7.8 Hz, 1H) 8.0 (d, J=8.3 Hz, 1H) 8.4 (d, J=8.3 Hz, 1H)

### Example 41

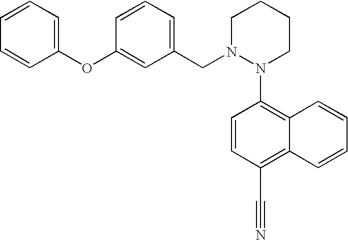

### 4-[2-{[3-(phenyloxy)phenyl]methyl}tetrahydro-1(2H)-pyridazinyl]-1-naphthalenecarbonitrile (C41)

From B1 and 3-(phenyloxy)benzaldehyde the title compound was isolated as a yellow solid.

1H NMR (500 MHz, DMSO-D6) δ ppm 1.8 (m, 4H) 3.1 (m, 2H) 3.4 (m, 2H) 3.9 (m, 2H) 6.5 (s, 1H) 6.7 (m, J=7.8 Hz, 1H) 6.7 (m, J=8.3 Hz, 2H) 7.0 (d, J=8.3 Hz, 1H) 7.1 (m, 3H) 7.3 (t, J=8.1 Hz, 2H) 7.4 (m, 1H) 7.6 (m, 1H) 7.9 (m, 2H) 8.3 (d, J=8.3 Hz, 1H)

### Example 42

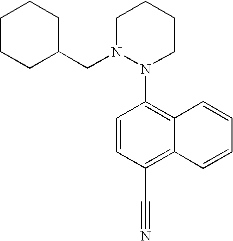

### 4-[2-(cyclohexylmethyl)tetrahydro-1(2H)-pyridazinyl]-1-naphthalenecarbonitrile (C42)

From B1 and cyclohexanecarbaldehyde the title compound was isolated as a yellow solid.

1H NMR (500 MHz, DMSO-D6) δ ppm 0.3 (m, 2H) 0.9 (m, 4H) 1.3 (m, 5H) 1.7 (m, 4H) 2.6 (m, 2H) 3.2 (m, 2H) 3.3 (m, 2H) 7.0 (d, J=8.3 Hz, 1H) 7.5 (t, J=7.1 Hz, 1H) 7.7 (t, J=7.1 Hz, 1H) 7.9 (m, 2H) 8.3 (d, J=8.3 Hz, 1H)

### Example 43

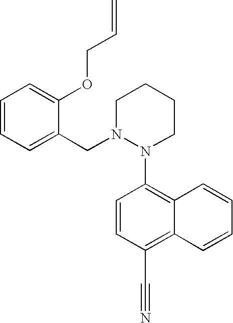

### 4-[2-{[2-(2-propen-1-yloxy)phenyl]methyl}tetrahydro-1(2H-pyridazinyl]-1-naphthalenecarbonitrile (C43)

From B1 and 2-(2-propen-1-yloxy)benzaldehyde the title compound was isolated as a yellow solid.

1H NMR (500 MHz, DMSO-D6) δ ppm 1.8 (m, 4H) 3.1 (m, 2H) 3.5 (m, 2H) 3.9 (m, 2H) 4.3 (m, J=4.9 Hz, 2H) 5.2 (m, 1H) 5.3 (m, 1H) 5.8 (t, 1H) 6.6 (t, J=7.3 Hz, 1H) 6.7 (d, J=5.9 Hz, 1H) 6.7 (d, J=8.3 Hz, 1H) 7.0 (m, 2H) 7.5 (t, J=7.1 Hz, 1H) 7.7 (t, J=7.1 Hz, 1H) 7.9 (m, 2H) 8.4 (d, J=8.3 Hz, 1H)

### Example 44

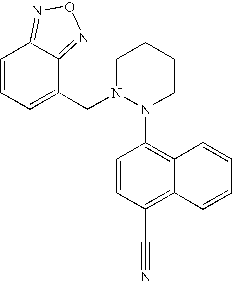

### 4-[2-(2,1,3-benzoxadiazol-4-ylmethyl)tetrahydro-1(2H)-pyridazinyl]-1-naphthalenecarbonitrile (C44)

From B1 and 2,1,3-benzoxadiazole-4-carbaldehyde the title compound was isolated as a yellow solid.

MS (m/z) ESI ES⁺=370

### Example 45

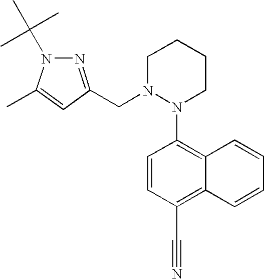

### 4-[2-{[1-(1,1-dimethylethyl)-5-methyl-1H-pyrazol-3-yl]methyl}tetrahydro-1(2H)-pyridazinyl]-1-naphthalenecarbonitrile (C45)

From B1 and 1-(1,1-dimethylethyl)-5-methyl-1H-pyrazole-3-carbaldehyde the title compound was isolated as a brown oil.

MS (m/z) ESI ES⁺=388

### Example 46

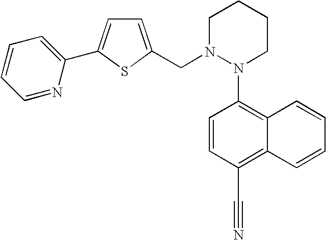

### 4-[2-{[5-(2-pyridinyl)-2-thienyl]methyl}tetrahydro-1(2H)-pyridazinyl]-1-naphthalenecarbonitrile (C46)

From B1 and 5-(2-pyridinyl)-2-thiophenecarbaldehyde the title compound was isolated as a yellow solid.

MS (m/z) ESI ES⁺=411

### Example 47

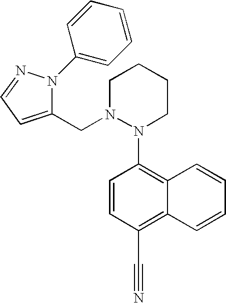

### 4-[2-[(1-phenyl-1H-pyrazol-5-yl)methyl]tetrahydro-1(2H)-pyridazinyl]-1-naphthalenecarbonitrile (C47)

From B1 and 1-phenyl-1H-pyrazole-5-carbaldehyde the title compound was isolated as a yellow solid.

MS (m/z) ESI ES⁺=394

### Example 48

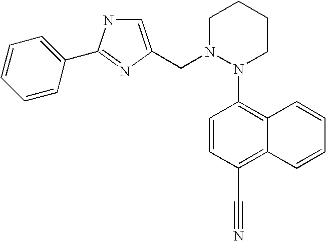

### 4-[2-[(2-phenyl-1H-imidazol-4-yl)methyl]tetrahydro-1(2H)-pyridazinyl]-1-naphthalenecarbonitrile (C48)

From B1 and 2-phenyl-1H-imidazol-4-carbaldehyde the title compound was isolated as a white solid.

MS (m/z) ESI ES⁺=394

### Example 49

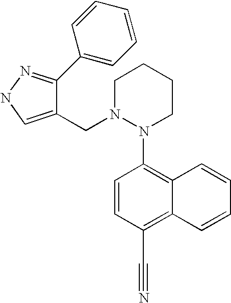

### 4-[2-[(3-phenyl-1H-pyrazol-4-yl)methyl]tetrahydro-1(2H)-pyridazinyl]-1-naphthalenecarbonitrile (C49)

From B1 and 3-phenyl-1H-pyrazol-4-carbaldehyde the title compound was isolated as a yellow solid.

MS (m/z) ESI ES⁺=394

### Example 50

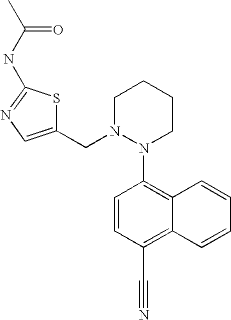

### N-(5-{[2-(4-cyano-1-naphthalenyl)tetrahydro-1(2H)-pyridazinyl]methyl}-1,3-thiazol-2-yl)acetamide (C50)

From B1 and N-(5-formyl-1,3-thiazol-2-yl)acetamide the title compound was isolated as a yellow solid.

MS (m/z) ESI ES⁺=392

### Example 51

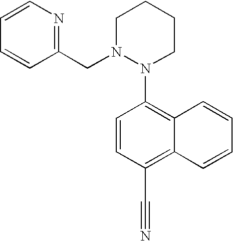

### 4-[2-(2-pyridinylmethyl)tetrahydro-1(2H)-pyridazinyl]-1-naphthalenecarbonitrile (C51)

From B1 and 2-pyridinecarbaldehyde the title compound was isolated as a yellow oil.

MS (m/z) ESI ES⁺=329

### Example 52

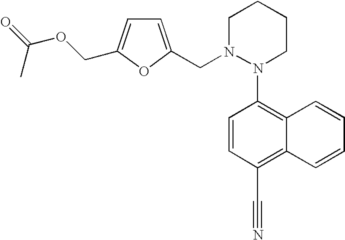

### (5-{[2-(4-cyano-1-naphthalenyl)tetrahydro-1(2H)-pyridazinyl]methyl}-2-furanyl)methyl acetate (C52)

From B1 and (5-formyl-2-furanyl)methyl acetate the title compound was isolated as a brown oil.

MS (m/z) ESI ES⁺=390

### Example 53

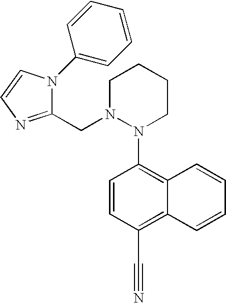

### 4-[2-[(1-phenyl-1H-imidazol-2-yl)methyl]tetrahydro-1(2H)-pyridazinyl]-1-naphthalenecarbonitrile (C53)

From B1 and 1-phenyl-1H-imidazol-2-carbaldehyde the title compound was isolated as a yellow oil.

MS (m/z) ESI ES⁺=394

### Example 54

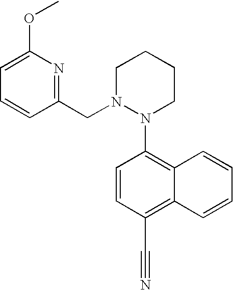

### 4-[2-{[6-(methyloxy)-2-pyridinyl]methyl}tetrahydro-1(2H)-pyridazinyl]-1-naphthalenecarbonitrile (C54)

From B1 and 6-(methyloxy)-2-pyridinecarbaldehyde the title compound was isolated as a brown solid.

MS (m/z) ESI ES⁺=359

### Example 55

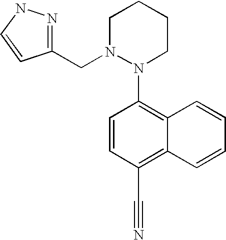

### 4-[2-(1H-pyrazol-3-ylmethyl)tetrahydro-1(2H)-pyridazinyl]-1-naphthalenecarbonitrile (C55)

From B1 and 1H-pyrazol-3-carbaldehyde the title compound was isolated as a yellow oil.

MS (m/z) ESI ES⁺=318

### Example 56

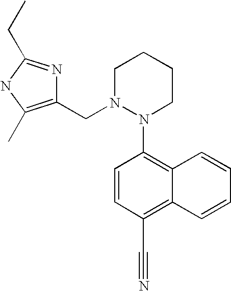

### 4-[2-[(2-ethyl-4-methyl-1H-imidazol-5-yl)methyl]tetrahydro-1(2H)-pyridazinyl]-1-naphthalenecarbonitrile (C56)

From B1 and 2-ethyl-4-methyl-1H-imidazol-5-carbaldehyde the title compound was isolated as a yellow solid.

MS (m/z) ESI ES⁺=360

### Example 57

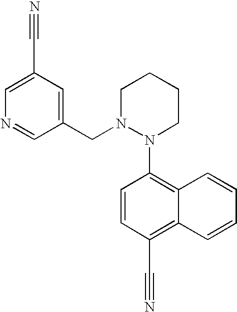

### 5-{[2-(4-cyano-1-naphthalenyl)tetrahydro-1(2H)-pyridazinyl]methyl}-3-pyridinecarbonitrile (C57)

From B1 and 5-formyl-3-pyridinecarbonitrile the title compound was isolated as a yellow solid.

MS (m/z) ESI ES⁺=354

### Example 58

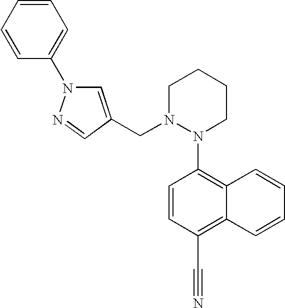

### 4-[2-[(1-phenyl-1H-pyrazol-4-yl)methyl]tetrahydro-1(2H)-pyridazinyl]-1-naphthalenecarbonitrile (C58)

From B1 and 1-phenyl-1H-pyrazol-4-carbaldehyde the title compound was isolated as a yellow solid.

MS (m/z) ESI ES⁺=394

### Example 59

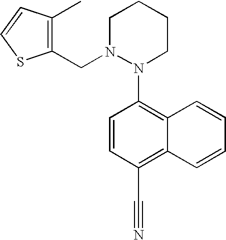

### 4-[2-[(3-methyl-2-thienyl)methyl]tetrahydro-1(2H)-pyridazinyl]1-naphthalenecarbonitrile (C59)

From B1 and 3-methyl-2-thiophenecarbaldehyde the title compound was isolated as a yellow solid.

MS (m/z) ESI ES⁺=348

### Example 60

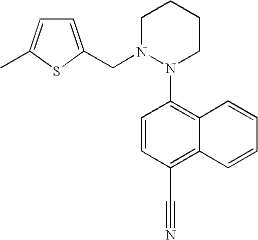

### 4-[2-[(5-methyl-2-thienyl)methyl]tetrahydro-1(2H)-pyridazinyl]-1-naphthalenecarbonitrile (C60)

From B1 and 5-methyl-2-thiophenecarbaldehyde the title compound was isolated as a yellow solid.

MS (m/z) ESI ES⁺=348

### Example 61

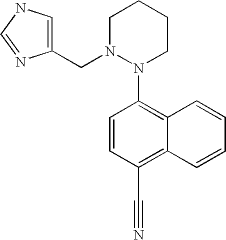

### 4-[2-(1H-imidazol-4-ylmethyl)tetrahydro-1(2H)-pyridazinyl]-1-naphthalenecarbonitrile (C61)

From B1 and 1H-imidazol-4-carbaldehyde the title compound was isolated as a yellow oil.

MS (m/z) ESI ES⁺=318

### Example 62

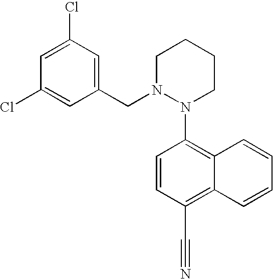

### 4-[2-[(3,5-dichlorophenyl)methyl]tetrahydro-1(2H)-pyridazinyl]-1-naphthalenecarbonitrile (C62)

From B1 and 3,5-dichlorobenzaldehyde the title compound was isolated as a yellow solid.

MS (m/z) APCI AP⁺=396

### Example 63

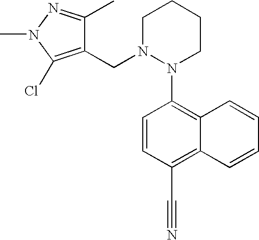

### 4-[2-[(5-chloro-1,3-dimethyl-1H-pyrazol-4-yl)methyl]tetrahydro-1(2H-pyridazinyl]-1-naphthalenecarbonitrile (C63)

From B1 and 5-chloro-1,3-dimethyl-1H-pyrazol-4-carbaldehyde the title compound was isolated as a yellow solid.

MS (m/z) APCI AP⁺=380

### Example 64

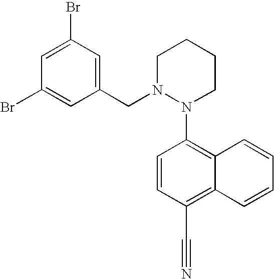

### 4-[2-[(3,5-dibromophenyl)methyl]tetrahydro-1(2H-pyridazinyl]-1-naphthalenecarbonitrile (C64)

From B1 and 3,5-dibromobenzaldehyde the title compound was isolated as a yellow solid.

MS (m/z) APCI AP⁺=486

### Example 65

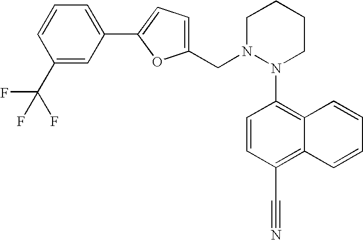

### 4-[2-({5-[3-(trifluoromethyl)phenyl]-2-furanyl}methyl)tetrahydro-1(2H)-pyridazinyl]-1-naphthalenecarbonitrile (C65)

From B1 and 5-[3-(trifluoromethyl)phenyl]-2-furancarbaldehyde the title compound was isolated as a red oil.

MS (m/z) APCI AP⁺=462

### Example 66

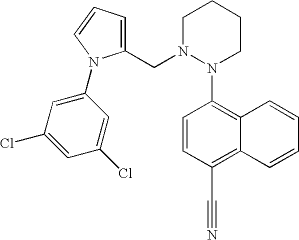

### 4-[2-{[1-(3,5-dichlorophenyl)-1H-pyrrol-2-yl]methyl}tetrahydro-1(2H)-pyridazinyl]-1-naphthalenecarbonitrile (C66)

From B1 and 3,5-dichlorophenyl)-1H-pyrrol-2-carbaldehyde the title compound was isolated as a purple solid.

MS (m/z) APCI AP⁺=461

### Example 67

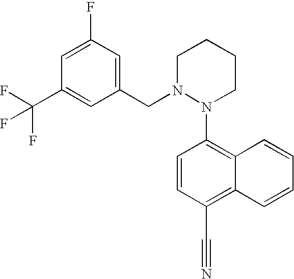

### 4-[2-{[3-fluoro-5-(trifluoromethyl)phenyl]methyl}tetrahydro-1(2H)-pyridazinyl]-1-naphthalenecarbonitrile (C67)

From B1 and 3-fluoro-5-(trifluoromethyl)benzaldehyde the title compound was isolated as a yellow oil.

MS (m/z) APCI AP⁺=414

### Example 68

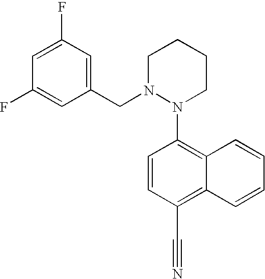

### 4-[2-[(3,5-difluorophenyl)methyl]tetrahydro-1(2H)-pyridazinyl]-1-naphthalenecarbonitrile (C68)

From B1 and 3,5-difluorobenzaldehyde the title compound was isolated as a brown solid.

MS (m/z) APCI AP⁺=364

### Example 69

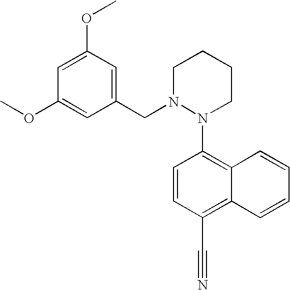

### 4-[2-{[3,5-bis(methyloxy)phenyl]methyl}tetrahydro-1(2H-pyridazinyl]-1-naphthalenecarbonitrile (C69)

From B1 and 3,5-bis(methyloxy)benzaldehyde the title compound was isolated as a yellow oil.

MS (m/z) APCI AP⁺=388

### Example 70

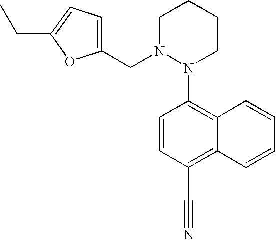

### 4-[2-[(5-ethyl-2-furanyl)methyl]tetrahydro-1(2H)-pyridazinyl]-1-naphthalenecarbonitrile (C70)

From B1 and 5-ethyl-2-furancarbaldehyde the title compound was isolated as a yellow oil.

MS (m/z) APCI AP⁺=388

### Example 71

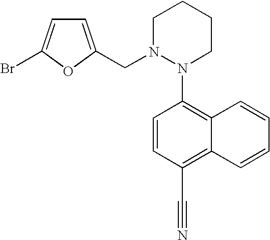

### 4-[2-[(5-bromo-2-furanyl)methyl]tetrahydro-1(2H)-pyridazinyl]-1-naphthalenecarbonitrile (C71)

From B1 and 5-bromo-2-furancarbaldehyde the title compound was isolated as a yellow oil.

MS (m/z) APCI AP⁺=398

### Example 72

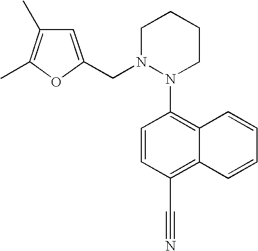

### 4-[2-[(4,5-dimethyl-2-furanyl)methyl]tetrahydro-1(2H)-pyridazinyl]-1-naphthalenecarbonitrile (C72)

From B1 and 4,5-dimethyl-2-furancarbaldehyde the title compound was isolated as a yellow solid.

1H NMR (500 MHz, DMSO-D6) δ ppm 1.6 (s, 3H) 1.7 (m, 2H) 1.8 (s, 3H) 1.8 (m, 2H) 3.2 (m, 2H) 3.4 (m, 2H) 6.8 (d, J=8.3 Hz, 1H) 7.5 (t, J=7.3 Hz, 1H) 7.6 (t, J=7.6 Hz, 1H) 7.8 (d, J=7.8 Hz, 1H) 7.9 (d, J=8.3 Hz, 1H) 8.4 (d, J=8.8 Hz, 1H)

### Example 73

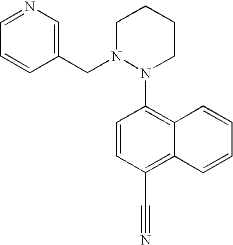

### 4-[2-(3-pyridinylmethyl)tetrahydro-1(2H)-pyridazinyl]-1-naphthalenecarbonitrile (C73)

From B1 and 3-pyridinecarbaldehyde the title compound was isolated as a yellow oil.

MS (m/z) ESI ES⁺=329

### Example 74

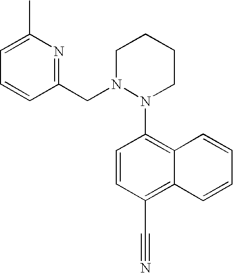

### 4-[2-[(6-methyl-2-pyridinyl)methyl]tetrahydro-1(2H)-pyridazinyl]-1-naphthalenecarbonitrile (C74)

From B1 and 6-methyl-2-pyridinecarbaldehyde the title compound was isolated as a yellow oil.

MS (m/z) ESI ES⁺=343

### Example 75

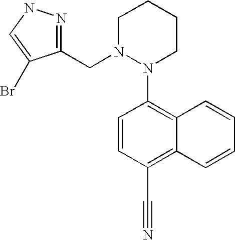

### 4-[2-[(4-bromo-1H-pyrazol-3-yl)methyl]tetrahydro-1(2H)-pyridazinyl]-1-naphthalenecarbonitrile (C75)

From B1 and 4-bromo-1H-pyrazol-3-carbaldehyde the title compound was isolated as a white solid.

MS (m/z) ESI ES⁺=397

### Example 76

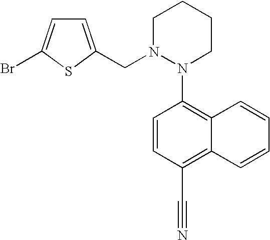

### 4-[2-[(5-bromo-2-thienyl)methyl]tetrahydro-1(2H)-pyridazinyl]-1-naphthalenecarbonitrile (C76)

From B1 and 5-bromo-2-thiophenecarbaldehyde the title compound was isolated as a yellow solid.

MS (m/z) APCI AP⁺=413

### Example 77

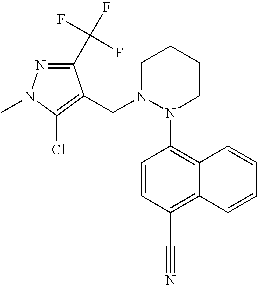

### 4-[2-{[5-chloro-1-methyl-3-(trifluoromethyl)-1H-pyrazol-4-yl]methyl}tetrahydro-1(2H)-pyridazinyl]-1-naphthalenecarbonitrile (C77)

From B1 and 5-chloro-1-methyl-3-(trifluoromethyl)-1H-pyrazol-4-carbaldehyde the title compound was isolated as a white solid.

MS (m/z) APCI AP⁺=434

### Example 78

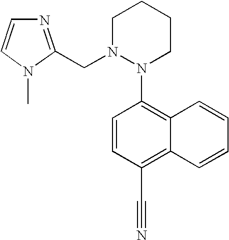

### 4-[2-[(1-methyl-1H-imidazol-2-yl)methyl]tetrahydro-1(2H)-pyridazinyl]-1-naphthalenecarbonitrile (C78)

From B1 and 1-methyl-1H-imidazol-2-carbaldehyde the title compound was isolated as a white solid.

MS (m/z) APCI AP⁺=332

### Example 79

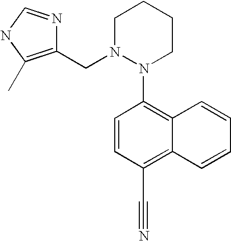

### 4-[2-[(4-methyl-1H-imidazol-5-yl)methyl]tetrahydro-1(2H)-pyridazinyl]-1-naphthalenecarbonitrile (C79)

From B1 and 4-methyl-1H-imidazol-5-carbaldehyde the title compound was isolated as a yellow solid.

MS (m/z) APCI AP⁺=332

### Example 80

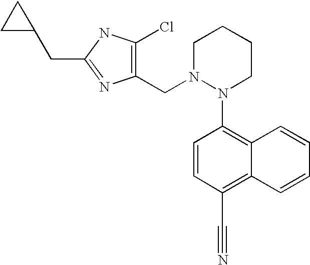

### 4-[2-{[4-chloro-2-(cyclopropylmethyl)-1H-imidazol-5-yl]methyl}tetrahydro-1(2H)-pyridazinyl]-1-naphthalenecarbonitrile (C80)

From B1 and 4-chloro-2-(cyclopropylmethyl)-1H-imidazol-5-carbaldehyde the title compound was isolated as a yellow solid.

MS (m/z) APCI AP⁺=406

### Example 81

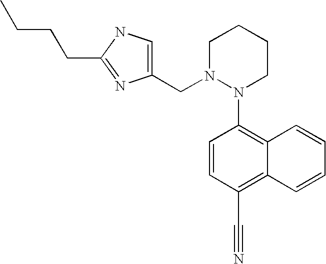

### 4-[2-[(2-butyl-1H-imidazol-4-yl)methyl]tetrahydro-1(2H)-pyridazinyl]-1-naphthalenecarbonitrile (C81)

From B1 and 2-butyl-1H-imidazol-4-carbaldehyde the title compound was isolated as a yellow oil.

MS (m/z) APCI AP⁺=374

### Example 82

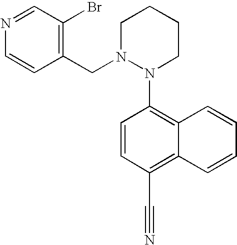

### 4-[2-[(3-bromo-4-pyridinyl)methyl]tetrahydro-1(2H-pyridazinyl]-1-naphthalenecarbonitrile (C82)

From B1 and 3-bromo-4-pyridinecarbaldehyde the title compound was isolated as a brown solid.

MS (m/z) APCI AP⁺=408

### Example 83

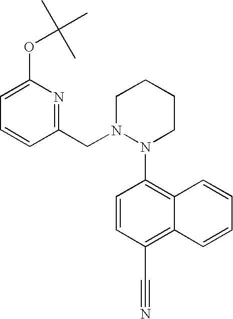

### 4-[2-({6-[(1,1-dimethylethyl)oxy]-2-pyridinyl}methyl)tetrahydro-1(2H)-pyridazinyl]-1-naphthalenecarbonitrile (C83)

From B1 and 6-[(1,1-dimethylethyl)oxy]-2-pyridinecarbaldehyde the title compound was isolated as a yellow oil.

MS (m/z) ESI ES⁺=401

### Example 84

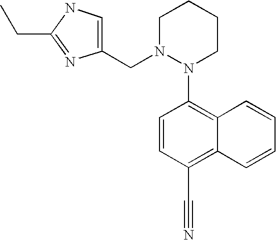

### 4-[2-[(2-ethyl-1H-imidazol-4-yl)methyl]tetrahydro-1(2H)-pyridazinyl]-1-naphthalenecarbonitrile (C84)

From B1 and 2-ethyl-1H-imidazol-4-carbaldehyde the title compound was isolated as a yellow oil,

MS (m/z) ESI ES⁺=346

### Example 85

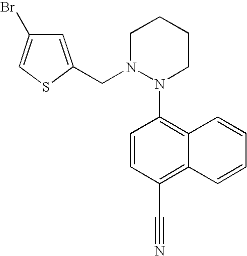

### 4-[2-[(4-bromo-2-thienyl)methyl]tetrahydro-1(2H)-pyridazinyl]-1-naphthalenecarbonitrile (C85)

From B1 and 4-bromo-2-thiophenecarbaldehyde the title compound was isolated as a yellow solid.

MS (m/z) ESI ES⁺=413

### Example 86

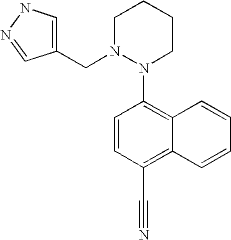

### 4-[2-(1H-pyrazol-4-ylmethyl)tetrahydro-1(2H)-pyridazinyl]-1-naphthalenecarbonitrile (C86)

From B1 and 1H-pyrazol-4-carbaldehyde the title compound was isolated as a yellow solid.

MS (m/z) ESI ES⁺=318

### Example 87

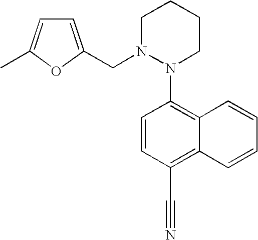

### 4-[2-[(5-methyl-2-furanyl)methyl]tetrahydro-1(2H)-pyridazinyl]-1-naphthalenecarbonitrile (C87)

From B1 and 5-methyl-2-furancarbaldehyde the title compound was isolated as a yellow oil.

MS (m/z) ESI ES⁺=332

### Example 88

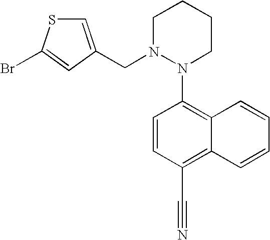

### 4-[2-[(5-bromo-3-thienyl)methyl]tetrahydro-1(2H)-pyridazinyl]-1-naphthalenecarbonitrile (C88)

From B1 and 5-bromo-3-thiophenecarbaldehyde the title compound was isolated as a yellow oil.

MS (m/z) ESI ES⁺=413

### Example 89

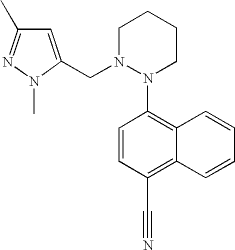

### 4-[2-[(1,3-dimethyl-1H-pyrazol-5-yl)methyl]tetrahydro-1(2H)-pyridazinyl]-1-naphthalenecarbonitrile (C89)

From B1 and 1,3-dimethyl-1H-pyrazol-5-carbaldehyde the title compound was isolated as a yellow solid.

MS (m/z) ESI ES⁺=346

### Example 90

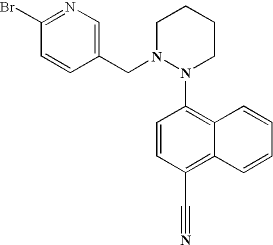

### 4-[2-[(6-bromo-3-pyridinyl)methyl]tetrahydro-1(2H)-pyridazinyl]-1-naphthalenecarbonitrile (C90)

From B1 and 6-bromo-3-pyridinecarbaldehyde the title compound was isolated as a yellow solid.

MS (m/z) ESI ES⁺=408

### Example 91

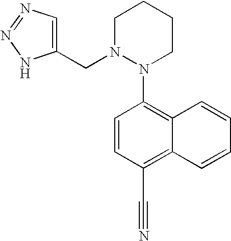

### 4-[2-(1H-1,2,3-triazol-4-ylmethyl)tetrahydro-1(2H)-pyridazinyl]-1-naphthalenecarbonitrile (C91)

From B1 and 1H-1,2,3-triazol-4-carbaldehyde the title compound was isolated as a yellow solid.

MS (m/z) ESI ES⁺=319

### Example 92

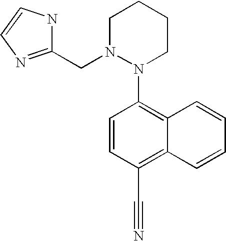

### 4-[2-(1H-imidazol-2-ylmethyl)tetrahydro-1(2H)-pyridazinyl]-1-naphthalenecarbonitrile (C92)

From B1 and 1H-imidazol-2-carbaldehyde the title compound was isolated as a yellow solid.

MS (m/z) ESI ES⁺=318

### Example 93

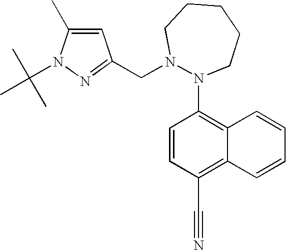

### 4-(2-{[1-(1,1-dimethylethyl)-5-methyl-1H-pyrazol-3-yl]methyl}hexahydro-1H-1,2-diazepin-1-yl)-1-naphthalenecarbonitrile (C93)

From B4 and 1-(1,1-dimethylethyl)-5-methyl-1H-pyrazol-3-carbaldehyde the title compound was isolated as a yellow oil.

MS (m/z) ESI ES⁺=402

### Example 94

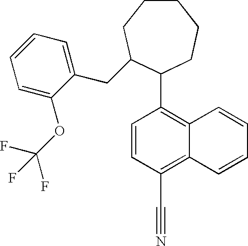

### 4-[2-({2-[(trifluoromethyl)oxy]phenyl}methyl)hexahydro-1H-1,2-diazepin-1-yl]-1-naphthalenecarbonitrile (C94)

From B4 and 2-[(trifluoromethyl)oxy]benzaldehyde the title compound was isolated as a brown oil.

MS (m/z) ESI ES⁺=426

### Example 95

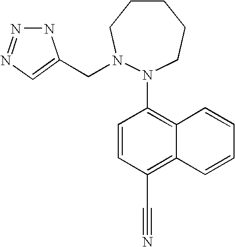

### 4-[2-(1H-1,2,3-triazol-4-ylmethyl)hexahydro-1H-1,2-diazepin-1-yl]-1-naphthalenecarbonitrile (C95)

From B4 and 1H-1,2,3-triazol-4-carbaldehyde the title compound was isolated as a yellow oil.

MS (m/z) ESI ES⁺=333

### Example 96

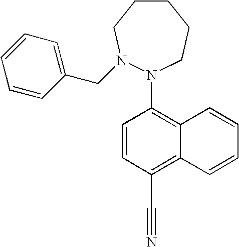

### 4-[2-(phenylmethyl)hexahydro-1H-1,2-diazepin-1-yl]-1-naphthalenecarbonitrile (C138)

From B4 and Benzaldehyde the Title Compound was Isolated as a Yellow Oil.

MS (m/z) ESI ES⁺=342

### Example 97

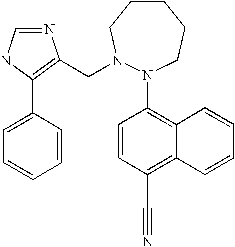

### 4-{2-[(4-phenyl-1H-imidazol-5-yl)methyl]hexahydro-1H-1,2-diazepin-1-yl}-1-naphthalenecarbonitrile (C97)

From B4 and 4-phenyl-1H-imidazol-5-carbaldehyde the title compound was isolated as a yellow solid.

MS (m/z) ESI ES⁺=408

### Example 98

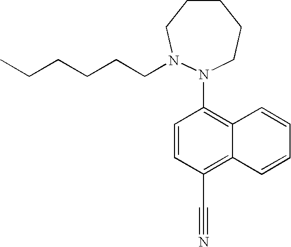

### 4-(2-hexylhexahydro-1H-1,2-diazepin-1-yl)-1-naphthalenecarbonitrile (C98)

From B4 and hexanal the title compound was isolated as a yellow oil.

MS (m/z) ESI ES⁺=336

### Example 99

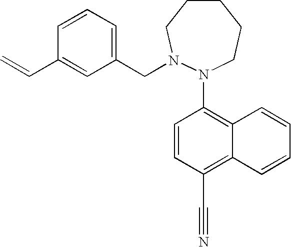

### 4-{2-[(3-ethenylphenyl)methyl]hexahydro-1H-1,2-diazepin-1-yl}-1-naphthalenecarbonitrile (C99)

From B4 and 3-ethenylbenzaldehyde the title compound was isolated as a yellow oil.

MS (m/z) ESI ES⁺=368

### Example 100

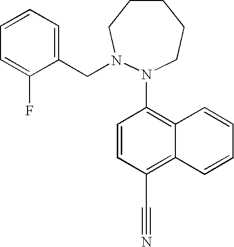

### 4-{2-[(2-fluorophenyl)methyl]hexahydro-1H-1,2-diazepin-1-yl}-1-naphthalenecarbonitrile (C100)

From B4 and 2-fluorobenzaldehyde the title compound was isolated as a yellow solid.

MS (m/z) ESI ES⁺=360

### Example 101

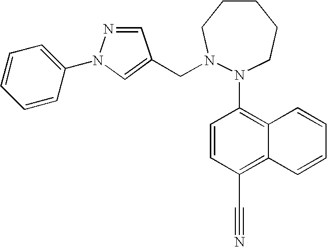

### 4-{2-[(1-phenyl-1H-pyrazol-4-yl)methyl]hexahydro-1H-1,2-diazepin-1-yl}-1-naphthalenecarbonitrile (C101)

From B4 and 1-phenyl-1H-pyrazol-4-carbaldehyde the title compound was isolated as a yellow oil.

MS (m/z) ESI ES⁺=408

### Example 102

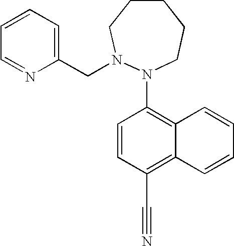

### 4-[2-(2-pyridinylmethyl)hexahydro-1H-1,2-diazepin-1-yl]-1-naphthalenecarbonitrile (C102)

From B4 and 2-Pyridinecarbaldehyde the Title Compound was Isolated as a Brown Oil.

MS (m/z) ESI ES⁺=343

### Example 103

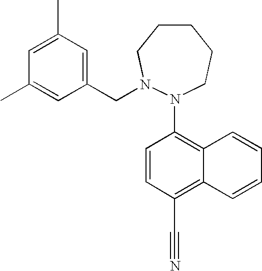

### 4-{2-[(3,5-dimethylphenyl)methyl]hexahydro-1H-1,2-diazepin-1-yl}-1-naphthalenecarbonitrile (C103)

From B4 and 3,5-dimethylbenzaldehyde the title compound was isolated as a yellow oil.

MS (m/z) ESI ES⁺=370

### Example 104

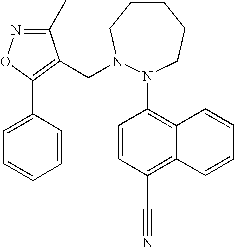

### 4-{2-[(3-methyl-5-phenyl-4-isoxazolyl)methyl]hexahydro-1H-1,2-diazepin-1-yl}-1-naphthalenecarbonitrile (C104)

From B4 and 3-methyl-5-phenyl-4-isoxazolecarbaldehyde the title compound was isolated as a yellow oil.

MS (m/z) ESI ES⁺=423

### Example 105

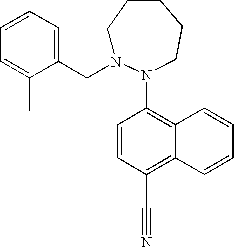

### 4-{2-[(2-methylphenyl)methyl]hexahydro-1H-1,2-diazepin-1-yl}-1-naphthalenecarbonitrile (C105)

From B4 and 2-methylbenzaldehyde the title compound was isolated as a yellow oil.

MS (m/z) ESI ES⁺=356

### Example 106

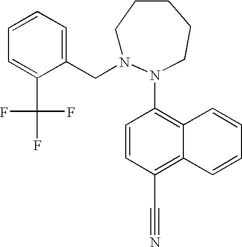

### 4-(2-{[2-(trifluoromethyl)phenyl]methyl}hexahydro-1H-1,2-diazepin-1-yl)-1-naphthalenecarbonitrile (C1068)

From B4 and 2-(trifluoromethyl)benzaldehyde the title compound was isolated as a yellow solid.

MS (m/z) ESI ES⁺=410

### Example 107

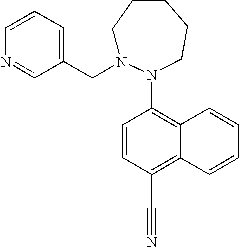

### 4-[2-(3-pyridinylmethyl)hexahydro-1H-1,2-diazepin-1-yl]-1-naphthalenecarbonitrile (C107)

From B4 and 3-pyridinecarbaldehyde the title compound was isolated as a yellow oil.

MS (m/z) ESI ES⁺=343

### Example 108

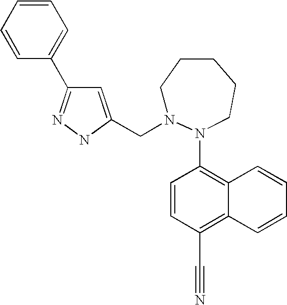

### 4-{2-[(3-phenyl-1H-pyrazol-5-yl)methyl]hexahydro-1H-1,2-diazepin-1-yl}-1-naphthalenecarbonitrile (C108)

From B4 and 3-phenyl-1H-pyrazol-5-carbaldehyde the title compound was isolated as a yellow oil.

MS (m/z) ESI ES⁺=408

### Example 109

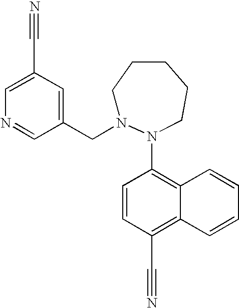

### 5-{[2-(4-cyano-1-naphthalenyl)hexahydro-1H-1,2-diazepin-1-yl]methyl}-3-pyridinecarbonitrile (C109)

From B4 and 5-formyl-3-pyridinecarbonitrile the title compound was isolated as a yellow oil.

MS (m/z) ESI ES⁺=368

### Example 110

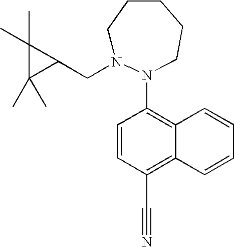

### 4-{2-[(2,2,3,3-tetramethylcyclopropyl)methyl]hexahydro-1H-1,2-diazepin-1-yl}-1-naphthalenecarbonitrile (C110)

From B4 and 2,2,3,3-tetramethylcyclopropanecarbaldehyde the title compound was isolated as a yellow oil.

MS (m/z) ESI ES⁺=362

### Example 111

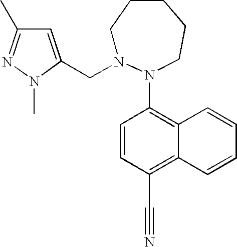

### 4-{2-[(1,3-dimethyl-1H-pyrazol-5-yl)methyl]hexahydro-1H-1,2-diazepin-1-yl}-1-naphthalenecarbonitrile (C111)

From B4 and 1,3-dimethyl-1H-pyrazol-5-carbaldehyde the title compound was isolated as a yellow oil.

MS (m/z) ESI ES⁺=360

### Example 112

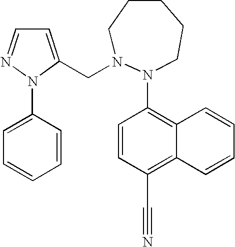

### 4-{2-[(1-phenyl-1H-pyrazol-5-yl)methyl]hexahydro-1H-1,2-diazepin-1-yl}-1-naphthalenecarbonitrile (C112)

From B4 and 1-phenyl-1H-pyrazol-5-carbaldehyde the title compound was isolated as a yellow oil.

MS (m/z) ESI ES⁺=408

### Example 113

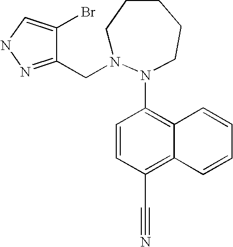

### 4-{2-[(4-bromo-1H-pyrazol-3-yl)methyl]hexahydro-1H-1,2-diazepin-1-yl}-1-naphthalenecarbonitrile (C113)

From B4 and 4-bromo-1H-pyrazol-3-carbaldehyde the title compound was isolated as a yellow oil.

MS (m/z) ESI ES⁺=411

### Example 114

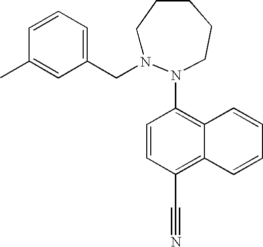

### 4-{2-[(3-methylphenyl)methyl]hexahydro-1H-1,2-diazepin-1-yl}-1-naphthalenecarbonitrile (C114)

From B4 and 3-methylbenzaldehyde the title compound was isolated as a yellow oil.

MS (m/z) ESI ES⁺=356

### Example 115

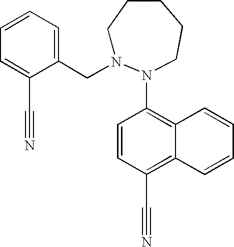

### 4-{2-[(2-cyanophenyl)methyl]hexahydro-1H-1,2-diazepin-1-yl}-1-naphthalenecarbonitrile (C115)

From B4 and 2-cyanobenzaldehyde the title compound was isolated as a green oil.

MS (m/z) ESI ES⁺=367

### Example 116

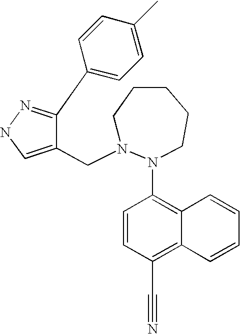

### 4-(2-{[3-(4-methylphenyl)-1H-pyrazol-4-yl]methyl}hexahydro-1H-1,2-diazepin-1-yl)-1-naphthalenecarbonitrile (C116)

From B4 and 3-(4-methylphenyl)-1H-pyrazol-4-carbaldehyde the title compound was isolated as a yellow oil.

MS (m/z) ESI ES⁺=422

### Example 117

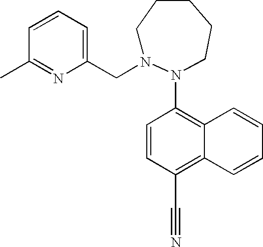

### 4-{2-[(6-methyl-2-pyridinyl)methyl]hexahydro-1H-1,2-diazepin-1-yl}-1-naphthalenecarbonitrile (C117)

From B4 and 6-methyl-2-pyridinecarbaldehyde the title compound was isolated as an orange oil.

MS (m/z) ESI ES⁺=357

### Example 118

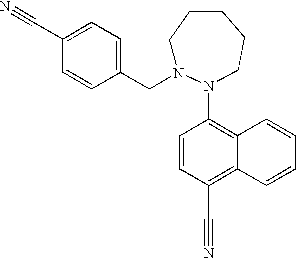

### 4-{2-[(4-cyanophenyl)methyl]hexahydro-1H-1,2-diazepin-1-yl}-1-naphthalenecarbonitrile (C18)

From B4 and 4-cyanobenzaldehyde the title compound was isolated as a yellow oil.

MS (m/z) ESI ES⁺=367

### Example 119

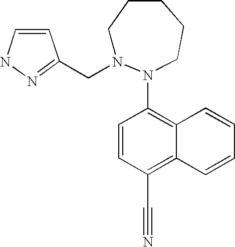

### 4-[2-(1H-pyrazol-3-ylmethyl)hexahydro-1H-1,2-diazepin-1-yl]-1-naphthalenecarbonitrile (C119)

From B4 and 1H-pyrazol-3-carbaldehyde the title compound was isolated as a yellow oil.

MS (m/z) ESI ES⁺=332

### Example 120

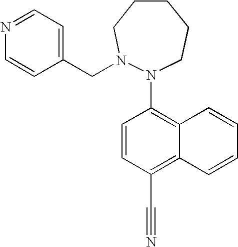

### 4-[2-(4-pyridinylmethyl)hexahydro-1H-1,2-diazepin-1-yl]-1-naphthalenecarbonitrile (C120)

From B4 and 4-pyridinecarbaldehyde the title compound was isolated as a brown solid.

MS (m/z) ESI ES⁺=343

### Example 121

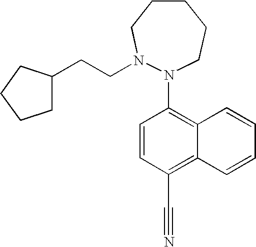

### 4-[2-(2-cyclopentylethyl)hexahydro-1H-1,2-diazepin-1-yl]-1-naphthalenecarbonitrile (C121)

From B4 and cyclopentylacetaldehyde the title compound was isolated as a yellow solid.

MS (m/z) ESI ES⁺=348

### Example 122

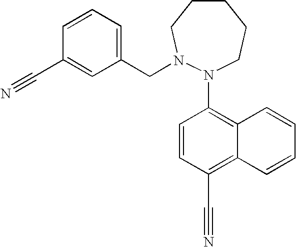

### 4-{2-[(3-cyanophenyl)methyl]hexahydro-1H-1,2-diazepin-1-yl}-1-naphthalenecarbonitrile (C122)

From B4 and 3-cyanobenzaldehyde the title compound was isolated as a yellow solid.

MS (m/z) ESI ES⁺=367

### Example 123

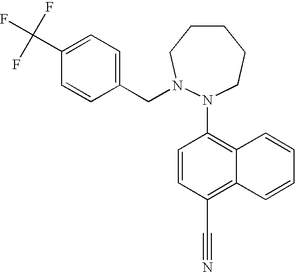

### 4-(2-{[4-(trifluoromethyl)phenyl]methyl}hexahydro-1H-1,2-diazepin-1-yl)-1-naphthalenecarbonitrile (C123)

From B4 and 4-(trifluoromethyl)benzaldehyde the title compound was isolated as a yellow oil.

MS (m/z) ESI ES⁺=410

### Example 124

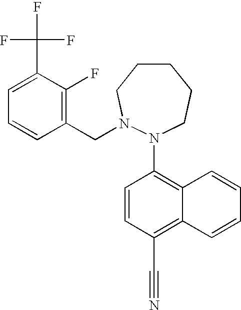

### 4-(2-{[2-fluoro-3-(trifluoromethyl)phenyl]methyl}hexahydro-1H-1,2-diazepin-1-yl)-1-naphthalenecarbonitrile (C124)

From B4 and 2-fluoro-3-(trifluoromethyl)benzaldehyde the title compound was isolated as a yellow oil.

MS (m/z) ESI ES⁺=428

### Example 125

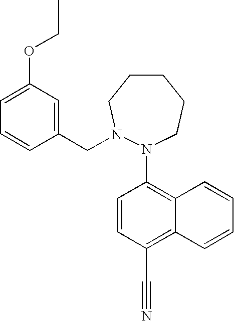

### 4-(2-{[3-(ethyloxy)phenyl]methyl}hexahydro-1H-1,2-diazepin-1-yl)-1-naphthalenecarbonitrile (C125)

From B4 and 3-(ethyloxy)benzaldehyde the title compound was isolated as a yellow oil.

MS (m/z) ESI ES⁺=386

### Example 126

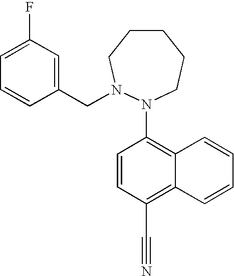

### 4-{2-[(3-fluorophenyl)methyl]hexahydro-1H-1,2-diazepin-1-yl}-1-naphthalenecarbonitrile (C126)

From B4 and 3-fluorobenzaldehyde the title compound was isolated as a yellow oil.

MS (m/z) ESI ES⁺=360

### Example 127

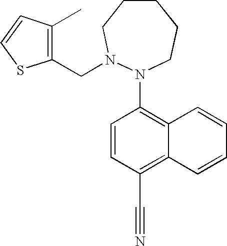

### 4-{2-[(3-methyl-2-thienyl)methyl]hexahydro-1H-1,2-diazepin-1-yl}-1-naphthalenecarbonitrile (C127)

From B4 and 3-methyl-2-thiophenecarbaldehyde the title compound was isolated as a yellow oil.

MS (m/z) ESI ES⁺=362

### Example 128

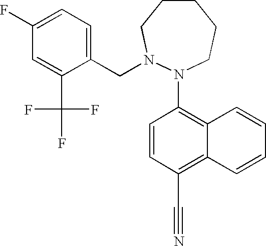

### 4-(2-{[4-fluoro-2-(trifluoromethyl)phenyl]methyl}hexahydro-1H-1,2-diazepin-1-yl)-1-naphthalenecarbonitrile (C128)

From B4 and 4-fluoro-2-(trifluoromethyl)benzaldehyde the title compound was isolated as a yellow oil.

MS (m/z) ESI ES⁺=428

### Example 129

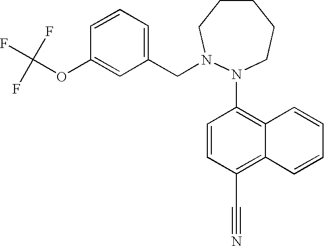

### 4-[2-({3-[(trifluoromethyl)oxy]phenyl}methyl)hexahydro-1H-1,2-diazepin-1-yl]-1-naphthalenecarbonitrile (C129)

From B4 and 3-[(trifluoromethyl)oxy]benzaldehyde the title compound was isolated as a yellow oil.

MS (m/z) ESI ES⁺=426

### Example 130

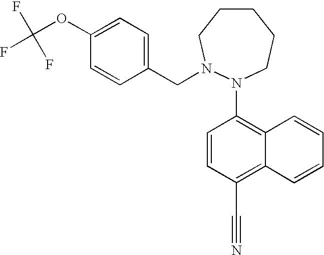

### 4-[2-({3-[(trifluoromethyl)oxy]phenyl}methyl)hexahydro-1H-1,2-diazepin-1-yl]-1-naphthalenecarbonitrile (C130)

From B4 and 4-[(trifluoromethyl)oxy]benzaldehyde the title compound was isolated as a yellow oil.

MS (m/z) ESI ES⁺=426

### Example 131

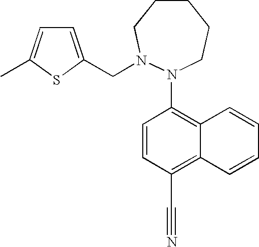

### 4-{2-[(5-methyl-2-thienyl)methyl]hexahydro-1H-1,2-diazepin-1-yl}-1-naphthalenecarbonitrile (C131)

From B4 and 5-methyl-2-thiophenecarbaldehyde the title compound was isolated as a yellow oil.

MS (m/z) ESI ES⁺=362

### Example 132

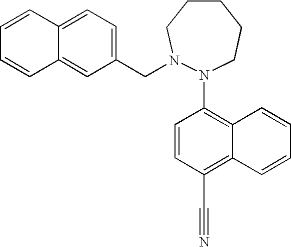

### 4-[2-(2-naphthalenylmethyl)hexahydro-1H-1,2-diazepin-1-yl]-1-naphthalenecarbonitrile (C132)

From B4 and 2-naphthalenecarbaldehyde the title compound was isolated as an orange oil.

MS (m/z) ESI ES⁺=392

### Example 133

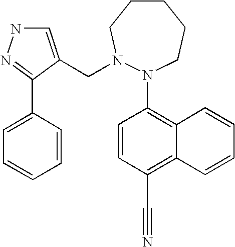

### 4-{2-[(3-phenyl-1H-pyrazol-4-yl)methyl]hexahydro-1H-1,2-diazepin-1-yl}-1-naphthalenecarbonitrile (C133)

From B4 and 3-phenyl-1H-pyrazol-4-carbaldehyde the title compound was isolated as a yellow oil.

MS (m/z) ESI ES⁺=408

### Example 134

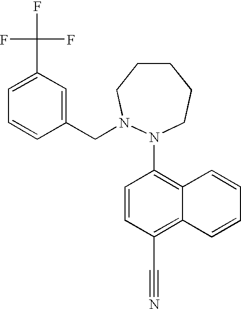

### 4-(2-{[3-(trifluoromethyl)phenyl]methyl}hexahydro-1H-1,2-diazepin-1-yl)-1-naphthalenecarbonitrile (C134)

From B4 and 3-(trifluoromethyl)benzaldehyde the title compound was isolated as a yellow oil.

MS (m/z) ESI ES⁺=410

### Example 135

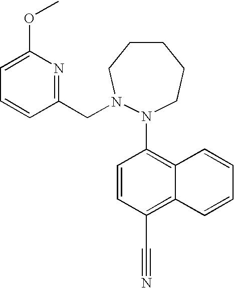

### 4-(2-{[6-(methyloxy)-2-pyridinyl]methyl}hexahydro-1H-1,2-diazepin-1-yl)-1-naphthalenecarbonitrile (C135)

From B4 and 6-(methyloxy)-2-pyridinecarbaldehyde the title compound was isolated as a yellow oil.

MS (m/z) ESI ES⁺=373

### Example 136

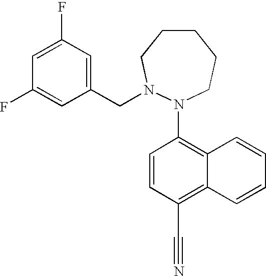

### 4-{2-[(3,5-difluorophenyl)methyl]hexahydro-1H-1,2-diazepin-1-yl}-1-naphthalenecarbonitrile (C136)

From B4 and 3,5-dlifluorobenzaldehyde the title compound was isolated as a yellow oil.

MS (m/z) ESI ES⁺=378

### Example 137

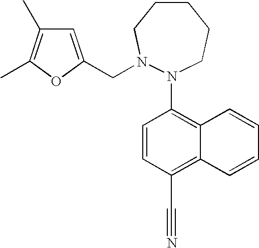

### 4-{2-[(4,5-dimethyl-2-furanyl)methyl]hexahydro-1H-1,2-diazepin-1-yl}-1-naphthalenecarbonitrile (C137)

From B4 and 4,5-dimethyl-2-furancarbaldehyde the title compound was isolated as a brown oil.

MS (m/z) ESI ES⁺=360

### Example 138

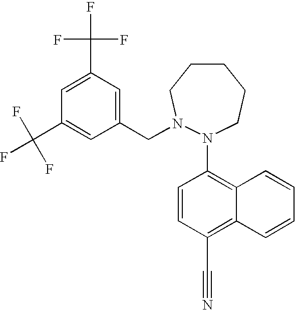

### 4-(2-{[3,5-bis(trifluoromethyl)phenyl]methyl}hexahydro-1H-1,2-diazepin-1-yl)-1-naphthalenecarbonitrile (C138)

From B4 and 3,5-bis(trifluoromethyl)benzaldehyde the title compound was isolated as a yellow oil.

MS (m/z) ESI ES⁺=478

### Example 139

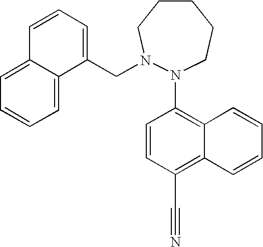

### 4-[2-(1-naphthalenylmethyl)hexahydro-1H-1,2-diazepin-1-yl]-1-naphthalenecarbonitrile (C139)

From B4 and 1-naphthalenecarbaldehyde the title compound was isolated as a yellow oil.

MS (m/z) ESI ES⁺=392

### Example 140

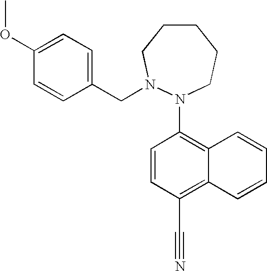

### 4-(2-{[4-(methyloxy)phenyl]methyl}hexahydro-1H-1,2-diazepin-1-yl)-1-naphthalenecarbonitrile (C140)

From B4 and 4-(methyloxy)benzaldehyde the title compound was isolated as a yellow oil.

MS (m/z) ESI ES⁺=372

### Example 141

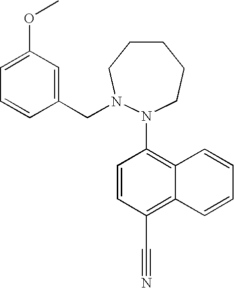

### 4-(2-{[3-(methyloxy)phenyl]methyl}hexahydro-1H-1,2-diazepin-1-yl)-1-naphthalenecarbonitrile (C141)

From B4 and 3-(methyloxy)benzaldehyde the title compound was isolated as a yellow oil.

MS (m/z) ESI ES⁺=372

### Example 142

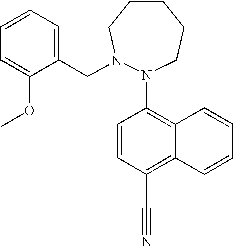

### 4-(2-{[2-(methyloxy)phenyl]methyl}hexahydro-1H-1,2-diazepin-1-yl)-1-naphthalenecarbonitrile (C142)

From B4 and 2-(methyloxy)benzaldehyde the title compound was isolated as a yellow oil.

MS (m/z) ESI ES⁺=372

### Example 143

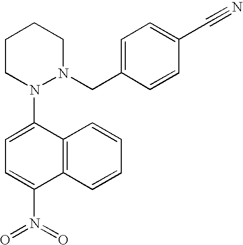

### 4-{[2-(4-nitro-1-naphthalenyl)tetrahydro-1(2H)-pyridazinyl]methyl}benzonitrile (C143)

From B2 and 4-cyanobenzaldehyde the title compound was obtained.

MS (m/z) ESI ES⁺=373

### Example 144

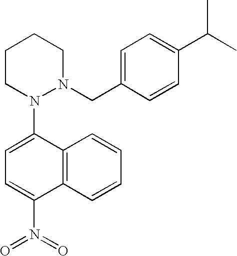

### 1-{[4-(1-methylethyl)phenyl]methyl}-2-(4-nitro-1-naphthalenyl)hexahydropyridazine (C144)

From B2 and 4-(1-methylethyl)benzaldehyde the title compound was obtained.

MS (m/z) ESI ES⁺=390

### Example 145

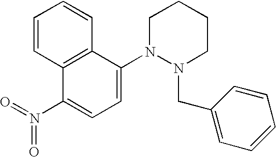

### 1-(4-nitro-1-naphthalenyl)-2-(phenylmethyl)hexahydropyridazine (C145)

From B2 and benzaldehyde the title compound was obtained.

1H NMR (400 MHz, CHLOROFORM-D) δ ppm 1.7 (m, 2H) 2.0 (m, 2H) 3.2 (m, 2H) 3.5 (m, 2H) 4.0 (s, 2H) 6.9 (m, 3H) 7.1 (m, 3H) 7.5 (m, 1H) 7.7 (m, 1H) 8.2 (d, J=8.6 Hz, 1H) 8.4 (m, 1H) 8.7 (m, 1H)

### Example 146

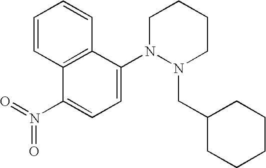

### 1-(cyclohexylmethyl)-2-(4-nitro-1-naphthalenyl)hexahydropyridazine (C146)

From B2 and cyclohexanecarbaldehyde the title compound was obtained.

MS (m/z) ESI ES⁺=354

### Example 147

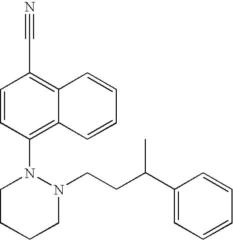

### 4-[2-(3-phenylbutyl)tetrahydro-1(2H)-pyridazinyl]-1-naphthalenecarbonitrile (C147)

From B4 and 3-phenylbutanal the title compound was isolated as a yellow oil.

1H NMR (300 MHz, METHANOL-D4) δ ppm 1.7 (m, 8H), 2.3 (m, 1H), 2.7 (m, 1H),

2.8 (m, 1H), 3.4 (m, 5H), 6.6 (m, 2H), 7.0 (m, 4H), 7.7 (m, 3H), 8.1 (d, J=8.3 Hz, 1H), 8.5 (d, J=8.6 Hz, 1H)

### Example 148

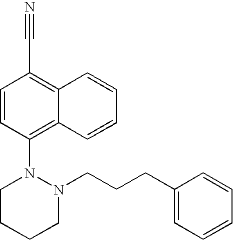

### 4-[2-(3-phenylpropyl)tetrahydro-1(2H-pyridazinyl]-1-naphthalenecarbonitrile (C148)

From B4 and 3-phenylpropanal the title compound was isolated as a yellow oil.

1H NMR (300 MHz, METHANOL-D4) 8 ppm 1.5 (m, 2H), 1.8 (m, 4H), 2.2 (t, J=7.5 Hz, 2H), 2.9 (t, J=6.9 Hz, 2H), 3.4 (m, 4H), 6.6 (m, 2H), 7.1 (m, 4H), 7.6 (m, 2H), 7.8 (d, J=8.0 Hz, 1H), 8.1 (d, J=8.3 Hz, 1H), 8.5 (d, J=8.6 Hz, 1H)

### Example 149

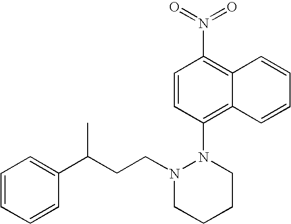

### 1-(4-nitro-1-naphthalenyl)-2-(3-phenylbutyl)hexahydropyridazine (C149)

From B2 and 3-phenylbutanal the title compound was isolated as a red oil.

MS (m/z) APCI AP⁺=390

### Example 150

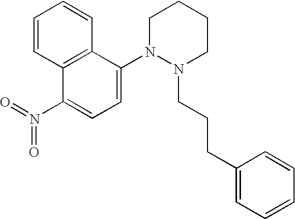

### 1-(4-nitro-1-naphthalenyl)-2-(3-phenylpropyl)hexahydropyridazine (C150)

From B2 and 3-phenylpropanal the title compound was isolated as a red oil.

1H NMR (300 MHz, METHANOL-D4) δ ppm 1.5 (m, 2H) 1.8 (m, 4H) 2.3 (t, J=7.5 Hz, 2H) 2.9 (t, J=6.8 Hz, 2H) 3.4 (m, 2H) 3.5 (m, 2H) 6.6 (m, 2H) 7.1 (m, 4H) 7.6 (m, 1H) 7.7 (m, 1H) 8.3 (d, J=8.6 Hz, 1H) 8.4 (d, J=9.1 Hz, 1H) 8.7 (d, J=9.1 Hz, 1H)

### Example 151

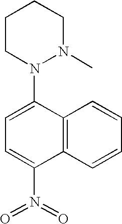

### 1-methyl-2-(4-nitro-1-naphthalenyl)hexahydropyridazine (C151)

From B2 and acetaldehyde the title compound was obtained.

MS (m/z) ESI ES⁺=272

### Example 152

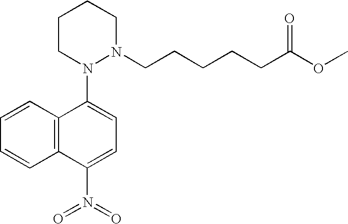

### Methyl 6-[2-(4-nitro-1-naphthalenyl)tetrahydro-1(2H)-pyridazinyl]hexanoate (C152)

From B2 and methyl 6-oxohexanoate the title compound was obtained.

1H NMR (300 MHz, CHLOROFORM-D) δ ppm 1.0 (m, 2H) 1.3 (m, 4H) 1.8 (m, 4H) 2.0 (m, 2H) 2.9 (t, J=6.9 Hz, 2H) 3.3 (m, 2H) 3.5 (m, 2H) 3.6 (s, 2H) 7.0 (d, J=8.8 Hz, 1H) 7.5 (m, 1H) 7.7 (m, 1H) 8.3 (d, J=9.4 Hz, 1H) 8.3 (d, J=8.6 Hz, 1H) 8.8 (d, J=8.3 Hz, 1H)

### Example 153

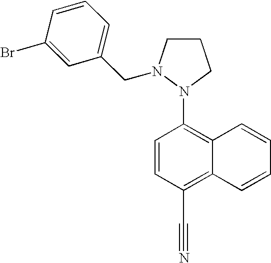

### 4-{2-[(3-bromophenyl)methyl]-1-pyrazolidinyl}-1-naphthalenecarbonitrile (C153)

From B5 and 3-bromobenzaldehyde the title compound was isolated as a yellow oil.

MS (m/z) ESI ES⁺=393

### Example 154

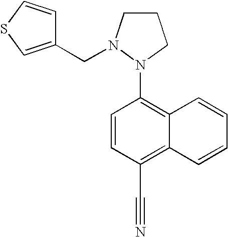

### 4-[2-(3-thienylmethyl)-1-pyrazolidinyl]-1-naphthalenecarbonitrile (C154)

From B5 and 3-thiophenecarbaldehyde the title compound was isolated as a yellow oil.

MS (m/z) ESI ES⁺=320

### Example 155

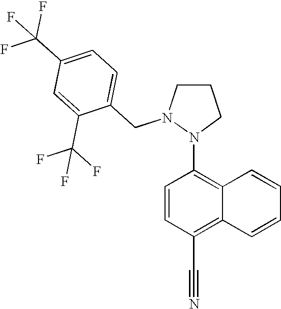

### 4-(2-{[2,4-bis(trifluoromethyl)phenyl]methyl}-1-pyrazolidinyl)-1-naphthalenecarbonitrile (C155)

From B5 and 2,4-bis(trifluoromethyl)benzaldehyde the title compound was isolated as a yellow solid.

MS (m/z) ESI ES⁺=450

### Example 156

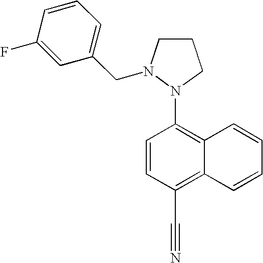

### 4-{2-[(3-fluorophenyl)methyl]-1-pyrazolidinyl}-1-naphthalenecarbonitrile (C156)

From B5 and 3-fluorobenzaldehyde the title compound was isolated as a yellow oil.

MS (m/z) ESI ES⁺=332

### Example 157

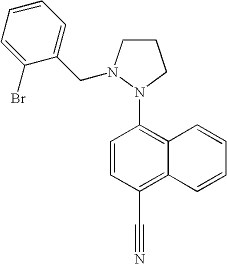

### 4-{2-[(2-bromophenyl)methyl]-1-pyrazolidinyl}-1-naphthalenecarbonitrile (C157)

From B5 and 2-bromobenzaldehyde the title compound was isolated as a yellow solid.

MS (m/z) ESI ES⁺=393

### Example 158

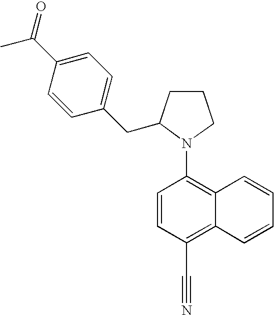

### 4-{2-[(4-acetylphenyl)methyl]-1-pyrazolidinyl}-1-naphthalenecarbonitrile (C158)

From B5 and 4-acetylbenzaldehyde the title compound was isolated as a yellow oil.

MS (m/z) ESI ES⁺=356

### Example 159

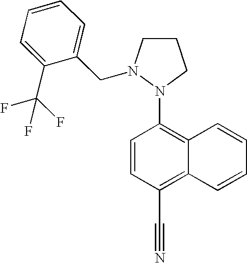

### 4-(2-{[2-(trifluoromethyl)phenyl]methyl}-1-pyrazolidinyl)-1-naphthalenecarbonitrile (C159)

From B5 and 2-(trifluoromethyl)benzaldehyde the title compound was isolated as a yellow oil.

MS (m/z) ESI ES⁺=382

### Example 160

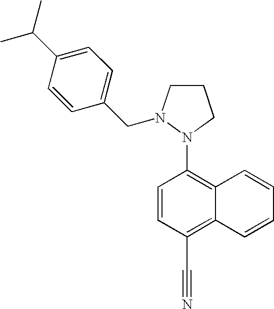

### 4-(2-{[4-(1-methylethyl)phenyl]methyl}-1-pyrazolidinyl)-1-naphthalenecarbonitrile (C160)

From B5 and 4-(1-methylethyl)benzaldehyde the title compound was isolated as a yellow oil.

MS (m/z) ESI ES⁺=356

### Example 161

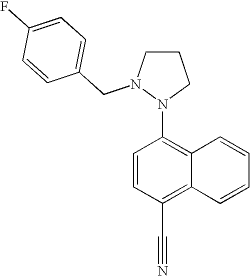

### 4-{2-[(4-fluorophenyl)methyl]-1-pyrazolidinyl}-1-naphthalenecarbonitrile (C161)

From B5 and 4-fluorobenzaldehyde the title compound was isolated as a yellow oil.

MS (m/z) ESI ES⁺=332

### Example 162

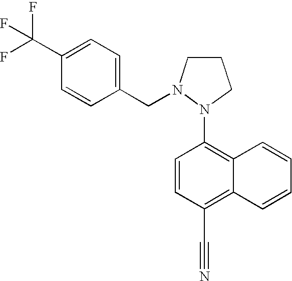

### 4-(2-{[4-(trifluoromethyl)phenyl]methyl}-1-pyrazolidinyl)-1-naphthalenecarbonitrile (C162)

From B5 and 4-(trifluoromethyl)benzaldehyde the title compound was isolated as a yellow oil.

MS (m/z) ESI ES⁺=382

### Example 163

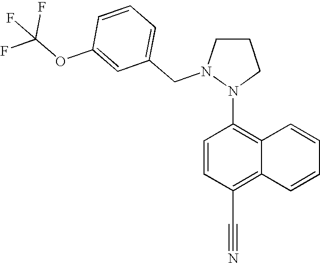

### 4-[2-({3-[(trifluoromethyl)oxy]phenyl}methyl)-1-pyrazolidinyl]-1-naphthalenecarbonitrile (C163)

From B5 and 3-[(trifluoromethyl)oxy]benzaldehyde the title compound was isolated as a yellow oil.

MS (m/z) ESI ES⁺=398

### Example 164

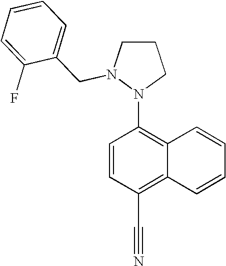

### 4-{2-[(2-fluorophenyl)methyl]-1-pyrazolidinyl}-1-naphthalenecarbonitrile (C164)

From B5 and 3-fluorobenzaldehyde the title compound was isolated as a yellow oil.

MS (m/z) ESI ES⁺=332

### Example 165

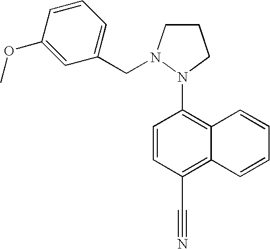

### 4-(2-{[3-(methyloxy)phenyl]methyl}-1-pyrazolidinyl)-1-naphthalenecarbonitrile (C165)

From B5 and 3-(methyloxy)benzaldehyde the title compound was isolated as a yellow oil.

MS (m/z) ESI ES⁺=344

### Example 166

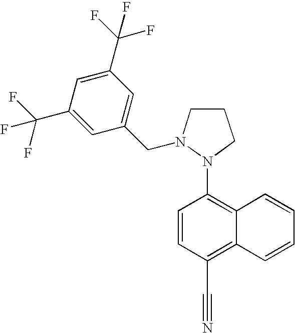

### 4-(2-{[3,5-bis(trifluoromethyl)phenyl]methyl}-1-pyrazolidinyl)-1-naphthalenecarbonitrile (C166)

From B5 and 3,5-bis(trifluoromethyl)benzaldehyde the title compound was isolated as a yellow oil.

MS (m/z) ESI ES⁺=450

### Example 167

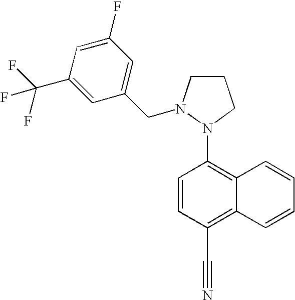

### 4-(2-{[3-fluoro-5-(trifluoromethyl)phenyl]methyl}-1-pyrazolidinyl)-1-naphthalenecarbonitrile (C167)

From B5 and 3-fluoro-5-(trifluoromethyl)benzaldehyde the title compound was isolated as a yellow oil.

MS (m/z) ESI ES⁺=400

### Example 168

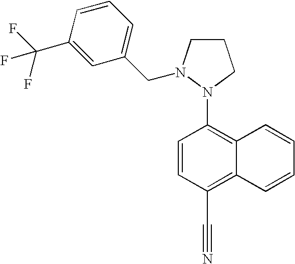

### 4-(2-{[3-(trifluoromethyl)phenyl]methyl}-1-pyrazolidinyl)-1-naphthalenecarbonitrile (C168)

From B5 and 3-(trifluoromethyl)benzaldehyde the title compound was isolated as a yellow oil.

MS (m/z) ESI ES⁺=382

### Example 169

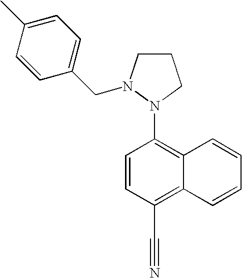

### 4-{2-[(4-methylphenyl)methyl]-1-pyrazolidinyl}-1-naphthalenecarbonitrile (C169)

From B5 and 4-methylbenzaldehyde the title compound was isolated as a yellow solid.

MS (m/z) ESI ES⁺=328

### Example 170

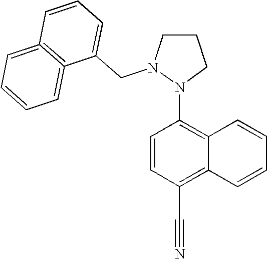

### 4-[2-(1-naphthalenylmethyl)-1-pyrazolidinyl]-1-naphthalenecarbonitrile (C170)

From B5 and 1-naphthalenecarbaldehyde the title compound was isolated as a yellow solid.

MS (m/z) ESI ES⁺=364

### Example 171

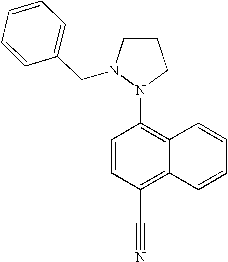

### 4-[2-(phenylmethyl)-1-pyrazolidinyl]-1-naphthalenecarbonitrile (C171)

From B5 and benzaldehyde the title compound was isolated as a yellow oil.

MS (m/z) ESI ES⁺=314

### Example 172

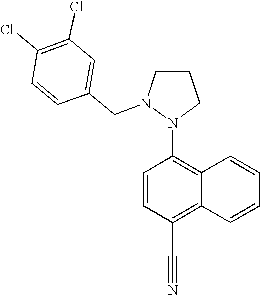

### 4-{2-[(3,4-dichlorophenyl)methyl]-1-pyrazolidinyl}-1-naphthalenecarbonitrile (C172)

From B5 and 3,4-dichlorobenzaldehyde the title compound was isolated as a yellow solid.

MS (m/z) ESI ES⁺=383

### Example 173

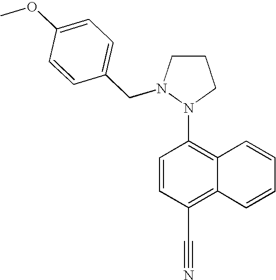

### 4-(2-{[4-(methyloxy)phenyl]methyl}-1-pyrazolidinyl)-1-naphthalenecarbonitrile (C173)

From B5 and 4-(methyloxy)benzaldehyde the title compound was isolated as a yellow solid.

MS (m/z) ESI ES⁺=344

### Example 174

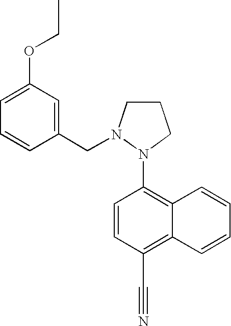

### 4-(2-{[3-(ethyloxy)phenyl]methyl}-1-pyrazolidinyl)-1-naphthalenecarbonitrile (C174)

From B5 and 3-(Ethyloxy)Benzaldehyde the Title Compound was Isolated as a Yellow Oil.

MS (m/z) ESI ES⁺=358

### Example 175

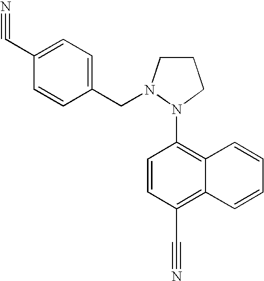

### 4-{2-[(4-cyanophenyl)methyl]-1-pyrazolidinyl}-1-naphthalenecarbonitrile (C175)

From B5 and 4-cyanobenzaldehyde the title compound was isolated as a yellow solid.

MS (m/z) ESI ES⁺=339

### Example 176

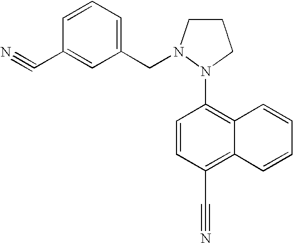

### 4-{2-[(3-cyanophenyl)methyl]-1-pyrazolidinyl}-1-naphthalenecarbonitrile (C176)

From B5 and 3-cyanobenzaldehyde the title compound was isolated as a white solid.

MS (m/z) ESI ES⁺=339

### Example 177

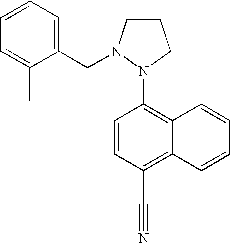

### 4-{2-[(2-methylphenyl)methyl]-1-pyrazolidinyl}-1-naphthalenecarbonitrile (C177)

From B5 and 2-methylbenzaldehyde the title compound was isolated as a white solid.

MS (m/z) ESI ES⁺=328

### Example 178

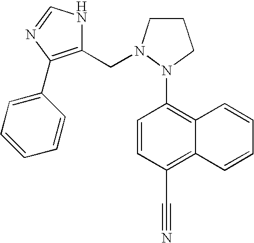

### 4-[2-[(4-phenyl-1H-imidazol-5-yl)methyl]tetrahydro-1(2H)-pyridazinyl]-1-naphthalenecarbonitrile (C178)

From B1 and 4-phenyl-1H-imidazol-5-carbaldehyde the title compound was isolated as a white solid.

MS (m/z) ESI ES⁺=394

### Example 179

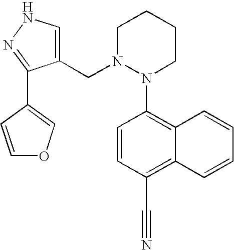

### 4-[2-{[3-(1-methylethyl)-1H-pyrazol-4-yl]methyl}tetrahydro-1(2H)-pyridazinyl]-1-naphthalenecarbonitrile (C179)

To a 250-ml round bottom flask equipped with a magnetic stir bar and nitrogen inlet was added ethyl 3-(3-furanyl)-3-oxopropanoate (1 g, 5.5 mmoles) followed by [bis(methyloxy)methyl]dimethylamine (10 ml). The reaction was allowed to stir at room temperature overnight. The volatiles were removed in vacuo. The crude product (5.5 mmoles) was used without characterization or purification. To this crude product was added acetic acid (10 ml) and hydrazine hydrate (0.83 g, 3 eq) and heated at 100° C. overnight. After cooling to room temperature, the volatiles were removed under reduced pressure. The residue was partitioned between ethyl acetate and 0.1N NaOH (pH˜10). The phases were separated and the organic fraction was washed twice with water, once with brine, dried over sodium sulfate, filtered and concentrated under reduced pressure to yield quantitative yield of crude pyrazole ester. This crude product was used without purification. The pyrazole ester (5.5 mmoles) in diethyl ether (5 ml) was added dropwise to a precooled (0° C.) suspension of lithium aluminumhydride (330 mg, 1.5 eq) in diethyl ether (10 ml). The reaction mixture was allowed to stir for 1 hr at room temperature at which point 0.4 ml of water was added very slowly, 0.4 ml of 5N NaOH, and 1.2 ml of water. This mixture was allowed to stir for 2 hrs resulting in the precipitation of a white solid. The reaction mixture was filtered through Celite and the salts were washed with copious amounts of ethyl acetate and methanol. The filtrate was concentrated to yield 540 mg (60% yield) of crude pyrazole alcohol. To the crude pyrazole alcohol was added acetone (10 ml) followed by manganese dioxide (2.9 g, 10 eq) and the reaction was stirred at 50° C. for 4 hrs. After cooling to room temperature the reaction was filtered through Celite and washed with acetone. The filtrate was concentrated to yield 300 mg (56% yield) of the pyrazole aldehyde.

The above aldehyde (100 mg, 2 eq) was coupled with B1(75 mg, 1 eq) via the reductive amination procedure outlined in Example 1 (C1) to yield 45 mg of the title compound.

MS (m/z) ESI ES⁺=384

The following compounds were synthesized according to a similar general procedure as used for C179 with the following exceptions: minimal amounts of anhydrous THF were used as the co-solvent in the LiAlH₄ reaction when solubility in Et₂O was low, with certain less-reactive substrates the LiAlH₄ reduction was allowed to proceed up to 48 hrs at 0° C., and, in certain cases, varying amounts of over-reduction of the ester to the Me analog was observed during the LiAlH₄ reaction:

### Example 180

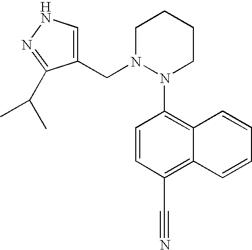

### 4-[2-{[3-(1-methylethyl)-1H-pyrazol-4-yl]methyl}tetrahydro-1(2H)-pyridazinyl]-1-naphthalenecarbonitrile (C180)

From B1 and ethyl 4-methyl-3-oxopentanoate the title compound was obtained.

MS (m/z) ESI ES⁺=360

### Example 181

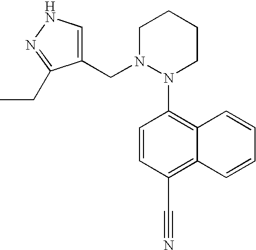

### 4-[2-[(3-ethyl-1H-pyrazol-4-yl)methyl]tetrahydro-1(2H)-pyridazinyl]-1-naphthalenecarbonitrile (C181)

From B1 and ethyl 3-oxopentanoate the title compound was obtained.

MS (m/z) ESI ES⁺=346

### Example 182

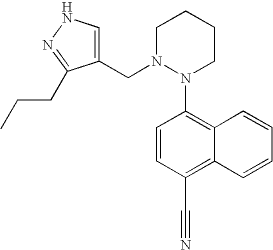

### 4-[2-[(3-propyl-1H-pyrazol-4-yl)methyl]tetrahydro-1(2H)-pyridazinyl]-1-naphthalenecarbonitrile (C182)

From B1 and ethyl 3-oxohexanoate the title compound was obtained.

MS (m/z) ESI ES⁺=360

### Example 183

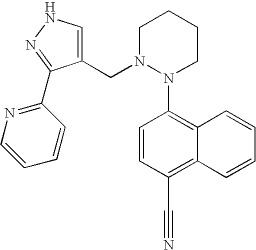

### 4-[2-{[3-(2-pyridinyl)-1H-pyrazol-4-yl]methyl}tetrahydro-1(2H)-pyridazinyl]-1-naphthalenecarbonitrile (C183)

From B1 and ethyl 3-oxo-3-(2-pyridinyl)propanoate the title compound was obtained.

MS (m/z) ESI ES⁺=395

### Example 184

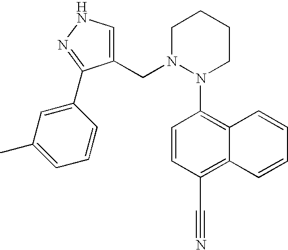

### 4-[2-{[3-(3-methylphenyl)-1H-pyrazol-4-yl]methyl}tetrahydro-1(2H)-pyridazinyl]-1-naphthalenecarbonitrile (C184)

From B1 and ethyl 3-(3-methylphenyl)-3-oxopropanoate the title compound was obtained.

MS (m/z) ESI ES⁺=408

The following compounds were synthesized according to a similar general procedure as used for Example 1 (C1):

### Example 185

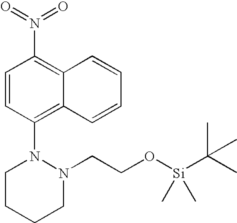

### E. 1-(2-{[(1,1-dimethylethyl)(dimethyl)silyl]oxy}ethyl)-2-(4-nitro-1-naphthalenyl)hexahydropyridazine (E1)

From B2 and {[(1,1-dimethylethyl)(dimethyl)silyl]oxy}acetaldehyde the title compound was obtained.

1H NMR (400 MHz, CHLOROFORM-D) δ ppm −0.1 (s, 6H) 0.8 (s, 9H) 1.8 (m, 4H) 3.0 (t, J=6.6 Hz, 2H) 3.3 (m, 2H) 3.4 (t, J=6.5 Hz, 2H) 3.5 (m, 2H) 7.0 (d, J=8.6 Hz, 1H) 7.5 (t, J=7.7 Hz, 1H) 7.6 (t, J=7.8 Hz, 1H) 8.3 (d, J=8.6 Hz, 2H) 8.7 (d, J=8.8 Hz, 1H)

### Example 186

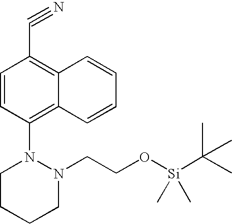

### 4-[2-(2-{([(1,1-dimethylethyl)(dimethyl)silyl]oxy}ethyl)tetrahydro-1(2H)-pyridazinyl]-1-naphthalenecarbonitrile (E2)

From B1 and {[(1,1-dimethylethyl)(dimethyl)silyl]oxy}acetaldehyde the title compound was obtained.

1H NMR (400 MHz, CHLOROFORM-D) δ ppm −0.2 (s, 6H) 0.7 (s, 9H) 1.8 (m, 4H) 2.9 (t, J=6.6 Hz, 2H) 3.3 (t, J=6.6 Hz, 2H) 3.3 (m, 2H) 3.4 (m, 2H) 6.9 (d, J=7.9 Hz, 1H) 7.5 (m, 1H) 7.6 (t, J=8.0 Hz, 1H) 7.8 (d, J=8.1 Hz, 1H) 8.1 (d, J=8.3 Hz, 1H) 8.3 (d, J=8.8 Hz, 1H)

### Cyclic Hydrazine Reductive Amination Using Phenylacetaldehydes

### Example 187

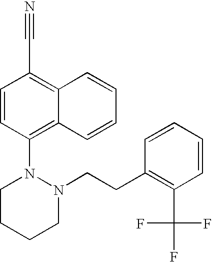

### 4-[2-{2-[2-(trifluoromethyl)phenyl]ethyl}tetrahydro-1(2H-pyridazinyl]-1-naphthalenecarbonitrile (C185)

To a 40 ml scintillation vial was added 0.067 ml of 2-[(trifluoromethyl)phenyl]ethanol (0.42 mmoles, 1 eq) and 5 ml dichloromethane. Then 3 g of 21 wt % pyridinium chlorochromate/silica gel (Silicycle, 21 wt %, 4 eq) was added and the reaction was shaken on an orbital shaker overnight. The reaction was filtered, washed with 3 ml diethyl ether and concentrated at room temperature at 200 mBar until approximately 3 ml of volume remained. The resulting aldehyde was neither isolated nor analyzed the crude product was used in the next step below. (100% yield was assumed.)

To a 40 ml scintillation vial was added 66 mg 4-(tetrahydro-1(2H)-pyridazinyl)-1-naphthalenecarbonitrile (B1) (0.278 mmoles, 1 eq), 3 ml ethanol, 1 ml acetic acid, and the above aldehyde solution, which contains a solution of the above aldehyde in 3 ml dichloromethane (0.42 mmoles, 1.5 eq). Then 400 mg (polystyrylmethyl)trimethylammoniumcyanoborohydride resin (Novabiochem, 4.3 mmol/g, 5 eq) was added and the reaction was shaken on an orbital shaker overnight. The reaction was filtered and concentrated in vacuo. The residue was purified via preparative HPLC (Phenomenex column Luna C18, 75 mm×30 mm, 5 micron), 50% acetonitrile/water (0.01% TFA) to 100% acetonitrile, to yield 36 mg of the title compound as a yellow oil.

1H NMR (400 MHz, METHANOL-D4)₆ ppm 1.8 (m, 4H), 2.6 (t, J=7.2 Hz, 2H), 3.1 (t, J=7.2 Hz, 2H), 3.4 (m, 2H), 3.4 (m, 2H), 7.0 (m, 1H), 7.0 (m, J=8.1 Hz, 1H), 7.1 (m, 2H), 7.4 (m, 1H), 7.4 (m, 1H), 7.6 (m, 1H), 7.8 (d, J=7.9 Hz, 1H), 8.0 (d, J=8.6 Hz, 1H), 8.3 (d, J=8.8 Hz, 1H)

The following compounds were synthesized according to the same general procedure as used to synthesize Example 187 (C185):

### Example 188

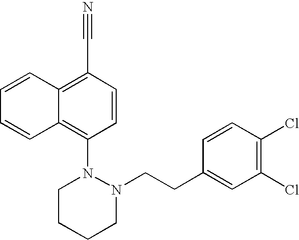

### 4-[2-[2-(3,4-dichlorophenyl)ethyl]tetrahydro-1(2H)-pyridazinyl]-1-naphthalenecarbonitrile (C186)

From B1 and 2-(3,4-dichlorophenyl)ethanol the title compound was isolated as an orange solid.

MS (m/z) APCI AP⁺=410

### Example 189

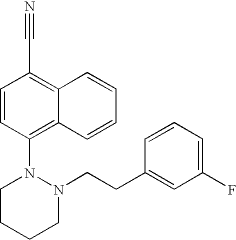

### 4-[2-[2-(3-fluorophenyl)ethyl]tetrahydro-1(2H)-pyridazinyl]-1-naphthalenecarbonitrile (C187)

From B1 and 2-(3-fluorophenyl)ethanol the title compound was isolated as an orange oil.

MS (m/z) APCI AP⁺=360

### Example 190

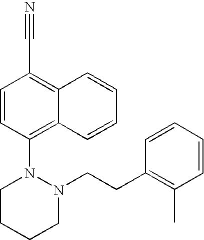

### 4-[2-[2-(2-methylphenyl)ethyl]tetrahydro-1(2H)-pyridazinyl]-1-naphthalenecarbonitrile (C188)

From B1 and 2-(2-methylphenyl)ethanol the title compound was isolated as an orange oil.

MS (m/z) APCI AP⁺=356

### Example 191

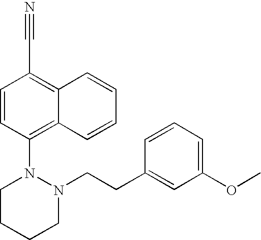

### 4-[2-{2-[3-(methyloxy)phenyl]ethyl}tetrahydro-1(2H)-pyridazinyl]-1-naphthalenecarbonitrile (C189)

From B1 and 2-[3-(methyloxy)phenyl]ethanol the title compound was isolated as an orange oil.

1H NMR (400 MHz, METHANOL-D4) δ ppm 1.8 (m, 4H), 2.4 (m, 2H), 3.2 (t, J=7.5 Hz, 2H), 3.5 (t, J=7.5 Hz, 2H), 3.6 (m, 3H), 6.2 (m, 1H), 6.3 (m, 1H), 6.5 (m, 1H), 6.9 (t, J=8.0 Hz, 1H), 7.1 (m, 1H), 7.4 (m, 1H), 7.6 (m, 1H), 7.8 (d, J=7.7 Hz, 1H), 8.0 (d, J=8.1 Hz, 1H), 8.3 (d, J=9.7 Hz, 1H)

### Example 192

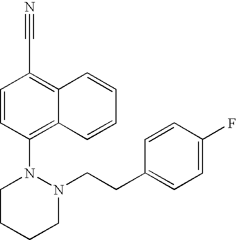

### 4-[2-[2-(4-fluorophenyl)ethyl]tetrahydro-1(2H)-pyridazinyl]-1-naphthalenecarbonitrile (C190)

From B1 and 2-(4-fluorophenyl)ethanol the title compound was isolated as an orange oil.

1H NMR (400 MHz, METHANOL-D4) δ ppm 1.8 (m, 4H), 2.4 (m, 2H), 3.1 (t, J=7.0 Hz, 2H), 3.3 (m, 2H), 3.4 (t, J=7.0 Hz, 2H), 6.6 (m, 2H), 6.7 (m, 2H), 7.0 (m, 1H), 7.4 (m, 1H), 7.6 (t, J=7.2 Hz, 1H), 7.8 (d, J=7.9 Hz, 1H), 8.0 (d, J=8.1 Hz, 1H), 8.2 (d, J=8.4 Hz, 1H)

### Example 193

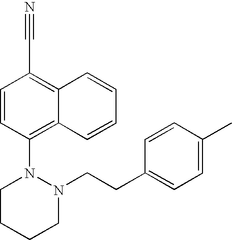

### 4-[2-[2-(4-methylphenyl)ethyl]tetrahydro-1(2H)-pyridazinyl]-1-naphthalenecarbonitrile (C191)

From B1 and 2-(4-methylphenyl)ethanol the title compound was isolated as an orange oil.

1H NMR (400 MHz, METHANOL-D4) δ ppm 1.8 (m, 4H), 2.2 (m, 3H), 2.4 (m, 2H), 3.1 (t, J=7.0 Hz, 2H), 3.3 (m, 2H), 3.4 (t, J=7.1 Hz, 2H), 6.6 (d, J=7.9 Hz, 2H), 6.7 (d, J=7.5 Hz, 2H), 7.0 (m, 1H), 7.4 (m, 1H), 7.6 (t, J=7.5 Hz, 1H), 7.8 (d, J=8.6 Hz, 1H), 8.0 (d, J=9.5 Hz, 1H), 8.2 (d, J=8.8 Hz, 1H)

### Example 194

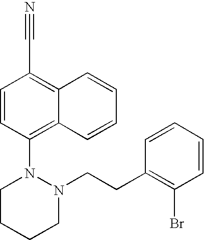

### 4-[2-[2-(2-bromophenyl)ethyl]tetrahydro-1(2H)-pyridazinyl]-1-naphthalenecarbonitrile (C192)

From B1 and 2-(2-bromophenyl)ethanol the title compound was isolated as a yellow oil.

1H NMR (400 MHz, METHANOL-D4) δ ppm 1.8 (m, 4H) 2.5 (t, J=7.3 Hz, 2H) 3.1 (t, J=7.2 Hz, 2H) 3.4 (m, 2H) 3.4 (m, 2H) 6.8 (m, 2H) 6.9 (m, 1H) 7.0 (d, J=7.9 Hz, 1H) 7.2 (m, 1H) 7.4 (m, 1H) 7.6 (m, 1H) 7.8 (d, J=7.9 Hz, 1H) 8.0 (d, J=8.4 Hz, 1H) 8.3 (d, J=8.8 Hz, 1H)

### Example 195

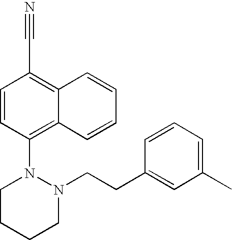

### 4-[2-[2-(3-methylphenyl)ethyl]tetrahydro-1(2H)-pyridazinyl]-1-naphthalenecarbonitrile (C193)

From B1 and 2-(3-methylphenyl)ethanol the title compound was isolated as an orange oil.

1H NMR (400 MHz, METHANOL-D4) δ ppm 1.8 (m, 4H), 2.1 (m, 3H), 2.4 (m, 2H), 3.1 (t, J=7.3 Hz, 2H), 3.4 (m, 2H), 3.5 (t, J=7.3 Hz, 2H), 6.5 (m, 1H), 6.6 (m, 1H), 6.7 (m, 1H), 6.8 (t, J=7.4 Hz, 1H), 7.1 (m, 1H), 7.4 (m, 1H), 7.6 (m, 1H), 7.8 (d, J=6.6 Hz, 1H), 8.0 (d, J=8.4 Hz, 1H), 8.3 (d, J=9.0 Hz, 1H)

### Example 196

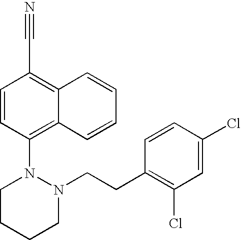

### 4-[2-[2-(2,4-dichlorophenyl)ethyl]tetrahydro-1(2H)-pyridazinyl]-1-naphthalenecarbonitrile (C194)

From B1 and 2-(2,4-dichlorophenyl)ethanol the title compound was isolated as a yellow oil.

1H NMR (400 MHz, METHANOL-D4) δ ppm 1.8 (m, 4H), 2.5 (t, J=6.6 Hz, 2H), 3.1 (t, J=6.5 Hz, 2H), 3.4 (m, 4H), 6.7 (m, 2H), 6.8 (d, J=1.8 Hz, 1H), 6.9 (m, J=8.1 Hz, 1H), 7.3 (m, 1H), 7.6 (m, 1H), 7.7 (d, J=7.9 Hz, 1H), 8.0 (d, J=8.2 Hz, 1H), 8.1 (d, J=8.2 Hz, 1H)

### Example 197

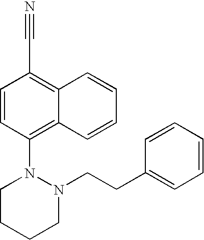

### 4-[2-(2-phenylethyl)tetrahydro-1(2H-pyridazinyl]-1-naphthalenecarbonitrile (C195)

From B1 and 2-(phenyl)ethanol the title compound was isolated as an orange oil.

1H NMR (400 MHz, METHANOL-D4) δ ppm 1.8 (m, 4H) 2.5 (m, 2H) 3.2 (t, J=7.3 Hz, 2H) 3.4 (m, 2H) 3.5 (m, 2H) 6.8 (m, 2H) 7.0 (m, 3H) 7.1 (m, 1H) 7.5 (m, 1H) 7.6 (m, 1H) 7.8 (d, J=8.4 Hz, 1H) 8.0 (d, J=8.6 Hz, 1H) 8.3 (d, J=8.2 Hz, 1H)

### Example 198

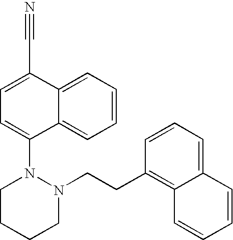

### 4-[2-[2-(1-naphthalenyl)ethyl]tetrahydro-1(2H-pyridazinyl]-1-naphthalenecarbonitrile (C196)

From B1 and 2-(1-naphthalenyl)ethanol the title compound was isolated as a red oil.

MS (m/z) APCI AP⁺=392

### Example 199

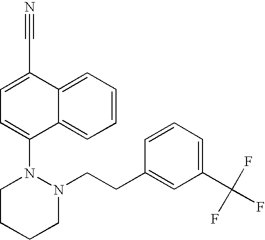

### 4-[2-{2-[3-(trifluoromethyl)phenyl]ethyl}tetrahydro-1(2H-pyridazinyl]-1-naphthalenecarbonitrile (C197)

From B1 and 2-[3-(trifluoromethyl)phenyl]ethanol the title compound was isolated as a red oil.

MS (m/z) APCI AP⁺=410

### Example 200

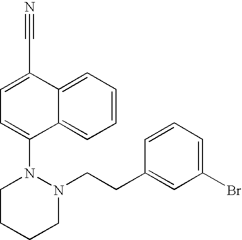

### 4-[2-[2-(3-bromophenyl)ethyl]tetrahydro-1(2H)-pyridazinyl]-1-naphthalenecarbonitrile (C198)

From B1 and 2-(3-bromophenyl)ethanol the title compound was isolated as an orange oil.

MS (m/z) APCI AP⁺=421

### Example 201

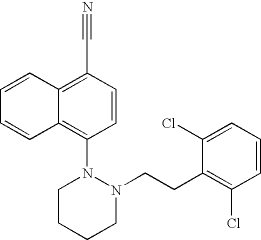

### 4-[2-[2-(2,6-dichlorophenyl)ethyl]tetrahydro-1(2H)-pyridazinyl]-1-naphthalenecarbonitrile (C199)

From B1 and 2-(2,6-dichlorophenyl)ethanol the title compound was isolated as a yellow oil.

MS (m/z) APCI AP⁺=410

### Example 202

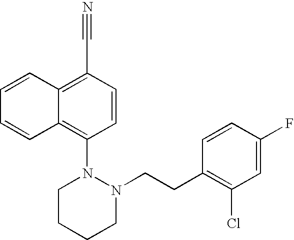

### 4-[2-[2-(2-chloro-4-fluorophenyl)ethyl]tetrahydro-1(2H)-pyridazinyl]-1-naphthalenecarbonitrile (C200)

From B1 and 2-(2-chloro-4-fluorophenyl)ethanol the title compound was isolated as a red oil.

MS (m/z) APCI AP⁺=394

### Example 203

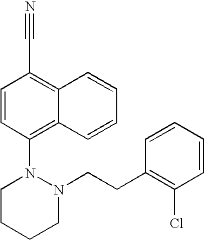

### 4-[2-[2-(2-chlorophenyl)ethyl]tetrahydro-1(2H)-pyridazinyl]-1-naphthalenecarbonitrile (C201)

From B1 and 2-(2-chlorophenyl)ethanol the title compound was isolated as a red oil.

MS (m/z) APCI AP⁺=376

### Example 204

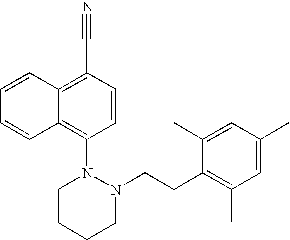

### 4-[2-[2-(2,4,6-trimethylphenyl)ethyl]tetrahydro-1(2H)-pyridazinyl]-1-naphthalenecarbonitrile (C202)

From B1 and 2-(2,4,6-trimethylphenyl)ethanol the title compound was isolated as an orange oil.

MS (m/z) APCI AP⁺=384

### Example 205

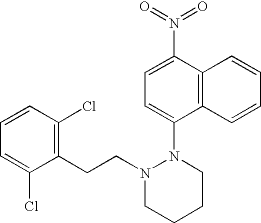

### 1-[2-(2,6-dichlorophenyl)ethyl]-2-(4-nitro-1-naphthalenyl)hexahydropyridazine (C203)

From B2 and 2-(2,6-Dichlorophenyl)Ethanol the Title Compound was Isolated as a Red Oil.

MS (m/z) APCI AP⁺=430

### Example 206

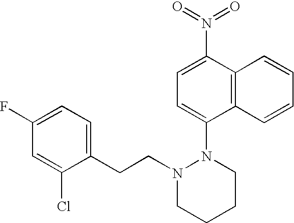

### 1-[2-(2-chloro-4-fluorophenyl)ethyl]-2-(4-nitro-1-naphthalenyl)hexahydropyridazine (C204)

From B2 and 2-(2-chloro-4-fluorophenyl)ethanol the title compound was isolated as a red oil.

MS (m/z) APCI AP⁺=414

### Example 207

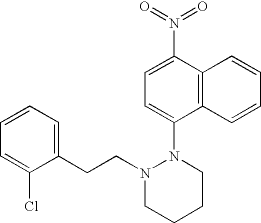

### 1-[2-(2-chlorophenyl)ethyl]-2-(4-nitro-1-naphthalenyl)hexahydropyridazine (C205)

From B2 and 2-(2-chlorophenyl)ethanol the title compound was isolated as a red oil.

MS (m/z) APCI AP⁺=396

### Example 208

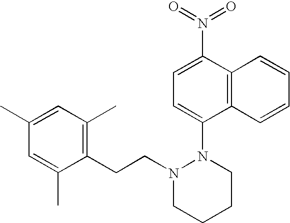

### 1-(4-nitro-1-naphthalenyl)-2-[2-(2,4,6-trimethylphenyl)ethyl]hexahydropyridazine (C206)

From B2 and 2-(2,4,6-trimethylphenyl)ethanol the title compound was isolated as a red oil.

MS (m/z) APCI AP⁺=404

### Example 209

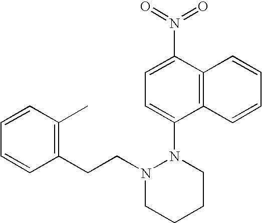

### 1-[2-(2-methylphenyl)ethyl]-2-(4-nitro-1-naphthalenyl)hexahydropyridazine (C207)

From B2 and 2-(2-methylphenyl)ethanol the title compound was isolated as a red oil.

MS (m/z) APCI AP⁺=376

### Example 210

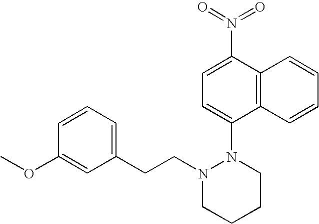

### 1-{2-[3-(methyloxy)phenyl]ethyl}-2-(4-nitro-1-naphthalenyl)hexahydropyridazine (C208)

From B2 and 2-[3-(methyloxy)phenyl]ethanol the title compound was isolated as a red oil.

MS (m/z) APCI AP⁺=392

### Example 211

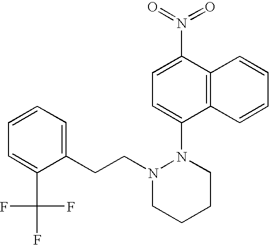

### 1-(4-nitro-1-naphthalenyl)-2-{2-[2-(trifluoromethyl)phenyl]ethyl}hexahydropyridazine (C209)

From B2 and 2-[2-(trifluoromethyl)phenyl]ethanol the title compound was isolated as a red oil.

MS (m/z) APCI AP⁺=430

### Example 212

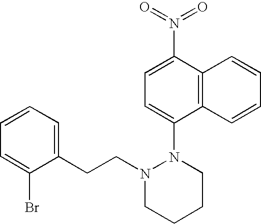

### 1-[2-(2-bromophenyl)ethyl]-2-(4-nitro-1-naphthalenyl)hexahydropyridazine (C210)

From B2 and 2-(2-bromophenyl)ethanol the title compound was isolated as a red oil.

MS (m/z) APCI AP⁺=441

### Example 213

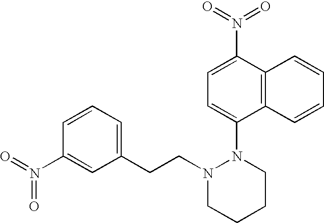

### 1-(4-nitro-1-naphthalenyl)-2-[2-(3-nitrophenyl)ethyl]hexahydropyridazine (C211)

From B2 and 2-(3-nitrophenyl)ethanol the title compound was isolated as a red oil MS (m/z) APCI AP⁺=407

### Example 214

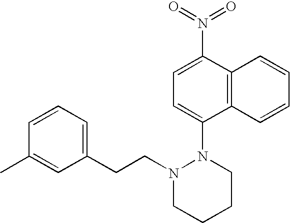

### 1-[2-(3-methylphenyl)ethyl]-2-(4-nitro-1-naphthalenyl)hexahydropyridazine (C212)

From B2 and 2-(3-methylphenyl)ethanol the title compound was isolated as a red oil.

MS (m/z) APCI AP⁺=376

### Example 215

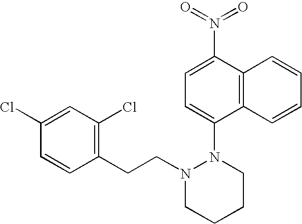

### 1-[2-(2,4-dichlorophenyl)ethyl]-2-(4-nitro-1-naphthalenyl)hexahydropyridazine (C213)

From B2 and 2-(2,4-dichlorophenyl)ethanol the title compound was isolated as a red oil.

MS (m/z) APCI AP⁺=430

### Example 216

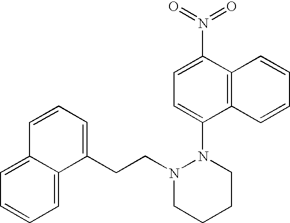

### 1-[2-(1-naphthalenyl)ethyl]-2-(4-nitro-1-naphthalenyl)hexahydropyridazine (C214)

From B2 and 2-(1-naphthalenyl)ethanol the title compound was isolated as an orange solid.

MS (m/z) APCI AP⁺=412

### Example 217

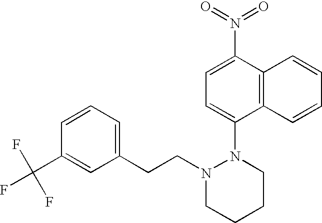

### 1-(4-nitro-1-naphthalenyl)-2-{2-[3-(trifluoromethyl)phenyl]ethyl}hexahydropyridazine (C215)

From B2 and 2-[3-(trifluoromethyl)phenyl]ethanol the title compound was isolated as an orange oil.

MS (m/z) APCI AP⁺=430

### Example 218

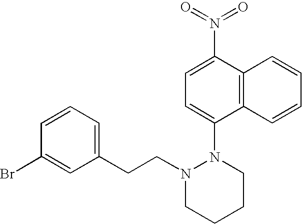

### 1-[2-(3-bromophenyl)ethyl]-2-(4-nitro-1-naphthalenyl)hexahydropyridazine (C216)

From B2 and 2-(3-bromophenyl)ethanol the title compound was isolated as an orange oil.

MS (m/z) APCI AP⁺=441

### Example 219

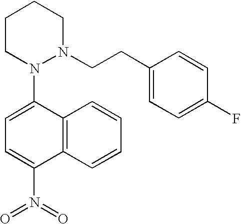

### 1-[2-(4-fluorophenyl)ethyl]-2-(4-nitro-1-naphthalenyl)hexahydropyridazine (C217)

From B2 and 2-(4-fluorophenyl)ethanol the title compound was obtained.

1H NMR (400 MHz, CHLOROFORM-D) 8 ppm 2.0 (m, 4H) 2.8 (m, 2H) 3.2 (m, 2H) 3.6 (m, 4H) 6.6 (dd, J=8.4, 5.3 Hz, 2H) 6.8 (m, 2H) 7.7 (m, 1H) 7.8 (m, 1H) 7.9 (m, 1H) 8.4 (d, J=8.4 Hz, 1H) 8.6 (d, J=8.6 Hz, 1H)

### Example 220

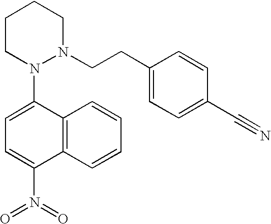

### 4-{2-[2-(4-nitro-1-naphthalenyl)tetrahydro-1(2H)-pyridazinyl]ethyl}benzonitrile (C218)

From B2 and 2-(4-cyanophenyl)ethanol the title compound was obtained.

1H NMR (400 MHz, CHLOROFORM-D) δ ppm 2.1 (m, 4H) 2.9 (m, 2H) 3.3 (m, 2H) 3.5 (m, 2H) 3.7 (m, 2H) 6.8 (d, J=8.3 Hz, 2H) 7.3 (d, J=8.1 Hz, 2H) 7.6 (t, J=7.6 Hz, 1H) 7.8 (m, 1H) 7.9 (m, 1H) 8.3 (d, J=8.3 Hz, 1H) 8.3 (d, J=8.4 Hz, 1H) 8.6 (d, J=8.6 Hz, 1H)

### Example 221

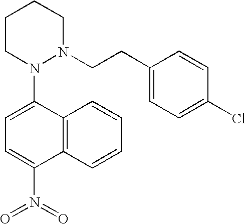

### 1-[2-(4-chlorophenyl)ethyl]-2-(4-nitro-1-naphthalenyl)hexahydropyridazine (C219)

From B2 and 2-(4-chlorophenyl)ethanol the title compound was obtained.

1H NMR (400 MHz, CHLOROFORM-D) δ ppm 2.0 (m, 4H) 2.7 (m, 2H) 3.2 (m, 2H) 3.6 (m, 4H) 6.6 (d, J=8.3 Hz, 2H) 7.0 (m, 2H) 7.6 (m, 1H) 7.7 (m, 2H) 8.3 (m, 2H) 8.6 (d, J=8.8 Hz, 1H)

### Example 222

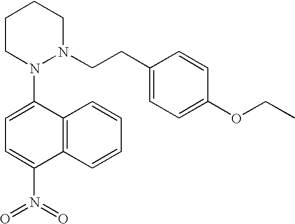

### 1-{2-[4-(ethyloxy)phenyl]ethyl}-2-(4-nitro-1-naphthalenyl)hexahydropyridazine (C220)

From B2 and 2-[4-(ethyloxy)phenyl]ethanol the title compound was obtained.

1H NMR (400 MHz, CHLOROFORM-D) δ ppm 1.4 (t, J=7.0 Hz, 3H) 1.8 (m, 4H) 2.5 (m, 2H) 3.1 (m, 2H) 3.3 (m, 2H) 3.6 (m, 2H) 3.9 (q, J=7.1 Hz, 2H) 6.6 (m, 2H) 6.8 (m, 2H) 7.0 (d, J=8.8 Hz, 1H) 7.4 (m, 1H) 7.6 (m, 1H) 8.2 (dd, J=8.7, 0.8 Hz, 1H) 8.3 (d, J=8.8 Hz, 1H) 8.8 (d, J=8.3 Hz, 1H)

### Example 223

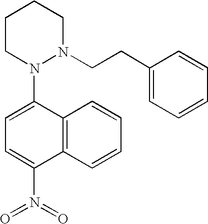

### 1-(4-nitro-1-naphthalenyl)-2-(2-phenylethyl)hexahydropyridazine (C221)

From B2 and 2-(phenyl)ethanol the title compound was obtained.

1H NMR (300 MHz, CHLOROFORM-D) δ ppm 1.8 (m, 4H) 2.6 (m, 2H) 3.2 (m, 2H)

3.3 (m, 2H) 3.6 (m, 2H) 6.9 (m, 2H) 7.0 (d, J=8.8 Hz, 1H) 7.1 (m, 3H) 7.5 (m, 1H) 7.7 (m, 1H) 8.2 (d, J=8.0 Hz, 1H) 8.3 (d, J=8.6 Hz, 1H) 8.8 (d, J=8.3 Hz, 1H)

### Cyclic Hydrazine Acylations

### Example 224

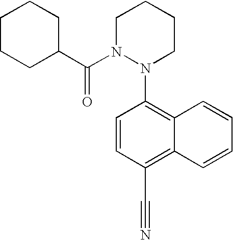

### D. 4-[2-(cyclohexylcarbonyl)tetrahydro-1(2H)-pyridazinyl]-1-naphthalenecarbonitrile (D1)

To a 40 ml scintillation vial was added 73 mg 4-(tetrahydro-1(2H)-pyridazinyl)-1-naphthalenecarbonitrile (B1) (0.31 mmoles, 1 eq) and 2 ml dichloromethane. Then 0.055 ml cyclohexanecarbonyl chloride (0.339 mmoles, 1.1 eq) was added followed by 0.064 ml triethylamine (0.462 mmoles, 1.5 eq). The reaction was shaken on an orbital shaker overnight. The reaction mixture was concentrated in vacuo and the residue was purified via preparative HPLC (Phenomenex column Luna C18, 75 mm×30 mm, 5 micron), 30% acetonitrile/water (0.01% TFA) to 100% acetonitrile, to yield 25 mg of the title compound as a yellow solid.

MS (m/z) ESI ES⁺=348

The following compounds were synthesized according to a similar general procedure as shown for D1:

### Example 225

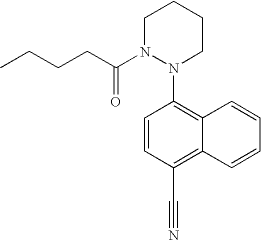

### 4-(2-pentanoyltetrahydro-1(2H)-pyridazinyl)-1-naphthalenecarbonitrile (D2)

From B1 and pentanoyl chloride the title compound was isolated as an orange oil.

MS (m/z) ESI ES⁺=322

### Example 226

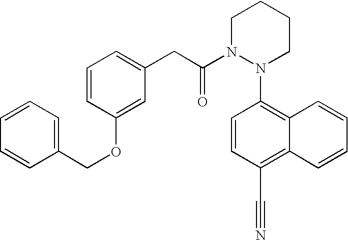

### 4-[2-({3-[(phenylmethyl)oxy]phenyl}acetyl)tetrahydro-1(2H)-pyridazinyl]-1-naphthalenecarbonitrile (D3)

From B1 and {3-[(phenylmethyl)oxy]phenyl}acetyl chloride the title compound was isolated as a yellow oil.

MS (m/z) ESI ES⁺=462

### Example 227

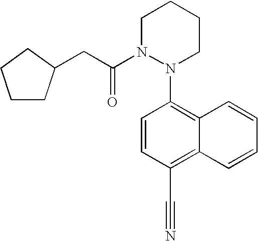

### 4-[2-(cyclopentylacetyl)tetrahydro-1(2H)-pyridazinyl]-1-naphthalenecarbonitrile (D4)

From B1 and cyclopentylacetyl chloride the title compound was isolated as an orange oil.

MS (m/z) ESI ES⁺=348

### Example 228

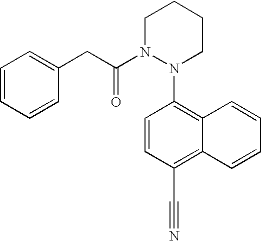

### 4-[2-(phenylacetyl)tetrahydro-1(2H)-pyridazinyl]-1-naphthalenecarbonitrile (D5)

From B1 and phenylacetyl chloride the title compound was isolated as an orange oil.

MS (m/z) ESI ES⁺=356

### Example 229

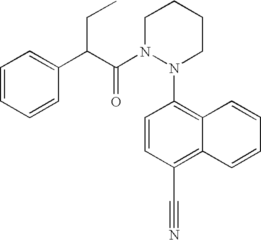

### 4-[2-(2-phenylbutanoyl)tetrahydro-1(2H)-pyridazinyl]-1-naphthalenecarbonitrile (D6)

From B1 and 2-phenylbutanoyl chloride the title compound was isolated as an orange oil.

MS (m/z) ESI ES⁺=384

### Example 230

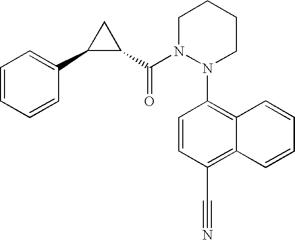

### 4-[2-{[(1S,2S)-2-phenylcyclopropyl]carbonyl}tetrahydro-1(2H)-pyridazinyl]-1-naphthalenecarbonitrile (D8)

From B1 and (1S,2S)-2-phenylcyclopropylcarbonyl chloride the title compound was isolated as an orange oil.

MS (m/z) ESI ES⁺=382

### Example 231

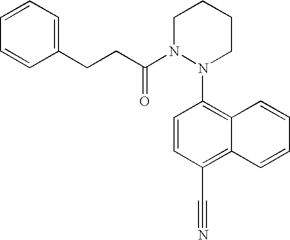

### 4-[2-(3-phenylpropanoyl)tetrahydro-1(2H-pyridazinyl]-1-naphthalenecarbonitrile (D9)

From B1 and phenylpropanoyl chloride the title compound was isolated as a yellow solid.

MS (m/z) ESI ES⁺=370

### Example 232

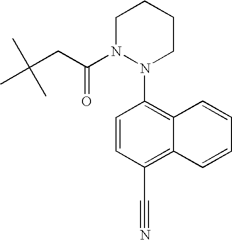

### 4-[2-(3,3-dimethylbutanoyl)tetrahydro-1(2H)-pyridazinyl]-1-naphthalenecarbonitrile (D10)

From B1 and 2-3,3-dimethylbutanoyl chloride the title compound was isolated as a white solid.

MS (m/z) ESI ES⁺=336

### Example 233

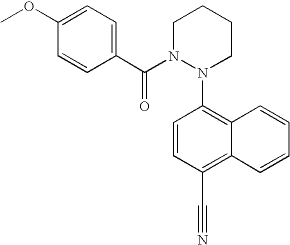

### 4-[2-{[4-(methyloxy)phenyl]carbonyl}tetrahydro-1(2H)-pyridazinyl]-1-naphthalenecarbonitrile (D1)

From B1 and 4-(methyloxy)benzoyl chloride the title compound was isolated as a white solid.

MS (m/z) APCI AP⁺=372

### Example 234

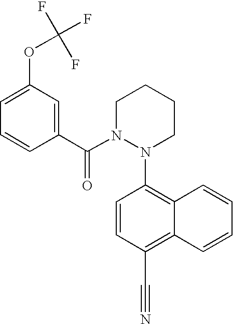

### 4-[2-({3-[(trifluoromethyl)oxy]phenyl}carbonyl)tetrahydro-1(2H)-pyridazinyl]-1-naphthalenecarbonitrile (D12)

From B1 and 3-[(trifluoromethyl)oxy]benzoyl chloride the title compound was isolated as a white solid.

MS (m/z) APCI AP⁺=426

### Example 235

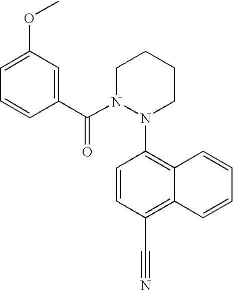

### 4-[2-{[3-(methyloxy)phenyl]carbonyl}tetrahydro-1(2H)-pyridazinyl]-1-naphthalenecarbonitrile (D13)

From B1 and 3-(methyloxy)benzoyl chloride the title compound was isolated as a white solid.

MS (m/z) APCI AP⁺=372

### Example 236

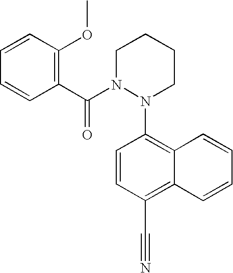

### 4-[2-{[2-(methyloxy)phenyl]carbonyl}tetrahydro-1(2H)-pyridazinyl]-1-naphthalenecarbonitrile (D14)

From B1 and 2-(methyloxy)benzoyl chloride the title compound was isolated as a white solid.

MS (m/z) APCI AP⁺=372

### Example 237

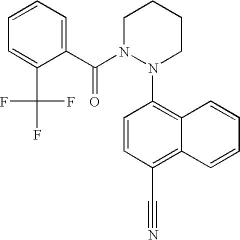

### 4-[2-{[2-(trifluoromethyl)phenyl]carbonyl}tetrahydro-1(2H)-pyridazinyl]-1-naphthalenecarbonitrile (D15)

From B1 and 2-(trifluoromethyl)benzoyl chloride the title compound was isolated as a white solid.

MS (m/z) APCI AP⁺=410

### Example 238

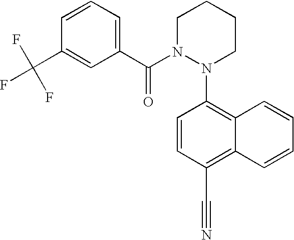

### 4-[2-{[3-(trifluoromethyl)phenyl]carbonyl}tetrahydro-1(2H)-pyridazinyl]-1-naphthalenecarbonitrile (D16)

From B1 and 3-(trifluoromethyl)benzoyl chloride the title compound was isolated as a white solid.

MS (m/z) APCI AP⁺=410

### Example 239

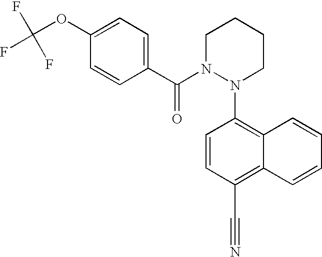

### 4-[2-({4-[(trifluoromethyl)oxy]phenyl}carbonyl)tetrahydro-1(2H)-pyridazinyl]-1-naphthalenecarbonitrile (D17)

From B1 and 4-[(trifluoromethyl)oxy]benzoyl chloride the title compound was isolated as a white solid.

MS (m/z) APCI AP⁺=426

### Example 240

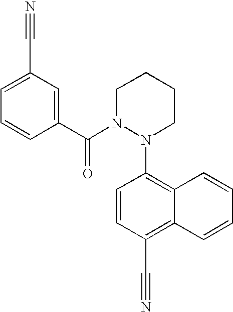

### 4-[2-[(3-cyanophenyl)carbonyl]tetrahydro-1(2H)-pyridazinyl]-1-naphthalenecarbonitrile (D18)

From B1 and 3-cyanobenzoyl chloride the title compound was isolated as a white solid.

MS (m/z) APCI AP⁺=367

### Example 241

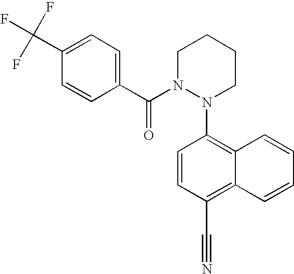

### 4-[2-{[4-(trifluoromethyl)phenyl]carbonyl}tetrahydro-1(2H)-pyridazinyl]-1-naphthalenecarbonitrile (D19)

From B1 and 4-(trifluoromethyl)benzoyl chloride the title compound was isolated as a white solid.

MS (m/z) APCI AP⁺=410

### Example 242

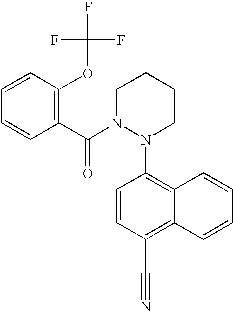

### 4-[2-({2-[(trifluoromethyl)oxy]phenyl}carbonyl)tetrahydro-1(2H)-pyridazinyl]-1-naphthalenecarbonitrile (D20)

From B1 and 2-[(trifluoromethyl)oxy]benzoyl chloride the title compound was isolated as a white solid.

MS (m/z) APCI AP⁺=426

### Example 243

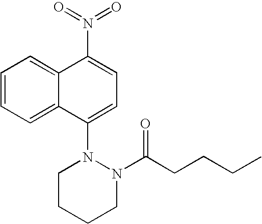

### 1-(4-nitro-1-naphthalenyl)-2-pentanoylhexahydropyridazine (D21)

From B2 and pentanoyl chloride the title compound was isolated as a yellow solid.

MS (m/z) APCI AP⁺=342

### Example 244

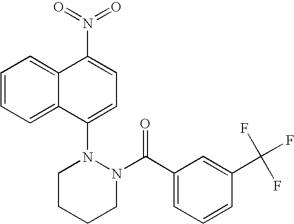

### 1-(4-nitro-1-naphthalenyl)-2-{[3-(trifluoromethyl)phenyl]carbonyl}hexahydropyridazine (D22)

From B2 and 3-(trifluoromethyl)benzoyl chloride the title compound was isolated as a yellow solid.

MS (m/z) APCI AP⁺=430

### Example 245

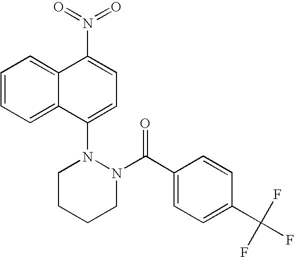

### 1-(4-nitro-1-naphthalenyl)-2-{[4-(trifluoromethyl)phenyl]carbonyl}hexahydropyridazine (D23)

From B2 and 4-(trifluoromethyl)benzoyl chloride the title compound was isolated as a yellow solid.

MS (m/z) APCI AP⁺=430

### Example 246

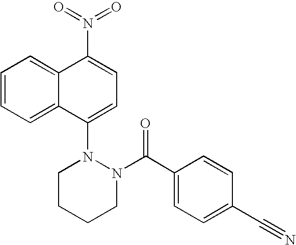

### 4-{[2-(4-nitro-1-naphthalenyl)tetrahydro-1(2H-pyridazinyl]carbonyl}benzonitrile (D24)

From B2 and 4-cyanobenzoyl chloride the title compound was isolated as a yellow solid.

MS (m/z) APCI AP⁺=387

### Example 247

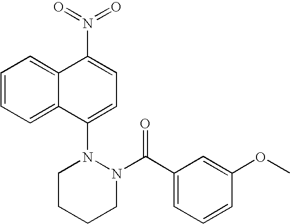

### 1-{[3-(methyloxy)phenyl]carbonyl}-2-(4-nitro-1-naphthalenyl)hexahydropyridazine (D25)

From B2 and 3-(methyloxy)benzoyl chloride the title compound was isolated as a yellow solid.

MS (m/z) APCI AP⁺=392

### Example 248

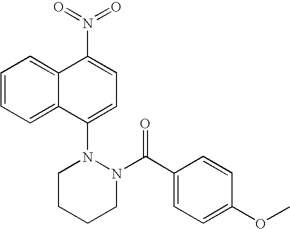

### 1-{[4-(methyloxy)phenyl]carbonyl}-2-(4-nitro-1-naphthalenyl)hexahydropyridazine (D26)

From B2 and 4-(methyloxy)benzoyl chloride the title compound was isolated as a yellow solid.

MS (m/z) APCI AP⁺=392

### Example 249

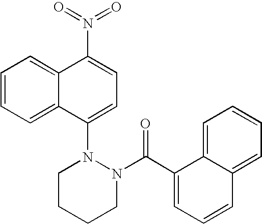

### 1-(1-naphthalenylcarbonyl)-2-(4-nitro-1-naphthalenyl)hexahydropyridazine (D27)

From B2 and 1-naphthalenecarbonyl chloride the title compound was isolated as a green solid.

MS (m/z) APCI AP⁺=412

### Example 250

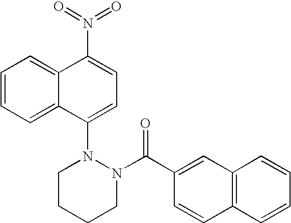

### 1-(2-naphthalenylcarbonyl)-2-(4-nitro-1-naphthalenyl)hexahydropyridazine (D28)

From B2 and 2-naphthalenecarbonyl chloride the title compound was isolated as a yellow solid.

MS (m/z) APCI AP⁺=412

### Example 251

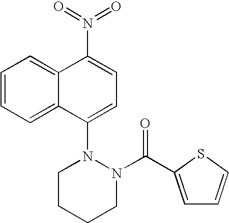

### 1-(4-nitro-1-naphthalenyl)-2-(2-thienylcarbonyl)hexahydropyridazine (D29)

From B2 and 2-thiophenecarbonyl chloride the title compound was isolated as a yellow solid.

MS (m/z) APCI AP⁺=368

### Example 252

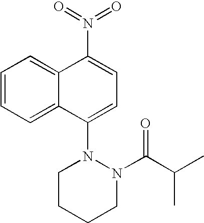

### 1-(2-methylpropanoyl)-2-(4-nitro-1-naphthalenyl)hexahydropyridazine (D30)

From B2 and isobutyryl chloride the title compound was isolated as a yellow solid.

MS (m/z) APCI AP⁺=328

### Example 253

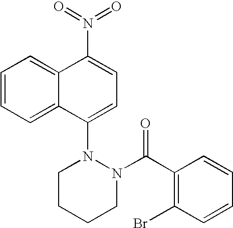

### 1-[(2-bromophenyl)carbonyl]-2-(4-nitro-1-naphthalenyl)hexahydropyridazine (D31)

From B2 and 2-bromobenzoyl chloride the title compound was isolated as a green solid.

MS (m/z) APCI AP⁺=441

### Example 254

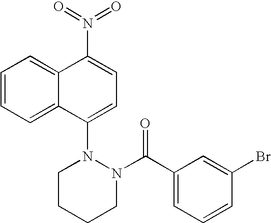

### 1-[(3-bromophenyl)carbonyl]-2-(4-nitro-1-naphthalenyl)hexahydropyridazine (D32)

From B2 and 3-bromobenzoyl chloride the title compound was isolated as a yellow solid.

MS (m/z) APCI AP⁺=441

### Example 255

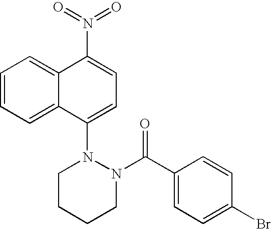

### 1-[(4-bromophenyl)carbonyl]-2-(4-nitro-1-naphthalenyl)hexahydropyridazine (D33)

From B2 and 4-bromobenzoyl chloride the title compound was isolated as a yellow solid.

MS (m/z) APCI AP⁺=441

### Example 256

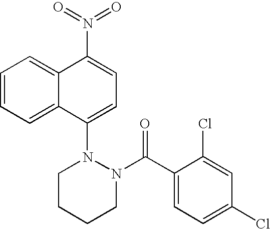

### 1-[(2,4-dichlorophenyl)carbonyl]-2-(4-nitro-1-naphthalenyl)hexahydropyridazine (D34)

From B2 and 2,4-dichlorobenzoyl chloride the title compound was isolated as a yellow solid.

MS (m/z) APCI AP⁺=430

### Example 257

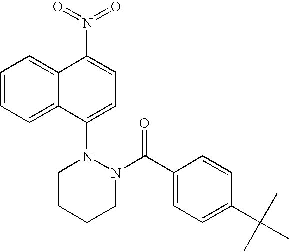

### 1-{[4-(1,1-dimethylethyl)phenyl]carbonyl}-2-(4-nitro-1-naphthalenyl)hexahydropyridazine (D35)

From B2 and 4-(1,1-dimethylethyl)benzoyl chloride the title compound was isolated as a yellow solid.

MS (m/z) APCI AP⁺=418

### Example 258

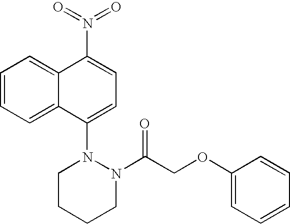

### 1-(4-nitro-1-naphthalenyl)-2-[(phenyloxy)acetyl]hexahydropyridazine (D36)

From B2 and (phenyloxy)acetyl chloride the title compound was isolated as a yellow solid.

MS (m/z) APCI AP⁺=392

### Example 259

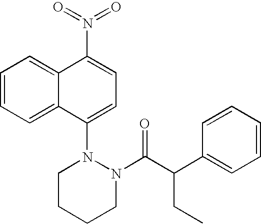

### 1-(4-nitro-1-naphthalenyl)-2-(2-phenylbutanoyl)hexahydropyridazine (D37)

From B2 and 2-phenylbutanoyl chloride the title compound was isolated as a yellow solid.

MS (m/z) APCI AP⁺=404

### Example 260

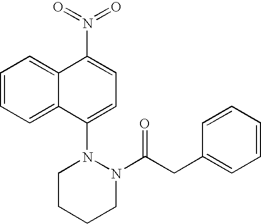

### 1-(4-nitro-1-naphthalenyl)-2-(phenylacetyl)hexahydropyridazine (D38)

From B2 and phenylacetyl chloride the title compound was isolated as a yellow solid.

MS (m/z) APCI AP⁺=376

### Example 261

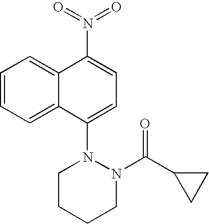

### 1-(cyclopropylcarbonyl)-2-(4-nitro-1-naphthalenyl)hexahydropyridazine (D39)

From B2 and cyclopropanecarbonyl chloride the title compound was isolated as an orange solid.

MS (m/z) APCI AP⁺=326

### Example 262

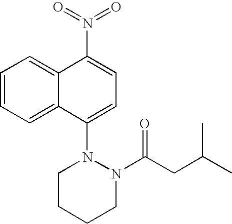

### 1-(3-methylbutanoyl)-2-(4-nitro-1-naphthalenyl)hexahydropyridazine (D40)

From B2 and 3-methylbutanoyl chloride the title compound was isolated as a yellow solid.

MS (m/z) APCI AP⁺=342

### Example 263

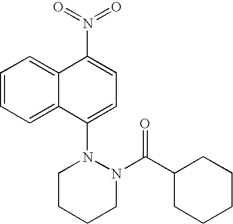

### 1-(cyclohexylcarbonyl)-2-(4-nitro-1-naphthalenyl)hexahydropyridazine (D41)

From B2 and cyclohexanecarbonyl chloride the title compound was isolated as a yellow solid.

MS (m/z) APCI AP⁺=368

### Example 264

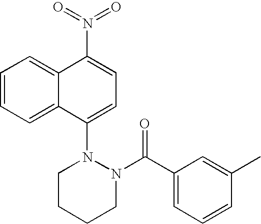

### 1-[(3-methylphenyl)carbonyl]-2-(4-nitro-1-naphthalenyl)hexahydropyridazine (D42)

From B2 and 3-methylbenzoyl chloride the title compound was isolated as a yellow solid.

MS (m/z) APCI AP⁺=376

### Example 265

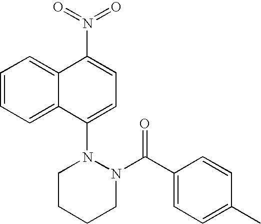

### 1-[(4-methylphenyl)carbonyl]-2-(4-nitro-1-naphthalenyl)hexahydropyridazine (D43)

From B2 and 4-methylbenzoyl chloride the title compound was isolated as a yellow solid.

MS (m/z) APCI AP⁺=376

### Example 266

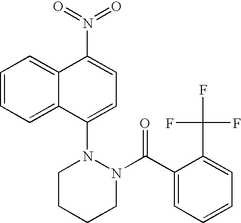

### 1-(4-nitro-1-naphthalenyl)-2-{[2-(trifluoromethyl)phenyl]carbonyl}hexahydropyridazine (D44)

From B2 and 2-(trifluoromethyl)benzoyl chloride the title compound was isolated as a green solid.

MS (m/z) APCI AP⁺=430

### Example 267

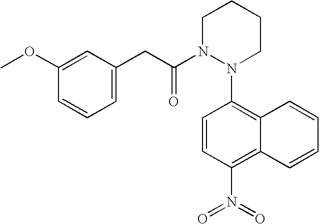

### 1-{[3-(methyloxy)phenyl]acetyl}-2-(4-nitro-1-naphthalenyl)hexahydropyridazine (D45)

From B2 and 3-(methyloxy)phenylacetyl chloride the title compound was isolated as a yellow solid.

MS (m/z) ES⁺=406

### Example 268

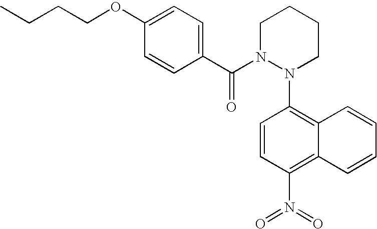

### 1-{[4-(butyloxy)phenyl]carbonyl}-2-(4-nitro-1-naphthalenyl)hexahydropyridazine (D46)

From B2 and 4-(butyloxy)benzoyl chloride the title compound was isolated as a yellow oil.

MS (m/z) ES⁺=434

### Example 269

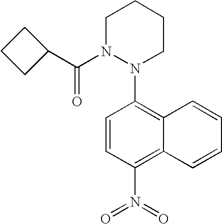

### 1-(cyclobutylcarbonyl)-2-(4-nitro-1-naphthalenyl)hexahydropyridazine (D47)

From B2 and cyclobutanecarbonyl chloride the title compound was isolated as an orange solid.

MS (m/z) ES⁺=340

### Example 270

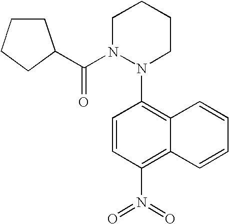

### 1-(cyclopentylcarbonyl)-2-(4-nitro-1-naphthalenyl)hexahydropyridazine (D48)

From B2 and cyclopentanecarbonyl chloride the title compound was isolated as a yellow solid.

MS (m/z) ES⁺=354

### Example 271

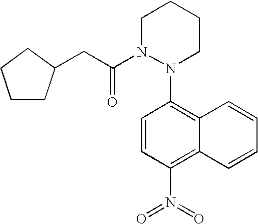

### 1-(cyclopentylacetyl)-2-(4-nitro-1-naphthalenyl)hexahydropyridazine (D49)

From B2 and cyclopentylacetyl chloride the title compound was isolated as a yellow solid.

MS (m/z) ES⁺=368

### Example 272

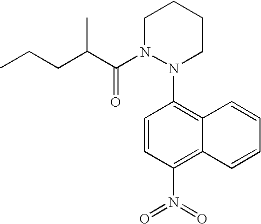

### 1-(2-methylpentanoyl)-2-(4-nitro-1-naphthalenyl)hexahydropyridazine (D50)

From B2 and 2-methylpentanoyl chloride the title compound was isolated as a yellow oil.

MS (m/z) ES⁺=356

### Example 273

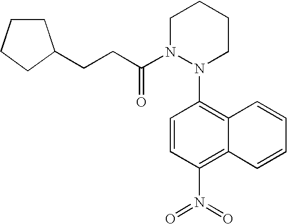

### 1-(3-cyclopentylpropanoyl)-2-(4-nitro-1-naphthalenyl)hexahydropyridazine (D51)

From B2 and cyclopentylpropanoyl chloride the title compound was isolated as a yellow oil.

MS (m/z) ES⁺=382

### Example 274

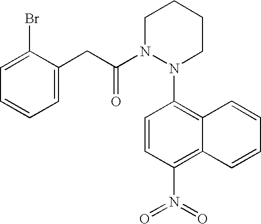

### 1-[(2-bromophenyl)acetyl]-2-(4-nitro-1-naphthalenyl)hexahydropyridazine (D52)

From B2 and 2-(bromophenyl)acetyl chloride the title compound was isolated as an orange oil.

MS (m/z) ES⁺=455

### Example 275

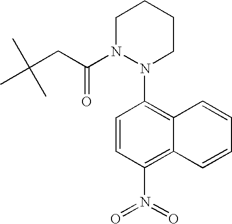

### 1-(3,3-dimethylbutanoyl)-2-(4-nitro-1-naphthalenyl)hexahydropyridazine (D53)

From B2 and 3,3-dimethylbutanoyl chloride the title compound was isolated as a yellow solid.

MS (m/z) ES⁺=356

### Example 276

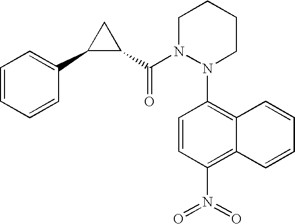

### 1-(4-nitro-1-naphthalenyl)-2-{[(1S,2S)-2-phenylcyclopropyl]carbonyl}hexahydropyridazine (D54)

From B2 and (1S,2S)-2-phenylcyclopropylcarbonyl chloride the title compound was isolated as an orange oil.

MS (m/z) ES⁺=402

### Example 277

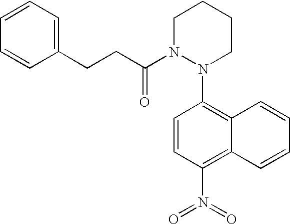

### 1-(4-nitro-1-naphthalenyl)-2-(3-phenylpropanoyl)hexahydropyridazine (D55)

From B2 and 3-phenylpropanoyl chloride the title compound was isolated as a yellow oil.

MS (m/z) ES⁺=390

### Example 278

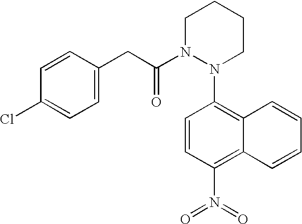

### 1-[(4-chlorophenyl)acetyl]-2-(4-nitro-1-naphthalenyl)hexahydropyridazine (D56)

From B2 and 4-(chlorophenyl)acetyl chloride the title compound was isolated as a yellow solid.

MS (m/z) ES⁺=410

### Example 279

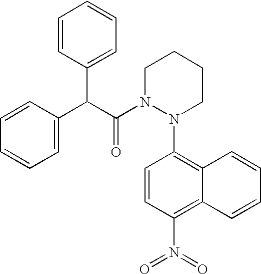

### 1-(diphenylacetyl)-2-(4-nitro-1-naphthalenyl)hexahydropyridazine (D57)

From B2 and diphenylacetyl chloride the title compound was isolated as a yellow solid.

MS (m/z) ES⁺=452

### Example 280

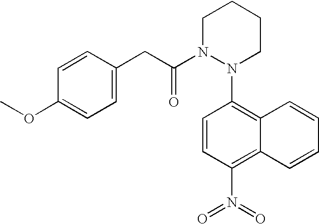

### 1-{[4-(methyloxy)phenyl]acetyl}-2-(4-nitro-1-naphthalenyl)hexahydropyridazine (D58)

From B2 and 4-(methyloxy)phenylacetyl chloride the title compound was isolated as a yellow solid.

MS (m/z) ES⁺=406

### Example 281

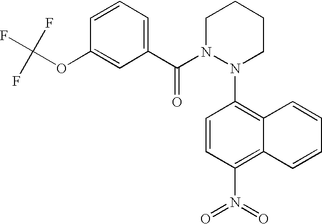

### 1-(4-nitro-1-naphthalenyl)-2-({3-[(trifluoromethyl)oxy]phenyl}carbonyl)hexahydropyridazine (D59)

From B2 and 3-[(trifluoromethyl)oxy]benzoyl chloride the title compound was isolated as a yellow solid.

MS (m/z) ES⁺=446

### Example 282

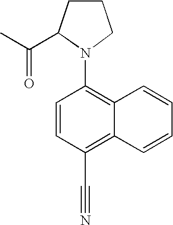

### 4-(2-acetyl-1-pyrazolidinyl)-1-naphthalenecarbonitrile (D60)

From B5 and acetic anhydride the title compound was isolated as a yellow solid.

MS (m/z) ESI ES⁺=266

### Example 283

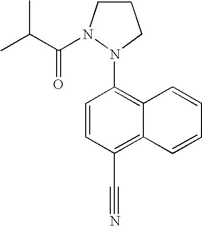

### 4-[2-(2-methylpropanoyl)-1-pyrazolidinyl]-1-naphthalenecarbonitrile (D61)

From B5 and isobutyryl chloride the title compound was isolated as a yellow solid.

MS (m/z) ESI ES⁺=294

### Example 284

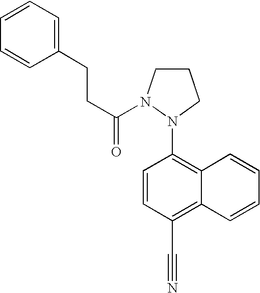

### 4-[2-(3-phenylpropanoyl)-1-pyrazolidinyl]-1-naphthalenecarbonitrile (D62)

From B5 and 3-phenylpropanoyl chloride the title compound was isolated as a yellow oil.

MS (m/z) ESI ES⁺=356

### Example 285

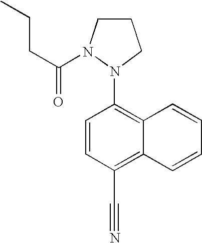

### 4-(2-butanoyl-1-pyrazolidinyl)-1-naphthalenecarbonitrile (D63)

From B5 and butanoyl chloride the title compound was isolated as a yellow solid.

MS (m/z) ESI ES⁺=294

### Example 286

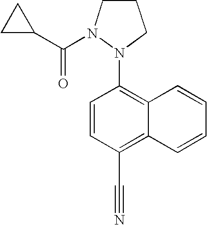

### 4-[2-(cyclopropylcarbonyl)-1-pyrazolidinyl]-1-naphthalenecarbonitrile (D64)

From B5 and cyclopropanecarbonyl chloride the title compound was isolated as a yellow solid.

MS (m/z) ESI ES⁺=292

### Example 287

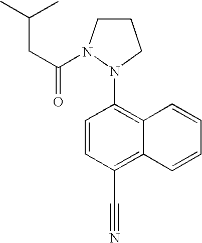

### 4-[2-(3-methylbutanoyl)-1-pyrazolidinyl]-1-naphthalenecarbonitrile (D65)

From B5 and 3-methylbutanoyl chloride the title compound was isolated as a yellow solid.

MS (m/z) ESI ES⁺=308

### Example 288

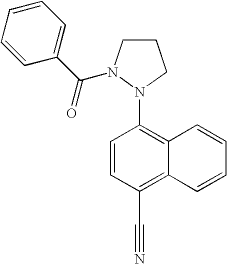

### 4-[2-(phenylcarbonyl)-1-pyrazolidinyl]-1-naphthalenecarbonitrile (D66)

From B5 and benzoyl chloride the title compound was isolated as a yellow solid.

MS (m/z) ESI ES⁺=328

### Example 289

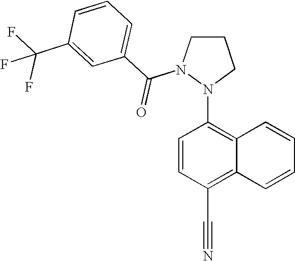

### 4-(2-{[3-(trifluoromethyl)phenyl]carbonyl}-1-pyrazolidinyl)-1-naphthalenecarbonitrile (D67)

From B5 and 3-(trifluoromethyl)benzoyl chloride the title compound was isolated as a yellow solid.

MS (m/z) ESI ES⁺=396

### Example 290

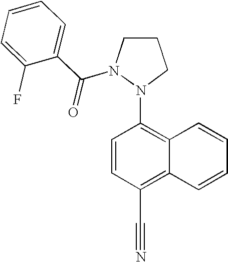

### 4-{2-[(2-fluorophenyl)carbonyl]-1-pyrazolidinyl}-1-naphthalenecarbonitrile (D68)

From B5 and 2-fluorobenzoyl chloride the title compound was isolated as a yellow solid.

MS (m/z) ESI ES⁺=346

### Example 291

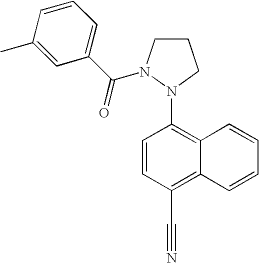

### 4-{2-[(3-methylphenyl)carbonyl]-1-pyrazolidinyl}-1-naphthalenecarbonitrile (D69)

From B5 and 3-methylbenzoyl chloride the title compound was isolated as a yellow solid.

MS (m/z) ESI ES⁺=342

### Example 292

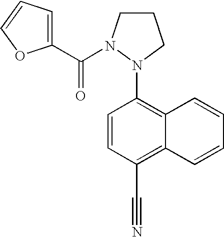

### 4-[2-(2-furanylcarbonyl)-1-pyrazolidinyl]-1-naphthalenecarbonitrile (D70)

From B5 and 2-furancarbonyl chloride the title compound was isolated as a yellow solid.

MS (m/z) ESI ES⁺=318

### Example 293

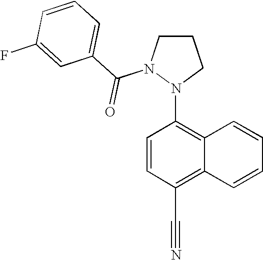

### 4-{2-[(3-fluorophenyl)carbonyl]-1-pyrazolidinyl}-1-naphthalenecarbonitrile (D71)

From B5 and 3-fluorobenzoyl chloride the title compound was isolated as a yellow solid.

MS (m/z) ESI ES⁺=346

### Example 294

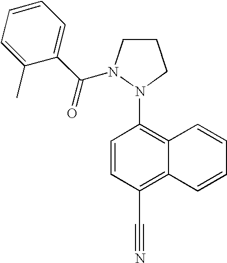

### 4-{2-[(2-methylphenyl)carbonyl]-1-pyrazolidinyl}-1-naphthalenecarbonitrile (D72)

From B5 and 2-methylbenzoyl chloride the title compound was isolated as a yellow solid.

MS (m/z) ESI ES⁺=342

### Example 295

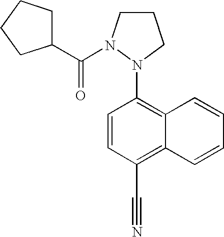

### 4-[2-(cyclopentylcarbonyl)-1-pyrazolidinyl]-1-naphthalenecarbonitrile (D73)

From B5 and cyclopentanecarbonyl chloride the title compound was isolated as a yellow solid.

MS (m/z) ESI ES⁺=320

### Example 296

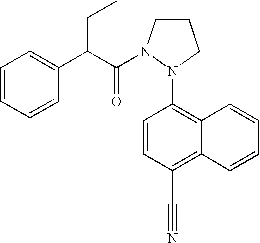

### 4-[2-(2-phenylbutanoyl)-1-pyrazolidinyl]-1-naphthalenecarbonitrile (D74)

From B5 and 2-phenylbutanoyl chloride the title compound was isolated as a white solid.

MS (m/z) ESI ES⁺=370

### Hexahydropyridazine HATU-mediated Amide Couplings

### Example 297

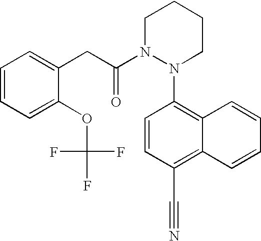

### 4-[2-({2-[(trifluoromethyl)oxy]phenyl}acetyl)tetrahydro-1(2H)-pyridazinyl]-1-naphthalenecarbonitrile (D75)

To a 40 ml scintillation vial was added 82 mg {2-[(trifluoromethyl)oxy]phenyl}acetic acid (0.371 mmoles, 1.1 eq), 2 ml dimethylformamide, and 0.065 ml diisopropylethyl amine (0.371 mmoles, 1.1 eq) followed by the addition of 141 mg O-(7-azabenzotriazol-1-yl)-N,N,N′,N′-tetramethyluronium hexafluorophosphate (HATU, 0.371 mmoles, 1.1 eq). The reaction mixture was shaken for 15 minutes on an orbital shaker at room temperature. Then 80 mg 4-(tetrahydro-1(2H)-pyridazinyl)-1-naphthalenecarbonitrile (B1) (0.337 mmoles, 1 eq) was added and the reaction was shaken on an orbital shaker overnight. The reaction mixture was partitioned between ethyl acetate and 1N NaOH and the organic fraction was concentrated in vacuo. The residue was purified via preparative HPLC (Phenomenex column Luna C18, 75 mm×30 mm, 5 micron), 30% acetonitrile/water (0.01% TFA) to 100% acetonitrile, to yield 66 mg of the title compound as a white solid.

MS (m/z) APCI AP⁺=440

The following compounds were synthesized according to a similar general procedure as described for D75:

### Example 298

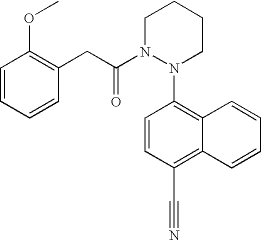

### 4-[2-{[2-(methyloxy)phenyl]acetyl}tetrahydro-1(2H-pyridazinyl]-1-naphthalenecarbonitrile (D76)

From B1 and [2-(methyloxy)phenyl]acetic acid the title compound was isolated as a white solid.

MS (m/z) APCI AP⁺=386

### Example 299

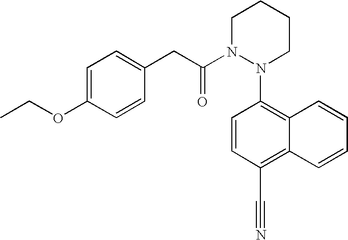

### 4-[2-{[4-(ethyloxy)phenyl]acetyl}tetrahydro-1(2H-pyridazinyl]-1-naphthalenecarbonitrile (D77)

From B1 and [4-(ethyloxy)phenyl]acetic acid the title compound was isolated as a yellow solid.

MS (m/z) APCI AP⁺=400

### Example 300

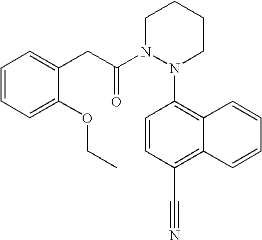

### 4-[2-{[2-(ethyloxy)phenyl]acetyl}tetrahydro-1(2H)-pyridazinyl]-1-naphthalenecarbonitrile (D78)

From B1 and [2-(ethyloxy)phenyl]acetic acid the title compound was isolated as a yellow oil.

MS (m/z) APCI AP⁺=400

### Example 301

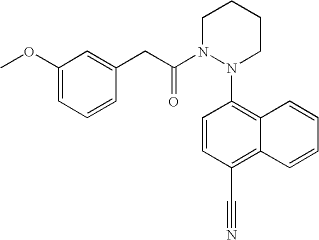

### 4-[2-{[3-(methyloxy)phenyl]acetyl}tetrahydro-1(2H)-pyridazinyl]1-naphthalenecarbonitrile (D79)

From B1 and [3-(methyloxy)phenyl]acetic acid the title compound was isolated as a yellow oil.

MS (m/z) APCI AP⁺=386

### Example 302

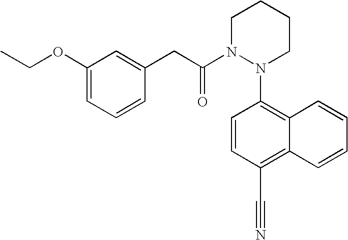

### 4-[2-{[3-(ethyloxy)phenyl]acetyl}tetrahydro-1(2H)-pyridazinyl]-1-naphthalenecarbonitrile (D80)

From B1 and [3-(ethyloxy)phenyl]acetic acid the title compound was isolated as a yellow oil.

MS (m/z) APCI AP⁺=400

### Example 303

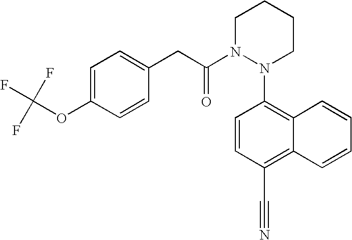

### 4-[2-({4-[(trifluoromethyl)oxy]phenyl}acetyl)tetrahydro-1(211)-pyridazinyl]-1-naphthalenecarbonitrile (D81)

From B1 and {4-[(trifluoromethyl)oxy]phenyl}acetic acid the title compound was isolated as a yellow oil.

MS (m/z) APCI AP⁺=440

### Example 304

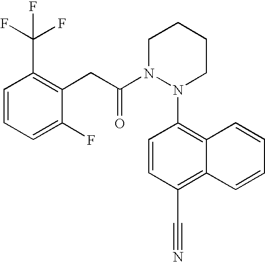

### 4-{[2-([2-fluoro-6-(trifluoromethyl)phenyl]acetyl}tetrahydro-1(2H)-pyridazinyl]-1-naphthalenecarbonitrile (D82)

From B1 and [2-fluoro-6-(trifluoromethyl)phenyl]acetic acid the title compound was isolated as a yellow oil.

MS (m/z) APCI AP⁺=442

### Example 305

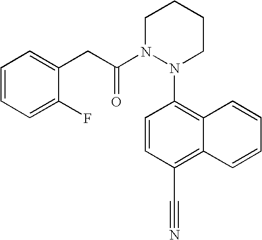

### 4-[2-[(2-fluorophenyl)acetyl]tetrahydro-1(2H)-pyridazinyl]-1-naphthalenecarbonitrile (D83)

From B1 and (2-fluorophenyl)acetic acid the title compound was isolated as a white solid.

MS (m/z) APCI AP⁺=374

### Example 306

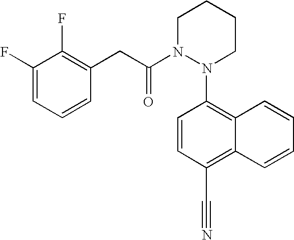

### 4-[2-[(2,3-difluorophenyl)acetyl]tetrahydro-1(2H)-pyridazinyl]-1-naphthalenecarbonitrile (D84)

From B1 and (2,3-difluorophenyl)acetic acid the title compound was isolated as a white solid.

MS (m/z) APCI AP⁺=392

### Example 307

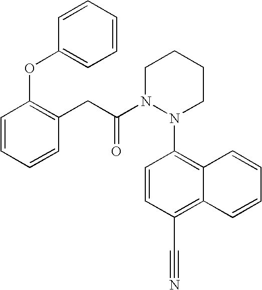

### 4-[2-{[2-(phenyloxy)phenyl]acetyl}tetrahydro-1(2H)-pyridazinyl]-1-naphthalenecarbonitrile (D85)

From B1 and [2-(phenyloxy)phenyl]acetic acid the title compound was isolated as a white solid.

MS (m/z) APCI AP⁺=448

### Example 308

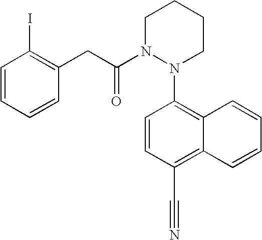

### 4-[2-[(2-iodophenyl)acetyl]tetrahydro-1(2H)-pyridazinyl]-1-naphthalenecarbonitrile (D86)

From B1 and (2-iodophenyl)acetic acid the title compound was isolated as a white solid.

MS (m/z) APCI AP⁺=482

### Example 309

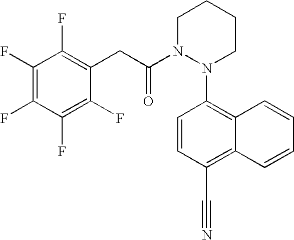

### 4-[2-[(pentafluorophenyl)acetyl]tetrahydro-1(2H)-pyridazinyl]-1-naphthalenecarbonitrile (D87)

From B1 and (pentafluorophenyl)acetic acid the title compound was isolated as a clear oil.

MS (m/z) APCI AP⁺=446

### Example 310

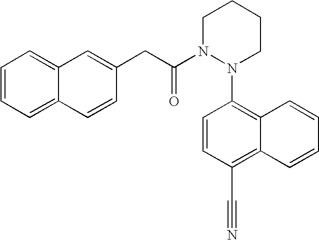

### 4-[2-(2-naphthalenylacetyl)tetrahydro-1(2H)-pyridazinyl]-1-naphthalenecarbonitrile (D88)

From B1 and (2-naphthalenyl)acetic acid the title compound was isolated as a white solid.

MS (m/z) APCI AP⁺=406

### Example 311

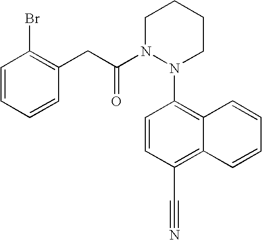

### 4-[2-[(2-bromophenyl)acetyl]tetrahydro-1(2H)-pyridazinyl]-1-naphthalenecarbonitrile (D89)

From B1 and (2-bromophenyl)acetic acid the title compound was isolated as a white solid.

MS (m/z) APCI AP⁺=435

### Example 312

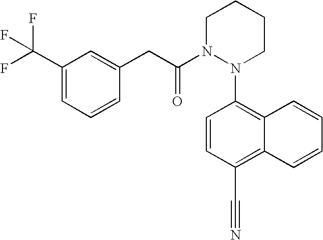

### 4-[2-{[3-(trifluoromethyl)phenyl]acetyl}tetrahydro-1(2H)-pyridazinyl]-1-naphthalenecarbonitrile (D90)

From B1 and [3-(trifluoromethyl)phenyl]acetic acid the title compound was isolated as a white solid.

MS (m/z) APCI AP⁺=424

### Example 313

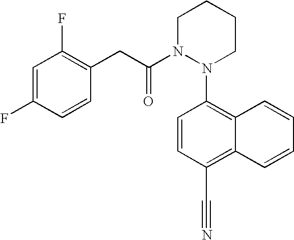

### 4-[2-[(2,4-difluorophenyl)acetyl]tetrahydro-1(2H-pyridazinyl]-1-naphthalenecarbonitrile (D91)

From B1 and (2,4-difluorophenyl)acetic acid the title compound was isolated as a white solid.

MS (m/z) APCI AP⁺=392

### Example 314

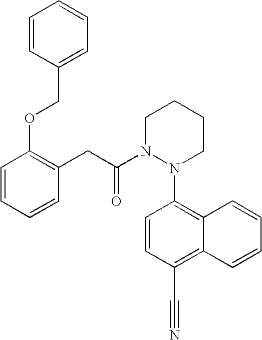

### 4-[2-({2-[(phenylmethyl)oxy]phenyl}acetyl)tetrahydro-1(2H-pyridazinyl]-1-naphthalenecarbonitrile (D92)

From B1 and {2-[phenylmethyl(oxy)]phenyl}acetic acid the title compound was isolated as a yellow oil.

MS (m/z) APCI AP⁺=462

### Example 315

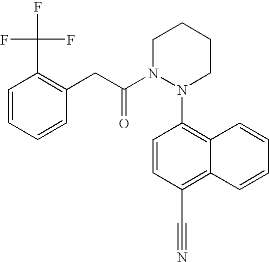

### 4-[2-{[2-(trifluoromethyl)phenyl]acetyl}tetrahydro-1(2H)-pyridazinyl]-1-naphthalenecarbonitrile (D93)

From B1 and [2-(trifluoromethyl)phenyl]acetic acid the title compound was isolated as a white solid.

MS (m/z) APCI AP⁺=424

### Example 316

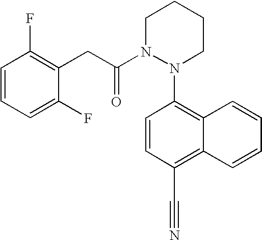

### 4-[2-[(2,6-difluorophenyl)acetyl]tetrahydro-1(2H)-pyridazinyl]-1-naphthalenecarbonitrile (D94)

From B1 and (2,6-difluorophenyl)acetic acid the title compound was isolated as a white solid.

MS (m/z) APCI AP⁺=392

### Example 317

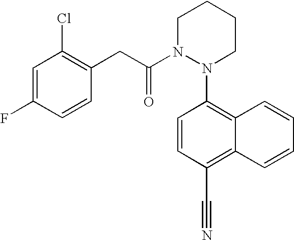

### 4-[2-[(2-chloro-4-fluorophenyl)acetyl]tetrahydro-1(2H)-pyridazinyl]-1-naphthalenecarbonitrile (D95)

From B1 and (2-chloro-4-fluorophenyl)acetic acid the title compound was isolated as a white solid.

MS (m/z) APCI AP⁺=408

### Example 318

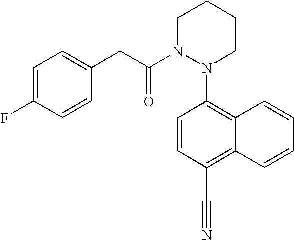

### 4-[2-[(4-fluorophenyl)acetyl]tetrahydro-1(2H-pyridazinyl]-1-naphthalenecarbonitrile (D96)

From B1 and (4-fluorophenyl)acetic acid the title compound was isolated as a white solid.

MS (m/z) APCI AP⁺=374

### Example 319

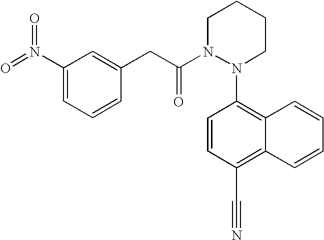

### 4-[2-[(3-nitrophenyl)acetyl]tetrahydro-1(2H)-pyridazinyl]-1-naphthalenecarbonitrile (D97)

From B1 and (3-nitrophenyl)acetic acid the title compound was isolated as a yellow solid.

MS (m/z) APCI AP⁺=401

### Example 320

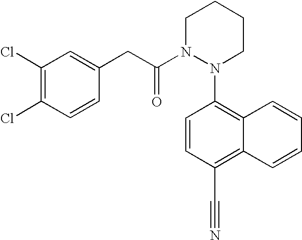

### 4-[2-[(3,4-dichlorophenyl)acetyl]tetrahydro-1(2H)-pyridazinyl]-1-naphthalenecarbonitrile (D98)

From B1 and (3,4-dichlorophenyl)acetic acid the title compound was isolated as a white solid.

MS (m/z) APCI AP⁺=424

### Example 322

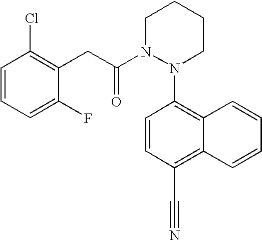

### 4-[2-[(2-chloro-6-fluorophenyl)acetyl]tetrahydro-1(2H)-pyridazinyl]-1-naphthalenecarbonitrile (D99)

From B1 and (2-chloro-6-fluorophenyl)acetic acid the title compound was isolated as a white solid.

MS (m/z) APCI AP⁺=408

### Example 323

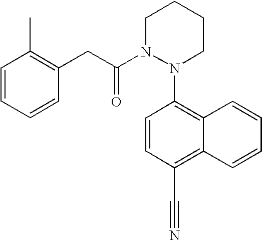

### 4-[2-[(2-methylphenyl)acetyl]tetrahydro-1(2H)-pyridazinyl]-1-naphthalenecarbonitrile (D100)

From B1 and (2-methylphenyl)acetic acid the title compound was isolated as a white solid.

MS (m/z) APCI AP⁺=370

### Example 324

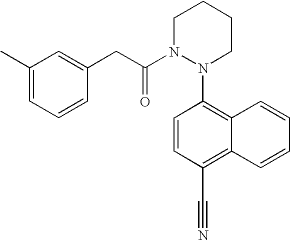

### 4-[2-[(3-methylphenyl)acetyl]tetrahydro-1(2H)-pyridazinyl]-1-naphthalenecarbonitrile (D101)

From B1 and (3-methylphenyl)acetic acid the title compound was isolated as a white solid.

MS (m/z) APCI AP⁺=370

### Example 325

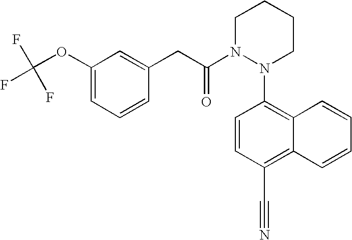

### 4-[2-({3-[(trifluoromethyl)oxy]phenyl}acetyl)tetrahydro-1(2H)-pyridazinyl]-1-naphthalenecarbonitrile (D102)

From B1 and {3-[(trifluoromethyl)oxy]phenyl}acetic acid the title compound was isolated as a white solid.

MS (m/z) APCI AP⁺=440

### Example 326

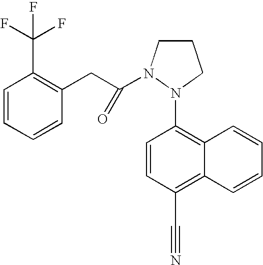

### 4-[2-({3-[(trifluoromethyl)oxy]phenyl}acetyl)-1-pyrazolidinyl]-1-naphthalenecarbonitrile (D103)

From B5 and [2-(trifluoromethyl)phenyl]acetic acid the title compound was isolated as a yellow solid.

MS (m/z) ESI ES⁺=410

### Example 327

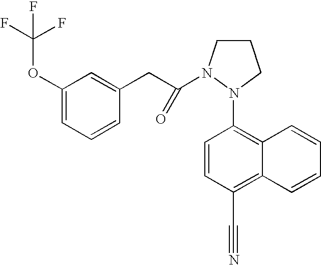

### 4-[2-({3-[(trifluoromethyl)oxy]phenyl}acetyl)-1-pyrazolidinyl]-1-naphthalenecarbonitrile (D104)

From B5 and {3-[(trifluoromethyl)oxy]phenyl}acetic acid the title compound was isolated as a yellow oil.

MS (m/z) ESI ES⁺=426

### Example 328

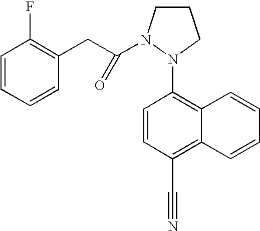

### 4-{2-[(2-fluorophenyl)acetyl]-1-pyrazolidinyl}-1-naphthalenecarbonitrile (D105)

From B5 and (2-fluorophenyl)acetic acid the title compound was isolated as a clear oil.

MS (m/z) ESI ES⁺=360

### Example 329

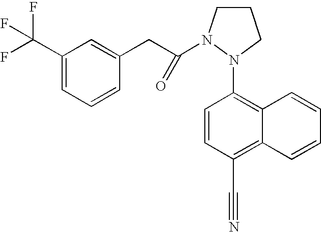

### 4-(2-{[3-(trifluoromethyl)phenyl]acetyl}-1-pyrazolidinyl)-1-naphthalenecarbonitrile (D106)

From B5 and [3-(trifluoromethyl)phenyl]acetic acid the title compound was isolated as a clear oil.

MS (m/z) ESI ES⁺=410

### Example 330

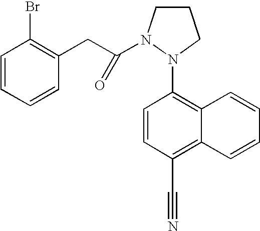

### 4-{2-[(2-bromophenyl)acetyl]-1-pyrazolidinyl}-1-naphthalenecarbonitrile (D107)

From B5 and (2-bromophenyl)acetic acid the title compound was isolated as a white solid.

MS (m/z) ESI ES⁺=421

### Example 331

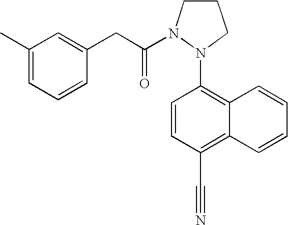

### 4-{2-[(3-methylphenyl)acetyl]-1-pyrazolidinyl}-1-naphthalenecarbonitrile (D108)

From B5 and (3-methylphenyl)acetic acid the title compound was isolated as a yellow oil.

MS (m/z) ESI ES⁺=356

### Example 332

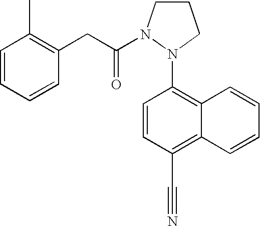

### 4-{2-[(2-methylphenyl)acetyl]-1-pyrazolidinyl}-1-naphthalenecarbonitrile (D109)

From B5 and (2-methylphenyl)acetic acid the title compound was isolated as a yellow oil.

MS (m/z) ESI ES⁺=356

### Example 333

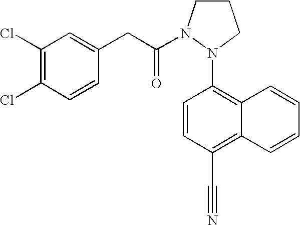

### 4-{2-[(3,4-dichlorophenyl)acetyl]-1-pyrazolidinyl}-1-naphthalenecarbonitrile (D110)

From B5 and (3,4-dichlorophenyl)acetic acid the title compound was isolated as a clear oil.

MS (m/z) ESI ES⁺=411

### Example 334

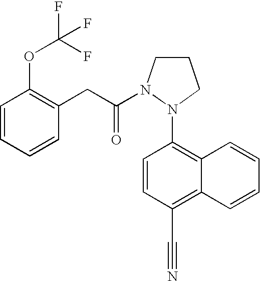

### 4-[2-({2-[(trifluoromethyl)oxy]phenyl}acetyl)-1-pyrazolidinyl]-1-naphthalenecarbonitrile (D111)

From B5 and {2-[(trifluoromethyl)oxy]phenyl}acetic acid the title compound was isolated as a white solid.

MS (m/z) ESI ES⁺=426

### Example 335

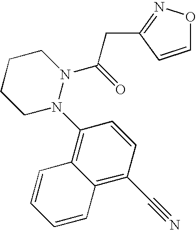

### 4-[2-(3-isoxazolylacetyl)tetrahydro-1(2H)-pyridazinyl]-1-naphthalenecarbonitrile (D112)

From B1 and (3-Isoxazolyl)Acetic Acid the Title Compound was Obtained.

MS (m/z) ESI ES⁺=347

### Example 336

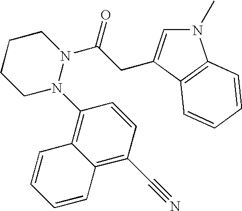

### 4-[2-[(1-methyl-1H-indol-3-yl)acetyl]tetrahydro-1(2H)-pyridazinyl]-1-naphthalenecarbonitrile (D113)

From B1 and (1-methyl-1H-indol-3-yl)acetic acid the title compound was obtained.

MS (m/z) ESI ES⁺=409

### Example 337

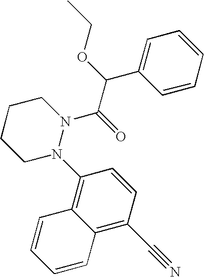

### 4-[2-[(ethyloxy)(phenyl)acetyl]tetrahydro-1(2H-pyridazinyl]-1-naphthalenecarbonitrile (D114)

From B1 and (ethyloxy)(phenyl)acetic acid the title compound was obtained.

MS (m/z) ESI ES⁺=400

### Example 338

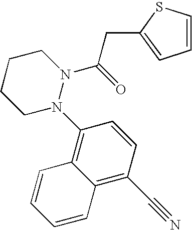

### 4-[2-(2-thienylacetyl)tetrahydro-1(2H)-pyridazinyl]-1-naphthalenecarbonitrile (D115)

From B1 and (2-thienyl)acetic acid the title compound was obtained.

MS (m/z) ESI ES⁺=362

### Example 339

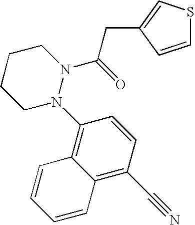

### 4-[2-(3-thienylacetyl)tetrahydro-1(2H)-pyridazinyl]-1-naphthalenecarbonitrile (D116)

From B1 and (3-thienyl)acetic acid the title compound was obtained.

MS (m/z) ESI ES⁺=362

### Example 340

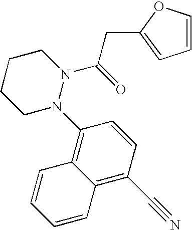

### 4-[2-(2-furanylacetyl)tetrahydro-1(2H)-pyridazinyl]-1-naphthalenecarbonitrile (D117)

From B1 and (2-furanyl)acetic acid the title compound was obtained.

MS (m/z) ESI ES⁺=346

### Hexahydropyridazine Amide Couplings: Mixed Anhydride Route

### Example 341

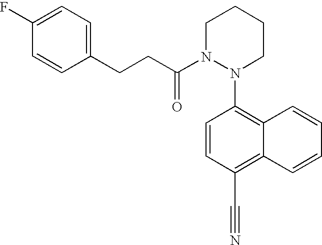

### 4-[2-[3-(4-fluorophenyl)propanoyl]tetrahydro-1(2H)-pyridazinyl]-1-naphthalenecarbonitrile (D118)

To a 40 ml scintillation vial was added 106 mg 3-(4-fluorophenyl)propanoic acid (0.695 mmoles, 1.0 eq), 1 ml dichloromethane, and 0.1 ml triethylamine (0.695 mmoles, 2.2 eq). This was followed by the addition of 0.083 ml isobutylchloroformate (0.695 mmoles, 2.2 eq). The reaction mixture was shaken for 15 minutes on an orbital shaker at room temperature. Then 75 mg 4-(tetrahydro-1(2H)-pyridazinyl)-1-naphthalenecarbonitrile (B1) (0.316 mmoles, 1 eq) in 1 ml dichloromethane was added and the reaction was shaken on an orbital shaker overnight. The reaction mixture was concentrated in vacuo. The residue was purified via preparative HPLC (Phenomenex column Luna C18, 75 mm×30 mm, 5 micron), 30% acetonitrile/water (0.01% TFA) to 100% acetonitrile, to yield 19 mg of the title compound as a clear oil.

MS (m/z) APCI AP⁺=388

The following compounds were synthesized according to a similar general procedure as described for D118:

### Example 342

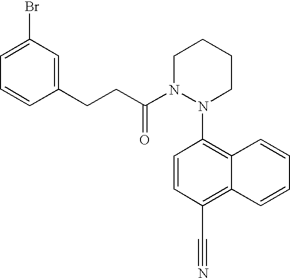

### 4-[2-[3-(3-bromophenyl)propanoyl]tetrahydro-1(2H)-pyridazinyl]-1-naphthalenecarbonitrile (D119)

From B1 and 3-(3-bromophenyl)propanoic acid the title compound was isolated as a white solid.

MS (m/z) APCI AP⁺=449

### Example 343

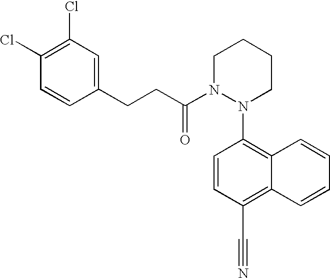

### 4-[2-[3-(3,4-dichlorophenyl)propanoyl]tetrahydro-1(2H)-pyridazinyl]-1-naphthalenecarbonitrile (D120)

From B1 and 3-(3,4-dichlorophenyl)propanoic acid the title compound was isolated as a white solid.

MS (m/z) APCI AP⁺=438

### Example 342

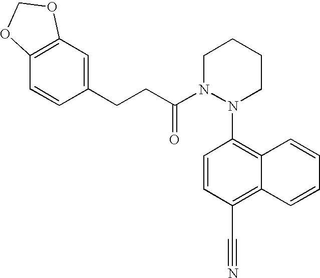

### 4-[2-[3-(1,3-benzodioxol-5-yl)propanoyl]tetrahydro-1(2H-pyridazinyl]-1-naphthalenecarbonitrile (D121)

From B1 and 3-(1,3-benzodioxol-5-yl)propanoic acid the title compound was isolated as a clear oil.

MS (m/z) APCI AP⁺=414

### Example 343

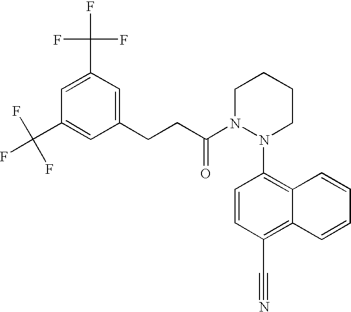

### 4-[2-{3-[3,5-bis(trifluoromethyl)phenyl]propanoyl}tetrahydro-1(2H)-pyridazinyl]-1-naphthalenecarbonitrile (D122)

From B1 and 3-[3,5-bis(trifluoromethyl)phenyl]propanoic acid the title compound was isolated as a clear oil.

MS (m/z) APCI AP⁺=506

### Example 344

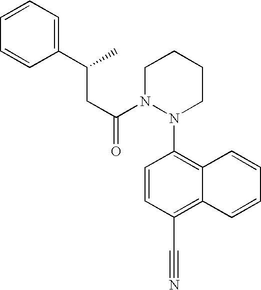

### 4-[2-[(3R)-3-phenylbutanoyl]tetrahydro-1(2H-pyridazinyl]-1-naphthalenecarbonitrile (D123)

From B1 and (3R)-3-phenylbutanoic acid the title compound was isolated as a clear oil.

MS (m/z) APCI AP⁺=384

### Example 345

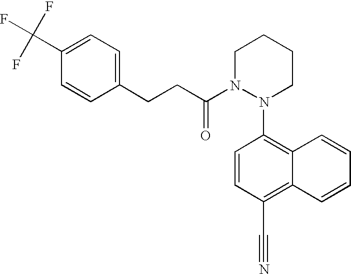

### 4-[2-{3-[4-(trifluoromethyl)phenyl]propanoyl}tetrahydro-1(2H-pyridazinyl]-1-naphthalenecarbonitrile (D124)

From B1 and 3-[4-(trifluoromethyl)phenyl]propanoic acid the title compound was isolated as a clear oil.

MS (m/z) APCI AP⁺=438

### Example 346

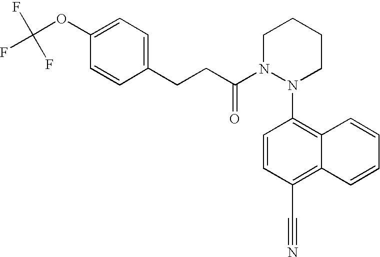

### 4-[2-(3-{4-[(trifluoromethyl)oxy]phenyl}propanoyl)tetrahydro-1(2H)-pyridazinyl]-1-naphthalenecarbonitrile (D125)

From B1 and 3-{[4-(trifluoromethyl)oxy]phenyl}propanoic acid the title compound was isolated as a clear oil.

MS (m/z) APCI AP⁺=454

### Example 347

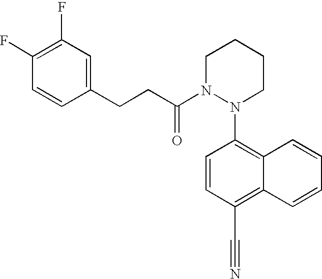

### 4-[2-[3-(3,4-difluorophenyl)propanoyl]tetrahydro-1(2H)-pyridazinyl]-1-naphthalenecarbonitrile (D126)

From B1 and 3-(3,4-difluorophenyl)propanoic acid the title compound was isolated as a white solid.

MS (m/z) APCI AP⁺=406

### Example 348

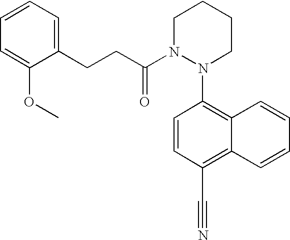

### 4-[2-{3-[2-(methyloxy)phenyl]propanoyl}tetrahydro-1(2H)-pyridazinyl]-1-naphthalenecarbonitrile (D127)

From B1 and 3-[2-(methyloxy)phenyl]propanoic acid the title compound was isolated as a clear oil.

MS (m/z) APCI AP⁺=400

### Example 349

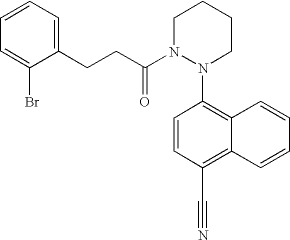

### 4-[2-[3-(2-bromophenyl)propanoyl]tetrahydro-1(2H)-pyridazinyl]-1-naphthalenecarbonitrile (D128)

From B1 and 3-(2-bromophenyl)propanoic acid the title compound was isolated as a clear oil.

MS (m/z) APCI AP⁺=449

### Example 350

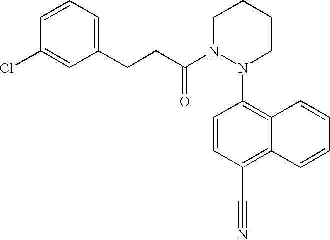

### 4-[2-[3-(3-chlorophenyl)propanoyl]tetrahydro-1(2H)-pyridazinyl]-1-naphthalenecarbonitrile (D129)

From B1 and 3-(3-chlorophenyl)propanoic acid the title compound was isolated as a white solid.

MS (m/z) APCI AP⁺=404

### Example 351

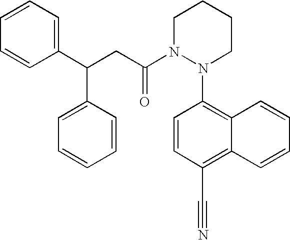

### 4-[2-(3,3-diphenylpropanoyl)tetrahydro-1(2H)-pyridazinyl]-1-naphthalenecarbonitrile (D130)

From B1 and 3,3-diphenylpropanoic acid the title compound was isolated as a white solid.

MS (m/z) APCI AP⁺=446

### Example 352

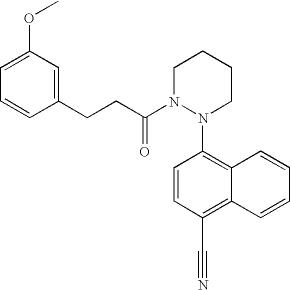

### 4-[2-{3-[3-(methyloxy)phenyl]propanoyl}tetrahydro-1(2H)-pyridazinyl]-1-naphthalenecarbonitrile (D131)

From B1 and 3-[3-(methyloxy)phenyl]propanoic acid the title compound was isolated as a clear oil.

MS (m/z) APCI AP⁺=400

### Example 353

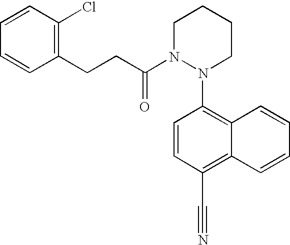

### 4-[2-[3-(2-chlorophenyl)propanoyl]tetrahydro-1(2H)-pyridazinyl]-1-naphthalenecarbonitrile (D132)

From B1 and 3-(2-chlorophenyl)propanoic acid the title compound was isolated as a white solid.

MS (m/z) APCI AP⁺=404

### Example 354

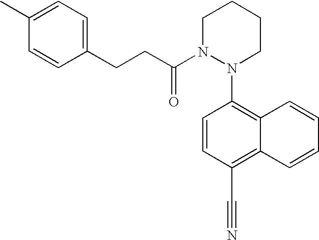

### 4-[2-[3-(4-methylphenyl)propanoyl]tetrahydro-1(2H)-pyridazinyl]-1-naphthalenecarbonitrile (D133)

From B1 and 3-(4-methylphenyl)propanoic acid the title compound was isolated as a clear oil.

MS (m/z) APCI AP⁺=384

### Example 355

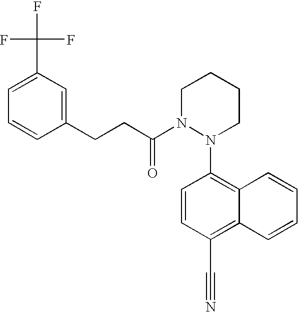

### 4-[2-{3-[3-(trifluoromethyl)phenyl]propanoyl}tetrahydro-1(2H)-pyridazinyl]-1-naphthalenecarbonitrile (D134)

From B1 and 3-[3-(trifluoromethyl)phenyl]propanoic acid the title compound was isolated as a clear oil.

MS (m/z) APCI AP⁺=438

### Example 356

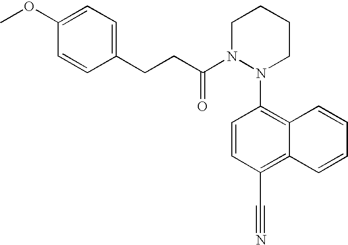

### 4-[2-{3-[4-(methyloxy)phenyl]propanoyl}tetrahydro-1(2H)-pyridazinyl]-1-naphthalenecarbonitrile (D135)

From B1 and 3-[4-(methyloxy)phenyl]propanoic acid the title compound was isolated as a clear oil.

MS (m/z) APCI AP⁺=400

### Example 357

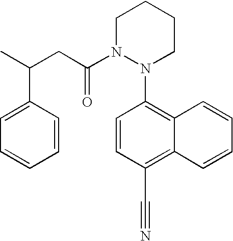

### 4-[2-(3-phenylbutanoyl)tetrahydro-1(2H)-pyridazinyl]-1-naphthalenecarbonitrile (D136)

From B1 and 3-phenylbutanoic acid the title compound was isolated as a white solid.

MS (m/z) APCI AP⁺=384

### Example 358

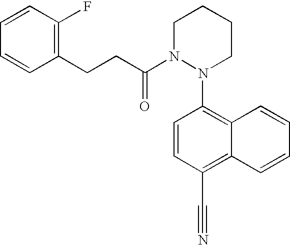

### 4-[2-[3-(2-fluorophenyl)propanoyl]tetrahydro-1(2H)-pyridazinyl]-1-naphthalenecarbonitrile (D137)

From B1 and 3-(2-fluorophenyl)propanoic acid the title compound was isolated as a clear oil.

MS (m/z) APCI AP⁺=388

### Example 359

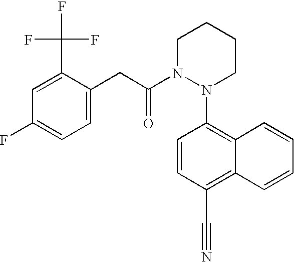

### 4-[2-{[4-fluoro-2-(trifluoromethyl)phenyl]acetyl}tetrahydro-1(2H-pyridazinyl]-1-naphthalenecarbonitrile (D138)

From B1 and [4-fluoro-2-(trifluoromethyl)phenyl]acetic acid the title compound was isolated as a white solid.

MS (m/z) APCI AP⁺=442

### Example 360

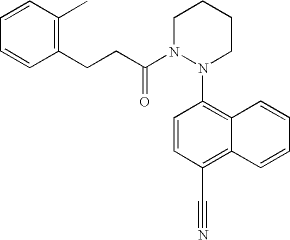

### 4-[2-[3-(2-methylphenyl)propanoyl]tetrahydro-1(2H)-pyridazinyl]-1-naphthalenecarbonitrile (D139)

From B1 and 3-(2-methylphenyl)propanoic acid the title compound was isolated as a clear oil.

MS (m/z) APCI AP⁺=384

### Example 361

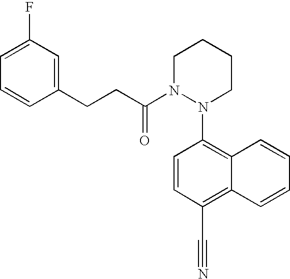

### 4-[2-[3-(3-fluorophenyl)propanoyl]tetrahydro-1(2H)-pyridazinyl]-1-naphthalenecarbonitrile (D140)

From B1 and 3-(3-fluorophenyl)propanoic acid the title compound was isolated as a white solid.

MS (m/z) APCI AP⁺=388

### Example 362

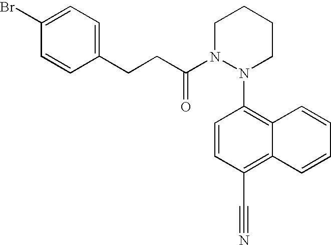

### 4-[2-[3-(4-bromophenyl)propanoyl]tetrahydro-1(2H-pyridazinyl]-1-naphthalenecarbonitrile (D141)

From B1 and 3-(4-bromophenyl)propanoic acid the title compound was isolated as a clear oil.

MS (m/z) APCI AP⁺=449

### Example 363

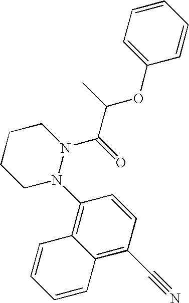

### 4-[2-[2-(phenyloxy)propanoyl]tetrahydro-1(2H)-pyridazinyl]-1-naphthalenecarbonitrile (D142)

From B1 and 2-(phenyloxy)propanoic acid the title compound was obtained.

MS (m/z) ESI ES⁺=386

### Example 364

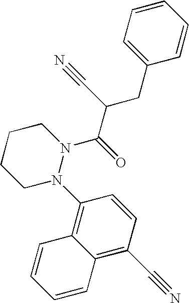

### 4-[2-(2-cyano-3-phenylpropanoyl)tetrahydro-1(2H)-pyridazinyl]-1-naphthalenecarbonitrile (D143)

From B1 and 2-cyano-3-phenylpropanoic acid the title compound was obtained.

MS (m/z) ESI ES⁺=395

### Example 365

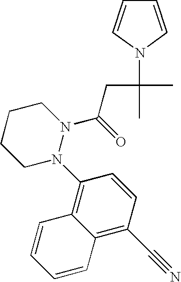

### 4-[2-[3-methyl-3-(1H-pyrrol-1-yl)butanoyl]tetrahydro-1(2H)-pyridazinyl]-1-naphthalenecarbonitrile (D144)

From B1 and 3-methyl-3-(1H-pyrrol-1-yl)butanoic acid the title compound was obtained.

MS (m/z) APCI AP⁻=385

### Example 366

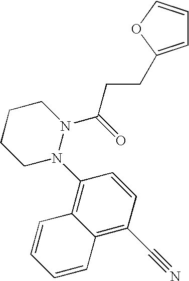

### 4-[2-[3-(2-furanyl)propanoyl]tetrahydro-1(2H)-pyridazinyl]-1-naphthalenecarbonitrile (D145)

From B1 and 3-(2-furanyl)propanoic acid the title compound was obtained.

MS (m/z) ESI ES⁺=360

### Example 367

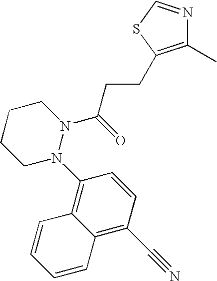

### 4-[2-[3-(4-methyl-1,3-thiazol-5-yl)propanoyl]tetrahydro-1(2H)-pyridazinyl]-1-naphthalenecarbonitrile (D146)

From B1 and 3-(4-methyl-1,3-thiazol-5-yl)propanoic acid the title compound was obtained.

MS (m/z) ESI ES⁺=391

### Example 368

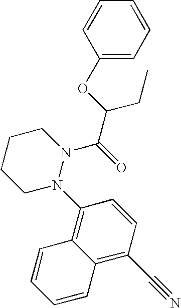

### 4-[2-[2-(phenyloxy)butanoyl]tetrahydro-1(2H)-pyridazinyl]-1-naphthalenecarbonitrile (D147)

From B1 and 2-phenoxybutanoic acid the title compound was obtained.

MS (m/z) ESI ES⁺=400

### Hexahydropyridazine Amide Couplings: Polystyrene-Linked Carbodiimide Method

### Example 369

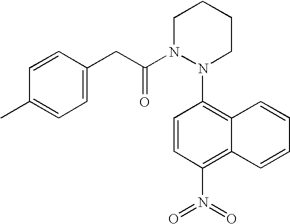

### 1-[(4-methylphenyl)acetyl]-2-(4-nitro-1-naphthalenyl)hexahydropyridazine (D147)

To a 40 ml scintillation vial was added 73 mg 1-(4-nitro-1-naphthalenyl)hexahydropyridazine (B2) (0.28 mmoles, 1 eq), 3 ml chloroform, and 55 mg (4-methylphenyl)acetic acid (0.37 mmoles, 1.3 eq). Then 410 mg polystyrene-linked carbodiimide resin (Argonaut Technologies, 1.28 mmol/g, 2 eq) was added and the reaction was heated at 60° C. on an orbital shaker overnight. After cooling to room temperature the reaction was filtered and concentrated in vacuo. The residue was purified via preparative HPLC (Phenomenex column Luna C18, 75 mm×30 mm, 5 micron), 30% acetonitrile/water (0.01% TFA) to 100% acetonitrile, to yield 69 mg of the title compound as a yellow solid.

MS (m/z) ES⁺=390

The following compounds were synthesized according to a similar general procedure as described for D147:

### Example 370

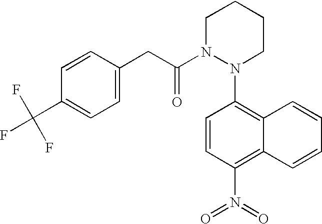

### 1-(4-nitro-1-naphthalenyl)-2-{[4-(trifluoromethyl)phenyl]acetyl}hexahydropyridazine (D148)

From B2 and [4-(trifluoromethyl)phenyl]acetic acid the title compound was isolated as a yellow solid.

MS (m/z) ESI ES⁺=444

### Example 371

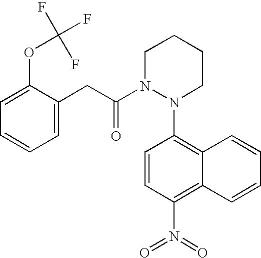

### 1-(4-nitro-1-naphthalenyl)-2-({2-[(trifluoromethyl)oxy]phenyl}acetyl)hexahydropyridazine (D149)

From B2 and {2-[(trifluoromethyl)oxy]phenyl}acetic acid the title compound was isolated as an orange oil.

MS (m/z) ESI ES⁺=460

### Example 372

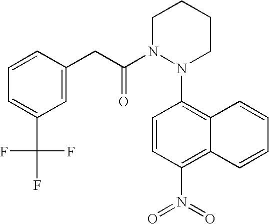

### 1-(4-nitro-1-naphthalenyl)-2-{[3-(trifluoromethyl)phenyl]acetyl}hexahydropyridazine (D150)

From B2 and [3-(trifluoromethyl)phenyl]acetic acid the title compound was isolated as a yellow oil.

MS (m/z) ESI ES⁺=444

### Example 373

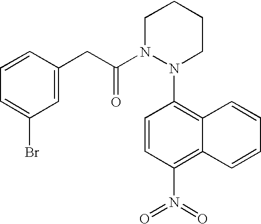

### 1-[(3-bromophenyl)acetyl]-2-(4-nitro-1-naphthalenyl)hexahydropyridazine (D151)

From B2 and (3-bromophenyl)acetic acid the title compound was isolated as a yellow oil.

MS (m/z) ESI ES⁺=455

### Example 374

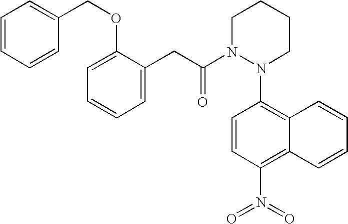

### 1-(4-nitro-1-naphthalenyl)-2-({2-[(phenylmethyl)oxy]phenyl}acetyl)hexahydropyridazine (D152)

From B2 and {2-[(phenylmethyl)oxy]phenyl}acetic acid the title compound was isolated as a yellow oil.

MS (m/z) ESI ES⁺=482

### Example 375

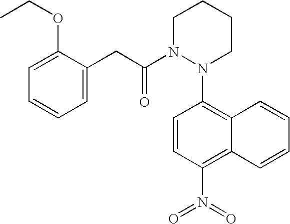

### 1-{[2-(ethyloxy)phenyl]acetyl}-2-(4-nitro-1-naphthalenyl)hexahydropyridazine (D153)

From B2 and [2-(ethyloxy)phenyl]acetic acid the title compound was isolated as a yellow solid.

MS (m/z) ESI ES⁺=420

### Example 376

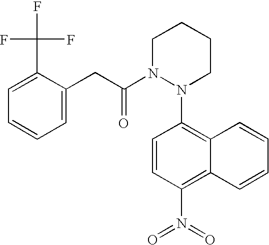

### 1-(4-nitro-1-naphthalenyl)-2-{[2-(trifluoromethyl)phenyl]acetyl}hexahydropyridazine (D154)

From B2 and [2-(trifluoromethyl)phenyl]acetic acid the title compound was isolated as a yellow solid.

MS (m/z) ESI ES⁺=444

### Example 377

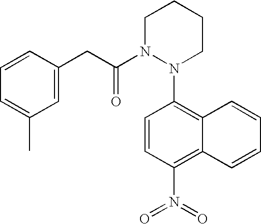

### 1-[(3-methylphenyl)acetyl]-2-(4-nitro-1-naphthalenyl)hexahydropyridazine (D155)

From B2 and (3-methylphenyl)acetic acid the title compound was isolated as an orange oil.

MS (m/z) ESI ES⁺=390

### Example 378

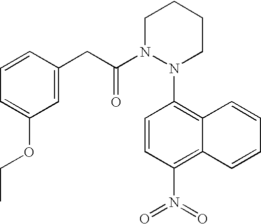

### 1-{[3-(ethyloxy)phenyl]acetyl}-2-(4-nitro-1-naphthalenyl)hexahydropyridazine (D156)

From B2 and [3-(ethyloxy)phenyl]acetic acid the title compound was isolated as a yellow oil.

MS (m/z) ESI ES⁺=420

### Example 379

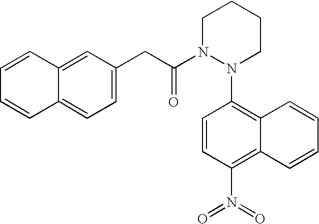

### 1-(2-naphthalenylacetyl)-2-(4-nitro-1-naphthalenyl)hexahydropyridazine (D157)

From B2 and (2-naphthalenyl)acetic acid the title compound was isolated as a yellow oil.

MS (m/z) ESI ES⁺=426

### Example 380

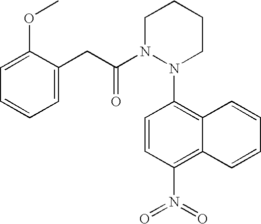

### 1-{[2-(methyloxy)phenyl]acetyl}-2-(4-nitro-1-naphthalenyl)hexahydropyridazine (D158)

From B2 and [2-(methyloxy)phenyl]acetic acid the title compound was isolated as an orange oil.

MS (m/z) ESI ES⁺=406

### Example 381

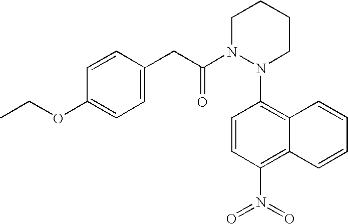

### 1-{[4-(ethyloxy)phenyl]acetyl}-2-(4-nitro-1-naphthalenyl)hexahydropyridazine (D159)

From B2 and [4-(ethyloxy)phenyl]acetic acid the title compound was isolated as a yellow solid.

MS (m/z) ESI ES⁺=420

### Example 382

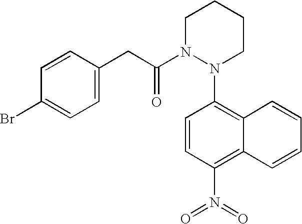

### 1-[(4-bromophenyl)acetyl]-2-(4-nitro-1-naphthalenyl)hexahydropyridazine (D160)

From B2 and (4-bromophenyl)acetic acid the title compound was isolated as a yellow solid.

MS (m/z) ESI ES⁺=455

### Example 383

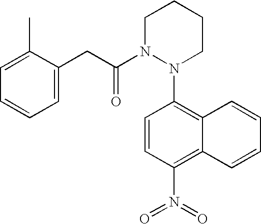

### 1-[(2-methylphenyl)acetyl]-2-(4-nitro-1-naphthalenyl)hexahydropyridazine (D161)

From B2 and (2-methylphenyl)acetic acid the title compound was isolated as a yellow oil.

MS (m/z) ESI ES⁺=390

### Example 384

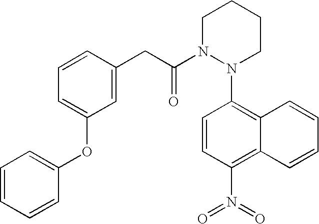

### 1-(4-nitro-1-naphthalenyl)-2-{[3-(phenyloxy)phenyl]acetyl}hexahydropyridazine (D162)

From B2 and [3-(phenyloxy)phenyl]acetic acid the title compound was isolated as a yellow oil.

MS (m/z) ESI ES⁺=468

### Example 385

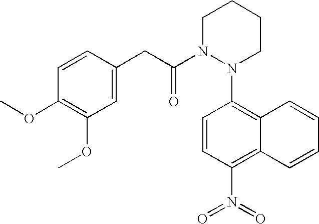

### 1-{[3,4-bis(methyloxy)phenyl]acetyl}-2-(4-nitro-1-naphthalenyl)hexahydropyridazine (D163)

From B2 and [3,4-(dimethyloxy)phenyl]acetic acid the title compound was isolated as a yellow solid.

MS (m/z) ES⁺=436 tert-Butyldimethylsilyl ether Deprotection

### Example 386

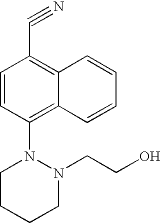

### F. 4-[2-(2-hydroxyethyl)tetrahydro-1(2H)-pyridazinyl]-1-naphthalenecarbonitrile (F1)

To a 40 ml scintillation vial was added 670 mg 4-[2-(2-{[(1,1-dimethylethyl)(dimethyl)silyl]oxy}ethyl)tetrahydro-1(2H)-pyridazinyl]-1-naphthalenecarbonitrile (C186) (1.69 mmoles, 1 eq), 10 ml ethanol, and 450 mg pyridinium p-toluenesulfonate (1.69 mmoles, 1 eq). The reaction mixture was heated at 70° C. overnight and, after cooling to room temperature, was concentrated in vacuo. The residue was purified via flash chromatography (20% ethyl acetate/hexanes to 50% ethyl acetate/hexanes) to yield 161 mg of the title compound.

1H NMR (400 MHz, CHLOROFORM-D) δ ppm 1.6 (m, 1H) 1.7 (m, 2H) 1.9 (m, 2H) 3.0 (m, 2H) 3.3 (m, 4H) 3.4 (m, 2H) 7.0 (d, J=8.1 Hz, 1H) 7.5 (m, 1H) 7.6 (t, J=7.1 Hz, 1H) 7.8 (d, J=7.9 Hz, 1H) 8.2 (d, J=8.4 Hz, 1H) 8.3 (d, J=8.2 Hz, 1H)

The following compounds were synthesized according to a similar general procedure as described for F1:

### Example 387

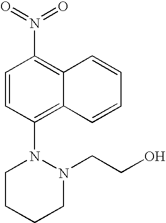

### 2-[2-(4-nitro-1-naphthalenyl)tetrahydro-1(2H-pyridazinyl]ethanol (F3)

From C185 the title compound was obtained.

1H NMR (400 MHz, CHLOROFORM-D) δ ppm 1.6 (m, 1H) 1.8 (m, 4H) 3.0 (m, 2H) 3.3 (m, 4H) 3.5 (m, 2H) 7.0 (d, J=8.4 Hz, 1H) 7.5 (t, J=7.6 Hz, 1H) 7.7 (t, J=7.9 Hz, 1H) 8.2 (d, J=8.4 Hz, 1H) 8.3 (d, J=8.6 Hz, 1H) 8.7 (d, J=8.8 Hz, 1H)

### Mitsonobu Reaction

### Example 388

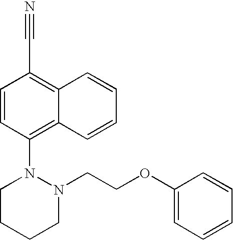

### G. 4-[2-[2-(phenyloxy)ethyl]tetrahydro-1(2H)-pyridazinyl]-1-naphthalenecarbonitrile (G1)

To a 40 ml scintillation vial was added 47 mg 4-[2-(2-hydroxyethyl)tetrahydro-1(2H)-pyridazinyl]-1-naphthalenecarbonitrile (F1) (0.166 mmoles, 1 eq), 2 ml dichloromethane, 62 mg triphenylphosphine (0.199 mmoles, 1.2 eq), and 20 mg phenol (0.183 mmoles, 1.1 eq). This was followed by the addition of 0.050 ml diisopropylazodicarboxylate (0.199 mmoles, 1.22 eq). The reaction mixture was shaken on an orbital shaker overnight at room temperature and then was concentrated in vacuo. The residue was purified via preparative HPLC (Phenomenex column Luna C18, 75 mm×30 mm, 5 micron), 50% acetonitrile/water (0.01% TFA) to 100% acetonitrile, to yield 16 mg of the title compound as a yellow oil.

1H NMR (400 MHz, METHANOL-D4) δ ppm 1.9 (m, 4H), 3.3 (t, J=5.7 Hz, 2H), 3.5 (m, 4H), 3.7 (t, J=5.5 Hz, 2H), 6.4 (d, J=7.9 Hz, 2H), 6.8 (m, 1H), 7.1 (m, 3H), 7.5 (m, 1H), 7.6 (m, 1H), 7.8 (d, J=8.1 Hz, 1H), 8.0 (d, J=8.4 Hz, 1H), 8.5 (d, J=8.6 Hz, 1H)

The following compounds were synthesized according to a similar general procedure as described for G1:

### Example 389

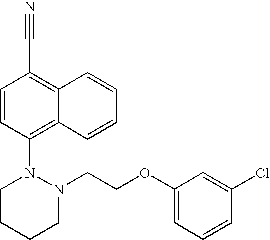

### 4-[2-{2-[(3-chlorophenyl)oxy]ethyl}tetrahydro-1(2H)-pyridazinyl]-1-naphthalenecarbonitrile (G2)

From F1 and 3-chlorophenol the title compound was isolated as a yellow oil.

MS (m/z) APCI AP⁺=392

### Example 390

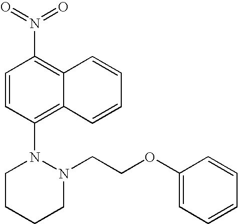

### 1-(4-nitro-1-naphthalenyl)-2-[2-(phenyloxy)ethyl]hexahydropyridazine (G3)

From F2 and phenol the title compound was isolated as a red oil.

MS (m/z) APCI AP⁺=378

### Example 391

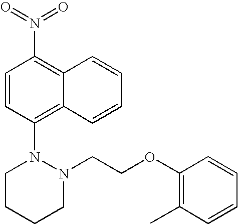

### 1-{2-[(2-methylphenyl)oxy]ethyl}-2-(4-nitro-1-naphthalenyl)hexahydropyridazine (G4)

From F2 and o-cresol the title compound was isolated as a red oil.

MS (m/z) APCI AP⁺=392

### Example 392

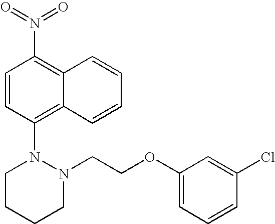

### 1-{2-[(3-chlorophenyl)oxy]ethyl}-2-(4-nitro-1-naphthalenyl)hexahydropyridazine (G5)

From F2 and 3-chlorophenol the title compound was isolated as a red oil.

MS (m/z) APCI AP⁺=412

### Example 393

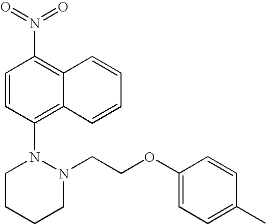

### 1-{2-[(4-methylphenyl)oxy]ethyl}-2-(4-nitro-1-naphthalenyl)hexahydropyridazine (G6)

From F2 and p-cresol the title compound was isolated as a red oil.

MS (m/z) APCI AP⁺=392

### Cyanation of Hexahydropyridazine

### Example 394

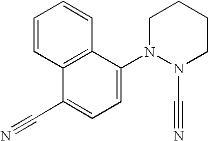

### 2-(4-cyano-1-naphthalenyl)tetrahydro-1(2H)-pyridazinecarbonitrile

To a 100 ml round bottom flask equipped with a magnetic stirbar and nitrogen flow was added 250 mg 4-(tetrahydro-1(2H)-pyridazinyl)-1-naphthalenecarbonitrile (B1) (1 mmoles, 1.0 eq) in 20 ml acetonitrile, 0.375 ml diisopropylethyl amine (2 mmoles, 2 eq), then cooled to 0° C. via an ice bath, at which point, 112 mg cyanogen bromide (1 mmole, 1 eq) was added. The reaction was allowed to warm gently to room temperature and stirred at rt for 18 hours. The solvent was removed under reduced and the resulting residue was purified by flash chromatography (eluted with 100% dichloromethane) to yield desired compound.

1H NMR (300 MHz, CHLOROFORM-D) δ ppm 2.0 (m, 2H) 2.1 (m, 2H) 3.6 (m, 2H) 3.7 (m, 2H) 7.2 (d, J=7.7 Hz, 61H) 7.7 (m, 2H) 7.9 (d, J=7.7 Hz, 1H) 8.1 (m, J=7.6, 1.0 Hz, 1H) 8.3 (m, J=7.5, 0.8 Hz, 1H)

The following compounds were synthesized according to a similar general procedure as described for Example 394:

### Example 395

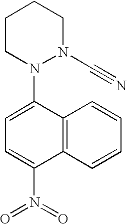

### 2-(4-nitro-1-naphthalenyl)tetrahydro-1(2H)-pyridazinecarbonitrile

From B2 the title compound was obtained.

1H NMR (400 MHz, CHLOROFORM-D) δ ppm 2.0 (m, 4H) 3.6 (m, 2H) 3.7 (m, 2H) 7.2 (d, J=8.2 Hz, 1H) 7.6 (m, 1H) 7.7 (m, 1H) 8.1 (d, J=8.6 Hz, 1H) 8.2 (d, J=8.4 Hz, 1H) 8.6 (d, J=8.8 Hz, 1H)

### Trifluoroethylation of Hexahydrodiazepine

### Example 396

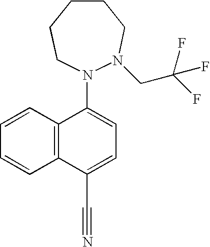

### 4-[2-(2,2,2-trifluoroethyl)hexahydro-1H-1,2-diazepin-1-yl]-1-naphthalenecarbonitrile

To a 50 ml round bottom flask equipped with a magnetic stirbar, an addition funnel, and nitrogen flow was added 250 mg 4-(hexahydro-1H-1,2-diazepin-1-yl)-1-naphthalenecarbonitrile (B4) (1 mmoles, 1.0 eq) in 5 ml 1,2-dichloroethane, then added 187 mg sodium cyanoborohydride (3 mmoles, 3.0 eq). The reaction mixture was cooled to 0° C. via an ice bath, at which point, 5 ml trifluoroacetic acid was added. Then trifluoroacetaldehyde hydrate was added dropwise and the reaction was allowed to warm gently to room temperature and stirred at rt for two days. To the reaction mixture was added 10 ml water and 20 ml dichloromethane. The phases were then separated and the organic phase was concentrated under reduced pressure to yield desired compound.

MS (m/z) ES⁺=334

The following compounds were synthesized according to a similar general procedure as used for C179 with the following exceptions: minimal amounts of anhydrous THF were used as the co-solvent in the LiAlH₄ reaction when solubility in Et₂O was low, with certain less-reactive substrates the LiAlH₄ reduction was allowed to proceed up to 48 hrs at 0° C., and, in certain cases, varying amounts of over-reduction of the ester to the Me analog was observed during the LiAlH₄ reaction:

### Example 397

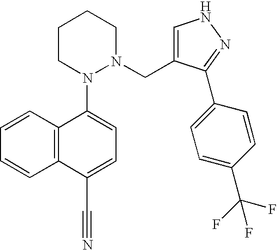

### 4-[2-({3-[4-(trifluoromethyl)phenyl]-1H-pyrazol-4-yl}methyl)tetrahydro-1(2H)-pyridazinyl]-1-naphthalenecarbonitrile (C222)

From (B1) and methyl 3-oxo-3-[4-(trifluoromethyl)phenyl]propanoate the title compound was obtained.

MS (m/z) APCI AP⁺=462

### Example 398

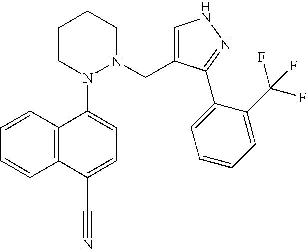

### 4-[2-({3-[2-(trifluoromethyl)phenyl]-1H-pyrazol-4-yl}methyl)tetrahydro-1(2H)-pyridazinyl]-1-naphthalenecarbonitrile (C223)

From (B1) and methyl 3-oxo-3-[2-(trifluoromethyl)phenyl]propanoate the title compound was obtained.

MS (m/z) APCI AP⁺=462

### Example 399

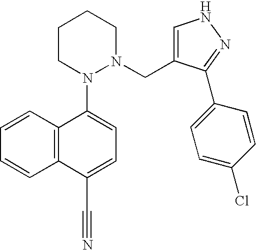

### 4-[2-{[3-(4-chlorophenyl)-1H-pyrazol-4-yl]methyl}tetrahydro-1(2H)-pyridazinyl]-1-naphthalenecarbonitrile (C224)

From (B1) and ethyl 3-oxo-3-[4-chlorophenyl]propanoate the title compound was obtained.

MS (m/z) APCI AP⁺=428

### Example 400

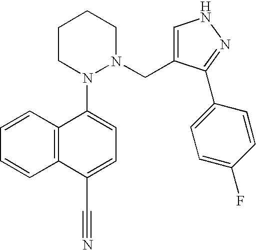

### 4-[2-{[3-(4-fluorophenyl)-1H-pyrazol-4-yl]methyl}tetrahydro-1(2H)-pyridazinyl]-1-naphthalenecarbonitrile (C225)

From (B1) and methyl 3-oxo-3-[4-fluorophenyl]propanoate the title compound was obtained.

MS (m/z) ESI ES⁺=412

### Example 401

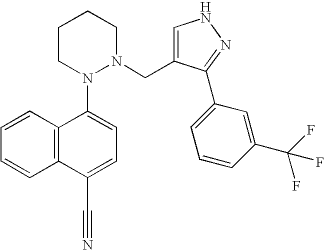

### 4-[2-({3-[3-(trifluoromethyl)phenyl]-1H-pyrazol-4-yl}methyl)tetrahydro-1(2H)-pyridazinyl]-1-naphthalenecarbonitrile (C226)

From (B1) and methyl 3-oxo-3-[3-(trifluoromethyl)phenyl]propanoate the title compound was obtained.

MS (m/z) ESI ES⁺=462

### Biological Section

Compounds of the current invention are preferably modulators of the glucocorticoid receptor. Activity mediated through oxosteroid nuclear receptors was determined using the following in vitro and in vivo assays.

### In Vitro Assays:

The following abbreviations and sources of materials are used

Fluormone PL Red—a commercially available PR fluoroprobe (PanVera Corp, Product No P2965)
Fluormone GS Red—a commercially available GR fluoroprobe (PanVera Corp, Product No P2894)
Fluormone AL Green—a commercially available AR fluoroprobe (PanVera Corp, Product No P3010)
PR-LBD—Purified human progesterone ligand binding domain tagged with Glutathione Transferase (PanVera Corp, Product No P2900)
GR—purified human glucocorticoid receptor (PanVera Corp, Product No P2812)
AR-LBD—Purified rat androgen ligand binding domain tagged with Glutathione Transferase (PanVera Corp, Product No P3009)
PR Screening Buffer—100 mM potassium phosphate (pH 7.4), 100 μG/ml bovine gamma globulin, 15% ethylene glycol, 0.02% NaN₃, 10% glycerol (PanVera Corp Product No P2967) with 0.1% w/v CHAPS
AR Screening Buffer—pH 7.5 containing protein stabilizing agents and glycerol (PanVera Corp Product No P3011)
GR Screening Buffer—100 mM potassium phosphate (pH 7.4), 200 mM Na₂MoO₂, 1 mM EDTA, 20% DMSO (PanVera Corp Product No P2814) with GR stabilizing peptide (100 μM) (PanVera Corp Product No P2815)
DTT—dithiothreitol (PanVera Corp Product No P2325)
Discovery Analyst—is an FP reader
DMSO—dimethylsulphoxide

### Glucocorticoid Receptor Fluorescence Polarization Assay (GR-FPA):

The glucocorticoid receptor fluorescence polarization assay is used to investigate the interaction of the compounds with the glucocorticoid receptor.

Compounds are added to the 384 well black plates to a final volume of 0.5 μL. Sufficient Fluormone GS Red and GR are defrosted on ice to give a final concentration of 1 nM and 4 nM, respectively. GR screening buffer is chilled to 4° C. prior to addition of DTT to give a final concentration of 1 mM. The Fluormone GS Red, and GR in GR Screening Buffer are added to compound plates to give a final volume of 10 μL. The assay is allowed to incubate at 4° C. for 12 hours. The plates are counted in a Discovery Analyst with suitable 535 nM excitation and 590 nM emission interference filters. Compounds that interact with the GR result in a lower fluorescence polarization reading. Test compounds are dissolved and diluted in DMSO. Compounds are assayed in singlicate, a four parameter curve fit of the following form being applied

$$y = \frac{a - d}{1 + {( \frac{x}{c} )}^{b}} + d$$

where a is the minimum, b is the Hill slope, c is the EC₅₀ and d is the maximum. Maximum and minimum values are compared to adhesion in the absence of compound and in the presence of 10⁻⁵M dexamethasone. Data is presented as the mean pIC₅₀ with the standard error of the mean of n experiments. Compounds with pIC₅₀ greater than 6.0 and a % max greater than 50 are preferred.

| | pIC50 in GR - FPA (as described |
|---|---|
| Example Number | above) |
| 1-401 | ≧5.8 |

### Progesterone Receptor Fluorescence Polarization Assay (PR-FPA):

The progesterone receptor fluorescence polarization assay is used to investigate the interaction of the compounds with the progesterone receptor.

Compounds are added to the 384 well black plates to a final volume of 0.5 μL. Sufficient Fluormone PL Red and PR-LBD are defrosted on ice to give a final concentration of 2 nM and 40 nM, respectively. PR screening buffer is chilled to 4° C. prior to addition of DTT to give a final concentration of 1 mM. The Fluormone PL Red and PR-LBD in PR Screening Buffer are added to compound plates to give a final volume of 10 μL. The assay is allowed to incubate at 20-22° C. for 2 hours. The plates are counted in a Discovery Analyst with suitable 535 nM excitation and 590 nM emission interference filters. Compounds that interact with the PR result in a lower fluorescence polarization reading. Test compounds are dissolved and diluted in DMSO. Compounds are assayed in singlicate, a four parameter curve fit of the following form being applied

$$y = \frac{a - d}{1 + {( \frac{x}{c} )}^{b}} + d$$

where a is the minimum, b is the Hill slope, c is the IC₅₀ and d is the maximum. Maximum and minimum values are compared to adhesion in the absence of compound and in the presence of 10⁻⁵M progesterone. Data is presented as the mean pIC₅₀ with the standard error of the mean of n experiments.

### Androgen Receptor Fluorescence Polarization Assay (AR-FPA):

The androgen receptor fluorescence polarization assay is used to investigate the interaction of the compounds with the androgen receptor.

Compounds are added to the 384 well black plates to a final volume of 0.5 μL. Sufficient Fluormone AL Green and AR-LBD are defrosted on ice to give a final concentration of 1 nM and 25 nM, respectively. AR screening buffer is chilled to 4° C. prior to addition of DTT to give a final concentration of 1 mM. The Fluormone AL Green and AR-LBD in AR Screening Buffer are added to compound plates to give a final volume of 10 μL. The assay is allowed to incubate at 20° C. for 5 hours. The plates are counted in a Discovery Analyst with suitable 485 nM excitation and 535 nM emission interference filters. Compounds that interact with the AR result in a lower fluorescence polarization reading. Test compounds are dissolved and diluted in DMSO. Compounds are assayed in singlicate, a four parameter curve fit of the following form being applied

$$y = \frac{a - d}{1 + {( \frac{x}{c} )}^{b}} + d$$

where a is the minimum, b is the Hill slope, c is the IC₅₀ and d is the maximum. Maximum and minimum values are compared to adhesion in the absence of compound and in the presence of 10⁻⁵M dihydrotestosterone. Data is presented as the mean pIC₅₀ with the standard error of the mean of n experiments.

### Cellular Tyrosine Aminotransferase Assay:

H4IIE-C3 cells were dispensed into a 96 well plate (40,000 cells/well) and were cultured in low glucose DMEM, 10% FBS, and 1× Pen/Strep. After three days, the cells were fed with an additional 200 μl of fresh serum-free media. After 24 hours of serum starvation, each well was treated either with vehicle, dexamethasone, or dexamethasone plus the appropriate GR ligand in serum free media. This treatment lasted 24 hours. The media was then removed and the cells were washed once with PBS, followed by the treatment with 50 μl of solubilization buffer (125 mM K₂HPO₄ pH=7.6, 11.0 mM EDTA pH=8.0, 0.5% Nonidet P-40, with 1.0 mM DTT added immediately before use), which was precooled to 4° C. This was followed by the 150-μl treatment of a premixed solution: 130 μl of an L-tyrosine solution [1.0 ml of L-tyrosine stock solution (63.13 mg L-tyrosine in 32 ml of 125 mM K₂HPO₄, 150 μl 10N KOH) and 0.6 ml of 125 mM KH₂PO₄], 10 ul of a 1 mM pyridoxyl phosphate solution (4.8 mg of pyridoxyl phosphate in 10 ml of 125 mM K₂HPO₄), and 10 μl of a 200 mM a-ketoglutarate solution (368 mg of a-ketoglutarate in 10 ml of 125 mM K₂HPO₄ and 150 μl of 10N KOH). The cells were incubated for 30 minutes at 37° C. At this point 3 μl of 10N KOH was added and incubated for and additional 30 minutes at 37° C. Each well was analyzed spectroscopically (Spectramax instrument) at 340 nM and IC₅₀s determined using GraphPad Prism 3.0. Compounds with pIC₅₀ greater than 5.0 are preferred.

### In Vivo Gluconeogenesis Mouse Model:

Nine to ten week old CD-1 male mice were fasted overnight on alpha-dry bedding. The model was separated into three groups: a vehicle group, a control group, and a treatment group. The procedure for each is shown below:

### Vehicle Group

After overnight fasting as described above, DMSO (5 ml/kg) was administered i.p. thirty minutes prior to saline (10 ml/kg) i.p. Blood glucose was monitored before DMSO administration and 1, 2, and 3 hours after saline administration via tail snips (10 ul) using a Glucometer Elite XL.

### Dexamethasone Group

After overnight fasting as described above, DMSO (5 ml/kg) was administered i.p., followed by dexamethasone phosphate in saline thirty minutes later (1 mg/kg, 10 ml/kg). Blood glucose was monitored before DMSO and 1, 2, and 3 hours after dexamethasone phosphate via tail snips (10 ul) using a Glucometer Elite XL.

### Treatment Group

After overnight fasting as described above, GR anatagonists (in DMSO, 5 ml/kg) were administered i.p. After thirty minutes, Dexamethasone phosphate in saline (1 mg/kg, 10 ml/kg) was administered i.p. Blood glucose was monitored before GR antagonist dosing and 1, 2, and 3 hours after Dexamethasone administration via tail snips (10 ul) using a Glucometer Elite XL.

Compounds that reduce or prevent dexamethasone-induced increases in blood glucose in the treatment group are preferred.

What is claimed is:

1. A compound of formula (I): 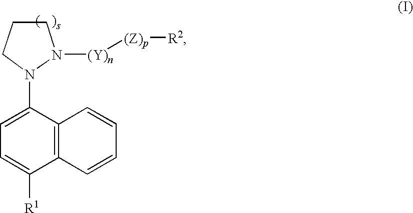
or a salt or solvate thereof, wherein
s is 1, 2, 3 or 4;
R¹ is cyano or nitro;
Y is —C(O)—;
Z is alkylene or —(R^{a})ₘO—;
R^{a} is alkylene;
m is 0 or 1;
n is 0 or 1;
p is 0 or 1;
R² is alkyl, cyano, cycloalkyl, substituted cycloalkyl, heterocycle, substituted heterocycle, aryl, substituted aryl, heteroaryl, substituted heteroaryl, haloalkyl, diphenylalkyl, alkylsilyl, amino, hydroxyl, —C(O)OCH₃, —CH(CN)CH₂Ph, —CH(OCH₂CH₃)Ph, or —NH(CH₂)₂Ph, wherein
when R² is substituted cycloalkyl, substituted aryl, substituted heteroaryl, or substituted heterocycle, each substituent is independently selected from the group consisting of alkyl, alkenyl, aryl, alkylaryl, alkylthio, alkoxy, alkenoxy, aryloxy, aralkoxy, halo, haloalkyl, haloaryl, haloalkoxy, haloalkylthio, haloalkylaryl, alkylsulfonyl, cyano, nitro, heterocycle, heteroaryl, cycloalkyl-alkylene, CH₃C(O)—, CH₃C(O)OCH₂—, and CH₃C(O)NH—.

2. A compound as claimed in claim 1, wherein s is 1, 2, or 3.

3. A compound as claimed in claim 1, wherein
s is 1;
R¹ is cyano or nitro;
Y is —C(O)—;
Z is alkylene;
n is 0 or 1;
p is 0 or 1;
R² is alkyl, cycloalkyl, heteroaryl, aryl, substituted aryl, haloalkyl, or amino, wherein
when R² is substituted aryl, each substituent is independently selected from the group consisting of alkyl, alkoxy, halo, haloalkyl, haloalkoxy, cyano, and CH₃C(O)—.

4. A compound as claimed in claim 1, wherein
s is 3;
R¹ is cyano or nitro;
Y is —C(O)—;
Z is alkylene;
n is 0 or 1;
p is 0 or 1;
R² is alkyl, cycloalkyl, substituted cycloalkyl, heterocycle, substituted heterocycle, aryl, substituted aryl, heteroaryl, substituted heteroaryl, or haloalkyl, wherein
when R² is substituted cycloalkyl, substituted aryl, substituted heteroaryl, or substituted heterocycle, each substituent is independently selected from the group consisting of alkyl, alkenyl, aryl, alkylaryl, alkoxy, halo, haloalkyl, haloalkoxy, and cyano.

5. A compound as claimed in claim 1, wherein
s is 2;
R¹ is cyano or nitro;
Y is —C(O)—;
Z is alkylene or —(R^{a})ₘO—,
R^{a} is alkylene;
m is 0 or 1;
n is 0 or 1;
p is 0 or 1;
R² is alkyl, cycloalkyl, substituted cycloalkyl, heterocycle, substituted heterocycle, aryl, substituted aryl, heteroaryl, substituted heteroaryl haloalkyl, diphenylalkyl, amino, hydroxyl, —C(O)OCH₃, —CH(CN)CH₂Ph, —CH(OCH₂CH₃)Ph, or —NH(CH₂)₂Ph, wherein
when R² is substituted cycloalkyl, substituted aryl, substituted heteroaryl, or substituted heterocycle, each substituent is independently selected from the group consisting of alkyl, alkenyl, aryl, alkylaryl, alkylthio, alkoxy, alkenoxy, aryloxy, aralkoxy, halo, haloalkyl, haloaryl, haloalkoxy, haloalkylthio, haloalkylaryl, alkylsulfonyl, cyano, nitro, heterocyclyl, cycloalkyl-alkylene, CH₃C(O)—, CH₃C(O)OCH₂—, and CH₃C(O)NH—.

6. A compound as claimed in claim 1, wherein
s is 2;
R¹ is cyano or nitro;
Y is —C(O)—;
Z is alkylene
n is 0 or 1;
p is 1;
R² is heterocycle, substituted aryl, or substituted heteroaryl, wherein
when R² is substituted aryl or substituted heteroaryl, each substituent is independently selected from the group consisting of alkyl, aryl, haloalkyl, haloalkoxy, cyano, and heteroaryl.

7. A compound as claimed in claim 1, wherein s is 2.

8. A compound as claimed in claim 1, wherein n is 0.

9. A compound as claimed in claim 1, wherein Y is —C(O)— and n is 1.

10. A compound as claimed in claim 1, wherein Z is alkylene and p is 1.

11. A compound as claimed in claim 10, wherein Z is methylene and p is 1.

12. A compound as claimed in claim 1, wherein R² is heterocycle, substituted aryl, or substituted heteroaryl, wherein
when R² is substituted aryl or substituted heteroaryl, each substituent is independently selected from the group consisting of alkyl, aryl, haloalkyl, haloalkoxy, cyano, and heteroaryl.

13. A compound as claimed in claim 1, wherein R¹ is cyano.

14. A compound of formula (IA): 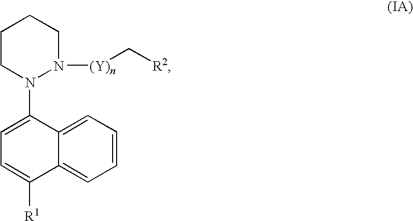
or a salt or solvate thereof, wherein
R¹ is cyano or nitro;
Y is —C(O)—;
n is 0 or 1;
R² is substituted aryl, heterocycle, or substituted heteroaryl, wherein
when R² is substituted aryl or substituted heteroaryl, each substituent is independently selected from the group consisting of alkyl, haloalkyl, haloalkoxy, heteroaryl, cyano and aryl.

15. A compound as claimed in claim 14, wherein R¹ is cyano.

16. A compound as claimed in claim 14, wherein n is 0.

17. A compound as claimed in claim 14, wherein Y is —C(O)— and n is 1.

18. A compound as claimed in claim 14, wherein Z is alkylene and p is 1.

19. A compound as claimed in claim 18, wherein Z is methylene and p is 1.

20. A compound selected from:
4-[2-(phenylmethyl)tetrahydro-1(2H)-pyridazinyl]-1-naphthalenecarbonitrile;
4-[2-{[4-(methyloxy)phenyl]methyl}tetrahydro-1(2H)-pyridazinyl]-1-naphthalenecarbonitrile;
4-[2-{[3-(ethyloxy)phenyl]methyl}tetrahydro-1(2H)-pyridazinyl]-1-naphthalenecarbonitrile;
4-[2-{[4-(ethyloxy)phenyl]methyl}tetrahydro-1(2H)-pyridazinyl]-1-naphthalenecarbonitrile;
4-[2-{[4-(butyloxy)phenyl]methyl}tetrahydro-1(2H)-pyridazinyl]-1-naphthalenecarbonitrile;
4-[2-[(4-ethylphenyl)methyl]tetrahydro-1(2H)-pyridazinyl]-1-naphthalenecarbonitrile;
4-[2-{[4-(1-methylethyl)phenyl]methyl}tetrahydro-1(2H)-pyridazinyl]-1-naphthalenecarbonitrile;
4-[2-[(2-bromophenyl)methyl]tetrahydro-1(2H)-pyridazinyl]-1-naphthalenecarbonitrile;
4-[2-[(3-bromophenyl)methyl]tetrahydro-1(2H)-pyridazinyl]-1-naphthalenecarbonitrile;
4-[2-[(4-bromophenyl)methyl]tetrahydro-1(2H)-pyridazinyl]-1-naphthalenecarbonitrile;
4-[2-[(2-chlorophenyl)methyl]tetrahydro-1(2H)-pyridazinyl]-1-naphthalenecarbonitrile;
4-[2-[(2-methylphenyl)methyl]tetrahydro-1(2H)-pyridazinyl]-1-naphthalenecarbonitrile;
4-[2-[(3-chlorophenyl)methyl]tetrahydro-1(2H)-pyridazinyl]-1-naphthalenecarbonitrile;
4-[2-[(4-chlorophenyl)methyl]tetrahydro-1(2H)-pyridazinyl]-1-naphthalenecarbonitrile;
4-[2-[(2-fluorophenyl)methyl]tetrahydro-1(2H)-pyridazinyl]-1-naphthalenecarbonitrile;
4-[2-[(3-fluorophenyl)methyl]tetrahydro-1(2H)-pyridazinyl]-1-naphthalenecarbonitrile;
4-[2-[(4-fluorophenyl)methyl]tetrahydro-1(2H)-pyridazinyl]-1-naphthalenecarbonitrile;
4-[2-[(2-cyanophenyl)methyl]tetrahydro-1(2H)-pyridazinyl]-1-naphthalenecarbonitrile;
4-[2-[(3-cyanophenyl)methyl]tetrahydro-1(2H)-pyridazinyl]-1-naphthalenecarbonitrile;
4-[2-[(4-cyanophenyl)methyl]tetrahydro-1(2H)-pyridazinyl]-1-naphthalenecarbonitrile;
4-[2-{[4-(methylthio)phenyl]methyl}tetrahydro-1(2H)-pyridazinyl]-1-naphthalenecarbonitrile;
4-[2-[(3,4-dichlorophenyl)methyl]tetrahydro-1(2H)-pyridazinyl]-1-naphthalenecarbonitrile;
4-[2-[(3-methylphenyl)methyl]tetrahydro-1(2H)-pyridazinyl]-1-naphthalenecarbonitrile;
4-[2-[(4-acetylphenyl)methyl]tetrahydro-1(2H)-pyridazinyl]-1-naphthalenecarbonitrile;
4-[2-{[4-(methylsulfonyl)phenyl]methyl}tetrahydro-1(2H)-pyridazinyl]-1-naphthalenecarbonitrile;
4-[2-{[4-(phenyloxy)phenyl]methyl}tetrahydro-1(2H)-pyridazinyl]-1-naphthalenecarbonitrile;
4-[2-[(4-butylphenyl)methyl]tetrahydro-1(2H)-pyridazinyl]-1-naphthalenecarbonitrile;
4-[2-({2-[(1,1-dimethylethyl)thio]phenyl}methyl)tetrahydro-1(2H)-pyridazinyl]-1-naphthalenecarbonitrile;
4-[2-({3-[(trifluoromethyl)thio]phenyl}methyl)tetrahydro-1(2H)-pyridazinyl]-1-naphthalenecarbonitrile;
4-[2-{[2,4-bis(trifluoromethyl)phenyl]methyl}tetrahydro-1(2H)-pyridazinyl]-1-naphthalenecarbonitrile;
4-[2-{[3,5-bis(trifluoromethyl)phenyl]methyl}tetrahydro-1(2H)-pyridazinyl]-1-naphthalenecarbonitrile;
4-[2-[(4-methylphenyl)methyl]tetrahydro-1(2H)-pyridazinyl]-1-naphthalenecarbonitrile;
4-[2-(1-naphthalenylmethyl)tetrahydro-1(2H)-pyridazinyl]-1-naphthalenecarbonitrile;
4-[2-(2-naphthalenylmethyl)tetrahydro-1(2H)-pyridazinyl]-1-naphthalenecarbonitrile;
4-[2-(3-thienylmethyl)tetrahydro-1(2H)-pyridazinyl]-1-naphthalenecarbonitrile;
4-[2-{[2,4-bis(methyloxy)phenyl]methyl}tetrahydro-1(2H)-pyridazinyl]-1-naphthalenecarbonitrile;
4-[2-{[2-(trifluoromethyl)phenyl]methyl}tetrahydro-1(2H)-pyridazinyl]-1-naphthalenecarbonitrile;
4-[2-{[4-(trifluoromethyl)phenyl]methyl}tetrahydro-1(2H)-pyridazinyl]-1-naphthalenecarbonitrile;
4-[2-{[2-(methyloxy)phenyl]methyl}tetrahydro-1(2H)-pyridazinyl]-1-naphthalenecarbonitrile;
4-[2-{[3-(methyloxy)phenyl]methyl}tetrahydro-1(2H)-pyridazinyl]-1-naphthalenecarbonitrile;
4-[2-{[3-(phenyloxy)phenyl]methyl}tetrahydro-1(2H)-pyridazinyl]-1-naphthalenecarbonitrile;
4-[2-(cyclohexylmethyl)tetrahydro-1(2H)-pyridazinyl]-1-naphthalenecarbonitrile;
4-[2-{[2-(2-propen-1-yloxy)phenyl]methyl}tetrahydro-1(2H)-pyridazinyl]-1-naphthalenecarbonitrile;
4-[2-(2,1,3-benzoxadiazol-4-ylmethyl)tetrahydro-1(2H)-pyridazinyl]-1-naphthalenecarbonitrile;
4-[2-{[1-(1,1-dimethylethyl)-5-methyl-1H-pyrazol-3-yl]methyl}tetrahydro-1(2H)-pyridazinyl]-1-naphthalenecarbonitrile;
4-[2-{[5-(2-pyridinyl)-2-thienyl]methyl}tetrahydro-1(2H)-pyridazinyl]-1-naphthalenecarbonitrile;
4-[2-[(1-phenyl-1H-pyrazol-5-yl)methyl]tetrahydro-1(2H)-pyridazinyl]-1-naphthalenecarbonitrile;
4-[2-[(2-phenyl-1H-imidazol-4-yl)methyl]tetrahydro-1(2H)-pyridazinyl]-1-naphthalenecarbonitrile;
4-[2-[(3-phenyl-1H-pyrazol-4-yl)methyl]tetrahydro-1(2H)-pyridazinyl]-1-naphthalenecarbonitrile;
N-(5-{[2-(4-cyano-1-naphthalenyl)tetrahydro-1(2H)-pyridazinyl]methyl}-1,3-thiazol-2-yl)acetamide;
4-[2-(2-pyridinylmethyl)tetrahydro-1(2H)-pyridazinyl]-1-naphthalenecarbonitrile;
(5-{[2-(4-cyano-1-naphthalenyl)tetrahydro-1(2H)-pyridazinyl]methyl}-2-furanyl)methyl acetate;
4-[2-[(1-phenyl-1H-imidazol-2-yl)methyl]tetrahydro-1(2H)-pyridazinyl]-1-naphthalenecarbonitrile;
4-[2-{[6-(methyloxy)-2-pyridinyl]methyl}tetrahydro-1(2H)-pyridazinyl]-1-naphthalenecarbonitrile;
4-[2-(1H-pyrazol-3-ylmethyl)tetrahydro-1(2H)-pyridazinyl]-1-naphthalenecarbonitrile;
4-[2-[(2-ethyl-4-methyl-1H-imidazol-5-yl)methyl]tetrahydro-1(2H)-pyridazinyl]-1-naphthalenecarbonitrile;
5-{[2-(4-cyano-1-naphthalenyl)tetrahydro-1(2H)-pyridazinyl]methyl}-3-pyridinecarbonitrile;
4-[2-[(1-phenyl-1H-pyrazol-4-yl)methyl]tetrahydro-1(2H)-pyridazinyl]-1-naphthalenecarbonitrile;
4-[2-[(3-methyl-2-thienyl)methyl]tetrahydro-1(2H)-pyridazinyl]-1-naphthalenecarbonitrile;
4-[2-[(5-methyl-2-thienyl)methyl]tetrahydro-1(2H)-pyridazinyl]-1-naphthalenecarbonitrile;
4-[2-(1H-imidazol-4-ylmethyl)tetrahydro-1(2H)-pyridazinyl]-1-naphthalenecarbonitrile;
4-[2-[(3,5-dichlorophenyl)methyl]tetrahydro-1(2H)-pyridazinyl]-1-naphthalenecarbonitrile;
4-[2-[(5-chloro-1,3-dimethyl-1H-pyrazol-4-yl)methyl]tetrahydro-1(2H)-pyridazinyl]-1-naphthalenecarbonitrile;
4-[2-[(3,5-dibromophenyl)methyl]tetrahydro-1(2H)-pyridazinyl]-1-naphthalenecarbonitrile;
4-[2-({5-[3-(trifluoromethyl)phenyl]-2-furanyl}methyl)tetrahydro-1(2H)-pyridazinyl]-1-naphthalenecarbonitrile;
4-[2-{[1-(3,5-dichlorophenyl)-1H-pyrrol-2-yl]methyl}tetrahydro-1(2H)-pyridazinyl]-1-naphthalenecarbonitrile;
4-[2-{[3-fluoro-5-(trifluoromethyl)phenyl]methyl}tetrahydro-1(2H)-pyridazinyl]-1-naphthalenecarbonitrile;
4-[2-[(3,5-difluorophenyl)methyl]tetrahydro-1(2H)-pyridazinyl]-1-naphthalenecarbonitrile;
4-[2-{[3,5-bis(methyloxy)phenyl]methyl}tetrahydro-1(2H)-pyridazinyl]-1-naphthalenecarbonitrile;
4-[2-[(5-ethyl-2-furanyl)methyl]tetrahydro-1(2H)-pyridazinyl]-1-naphthalenecarbonitrile;
4-[2-[(5-bromo-2-furanyl)methyl]tetrahydro-1(2H)-pyridazinyl]-1-naphthalenecarbonitrile;
4-[2-[(4,5-dimethyl-2-furanyl)methyl]tetrahydro-1(2H)-pyridazinyl]-1-naphthalenecarbonitrile;
4-[2-(3-pyridinylmethyl)tetrahydro-1(2H)-pyridazinyl]-1-naphthalenecarbonitrile
4-[2-[(6-methyl-2-pyridinyl)methyl]tetrahydro-1(2H-pyridazinyl]-1-naphthalenecarbonitrile;
4-[2-[(4-bromo-1H-pyrazol-3-yl)methyl]tetrahydro-1(2H)-pyridazinyl]-1-naphthalenecarbonitrile;
4-[2-[(5-bromo-2-thienyl)methyl]tetrahydro-1(2H)-pyridazinyl]-1-naphthalenecarbonitrile;
4-[2-{[5-chloro-1-methyl-3-(trifluoromethyl)-1H-pyrazol-4-yl]methyl}tetrahydro-1(2H)-pyridazinyl]-1-naphthalenecarbonitrile;
4-[2-[(1-methyl-1H-imidazol-2-yl)methyl]tetrahydro-1(2H)-pyridazinyl]-1-naphthalenecarbonitrile;
4-[2-[(4-methyl-1H-imidazol-5-yl)methyl]tetrahydro-1(2H)-pyridazinyl]-1-naphthalenecarbonitrile;
4-[2-{[4-chloro-2-(cyclopropylmethyl)-1H-imidazol-5-yl]methyl}tetrahydro-1(2H)-pyridazinyl]-1-naphthalenecarbonitrile;
4-[2-[(2-butyl-1H-imidazol-4-yl)methyl]tetrahydro-1(2H)-pyridazinyl]-1-naphthalenecarbonitrile;
4-[2-[(3-bromo-4-pyridinyl)methyl]tetrahydro-1(2H)-pyridazinyl]-1-naphthalenecarbonitrile;
4-[2-({6-[(1,1-dimethylethyl)oxy]-2-pyridinyl}methyl)tetrahydro-1(2H)-pyridazinyl]-1-naphthalenecarbonitrile;
4-[2-[(2-ethyl-1H-imidazol-4-yl)methyl]tetrahydro-1(2H)-pyridazinyl]-1-naphthalenecarbonitrile;
4-[2-[(4-bromo-2-thienyl)methyl]tetrahydro-1(2H)-pyridazinyl]-1-naphthalenecarbonitrile;
4-[2-(1H-pyrazol-4-ylmethyl)tetrahydro-1(2H)-pyridazinyl]-1-naphthalenecarbonitrile;
4-[2-[(5-methyl-2-furanyl)methyl]tetrahydro-1(2H)-pyridazinyl]-1-naphthalenecarbonitrile;
4-[2-[(5-bromo-3-thienyl)methyl]tetrahydro-1(2H)-pyridazinyl]-1-naphthalenecarbonitrile;
4-[2-[(1,3-dimethyl-1H-pyrazol-5-yl)methyl]tetrahydro-1(2H)-pyridazinyl]-1-naphthalenecarbonitrile;
4-[2-[(6-bromo-3-pyridinyl)methyl]tetrahydro-1(2H)-pyridazinyl]-1-naphthalenecarbonitrile;
4-[2-(1H-1,2,3-triazol-4-ylmethyl)tetrahydro-1(2H)-pyridazinyl]-1-naphthalenecarbonitrile;
4-[2-(1H-imidazol-2-ylmethyl)tetrahydro-1(2H)-pyridazinyl]-1-naphthalenecarbonitrile;
4-(2-{[1-(1,1-dimethylethyl)-5-methyl-1H-pyrazol-3-yl]methyl}hexahydro-1H-1,2-diazepin-1-yl)-1-naphthalenecarbonitrile;
4-[2-({2-[(trifluoromethyl)oxy]phenyl}methyl)hexahydro-1H-1,2-diazepin-1-yl]-1-naphthalenecarbonitrile;
4-[2-(1H-1,2,3-triazol-4-ylmethyl)hexahydro-1H-1,2-diazepin-1-yl]-1-naphthalenecarbonitrile;
4-[2-(phenylmethyl)hexahydro-1H-1,2-diazepin-1-yl]-1-naphthalenecarbonitrile;
4-{2-[(4-phenyl-1H-imidazol-5-yl)methyl]hexahydro-1H-1,2-diazepin-1-yl}-1-naphthalenecarbonitrile;
4-(2-hexylhexahydro-1H-1,2-diazepin-1-yl)-1-naphthalenecarbonitrile;
4-{2-[(3-ethenylphenyl)methyl]hexahydro-1H-1,2-diazepin-1-yl}-1-naphthalenecarbonitrile;
4-{2-[(2-fluorophenyl)methyl]hexahydro-1H-1,2-diazepin-1-yl}-1-naphthalenecarbonitrile;
4-{2-[(1-phenyl-1H-pyrazol-4-yl)methyl]hexahydro-1H-1,2-diazepin-1-yl}-1-naphthalenecarbonitrile;
4-[2-(2-pyridinylmethyl)hexahydro-1H-1,2-diazepin-1-yl]-1-naphthalenecarbonitrile;
4-{2-[(3,5-dimethylphenyl)methyl]hexahydro-1H-1,2-diazepin-1-yl}-1-naphthalenecarbonitrile;
4-{2-[(3-methyl-5-phenyl-4-isoxazolyl)methyl]hexahydro-1H-1,2-diazepin-1-yl}-1-naphthalenecarbonitrile;
4-{2-[(2-methylphenyl)methyl]hexahydro-1H-1,2-diazepin-1-yl}-1-naphthalenecarbonitrile;
4-(2-{[2-(trifluoromethyl)phenyl]methyl}hexahydro-1H-1,2-diazepin-1-yl)-1-naphthalenecarbonitrile;
4-[2-(3-pyridinylmethyl)hexahydro-1H-1,2-diazepin-1-yl]-1-naphthalenecarbonitrile;
4-{2-[(3-phenyl-1H-pyrazol-5-yl)methyl]hexahydro-1H-1,2-diazepin-1-yl}-1-naphthalenecarbonitrile;
5-{[2-(4-cyano-1-naphthalenyl)hexahydro-1H-1,2-diazepin-1-yl]methyl}-3-pyridinecarbonitrile;
4-{2-[(2,2,3,3-tetramethylcyclopropyl)methyl]hexahydro-1H-1,2-diazepin-1-yl}-1-naphthalenecarbonitrile;
4-{2-[(1,3-dimethyl-1H-pyrazol-5-yl)methyl]hexahydro-1H-1,2-diazepin-1-yl}-1-naphthalenecarbonitrile;
4-{2-[(1-phenyl-1H-pyrazol-5-yl)methyl]hexahydro-1H-1,2-diazepin-1-yl}-1-naphthalenecarbonitrile;
4-{2-[(4-bromo-1H-pyrazol-3-yl)methyl]hexahydro-1H-1,2-diazepin-1-yl}-1-naphthalenecarbonitrile;
4-{2-[(3-methylphenyl)methyl]hexahydro-1H-1,2-diazepin-1-yl}-1-naphthalenecarbonitrile;
4-{2-[(2-cyanophenyl)methyl]hexahydro-1H-1,2-diazepin-1-yl}-1-naphthalenecarbonitrile;
4-(2-{[3-(4-methylphenyl)-1H-pyrazol-4-yl]methyl}hexahydro-1H-1,2-diazepin-1-yl)-1-naphthalenecarbonitrile;
4-{2-[(6-methyl-2-pyridinyl)methyl]hexahydro-1H-1,2-diazepin-1-yl}-1-naphthalenecarbonitrile;
4-{2-[(4-cyanophenyl)methyl]hexahydro-1H-1,2-diazepin-1-yl}-1-naphthalenecarbonitrile;
4-[2-(1H-pyrazol-3-ylmethyl)hexahydro-1H-1,2-diazepin-1-yl]-1-naphthalenecarbonitrile;
4-[2-(4-pyridinylmethyl)hexahydro-1H-1,2-diazepin-1-yl]-1-naphthalenecarbonitrile;
4-[2-(2-cyclopentylethyl)hexahydro-1H-1,2-diazepin-1-yl]-1-naphthalenecarbonitrile;
4-{2-[(3-cyanophenyl)methyl]hexahydro-1H-1,2-diazepin-1-yl}-1-naphthalenecarbonitrile;
4-(2-{[4-(trifluoromethyl)phenyl]methyl}hexahydro-1H-1,2-diazepin-1-yl)-1-naphthalenecarbonitrile;
4-(2-{[2-fluoro-3-(trifluoromethyl)phenyl]methyl}hexahydro-1H-1,2-diazepin-1-yl)-1-naphthalenecarbonitrite;
4-(2-{[3-(ethyloxy)phenyl]methyl}hexahydro-1H-1,2-diazepin-1-yl)-1-naphthalenecarbonitrile;
4-{2-[(3-fluorophenyl)methyl]hexahydro-1H-1,2-diazepin-1-yl}-1-naphthalenecarbonitrile;
4-{2-[(3-methyl-2-thienyl)methyl]hexahydro-1H-1,2-diazepin-1-yl}-1-naphthalenecarbonitrile;
4-(2-{[4-fluoro-2-(trifluoromethyl)phenyl]methyl}hexahydro-1H-1,2-diazepin-1-yl)-1-naphthalenecarbonitrile;
4-[2-({3-[(trifluoromethyl)oxy]phenyl}methyl)hexahydro-1H-1,2-diazepin-1-yl]-1-naphthalenecarbonitrile;
4-[2-({3-[(trifluoromethyl)oxy]phenyl}methyl)hexahydro-1H-1,2-diazepin-1-yl]-1-naphthalenecarbonitrile;
4-{2-[(5-methyl-2-thienyl)methyl]hexahydro-1H-1,2-diazepin-1-yl}-1-naphthalenecarbonitrile;
4-[2-(2-naphthalenylmethyl)hexahydro-1H-1,2-diazepin-1-yl]-1-naphthalenecarbonitrile;
4-{2-[(3-phenyl-1H-pyrazol-4-yl)methyl]hexahydro-1H-1,2-diazepin-1-yl}-1-naphthalenecarbonitrile;
4-(2-{[3-(trifluoromethyl)phenyl]methyl}hexahydro-1H-1,2-diazepin-1-yl)-1-naphthalenecarbonitrile;
4-(2-{[6-(methyloxy)-2-pyridinyl]methyl}hexahydro-1H-1,2-diazepin-1-yl)-1-naphthalenecarbonitrile;
4-{2-[(3,5-difluorophenyl)methyl]hexahydro-1H-1,2-diazepin-1-yl}-1-naphthalenecarbonitrile;
4-{2-[(4,5-dimethyl-2-furanyl)methyl]hexahydro-1H-1,2-diazepin-1-yl}-1-naphthalenecarbonitrile;
4-(2-{[3,5-bis(trifluoromethyl)phenyl]methyl}hexahydro-1H-1,2-diazepin-1-yl)-1-naphthalenecarbonitrile;
4-[2-(1-naphthalenylmethyl)hexahydro-1H-1,2-diazepin-1-yl]-1-naphthalenecarbonitrile;
4-(2-{[4-(methyloxy)phenyl]methyl}hexahydro-1H-1,2-diazepin-1-yl)-1-naphthalenecarbonitrile;
4-(2-{[3-(methyloxy)phenyl]methyl}hexahydro-1H-1,2-diazepin-1-yl)-1-naphthalenecarbonitrile;
4-(2-{[2-(methyloxy)phenyl]methyl}hexahydro-1H-1,2-diazepin-1-yl)-1-naphthalenecarbonitrile;
4-{[2-(4-nitro-1-naphthalenyl)tetrahydro-1(2H)-pyridazinyl]methyl}benzonitrile;
1-{[4-(1-methylethyl)phenyl]methyl}-2-(4-nitro-1-naphthalenyl)hexahydropyridazine;
1-(4-nitro-1-naphthalenyl)-2-(phenylmethyl)hexahydropyridazine;
1-(cyclohexylmethyl)-2-(4-nitro-1-naphthalenyl)hexahydropyridazine;
4-[2-(3-phenylbutyl)tetrahydro-1(2H)-pyridazinyl]-1-naphthalenecarbonitrile;
4-[2-(3-phenylpropyl)tetrahydro-1(2H)-pyridazinyl]-1-naphthalenecarbonitrile;
1-(4-nitro-1-naphthalenyl)-2-(3-phenylbutyl)hexahydropyridazine;
1-(4-nitro-1-naphthalenyl)-2-(3-phenylpropyl)hexahydropyridazine;
1-methyl-2-(4-nitro-1-naphthalenyl)hexahydropyridazine;
Methyl 6-[2-(4-nitro-1-naphthalenyl)tetrahydro-1(2H)-pyridazinyl]hexanoate;
4-{2-[(3-bromophenyl)methyl]-1-pyrazolidinyl}-1-naphthalenecarbonitrile;
4-[2-(3-thienylmethyl)-1-pyrazolidinyl]-1-naphthalenecarbonitrile;
4-(2-{[2,4-bis(trifluoromethyl)phenyl]methyl}-1-pyrazolidinyl)-1-naphthalenecarbonitrile;
4-{2-[(3-fluorophenyl)methyl]-1-pyrazolidinyl}-1-naphthalenecarbonitrile;
4-{2-[(2-bromophenyl)methyl]-1-pyrazolidinyl}-1-naphthalenecarbonitrile;
4-{2-[(4-acetylphenyl)methyl]-1-pyrazolidinyl}-1-naphthalenecarbonitrile;
4-(2-{[2-(trifluoromethyl)phenyl]methyl}-1-pyrazolidinyl)-1-naphthalenecarbonitrile;
4-(2-{[4-(1-methylethyl)phenyl]methyl}-1-pyrazolidinyl)-1-naphthalenecarbonitrile;
4-{2-[(4-fluorophenyl)methyl]-1-pyrazolidinyl}-1-naphthalenecarbonitrile;
4-(2-{[4-(trifluoromethyl)phenyl]methyl}-1-pyrazolidinyl)-1-naphthalenecarbonitrile;
4-[2-({3-[(trifluoromethyl)oxy]phenyl}methyl)-1-pyrazolidinyl]-1-naphthalenecarbonitrile;
4-{2-[(2-fluorophenyl)methyl]-1-pyrazolidinyl)-1-naphthalenecarbonitrile;
4-(2-([3-(methyloxy)phenyl]methyl}-1-pyrazolidinyl)-1-naphthalenecarbonitrile;
4-(2-{[3,5-bis(trifluoromethyl)phenyl]methyl}-1-pyrazolidinyl)-1-naphthalenecarbonitrile;
4-(2-{[3-fluoro-5-(trifluoromethyl)phenyl]methyl}-1-pyrazolidinyl)-1-naphthalenecarbonitrile;
4-(2-{[3-(trifluoromethyl)phenyl]methyl}-1-pyrazolidinyl)-1-naphthalenecarbonitrile;
4-{2-[(4-methylphenyl)methyl]-1-pyrazolidinyl}-1-naphthalenecarbonitrile;
4-[2-(1-naphthalenylmethyl)-1-pyrazolidinyl]-1-naphthalenecarbonitrile;
4-[2-(phenylmethyl)-1-pyrazolidinyl]-1-naphthalenecarbonitrile;
4-{2-[(3,4-dichlorophenyl)methyl]-1-pyrazolidinyl}-1-naphthalenecarbonitrile;
4-(2-{[4-(methyloxy)phenyl]methyl}-1-pyrazolidinyl)-1-naphthalenecarbonitrile;
4-(2-{[3-(ethyloxy)phenyl]methyl}-1-pyrazolidinyl)-1-naphthalenecarbonitrile;
4-{2-[(4-cyanophenyl)methyl]-1-pyrazolidinyl}-1-naphthalenecarbonitrile;
4-{2-[(3-cyanophenyl)methyl]-1-pyrazolidinyl}-1-naphthalenecarbonitrile;
4-{2-[(2-methylphenyl)methyl]-1-pyrazolidinyl}-1-naphthalenecarbonitrile;
4-[2-[(4-phenyl-1H-imidazol-5-yl)methyl]tetrahydro-1(2H)-pyridazinyl]-1-naphthalenecarbonitrile;
4-[2-{[3-(1-methylethyl)-1H-pyrazol-4-yl]methyl}tetrahydro-1(2H)-pyridazinyl]-1-naphthalenecarbonitrile;
4-[2-{[3-(1-methylethyl)-1H-pyrazol-4-yl]methyl}tetrahydro-1(2H)-pyridazinyl]-1-naphthalenecarbonitrile;
4-[2-[(3-ethyl-1H-pyrazol-4-yl)methyl]tetrahydro-1(2H)-pyridazinyl]-1-naphthalenecarbonitrile;
4-[2-[(3-propyl-1H-pyrazol-4-yl)methyl]tetrahydro-1(2H)-pyridazinyl]-1-naphthalenecarbonitrile;
4-[2-{[3-(2-pyridinyl)-1H-pyrazol-4-yl]methyl}tetrahydro-1(2H)-pyridazinyl]-1-naphthalenecarbonitrile;
4-[2-{[3-(3-methylphenyl)-1H-pyrazol-4-yl]methyl}tetrahydro-1(2H)-pyridazinyl]-1-naphthalenecarbonitrile;
1-(2-{[(1,1-dimethylethyl)(dimethyl)silyl]oxy}ethyl)-2-(4-nitro-1-naphthalenyl)hexahydropyridazine;
4-[2-(2-{[(1,1-dimethylethyl)(dimethyl)silyl]oxy}ethyl)tetrahydro-1(2H)-pyridazinyl]-1-naphthalenecarbonitrile;
4-[2-{2-[2-(trifluoromethyl)phenyl]ethyl}tetrahydro-1(2H)-pyridazinyl]-1-naphthalenecarbonitrile;
4-[2-[2-(3,4-dichlorophenyl)ethyl]tetrahydro-1(2H)-pyridazinyl]-1-naphthalenecarbonitrile;
4-[2-[2-(3-fluorophenyl)ethyl]tetrahydro-1(2H)-pyridazinyl]-1-naphthalenecarbonitrile;
4-[2-[2-(2-methylphenyl)ethyl]tetrahydro-1(2H)-pyridazinyl]-1-naphthalenecarbonitrile;
4-[2-{2-[3-(methyloxy)phenyl]ethyl}tetrahydro-1(2H)-pyridazinyl]-1-naphthalenecarbonitrile;
4-[2-[2-(4-fluorophenyl)ethyl]tetrahydro-1(2H)-pyridazinyl]-1-naphthalenecarbonitrile;
4-[2-[2-(4-methylphenyl)ethyl]tetrahydro-1(2H)-pyridazinyl]-1-naphthalenecarbonitrile;
4-[2-[2-(2-bromophenyl)ethyl]tetrahydro-1(2H)-pyridazinyl]-1-naphthalenecarbonitrile;
4-[2-[2-(3-methylphenyl)ethyl]tetrahydro-1(2H)-pyridazinyl]-1-naphthalenecarbonitrile;
4-[2-[2-(2,4-dichlorophenyl)ethyl]tetrahydro-1(2H)-pyridazinyl]-1-naphthalenecarbonitrile;
4-[2-(2-phenylethyl)tetrahydro-1(2H)-pyridazinyl]-1-naphthalenecarbonitrile;
4-[2-[2-(1-naphthalenyl)ethyl]tetrahydro-1(2H)-pyridazinyl]-1-naphthalenecarbonitrile;
4-[2-{2-[3-(trifluoromethyl)phenyl]ethyl}tetrahydro-1(2H)-pyridazinyl]-1-naphthalenecarbonitrile;
4-[2-[2-(3-bromophenyl)ethyl]tetrahydro-1(2H)-pyridazinyl]-1-naphthalenecarbonitrile;
4-[2-[2-(2,6-dichlorophenyl)ethyl]tetrahydro-1(2H)-pyridazinyl]-1-naphthalenecarbonitrile;
4-[2-[2-(2-chloro-4-fluorophenyl)ethyl]tetrahydro-1(2H)-pyridazinyl]-1-naphthalenecarbonitrile;
4-[2-[2-(2-chlorophenyl)ethyl]tetrahydro-1(2H)-pyridazinyl]-1-naphthalenecarbonitrile;
4-[2-[2-(2,4,6-trimethylphenyl)ethyl]tetrahydro-1(2H)-pyridazinyl]-1-naphthalenecarbonitrile;
1-[2-(2,6-dichlorophenyl)ethyl]-2-(4-nitro-1-naphthalenyl)hexahydropyridazine;
1-[2-(2-chloro-4-fluorophenyl)ethyl]-2-(4-nitro-1-naphthalenyl)hexahydropyridazine;
1-[2-(2-chlorophenyl)ethyl]-2-(4-nitro-1-naphthalenyl)hexahydropyridazine;
1-(4-nitro-1-naphthalenyl)-2-[2-(2,4,6-trimethylphenyl)ethyl]hexahydropyridazine
1-[2-(2-methylphenyl)ethyl]-2-(4-nitro-1-naphthalenyl)hexahydropyridazine;
1-{2-[3-(methyloxy)phenyl]ethyl}-2-(4-nitro-1-naphthalenyl)hexahydropyridazine;
1-(4-nitro-1-naphthalenyl)-2-{2-[2-(trifluoromethyl)phenyl]ethyl}hexahydropyridazine;
1-[2-(2-bromophenyl)ethyl]-2-(4-nitro-1-naphthalenyl)hexahydropyridazine;
1-(4-nitro-1-naphthalenyl)-2-[2-(3-nitrophenyl)ethyl]hexahydropyridazine;
1-[2-(3-methylphenyl)ethyl]-2-(4-nitro-1-naphthalenyl)hexahydropyridazine;
1-[2-(2,4-dichlorophenyl)ethyl]-2-(4-nitro-1-naphthalenyl)hexahydropyridazine;
1-[2-(1-naphthalenyl)ethyl]-2-(4-nitro-1-naphthalenyl)hexahydropyridazine;
1-(4-nitro-1-naphthalenyl)-2-{2-[3-(trifluoromethyl)phenyl]ethyl}hexahydropyridazine;
1-[2-(3-bromophenyl)ethyl]-2-(4-nitro-1-naphthalenyl)hexahydropyridazine;
1-[2-(4-fluorophenyl)ethyl]-2-(4-nitro-1-naphthalenyl)hexahydropyridazine;
4-{2-[2-(4-nitro-1-naphthalenyl)tetrahydro-1(2H)-pyridazinyl]ethyl}benzonitrile;
1-[2-(4-chlorophenyl)ethyl]-2-(4-nitro-1-naphthalenyl)hexahydropyridazine;
1-{2-[4-(ethyloxy)phenyl]ethyl}-2-(4-nitro-1-naphthalenyl)hexahydropyridazine;
1-(4-nitro-1-naphthalenyl)-2-(2-phenylethyl)hexahydropyridazine;
4-[2-(cyclohexylcarbonyl)tetrahydro-1(2H)-pyridazinyl]-1-naphthalenecarbonitrile;
4-(2-pentanoyltetrahydro-1(2H)-pyridazinyl)-1-naphthalenecarbonitrile;
4-[2-({3-[(phenylmethyl)oxy]phenyl}acetyl)tetrahydro-1(2H)-pyridazinyl]-1-naphthalenecarbonitrile;
4-[2-(cyclopentylacetyl)tetrahydro-1(2H)-pyridazinyl]-1-naphthalenecarbonitrile;
4-[2-(phenylacetyl)tetrahydro-1(2H)-pyridazinyl]-1-naphthalenecarbonitrile;
4-[2-(2-phenylbutanoyl)tetrahydro-1(2H)-pyridazinyl]-1-naphthalenecarbonitrile;
4-[2-{[(1S,2S)-2-phenylcyclopropyl]carbonyl}tetrahydro-1(2H)-pyridazinyl]-1-naphthalenecarbonitrile;
4-[2-(3-phenylpropanoyl)tetrahydro-1(2H)-pyridazinyl]-1-naphthalenecarbonitrile;
4-[2-(3,3-dimethylbutanoyl)tetrahydro-1(2H)-pyridazinyl]-1-naphthalenecarbonitrile;
4-[2-{[4-(methyloxy)phenyl]carbonyl}tetrahydro-1(2H)-pyridazinyl]-1-naphthalenecarbonitrile;
4-[2-({3-[(trifluoromethyl)oxy]phenyl}carbonyl)tetrahydro-1(2H)-pyridazinyl]-1-naphthalenecarbonitrile;
4-[2-{[3-(methyloxy)phenyl]carbonyl}tetrahydro-1(2H)-pyridazinyl]-1-naphthalenecarbonitrile;
4-[2-{[2-(methyloxy)phenyl]carbonyl}tetrahydro-1(2H)-pyridazinyl]-1-naphthalenecarbonitrile;
4-[2-{[2-(trifluoromethyl)phenyl]carbonyl}tetrahydro-1(2H)-pyridazinyl]-1-naphthalenecarbonitrile;
4-[2-{[3-(trifluoromethyl)phenyl]carbonyl}tetrahydro-1(2H)-pyridazinyl]-1-naphthalenecarbonitrile;
4-[2-({4-[(trifluoromethyl)oxy]phenyl}carbonyl)tetrahydro-1(2H)-pyridazinyl]-1-naphthalenecarbonitrile;
4-[2-[(3-cyanophenyl)carbonyl]tetrahydro-1(2H)-pyridazinyl]-1-naphthalenecarbonitrile;
4-[2-{[4-(trifluoromethyl)phenyl]carbonyl}tetrahydro-1(2H)-pyridazinyl]-1-naphthalenecarbonitrile;
4-[2-({2-[(trifluoromethyl)oxy]phenyl}carbonyl)tetrahydro-1(2H)-pyridazinyl]-1-naphthalenecarbonitrile;
1-(4-nitro-1-naphthalenyl)-2-pentanoylhexahydropyridazine;
1-(4-nitro-1-naphthalenyl)-2-{[3-(trifluoromethyl)phenyl]carbonyl}hexahydropyridazine;
1-(4-nitro-1-naphthalenyl)-2-{[4-(trifluoromethyl)phenyl]carbonyl}hexahydropyridazine;
4-{[2-(4-nitro-1-naphthalenyl)tetrahydro-1(2H)-pyridazinyl]carbonyl}benzonitrile;
1-{[3-(methyloxy)phenyl]carbonyl}-2-(4-nitro-1-naphthalenyl)hexahydropyridazine;
1-{[4-(methyloxy)phenyl]carbonyl}-2-(4-nitro-1-naphthalenyl)hexahydropyridazine;
1-(1-naphthalenylcarbonyl)-2-(4-nitro-1-naphthalenyl)hexahydropyridazine;
1-(2-naphthalenylcarbonyl)-2-(4-nitro-1-naphthalenyl)hexahydropyridazine;
1-(4-nitro-1-naphthalenyl)-2-(2-thienylcarbonyl)hexahydropyridazine;
1-(2-methylpropanoyl)-2-(4-nitro-1-naphthalenyl)hexahydropyridazine;
1-[(2-bromophenyl)carbonyl]-2-(4-nitro-1-naphthalenyl)hexahydropyridazine;
1-[(3-bromophenyl)carbonyl]-2-(4-nitro-1-naphthalenyl)hexahydropyridazine;
1-[(4-bromophenyl)carbonyl]-2-(4-nitro-1-naphthalenyl)hexahydropyridazine;
1-[(2,4-dichlorophenyl)carbonyl]-2-(4-nitro-1-naphthalenyl)hexahydropyridazine;
1-{[4-(1,1-dimethylethyl)phenyl]carbonyl}-2-(4-nitro-1-naphthalenyl)hexahydropyridazine;
1-(4-nitro-1-naphthalenyl)-2-[(phenyloxy)acetyl]hexahydropyridazine;
1-(4-nitro-1-naphthalenyl)-2-(2-phenylbutanoyl)hexahydropyridazine;
1-(4-nitro-1-naphthalenyl)-2-(phenylacetyl)hexahydropyridazine;
1-(cyclopropylcarbonyl)-2-(4-nitro-1-naphthalenyl)hexahydropyridazine;
1-(3-methylbutanoyl)-2-(4-nitro-1-naphthalenyl)hexahydropyridazine;
1-(cyclohexylcarbonyl)-2-(4-nitro-1-naphthalenyl)hexahydropyridazine;
1-[(3-methylphenyl)carbonyl]-2-(4-nitro-1-naphthalenyl)hexahydropyridazine;
1-[(4-methylphenyl)carbonyl]-2-(4-nitro-1-naphthalenyl)hexahydropyridazine;
1-(4-nitro-1-naphthalenyl)-2-{[2-(trifluoromethyl)phenyl]carbonyl}hexahydropyridazine;
1-{[3-(methyloxy)phenyl]acetyl}-2-(4-nitro-1-naphthalenyl)hexahydropyridazine;
1-{[4-(butyloxy)phenyl]carbonyl}-2-(4-nitro-1-naphthalenyl)hexahydropyridazine;
1-(cyclobutylcarbonyl)-2-(4-nitro-1-naphthalenyl)hexahydropyridazine;
1-(cyclopentylcarbonyl)-2-(4-nitro-1-naphthalenyl)hexahydropyridazine;
1-(cyclopentylacetyl)-2-(4-nitro-1-naphthalenyl)hexahydropyridazine;
1-(2-methylpentanoyl)-2-(4-nitro-1-naphthalenyl)hexahydropyridazine;
1-(3-cyclopentylpropanoyl)-2-(4-nitro-1-naphthalenyl)hexahydropyridazine;
1-[(2-bromophenyl)acetyl]-2-(4-nitro-1-naphthalenyl)hexahydropyridazine;
1-(3,3-dimethylbutanoyl)-2-(4-nitro-1-naphthalenyl)hexahydropyridazine;
1-(4-nitro-1-naphthalenyl)-2-{[(1S,2S)-2-phenylcyclopropyl]carbonyl}hexahydropyridazine;
1-(4-nitro-1-naphthalenyl)-2-(3-phenylpropanoyl)hexahydropyridazine;
1-[(4-chlorophenyl)acetyl]-2-(4-nitro-1-naphthalenyl)hexahydropyridazine;
1-(diphenylacetyl)-2-(4-nitro-1-naphthalenyl)hexahydropyridazine;
1-{[4-(methyloxy)phenyl]acetyl}-2-(4-nitro-1-naphthalenyl)hexahydropyridazine;
1-(4-nitro-1-naphthalenyl)-2-({3-[(trifluoromethyl)oxy]phenyl}carbonyl)hexahydropyridazine;
4-(2-acetyl-1-pyrazolidinyl)-1-naphthalenecarbonitrile;
4-[2-(2-methylpropanoyl)-1-pyrazolidinyl]-1-naphthalenecarbonitrile;
4-[2-(3-phenylpropanoyl)-1-pyrazolidinyl]-1-naphthalenecarbonitrile;
4-(2-butanoyl-1-pyrazolidinyl)-1-naphthalenecarbonitrile;
4-[2-(cyclopropylcarbonyl)-1-pyrazolidinyl]-1-naphthalenecarbonitrile;
4-[2-(3-methylbutanoyl)-1-pyrazolidinyl]-1-naphthalenecarbonitrile;
4-[2-(phenylcarbonyl)-1-pyrazolidinyl]-1-naphthalenecarbonitrile;
4-(2-{[3-(trifluoromethyl)phenyl]carbonyl}-1-pyrazolidinyl)-1-naphthalenecarbonitrile;
4-{2-[(2-fluorophenyl)carbonyl]-1-pyrazolidinyl}-1-naphthalenecarbonitrile;
4-{2-[(3-methylphenyl)carbonyl]-1-pyrazolidinyl}-1-naphthalenecarbonitrile;
4-[2-(2-furanylcarbonyl)-1-pyrazolidinyl]-1-naphthalenecarbonitrile;
4-{2-[(3-fluorophenyl)carbonyl]-1-pyrazolidinyl}-1-naphthalenecarbonitrile;
4-{2-[(2-methylphenyl)carbonyl]-1-pyrazolidinyl}-1-naphthalenecarbonitrile;
4-[2-(cyclopentylcarbonyl)-1-pyrazolidinyl]-1-naphthalenecarbonitrile;
4-[2-(2-phenylbutanoyl)-1-pyrazolidinyl]-1-naphthalenecarbonitrile;
4-[2-({2-[(trifluoromethyl)oxy]phenyl}acetyl)tetrahydro-1(2H)-pyridazinyl]-1-naphthalenecarbonitrile;
4-[2-{[2-(methyloxy)phenyl]acetyl}tetrahydro-1(2H)-pyridazinyl]-1-naphthalenecarbonitrile;
4-[2-{[4-(ethyloxy)phenyl]acetyl}tetrahydro-1(2H)-pyridazinyl]-1-naphthalenecarbonitrile;
4-[2-{[2-(ethyloxy)phenyl]acetyl}tetrahydro-1(2H)-pyridazinyl]-1-naphthalenecarbonitrile;
4-[2-{[3-(methyloxy)phenyl]acetyl}tetrahydro-1(2H)-pyridazinyl]-1-naphthalenecarbonitrile;
4-[2-{[3-(ethyloxy)phenyl]acetyl}tetrahydro-1(2H)-pyridazinyl]-1-naphthalenecarbonitrile;
4-[2-({4-[(trifluoromethyl)oxy]phenyl}acetyl)tetrahydro-1(2H)-pyridazinyl]-1-naphthalenecarbonitrile;
4-[2-{[2-fluoro-6-(trifluoromethyl)phenyl]acetyl}tetrahydro-1(2H)-pyridazinyl]-1-naphthalenecarbonitrile;
4-[2-[(2-fluorophenyl)acetyl]tetrahydro-1(2H)-pyridazinyl]-1-naphthalenecarbonitrile;
4-[2-[(2,3-difluorophenyl)acetyl]tetrahydro-1(2H)-pyridazinyl]-1-naphthalenecarbonitrile;
4-[2-{[2-(phenyloxy)phenyl]acetyl}tetrahydro-1(2H)-pyridazinyl]-1-naphthalenecarbonitrile;
4-[2-[(2-iodophenyl)acetyl]tetrahydro-1(2H)-pyridazinyl]-1-naphthalenecarbonitrile;
4-[2-[(pentafluorophenyl)acetyl]tetrahydro-1(2H)-pyridazinyl]-1-naphthalenecarbonitrile;
4-[2-(2-naphthalenyl)acetyl)tetrahydro-1(2H)-pyridazinyl]-1-naphthalenecarbonitrile;
4-[2-[(2-bromophenyl)acetyl]tetrahydro-1(2H-pyridazinyl]-1-naphthalenecarbonitrile;
4-[2-{[3-(trifluoromethyl)phenyl]acetyl}tetrahydro-1(2H)-pyridazinyl]-1-naphthalenecarbonitrile;
4-[2-[(2,4-difluorophenyl)acetyl]tetrahydro-1(2H)-pyridazinyl]-1-naphthalenecarbonitrile;
4-[2-({2-[(phenylmethyl)oxy]phenyl}acetyl)tetrahydro-1(2H)-pyridazinyl]-1-naphthalenecarbonitrile;
4-[2-{[2-(trifluoromethyl)phenyl]acetyl}tetrahydro-1(2H)-pyridazinyl]-1-naphthalenecarbonitrile;
4-[2-[(2,6-difluorophenyl)acetyl]tetrahydro-1(2H)-pyridazinyl]-1-naphthalenecarbonitrile;
4-[2-[(2-chloro-4-fluorophenyl)acetyl]tetrahydro-1(2H)-pyridazinyl]-1-naphthalenecarbonitrile;
4-[2-[(4-fluorophenyl)acetyl]tetrahydro-1(2H)-pyridazinyl]-1-naphthalenecarbonitrile;
4-[2-[(3-nitrophenyl)acetyl]tetrahydro-1(2H)-pyridazinyl]-1-naphthalenecarbonitrile;
4-[2-[(3,4-dichlorophenyl)acetyl]tetrahydro-1(2H)-pyridazinyl]-1-naphthalenecarbonitrile;
4-[2-[(2-chloro-6-fluorophenyl)acetyl]tetrahydro-1(2H)-pyridazinyl]-1-naphthalenecarbonitrile;
4-[2-[(2-methylphenyl)acetyl]tetrahydro-1(2H)-pyridazinyl]-1-naphthalenecarbonitrile;
4-[2-[(3-methylphenyl)acetyl]tetrahydro-1(2H)-pyridazinyl]-1-naphthalenecarbonitrile;
4-[2-({3-[(trifluoromethyl)oxy]phenyl}acetyl)tetrahydro-1(2H)-pyridazinyl]-1-naphthalenecarbonitrile;
4-[2-({3-[(trifluoromethyl)oxy]phenyl}acetyl)-1-pyrazolidinyl]-1-naphthalenecarbonitrile;
4-[2-({3-[(trifluoromethyl)oxy]phenyl}acetyl)-1-pyrazolidinyl]-1-naphthalenecarbonitrile;
4-{2-[(2-fluorophenyl)acetyl]-1-pyrazolidinyl}-1-naphthalenecarbonitrile;
4-(2-{[3-(trifluoromethyl)phenyl]acetyl}-1-pyrazolidinyl)-1-naphthalenecarbonitrile;
4-{2-[(2-bromophenyl)acetyl]-1-pyrazolidinyl}-1-naphthalenecarbonitrile;
4-{2-[(3-methylphenyl)acetyl]-1-pyrazolidinyl}-1-naphthalenecarbonitrile;
4-{2-[(2-methylphenyl)acetyl]-1-pyrazolidinyl}-1-naphthalenecarbonitrile;
4-{2-[(3,4-dichlorophenyl)acetyl]-1-pyrazolidinyl}-1-naphthalenecarbonitrile;
4-[2-({2-[(trifluoromethyl)oxy]phenyl}acetyl)-1-pyrazolidinyl]-1-naphthalenecarbonitrile;
4-[2-(3-isoxazolylacetyl)tetrahydro-1(2H)-pyridazinyl]-1-naphthalenecarbonitrile;
4-[2-[(1-methyl-1H-indol-3-yl)acetyl]tetrahydro-1(2H)-pyridazinyl]-1-naphthalenecarbonitrile;
4-[2-[(ethyloxy)(phenyl)acetyl]tetrahydro-1(2H)-pyridazinyl]-1-naphthalenecarbonitrile;
4-[2-(2-thienylacetyl)tetrahydro-1(2H)-pyridazinyl]-1-naphthalenecarbonitrile;
4-[2-(3-thienylacetyl)tetrahydro-1(2H)-pyridazinyl]-1-naphthalenecarbonitrile;
4-[2-(2-furanylacetyl)tetrahydro-1(2H)-pyridazinyl]-1-naphthalenecarbonitrile;
4-[2-[3-(4-fluorophenyl)propanoyl]tetrahydro-1(2H)-pyridazinyl]-1-naphthalenecarbonitrile;
4-[2-[3-(3-bromophenyl)propanoyl]tetrahydro-1(2H)-pyridazinyl]-1-naphthalenecarbonitrile;
4-[2-[3-(3,4-dichlorophenyl)propanoyl]tetrahydro-1(2H)-pyridazinyl]-1-naphthalenecarbonitrile;
4-[2-[3-(1,3-benzodioxol-5-yl)propanoyl]tetrahydro-1(2H)-pyridazinyl]-1-naphthalenecarbonitrile;
4-[2-{3-[3,5-bis(trifluoromethyl)phenyl]propanoyl}tetrahydro-1(2H)-pyridazinyl]-1-naphthalenecarbonitrile;
4-[2-[(3R)-3-phenylbutanoyl]tetrahydro-1(2H)-pyridazinyl]-1-naphthalenecarbonitrile;
4-[2-{3-[4-(trifluoromethyl)phenyl]propanoyl}tetrahydro-1(2H)-pyridazinyl]-1-naphthalenecarbonitrile;
4-[2-(3-{4-[(trifluoromethyl)oxy]phenyl}propanoyl)tetrahydro-1(2H)-pyridazinyl]-1-naphthalenecarbonitrile;
4-[2-[3-(3,4-difluorophenyl)propanoyl]tetrahydro-1(2H)-pyridazinyl]-1-naphthalenecarbonitrile;
4-[2-{3-[2-(methyloxy)phenyl]propanoyl}tetrahydro-1(2H)-pyridazinyl]-1-naphthalenecarbonitrile;
4-[2-[3-(2-bromophenyl)propanoyl]tetrahydro-1(2H)-pyridazinyl]-1-naphthalenecarbonitrile;
4-[2-[3-(3-chlorophenyl)propanoyl]tetrahydro-1(2H)-pyridazinyl]-1-naphthalenecarbonitrile;
4-[2-(3,3-diphenylpropanoyl)tetrahydro-1(2H)-pyridazinyl]-1-naphthalenecarbonitrile
4-[2-{3-[3-(methyloxy)phenyl]propanoyl}tetrahydro-1(2H)-pyridazinyl]-1-naphthalenecarbonitrile;
4-[2-[3-(2-chlorophenyl)propanoyl]tetrahydro-1(2H)-pyridazinyl]-1-naphthalenecarbonitrile;
4-[2-[3-(4-methylphenyl)propanoyl]tetrahydro-1(2H)-pyridazinyl]-1-naphthalenecarbonitrile;
4-[2-{3-[3-(trifluoromethyl)phenyl]propanoyl}tetrahydro-1(2H)-pyridazinyl]-1-naphthalenecarbonitrile;
4-[2-{3-[4-(methyloxy)phenyl]propanoyl}tetrahydro-1(2H)-pyridazinyl]-1-naphthalenecarbonitrile;
4-[2-(3-phenylbutanoyl)tetrahydro-1(2H)-pyridazinyl]-1-naphthalenecarbonitrile;
4-[2-[3-(2-fluorophenyl)propanoyl]tetrahydro-1(2H)-pyridazinyl]-1-naphthalenecarbonitrile;
4-[2-{[4-fluoro-2-(trifluoromethyl)phenyl]acetyl}tetrahydro-1(2H)-pyridazinyl]-1-naphthalenecarbonitrile;
4-[2-[3-(2-methylphenyl)propanoyl]tetrahydro-1(2H)-pyridazinyl]-1-naphthalenecarbonitrile;
4-[2-[3-(3-fluorophenyl)propanoyl]tetrahydro-1(2H)-pyridazinyl]-1-naphthalenecarbonitrile;
4-[2-[3-(4-bromophenyl)propanoyl]tetrahydro-1(2H)-pyridazinyl]-1-naphthalenecarbonitrile;
4-[2-[2-(phenyloxy)propanoyl]tetrahydro-1(2H)-pyridazinyl]-1-naphthalenecarbonitrile;
4-[2-(2-cyano-3-phenylpropanoyl)tetrahydro-1(2H)-pyridazinyl]-1-naphthalenecarbonitrile;
4-[2-[3-methyl-3-(1H-pyrrol-1-yl)butanoyl]tetrahydro-1(2H)-pyridazinyl]-1-naphthalenecarbonitrile;
4-[2-[3-(2-furanyl)propanoyl]tetrahydro-1(2H)-pyridazinyl]-1-naphthalenecarbonitrile;
4-[2-[3-(4-methyl-1,3-thiazol-5-yl)propanoyl]tetrahydro-1(2H)-pyridazinyl]-1-naphthalenecarbonitrile;
4-[2-[2-(phenyloxy)butanoyl]tetrahydro-1(2H)-pyridazinyl]-1-naphthalenecarbonitrile;
1-[(4-methylphenyl)acetyl]-2-(4-nitro-1-naphthalenyl)hexahydropyridazine;
1-(4-nitro-1-naphthalenyl)-2-{[4-(trifluoromethyl)phenyl]acetyl}hexahydropyridazine;
1-(4-nitro-1-naphthalenyl)-2-({2-[(trifluoromethyl)oxy]phenyl}acetyl)hexahydropyridazine;
1-(4-nitro-1-naphthalenyl)-2-{[3-(trifluoromethyl)phenyl]acetyl}hexahydropyridazine;
1-[(3-bromophenyl)acetyl]-2-(4-nitro-1-naphthalenyl)hexahydropyridazine;
1-(4-nitro-1-naphthalenyl)-2-({2-[(phenylmethyl)oxy]phenyl}acetyl)hexahydropyridazine;
1-{[2-(ethyloxy)phenyl]acetyl}-2-(4-nitro-1-naphthalenyl)hexahydropyridazine;
1-(4-nitro-1-naphthalenyl)-2-{[2-(trifluoromethyl)phenyl]acetyl}hexahydropyridazine;
1-[(3-methylphenyl)acetyl]-2-(4-nitro-1-naphthalenyl)hexahydropyridazine;
1-{[3-(ethyloxy)phenyl]acetyl}-2-(4-nitro-1-naphthalenyl)hexahydropyridazine;
1-(2-naphthalenylacetyl)-2-(4-nitro-1-naphthalenyl)hexahydropyridazine;
1-{[2-(methyloxy)phenyl]acetyl}-2-(4-nitro-1-naphthalenyl)hexahydropyridazine;
1-{[4-(ethyloxy)phenyl]acetyl}-2-(4-nitro-1-naphthalenyl)hexahydropyridazine;
1-[(4-bromophenyl)acetyl]-2-(4-nitro-1-naphthalenyl)hexahydropyridazine;
1-[(2-methylphenyl)acetyl]-2-(4-nitro-1-naphthalenyl)hexahydropyridazine;
1-(4-nitro-1-naphthalenyl)-2-{[3-(phenyloxy)phenyl]acetyl}hexahydropyridazine;
1-{[3,4-bis(methyloxy)phenyl]acetyl}-2-(4-nitro-1-naphthalenyl)hexahydropyridazine;
4-[2-(2-hydroxyethyl)tetrahydro-1(2H)-pyridazinyl]-1-naphthalenecarbonitrile;
2-[2-(4-nitro-1-naphthalenyl)tetrahydro-1(2H)-pyridazinyl]ethanol;
4-[2-[2-(phenyloxy)ethyl]tetrahydro-1(2H)-pyridazinyl]-1-naphthalenecarbonitrile;
4-[2-{2-[(3-chlorophenyl)oxy]ethyl}tetrahydro-1(2H)-pyridazinyl]-1-naphthalenecarbonitrile;
1-(4-nitro-1-naphthalenyl)-2-[2-(phenyloxy)ethyl]hexahydropyridazine;
1-{2-[(2-methylphenyl)oxy]ethyl}-2-(4-nitro-1-naphthalenyl)hexahydropyridazine;
1-{2-[(3-chlorophenyl)oxy]ethyl}-2-(4-nitro-1-naphthalenyl)hexahydropyridazine;
1-{2-[(4-methylphenyl)oxy]ethyl}-2-(4-nitro-1-naphthalenyl)hexahydropyridazine;
2-(4-cyano-1-naphthalenyl)tetrahydro-1(2H)-pyridazinecarbonitrile;
2-(4-nitro-1-naphthalenyl)tetrahydro-1(2H)-pyridazinecarbonitrile;
4-[2-(2,2,2-trifluoroethyl)hexahydro-1H-1,2-diazepin-1-yl]-1-naphthalenecarbonitrile;
4-[2-({3-[4-(trifluoromethyl)phenyl]-1H-pyrazol-4-yl}methyl)tetrahydro-1(2H)-pyridazinyl]-1-naphthalenecarbonitrile;
4-[2-({3-[2-(trifluoromethyl)phenyl]-1H-pyrazol-4-yl}methyl)tetrahydro-1(2H)-pyridazinyl]-1-naphthalenecarbonitrile;
4-[2-{[3-(4-chlorophenyl)-1H-pyrazol-4-yl]methyl}tetrahydro-1(2H)-pyridazinyl]-1-naphthalenecarbonitrile;
4-[2-{[3-(4-fluorophenyl)-1H-pyrazol-4-yl]methyl}tetrahydro-1(2H)-pyridazinyl]-1-naphthalenecarbonitrile;
4-[2-({3-[3-(trifluoromethyl)phenyl]-1H-pyrazol-4-yl}methyl)tetrahydro-1(2H)-pyridazinyl]-1-naphthalenecarbonitrile;
and salts and solvates thereof.

21. A compound selected from:
4-[2-{[3,5-bis(trifluoromethyl)phenyl]methyl}tetrahydro-1(2H)-pyridazinyl]-1-naphthalenecarbonitrile;
4-[2-[(3-phenyl-1H-pyrazol-4-yl)methyl]tetrahydro-1(2H)-pyridazinyl]-1-naphthalenecarbonitrile;
4-[2-{[1-(1,1-dimethylethyl)-5-methyl-1H-pyrazol-3-yl]methyl}tetrahydro-1(2H)-pyridazinyl]-1-naphthalenecarbonitrile;
4-[2-[(6-methyl-2-pyridinyl)methyl]tetrahydro-1(2H)-pyridazinyl]-1-naphthalenecarbonitrile;
4-[2-[(4-phenyl-1H-imidazol-5-yl)methyl]tetrahydro-1(2H)-pyridazinyl]-1-naphthalenecarbonitrile;
1-(4-nitro-1-naphthalenyl)-2-({2-[(trifluoromethyl)oxy]phenyl}acetyl)hexahydropyridazine;
4-[2-(1H-pyrazol-3-ylmethyl)tetrahydro-1(2H)-pyridazinyl]-1-naphthalenecarbonitrile;
4-[2-[(3-cyanophenyl)methyl]tetrahydro-1(2H)-pyridazinyl]-1-naphthalenecarbonitrile;
4-[2-{[3-(3-furanyl)-1H-pyrazol-4-yl]methyl}tetrahydro-1(2H)-pyridazinyl]-1-naphthalenecarbonitrile;
4-[2-{[3-(1-methylethyl)-1H-pyrazol-4-yl]methyl}tetrahydro-1(2H)-pyridazinyl]-1-naphthalenecarbonitrile;
and salts and solvates thereof.

22. (canceled)

23. A pharmaceutical composition comprising a compound according to claim 1.

24-26. (canceled)

27. A method for the treatment of type 2 diabetes, type 1 diabetes, hyperglycemia, insulin resistance, metabolic syndrome X, diabetic dyslipidemia, bipolar disorder (manic depression), drug dependency, sleep disorders, schizophrenia, obsessive-compulsive disorder, post-traumatic stress disorder, social anxiety disorder, and generalized anxiety disorder comprising the administration of a compound according to claim 1.

28. A method for the treatment of conditions or disorders that respond to glucocorticoid receptor modulation comprising the administration of a compound according to claim 1.

29. (canceled)

30. (canceled)
